(19) United States
(12) Patent Application Publication
(10) Pub. No.: US 2026/0139331 A1
(43) Pub. Date: May 21, 2026

(54) HIGH-THROUGHPUT METHODS FOR ISOLATING AND CHARACTERIZING AMMONIUM-EXCRETING MUTANT LIBRARIES GENERATED BY CHEMICAL MUTAGENESIS

(71) Applicant: Pivot Bio, Inc., Minnetonka, MN (US)

(72) Inventors: Min-Hyung RYU, Cambridge, MA (US); Bilge Ozaydin ESKIYENENTURK, Berkeley, CA (US); Alvin TAMSIR, Alamo, CA (US)

(21) Appl. No.: 19/454,783

(22) Filed: Jan. 21, 2026

Related U.S. Application Data
(62) Division of application No. 17/604,119.
(60) Provisional application No. 62/838,780, filed on Apr. 25, 2019.

(51) Int. Cl. C12Q 1/6897; A01N 63/20; C12N 1/205; C12Q 1/689

(52) U.S. Cl. CPC C12Q 1/6897; A01N 63/20; C12N 1/205; C12Q 1/689

(57) ABSTRACT

The present disclosure provides high-throughput methods for rapidly mutagenizing, screening, and targeting candidate microbes that are capable of fixing atmospheric nitrogen in the presence of exogenous nitrogen. The methods utilize a microbial biosensor capable of detecting the presence/absence of ammonium and/or glutamine in a composition and signaling with a fluorescent reporter. The present disclosure further utilizes rapid visual detection assays capable of processing thousands of candidate microbes. The disclosed methods and biosensor can be used to identify mutant bacteria with improved nitrogen fixing capabilities. Mutant bacteria with improved nitrogen fixing capabilities are also disclosed, as well as methods of utilizing these novel bacteria to provide fixed nitrogen to a plant.

### CROSS-REFERENCE TO RELATED APPLICATIONS

This application is a divisional of U.S. application Ser. No. 17/604,119, filed Oct. 15, 2021, which is a U.S. National Stage application under 35 U.S.C. § 371 of International PCT Application No. PCT/US2020/029831, filed Apr. 24, 2020, which claims the benefit of priority to U.S. Provisional Application No. 62/838,780, filed Apr. 25, 2019, each of which is herein incorporated by reference in its entirety for all purposes.

### REFERENCE TO AN ELECTRONIC SEQUENCE LISTING

The contents of the electronic sequence listing (PIVO_014_02US_SeqList_ST26.xml; Size: 87,946 bytes; and Date of Creation: Jan. 20, 2026) are herein incorporated by reference in their entirety.

### BACKGROUND OF THE DISCLOSURE

Techniques used in the state of the art to randomly mutagenize microbes and evaluate and select for an increased ability to fix atmospheric nitrogen in the presence of exogenous nitrogen are slow and not capable of being scaled to mutagenize and select potential candidate microbes given that gauging nitrogenase activity and ammonium excretion are slow processes. There is a clear need for a rapid screen to determine whether a single microbe or a million microbes are capable of fixing nitrogen, and more importantly, doing so in the presence of exogenous nitrogen.

The compositions and methods of using the compositions of the present disclosure solve this bottleneck in the discovery process by combining large-scale microbial mutagenesis with a screening process that relies on a modified microbe to act as a biosensor for screening for the presence or absence of free ammonium and/or glutamine while co-culturing the biosensor with the mutagenized microbes. These methods are further paired with high-throughput assays that rapidly identify candidate microbes that fix atmospheric nitrogen.

### SUMMARY OF THE DISCLOSURE

In one aspect, provided herein is a biosensor capable of detecting the presence of ammonium in a composition, the biosensor comprising: a bacterium comprising: (a) a nucleic acid sequence encoding a reporter molecule, (b) a promoter or fragment thereof selected from the Nif regulon operably linked to the nucleic acid sequence of (a), (c) a nucleic acid sequence encoding an inhibitory nitrogen fixation regulatory protein, and (d) a nucleic acid sequence encoding a positive nitrogen fixation regulatory protein; wherein the bacterium expresses the reporter molecule in the absence of ammonium. In some cases, the reporter molecule is a fluorescent protein, functional fragment, and/or fusions thereof. In some cases, the fluorescent protein is a GFP, RFP, YFP, CFP, or functional variant or fragment thereof. In some cases, the fluorescent protein is GFP. In some cases, the GFP is a superfolder GFP. In some cases, the promoter or fragment thereof is selected from the nifHDK operon. In some cases, the promoter is a nifH promoter. In some cases, the inhibitory nitrogen fixation regulatory protein is NifL. In some cases, the positive nitrogen fixation regulatory protein is NifA. In some cases, the bacterium in Escherichia coli.

In another aspect, provided herein is a method of detecting the presence of ammonium in a composition, the method comprising: (a) inoculating a composition with a biosensor, wherein the biosensor comprises a bacterium, which comprises: (i) a nucleic acid sequence encoding a reporter molecule, (ii) a promoter or fragment thereof selected from the Nif regulon operably linked to the nucleic acid sequence of (i), (iii) a nucleic acid sequence encoding an inhibitory nitrogen fixation regulatory protein, and (iv) a nucleic acid sequence encoding a positive nitrogen fixation regulatory protein; wherein the bacterium expresses the reporter molecule in the absence of ammonium; (b) exposing the inoculated composition to a stimulus sufficient to activate the reporter molecule; and (c) detecting reporter molecule output of the inoculated composition following (b), as compared to a control composition. In some cases, the method further comprises quantifying the amount of ammonium in the composition based upon the detected reporter molecule output. In some cases, the stimulus is selected from the group consisting of a chemical stimulus, a physical stimulus, a genetic stimulus and an energy stimulus. In some cases, the stimulus is light. In some cases, the reporter molecule is a fluorescent protein, functional fragment, and/or fusions thereof. In some cases, the fluorescent protein is a GFP, RFP, YFP, CFP, or functional variant or fragment thereof. In some cases, the fluorescent protein is GFP. In some cases, the GFP is a superfolder GFP. In some cases, step (b) entails exposing the inoculated composition to light excitation sufficient to fluoresce the fluorescent protein, functional fragment, and/or fusions thereof. In some cases, step (c) entails detecting intensity of fluorescent output of the inoculated composition following (b), as compared to the control composition. In some cases, the fluorescence is detected with a flow cytometer, a plate reader, or fluorescence-activated droplet sorting. In some cases, the promoter or fragment thereof is selected from the nifHDK operon. In some cases, the promoter is a nifH promoter. In some cases, the inhibitory nitrogen fixation regulatory protein is NifL. In some cases, the positive nitrogen fixation regulatory protein is NifA.

In one aspect, provided herein is a method for identification of bacterial mutants capable of fixing atmospheric nitrogen, the method comprising: (a) exposing a population of bacteria to a mutagen in a microbial composition; (b) co-culturing the microbial composition with a biosensor, wherein the biosensor comprises a bacterium, which comprises: (i) a nucleic acid sequence encoding a reporter molecule, (ii) a promoter or fragment thereof selected from the Nif regulon operably linked to the nucleic acid sequence of (i), (iii) a nucleic acid sequence encoding an inhibitory nitrogen fixation regulatory protein, and (iv) a nucleic acid sequence encoding a positive nitrogen fixation regulatory protein, wherein the bacterium expresses the reporter molecule in the absence of ammonium; (c) exposing the co-cultured composition to a stimulus sufficient to activate the reporter molecule; and (d) identifying bacterial mutants capable of fixing atmospheric nitrogen as those that result in decreased or no reporter molecule output as compared to a control. In some cases, the reporter molecule output of the co-cultured composition is detected following (c), as compared to a control composition. In some cases, the control composition comprises microbes that do not express the reporter molecule. In some cases, the control composition comprises microbes that express a functionally deleted variant of the reporter molecule. In some cases, the stimulus is selected from the group consisting of a chemical stimulus, a physical stimulus, a genetic stimulus and an energy stimulus. In some cases, the stimulus is light. In some cases, the reporter molecule is a fluorescent protein, functional fragment, and/or fusions thereof. In some cases, the fluorescent protein is a GFP, RFP, YFP, CFP, or functional variant or fragment thereof. In some cases, the fluorescent protein is GFP. In some cases, the GFP is a superfolder GFP. In some cases, step (c) entails exposing the inoculated composition to light excitation sufficient to fluoresce the fluorescent protein, functional fragment, and/or fusions thereof. In some cases, the fluorescence is detected with a flow cytometer, a plate reader, or fluorescence-activated droplet sorting. In some cases, the method further comprises isolating the bacterial mutants capable of fixing atmospheric nitrogen identified in (d). In some cases, the isolated bacterial mutants comprise a bacterium selected from a bacterium deposited as PTA-126709, a bacterium deposited as PTA-126710, a bacterium deposited as PTA-126711, a bacterium deposited as PTA-126712, a bacterium deposited as PTA-126713, a bacterium deposited as PTA-126714, a bacterium deposited as PTA-126715, a bacterium deposited as PTA-126716, a bacterium deposited as PTA-126717, a bacterium deposited as PTA-126718, a bacterium deposited as PTA-126719, a bacterium deposited as PTA-126720, a bacterium deposited as PTA-126721, a bacterium deposited as PTA-126722, a bacterium deposited as PTA-126723, a bacterium deposited as PTA-126724, a bacterium deposited as PTA-126725, a bacterium deposited as PTA-126726 and combinations thereof. In some cases, the isolated bacterial mutants comprise a bacterium comprising at least one genetic variation introduced into a member selected from the group consisting of: nifA, nifL, ntrB, ntrC, polynucleotide encoding glutamine synthetase, glnA, glnB, glnK, drat, amtB, polynucleotide encoding glutaminase, glnD, glnE, nifJ, nifH, nifD, nifK, nifY, nifE, nifN, nifU, nifS, nifV, nifW, nifZ, nifM, nifF, nifB, nifQ, a gene associated with biosynthesis of a nitrogenase enzyme, or combinations thereof. In some cases, the isolated bacterial mutants comprise a bacterium comprising at least one genetic variation in glnA. In some cases, the at least one genetic variation in glnA comprises at least one nucleotide substitution at nucleotide position 198, 469, 515, 521, 566, 622, 652, 764, 802, 986 and/or 1015 of a Klebsiella glnA gene or at a homologous nucleotide position in a homolog thereof. In some cases, the Klebsiella glnA gene comprises a nucleic acid sequence of SEQ ID NO: 1. In some cases, the homolog of the Klebsiella glnA gene is a Kosakonia glnA gene. In some cases, the homolog of the Klebsiella glnA gene comprises a nucleic acid sequence of SEQ ID NO: 2. In some cases, the homolog of the Klebsiella glnA gene is a Metakosakonia glnA gene. In some cases, the homolog of the Klebsiella glnA gene comprises a nucleic acid sequence of SEQ ID NO: 3. In some cases, the glnA gene comprising the at least one genetic variation comprises a nucleic acid sequence selected from the group consisting of SEQ ID NOs 6-13 and 15-19. In some cases, expression of the glnA gene comprising the at least one genetic variation produces a GlnA protein comprising at least one amino acid substitution at amino acid position 66, 157, 172, 174, 189, 208, 218, 255, 268, 329 and/or 339 of a Klebsiella GlnA protein or at a homologous amino acid position in a homolog thereof. In some cases, expression of the glnA gene comprising the at least one genetic variation produces a GlnA protein comprising at least one amino acid substitution selected from the group consisting of M66I, M66V, G157C, H172L, P174L, S189F, E208K, G218S, T255I, G268S, A329V and/or N339D of a Klebsiella GlnA protein and identical amino acid substitutions at homologous amino acid positions in a homolog thereof. In some cases, the Klebsiella GlnA protein comprises an amino acid sequence of SEQ ID NO: 24. In some cases, the homolog of the Klebsiella GlnA protein is a Kosakonia GlnA protein. In some cases, the homolog of the Klebsiella GlnA protein comprises an amino acid sequence of SEQ ID NO: 25. In some cases, the homolog of the Klebsiella GlnA protein is a Metakosakonia GlnA protein. In some cases, the homolog of the Klebsiella GlnA protein comprises an amino acid sequence of SEQ ID NO: 26. In some cases, the GlnA protein comprises an amino acid sequence selected from the group consisting of SEQ ID NOs 29-36 and 38-42. In some cases, the at least one genetic variation in glnA comprises at least one nucleotide substitution at nucleotide position 308, 487, 511 and/or 657 of a Paraburkholderia glnA gene or in a homologous nucleotide position in a homolog thereof. In some cases, the Paraburkholderia glnA gene comprises a nucleic acid sequence of SEQ ID NO: 4. In some cases, the glnA gene comprising the at least one genetic variation comprises a nucleic acid sequence selected from the group consisting of SEQ ID NOs 20-23. In some cases, expression of the glnA gene comprising the at least one genetic variation produces a GlnA protein comprising at least one amino acid substitution at amino acid position 103, 163, 171 and/or 219 of a Paraburkholderia GlnA protein or in a homologous position in a homolog thereof. In some cases, expression of the glnA gene comprising the at least one genetic variation produces a GlnA protein comprising at least one amino acid substitution selected from the group consisting of Y103C, S163P, N171D and/or Q219H of a Paraburkholderia GlnA protein and identical amino acid substitutions in homologous amino acid positions in a homolog thereof. In some cases, the Paraburkholderia GlnA protein comprises an amino acid sequence of SEQ ID NO: 27. In some cases, the GlnA protein comprises an amino acid sequence selected from the group consisting of SEQ ID NOs 43-46. In some cases, the isolated bacterial mutants comprise a bacterium comprising at least one genetic variation in glnE. In some cases, the at least one genetic variation in glnE comprises at least one nucleotide substitution at nucleotide position 965 and/or 2838 of a Klebsiella glnE gene or at a homologous nucleotide position in a homolog thereof. In some cases, the at least one genetic variation in glnE comprises at least one nucleotide substitution at nucleotide position 965 and 2838 of a Klebsiella glnE gene or at a homologous nucleotide position in a homolog thereof. In some cases, the Klebsiella glnE gene comprises a nucleic acid sequence of SEQ ID NO: 5. In some cases, the glnE gene comprising the at least one genetic variation comprises a nucleic acid sequence of SEQ ID NO: 14. In some cases, expression of the glnE gene comprising the at least one genetic variation produces a GlnE protein comprising at least one amino acid substitution at amino acid position 322 and/or 746 of a Klebsiella GlnE protein or at a homologous amino acid position in a homolog thereof. In some cases, expression of the glnE gene comprising the at least one genetic variation produces a GlnE protein comprising at least one amino acid substitution at amino acid position 322 and 746 of a Klebsiella GlnE protein or at a homologous amino acid position in a homolog thereof. In some cases, the amino acid substitution at position 322 or the homologous amino acid position is a G to E substitution. In some cases, the amino acid substitution at position 746 or the homologous amino acid position is a G to D substitution. In some cases, the Klebsiella GlnE protein comprises an amino acid sequence of SEQ ID NO: 28. In some cases, the GlnE protein comprises an amino acid sequence of SEQ ID NO: 37.

In one aspect, provided herein is a method for high-throughput identification of one or more bacterial mutants capable of fixing atmospheric nitrogen, the method comprising: (a) exposing a population of bacteria to a mutagen; (b) transferring the population of bacteria in (a) into one or more samples comprising a medium; (c) co-culturing a biosensor within each of the one or more samples in (b), wherein the biosensor comprises a bacterium, which comprises: (i) a nucleic acid sequence encoding a reporter molecule, (ii) a promoter or fragment thereof selected from the Nif regulon operably linked to the nucleic acid sequence of (i), (iii) a nucleic acid sequence encoding an inhibitory nitrogen fixation regulatory protein, and (iv) a nucleic acid sequence encoding a positive nitrogen fixation regulatory protein; wherein the bacterium expresses the reporter molecule in the absence of ammonium; (d) exposing each of the one or more samples in (c) to a stimulus sufficient to activate the reporter molecule; and (e) identifying from the one or more samples in (d) one or more bacterial mutants capable of fixing atmospheric nitrogen as those that result in decreased output of the reporter molecule as compared to a control. In some cases, the output of the reporter molecule of the one or more samples in (d) is measured following (d), as compared to a control composition. In some cases, the control composition comprises microbes that do not express the reporter molecule. In some cases, the control composition comprises microbes that express a functionally deleted variant of the reporter molecule. In some cases, the stimulus is selected from the group consisting of a chemical stimulus, a physical stimulus, a genetic stimulus and an energy stimulus. In some cases, the stimulus is light. In some cases, the reporter molecule is a fluorescent protein, functional fragment, and/or fusions thereof. In some cases, the fluorescent protein is a GFP, RFP, YFP, CFP, or functional variant or fragment thereof. In some cases, the fluorescent protein is GFP. In some cases, the GFP is a superfolder GFP. In some cases, step (d) entails exposing the inoculated composition to light excitation sufficient to fluoresce the fluorescent protein, functional fragment, and/or fusions thereof. In some cases, the fluorescence is detected with a flow cytometer, a plate reader, or fluorescence-activated droplet sorting. In some cases, the method further comprises isolating the bacterial mutants capable of fixing atmospheric nitrogen. In some cases, the isolated bacterial mutants comprise a bacterium selected from a bacterium deposited as PTA-126709, a bacterium deposited as PTA-126710, a bacterium deposited as PTA-126711, a bacterium deposited as PTA-126712, a bacterium deposited as PTA-126713, a bacterium deposited as PTA-126714, a bacterium deposited as PTA-126715, a bacterium deposited as PTA-126716, a bacterium deposited as PTA-126717, a bacterium deposited as PTA-126718, a bacterium deposited as PTA-126719, a bacterium deposited as PTA-126720, a bacterium deposited as PTA-126721, a bacterium deposited as PTA-126722, a bacterium deposited as PTA-126723, a bacterium deposited as PTA-126724, a bacterium deposited as PTA-126725, a bacterium deposited as PTA-126726 and combinations thereof. In some cases, the isolated bacterial mutants comprise a bacterium comprising at least one genetic variation introduced into a member selected from the group consisting of: nifA, nifL, ntrB, ntrC, polynucleotide encoding glutamine synthetase, glnA, glnB, glnK, drat, amtB, polynucleotide encoding glutaminase, glnD, glnE, nifJ, nifH, nifD, nifK, nifY, nifE, nifN, nifU, nifS, nifV, nifW, nifZ, nifM, nifF, nifB, nifQ, a gene associated with biosynthesis of a nitrogenase enzyme, or combinations thereof. In some cases, the isolated bacterial mutants comprise a bacterium comprising at least one genetic variation in glnA. In some cases, the at least one genetic variation in glnA comprises at least one nucleotide substitution at nucleotide position 198, 469, 515, 521, 566, 622, 652, 764, 802, 986 and/or 1015 of a Klebsiella glnA gene or at a homologous nucleotide position in a homolog thereof. In some cases, the Klebsiella glnA gene comprises a nucleic acid sequence of SEQ ID NO: 1. In some cases, the homolog of the Klebsiella glnA gene is a Kosakonia glnA gene. In some cases, the homolog of the Klebsiella glnA gene comprises a nucleic acid sequence of SEQ ID NO: 2. In some cases, the homolog of the Klebsiella glnA gene is a Metakosakonia glnA gene. In some cases, the homolog of the Klebsiella glnA gene comprises a nucleic acid sequence of SEQ ID NO: 3. In some cases, the glnA gene comprising the at least one genetic variation comprises a nucleic acid sequence selected from the group consisting of SEQ ID NOs 6-13 and 15-19. In some cases, expression of the glnA gene comprising the at least one genetic variation produces a GlnA protein comprising at least one amino acid substitution at amino acid position 66, 157, 172, 174, 189, 208, 218, 255, 268, 329 and/or 339 of a Klebsiella GlnA protein or at a homologous amino acid position in a homolog thereof. In some cases, expression of the glnA gene comprising the at least one genetic variation produces a GlnA protein comprising at least one amino acid substitution selected from the group consisting of M66I, M66V, G157C, H172L, P174L, S189F, E208K, G218S, T255I, G268S, A329V and/or N339D of a Klebsiella GlnA protein and identical amino acid substitutions at homologous amino acid positions in a homolog thereof. In some cases, the Klebsiella GlnA protein comprises an amino acid sequence of SEQ ID NO: 24. In some cases, the homolog of the Klebsiella GlnA protein is a Kosakonia GlnA protein. In some cases, the homolog of the Klebsiella GlnA protein comprises an amino acid sequence of SEQ ID NO: 25. In some cases, the homolog of the Klebsiella GlnA protein is a Metakosakonia GlnA protein. In some cases, the homolog of the Klebsiella GlnA protein comprises an amino acid sequence of SEQ ID NO: 26. In some cases, the GlnA protein comprises an amino acid sequence selected from the group consisting of SEQ ID NOs 29-36 and 38-42. In some cases, the at least one genetic variation in glnA comprises at least one nucleotide substitution at nucleotide position 308, 487, 511 and/or 657 of a Paraburkholderia glnA gene or in a homologous nucleotide position in a homolog thereof. In some cases, the Paraburkholderia glnA gene comprises a nucleic acid sequence of SEQ ID NO: 4. In some cases, the glnA gene comprising the at least one genetic variation comprises a nucleic acid sequence selected from the group consisting of SEQ ID NOs 20-23. In some cases, expression of the glnA gene comprising the at least one genetic variation produces a GlnA protein comprising at least one amino acid substitution at amino acid position 103, 163, 171 and/or 219 of a Paraburkholderia GlnA protein or in a homologous position in a homolog thereof. In some cases, expression of the glnA gene comprising the at least one genetic variation produces a GlnA protein comprising at least one amino acid substitution selected from the group consisting of Y103C, S163P, N171D and/or Q219H of a Paraburkholderia GlnA protein and identical amino acid substitutions in homologous amino acid positions in a homolog thereof. In some cases, the Paraburkholderia GlnA protein comprises an amino acid sequence of SEQ ID NO: 27. In some cases, the GlnA protein comprises an amino acid sequence selected from the group consisting of SEQ ID NOs 43-46. In some cases, the isolated bacterial mutants comprise at least one genetic variation in glnE. In some cases, the at least one genetic variation in glnE comprises at least one nucleotide substitution at nucleotide position 965 and/or 2838 of a Klebsiella glnE gene or at a homologous nucleotide position in a homolog thereof. In some cases, the at least one genetic variation in glnE comprises at least one nucleotide substitution at nucleotide position 965 and 2838 of a Klebsiella glnE gene or at a homologous nucleotide position in a homolog thereof. In some cases, the Klebsiella glnE gene comprises a nucleic acid sequence of SEQ ID NO: 5. In some cases, the glnE gene comprising the at least one genetic variation comprises a nucleic acid sequence of SEQ ID NO: 14. In some cases, expression of the glnE gene comprising the at least one genetic variation produces a GlnE protein comprising at least one amino acid substitution at amino acid position 322 and/or 746 of a Klebsiella GlnE protein or at a homologous amino acid position in a homolog thereof. In some cases, expression of the glnE gene comprising the at least one genetic variation produces a GlnE protein comprising at least one amino acid substitution at amino acid position 322 and 746 of a Klebsiella GlnE protein or at a homologous amino acid position in a homolog thereof. In some cases, the amino acid substitution at position 322 or the homologous amino acid position is a G to E substitution. In some cases, the amino acid substitution at position 746 or the homologous amino acid position is a G to D substitution. In some cases, the Klebsiella GlnE protein comprises an amino acid sequence of SEQ ID NO: 28. In some cases, the GlnE protein comprises an amino acid sequence of SEQ ID NO: 37. In some cases, the population of bacteria comprises at least 100,000 bacteria, and wherein the method for high-throughput identification is completed in less than four hours. In some cases, the promoter or fragment thereof is selected from the nifHDK operon. In some cases, the promoter is a nifH promoter. In some cases, the inhibitory nitrogen fixation regulatory protein is NifL. In some cases, the positive nitrogen fixation regulatory protein is NifA. In some cases, the mutagen is selected from mitomycin C (MMC), N-methyl-N-nitrosourea (MNU), nitrous acid (NA), diepoxybutane (DEB), 1,2,7,8-diepoxyoctane (DEO), ethyl methane sulfonate (EMS), methyl methane sulfonate (MMS), N-methyl-N′-nitro-N-nitrosoguanidine (MNNG), 4-nitroquinoline 1-oxide (4-NQO), 2-methyloxy-6-chloro-9(3-[ethyl-2-chloroethyl]-aminopropylamin)-acridinedihydrochloride (ICR-170), 2-amino purine (2AP), and hydroxylamine (HA). In some cases, the mutagen is EMS. In some cases, the population of bacteria is selected from wild-type bacteria, transgenic mutant bacteria, non-intergeneric mutant bacteria and intergeneric mutant bacteria.

In one aspect, provided herein is a method for identifying novel bacterial genes, pathways, and/or regulatory elements involved in fixing atmospheric nitrogen, the method comprising: (a) exposing a population of bacteria to a mutagen in a microbial composition; (b) inoculating the microbial composition with a biosensor, wherein the biosensor comprises a bacterium, which comprises: (i) a nucleic acid sequence encoding a reporter molecule, (ii) a promoter or fragment thereof selected from the Nif regulon operably linked to the nucleic acid sequence of (i), (iii) a nucleic acid sequence encoding an inhibitory nitrogen fixation regulatory protein, and (iv) a nucleic acid sequence encoding a positive nitrogen fixation regulatory protein; wherein the bacterium expresses the reporter molecule in the absence of ammonium; (c) exposing the inoculated composition to a stimulus sufficient to activate the reporter molecule, (d) measuring the output of the reporter molecule of the inoculated composition following (c), as compared to a control composition; (e) isolating bacteria from inoculated compositions that exhibit a decreased output of the reporter molecule as compared to the control composition as bacteria capable of fixing atmospheric nitrogen; (f) sequencing the genome(s) of the bacteria capable of fixing atmospheric nitrogen from (e); and (g) identifying genes, pathways, and/or regulatory elements that contain mutations from the sequenced genomes. In some cases, the control composition comprises microbes that do not express the reporter molecule. In some cases, the control composition comprises microbes that express a functionally deleted variant of the reporter molecule. In some cases, the stimulus is selected from the group consisting of a chemical stimulus, a physical stimulus, a genetic stimulus and an energy stimulus. In some cases, the stimulus is light. In some cases, the reporter molecule is a fluorescent protein, functional fragment, and/or fusions thereof. In some cases, the fluorescent protein is a GFP, RFP, YFP, CFP, or functional variant or fragment thereof. In some cases, the fluorescent protein is GFP. In some cases, the GFP is a superfolder GFP. In some cases, step (c) entails exposing the inoculated composition to light excitation sufficient to fluoresce the fluorescent protein, functional fragment, and/or fusions thereof. In some cases, the fluorescence is detected with a flow cytometer, a plate reader, or fluorescence-activated droplet sorting. In some cases, the method further comprises determining whether the genes, pathways, and/or regulatory elements contain mutations known to be associated with the fixation of atmospheric nitrogen. In some cases, the identified mutations comprise at least one mutation in glnA or glnE. In some cases, the at least one mutation in glnA comprises at least one nucleotide substitution at nucleotide position 198, 469, 515, 521, 566, 622, 652, 764, 802, 986 and/or 1015 of a Klebsiella glnA gene or at a homologous nucleotide position in a homolog thereof. In some cases, the Klebsiella glnA gene comprises a nucleic acid sequence of SEQ ID NO: 1. In some cases, the homolog of the Klebsiella glnA gene is a Kosakonia glnA gene. In some cases, the homolog of the Klebsiella glnA gene comprises a nucleic acid sequence of SEQ ID NO: 2. In some cases, the homolog of the Klebsiella glnA gene is a Metakosakonia glnA gene. In some cases, the homolog of the Klebsiella glnA gene comprises a nucleic acid sequence of SEQ ID NO: 3. In some cases, the glnA gene comprising the at least one genetic variation comprises a nucleic acid sequence selected from the group consisting of SEQ ID NOs 6-13 and 15-19. In some cases, expression of the glnA gene comprising the at least one genetic variation produces a GlnA protein comprising at least one amino acid substitution at amino acid position 66, 157, 172, 174, 189, 208, 218, 255, 268, 329 and/or 339 of a Klebsiella GlnA protein or at a homologous amino acid position in a homolog thereof. In some cases, expression of the glnA gene comprising the at least one genetic variation produces a GlnA protein comprising at least one amino acid substitution selected from the group consisting of M66I, M66V, G157C, H172L, P174L, S189F, E208K, G218S, T255I, G268S, A329V and/or N339D of a Klebsiella GlnA protein and identical amino acid substitutions at homologous amino acid positions in a homolog thereof. In some cases, the Klebsiella GlnA protein comprises an amino acid sequence of SEQ ID NO: 24. In some cases, the homolog of the Klebsiella GlnA protein is a Kosakonia GlnA protein. In some cases, the homolog of the Klebsiella GlnA protein comprises an amino acid sequence of SEQ ID NO: 25. In some cases, the homolog of the Klebsiella GlnA protein is a Metakosakonia GlnA protein. In some cases, the homolog of the Klebsiella GlnA protein comprises an amino acid sequence of SEQ ID NO: 26. In some cases, the GlnA protein comprises an amino acid sequence selected from the group consisting of SEQ ID NOs 29-36 and 38-42. In some cases, the at least one genetic variation in glnA comprises at least one nucleotide substitution at nucleotide position 308, 487, 511 and/or 657 of a Paraburkholderia glnA gene or in a homologous nucleotide position in a homolog thereof. In some cases, the Paraburkholderia glnA gene comprises a nucleic acid sequence of SEQ ID NO: 4. In some cases, the glnA gene comprising the at least one genetic variation comprises a nucleic acid sequence selected from the group consisting of SEQ ID NOs 20-23. In some cases, expression of the glnA gene comprising the at least one genetic variation produces a GlnA protein comprising at least one amino acid substitution at amino acid position 103, 163, 171 and/or 219 of a Paraburkholderia GlnA protein or in a homologous position in a homolog thereof. In some cases, expression of the glnA gene comprising the at least one genetic variation produces a GlnA protein comprising at least one amino acid substitution selected from the group consisting of Y103C, S163P, N171D and/or Q219H of a Paraburkholderia GlnA protein and identical amino acid substitutions in homologous amino acid positions in a homolog thereof. In some cases, the Paraburkholderia GlnA protein comprises an amino acid sequence of SEQ ID NO: 27. In some cases, the GlnA protein comprises an amino acid sequence selected from the group consisting of SEQ ID NOs 43-46. In some cases, the at least one genetic variation in glnE comprises at least one nucleotide substitution at nucleotide position 965 and/or 2838 of a Klebsiella glnE gene or at a homologous nucleotide position in a homolog thereof. In some cases, the at least one genetic variation in glnE comprises at least one nucleotide substitution at nucleotide position 965 and 2838 of a Klebsiella glnE gene or at a homologous nucleotide position in a homolog thereof. In some cases, the Klebsiella glnE gene comprises a nucleic acid sequence of SEQ ID NO: 5. In some cases, the glnE gene comprising the at least one genetic variation comprises a nucleic acid sequence of SEQ ID NO: 14. In some cases, expression of the glnE gene comprising the at least one genetic variation produces a GlnE protein comprising at least one amino acid substitution at amino acid position 322 and/or 746 of a Klebsiella GlnE protein or at a homologous amino acid position in a homolog thereof. In some cases, expression of the glnE gene comprising the at least one genetic variation produces a GlnE protein comprising at least one amino acid substitution at amino acid position 322 and 746 of a Klebsiella GlnE protein or at a homologous amino acid position in a homolog thereof. In some cases, the amino acid substitution at position 322 or the homologous amino acid position is a G to E substitution. In some cases, the amino acid substitution at position 746 or the homologous amino acid position is a G to D substitution. In some cases, the Klebsiella GlnE protein comprises an amino acid sequence of SEQ ID NO: 28. In some cases, the GlnE protein comprises an amino acid sequence of SEQ ID NO: 37. In some cases, the promoter or fragment thereof is selected from the nifHDK operon. In some cases, the promoter is a nifH promoter. In some cases, the inhibitory nitrogen fixation regulatory protein is NifL. In some cases, the positive nitrogen fixation regulatory protein is NifA. In some cases, the mutagen is selected from mitomycin C (MMC), N-methyl-N-nitrosourea (MNU), nitrous acid (NA), diepoxybutane (DEB), 1,2,7,8-diepoxyoctane (DEO), ethyl methane sulfonate (EMS), methyl methane sulfonate (MMS), N-methyl-N′-nitro-N-nitrosoguanidine (MNNG), 4-nitroquinoline 1-oxide (4-NQO), 2-methyloxy-6-chloro-9(3-[ethyl-2-chloroethyl]-aminopropylamin)-acridinedihydrochloride (ICR-170), 2-amino purine (2AP), and hydroxylamine (HA). In some cases, the mutagen is EMS. In some cases, the population of bacteria is selected from wild-type bacteria, transgenic mutant bacteria, non-intergeneric mutant bacteria and intergeneric mutant bacteria.

In another aspect, provided herein is a method for identification of bacterial mutants capable of fixing atmospheric nitrogen, the method comprising: (a) exposing a population of bacteria to a mutagen; (b) exposing the population of bacteria exposed to the mutagen in (a) to a diazotrophic growth inhibitor in a microbial composition; (c) co-culturing the microbial composition with a biosensor, wherein the biosensor comprises a bacterium, which comprises: (i) a nucleic acid sequence encoding a reporter molecule, (ii) a promoter or fragment thereof selected from the Nif regulon operably linked to the nucleic acid sequence of (i), (iii) a nucleic acid sequence encoding an inhibitory nitrogen fixation regulatory protein, and (iv) a nucleic acid sequence encoding a positive nitrogen fixation regulatory protein, wherein the bacterium expresses the reporter molecule in the absence of ammonium; (d) exposing the co-cultured composition to a stimulus sufficient to activate the reporter molecule; and (e) identifying bacterial mutants capable of fixing atmospheric nitrogen as those that result in decreased or no output of the reporter molecule, as compared to a control. In some cases, the diazotrophic growth inhibitor is ethylenediamine (EDA) or methylammonium. In some cases, the output of the reporter molecule of the one or more samples in (d) is measured following (d), as compared to a control composition. In some cases, the control composition comprises microbes that do not express the reporter molecule. In some cases, the control composition comprises microbes that express a functionally deleted variant of the reporter molecule. In some cases, the stimulus is selected from the group consisting of a chemical stimulus, a physical stimulus, a genetic stimulus and an energy stimulus. In some cases, the stimulus is light. In some cases, the reporter molecule is a fluorescent protein, functional fragment, and/or fusions thereof. In some cases, the fluorescent protein is a GFP, RFP, YFP, CFP, or functional variant or fragment thereof. In some cases, the fluorescent protein is GFP. In some cases, the GFP is a superfolder GFP. In some cases, step (d) entails exposing the inoculated composition to light excitation sufficient to fluoresce the fluorescent protein, functional fragment, and/or fusions thereof. In some cases, the fluorescence is detected with a flow cytometer, a plate reader, or fluorescence-activated droplet sorting. In some cases, the method further comprises isolating the bacterial mutants identified as capable of fixing atmospheric nitrogen. In some cases, the isolated bacterial mutants comprise a bacterium selected from a bacterium deposited as PTA-126709, a bacterium deposited as PTA-126710, a bacterium deposited as PTA-126711, a bacterium deposited as PTA-126712, a bacterium deposited as PTA-126713, a bacterium deposited as PTA-126714, a bacterium deposited as PTA-126715, a bacterium deposited as PTA-126716, a bacterium deposited as PTA-126717, a bacterium deposited as PTA-126718, a bacterium deposited as PTA-126719, a bacterium deposited as PTA-126720, a bacterium deposited as PTA-126721, a bacterium deposited as PTA-126722, a bacterium deposited as PTA-126723, a bacterium deposited as PTA-126724, a bacterium deposited as PTA-126725, a bacterium deposited as PTA-126726 and combinations thereof. In some cases, the isolated bacterial mutants comprise bacteria which comprise at least one genetic variation introduced into a member selected from the group consisting of: nifA, nifL, ntrB, ntrC, polynucleotide encoding glutamine synthetase, glnA, glnB, glnK, drat, amtB, polynucleotide encoding glutaminase, glnD, glnE, nifJ, nifH, nifD, nifK, nifY, nifE, nifN nifU, nifS, nifV, nifW, nif, nifM, nifF, nifB, nifQ, a gene associated with biosynthesis of a nitrogenase enzyme, or combinations thereof. In some cases, the isolated bacterial mutants comprise a bacterium comprising a genetic variation in glnA. In some cases, the at least one genetic variation in glnA comprises at least one nucleotide substitution at nucleotide position 198, 469, 515, 521, 566, 622, 652, 764, 802, 986 and/or 1015 of a Klebsiella glnA gene or at a homologous nucleotide position in a homolog thereof. In some cases, the Klebsiella glnA gene comprises a nucleic acid sequence of SEQ ID NO: 1. In some cases, the homolog of the Klebsiella glnA gene is a Kosakonia glnA gene. In some cases, the homolog of the Klebsiella glnA gene comprises a nucleic acid sequence of SEQ ID NO: 2. In some cases, the homolog of the Klebsiella glnA gene is a Metakosakonia glnA gene. In some cases, the homolog of the Klebsiella glnA gene comprises a nucleic acid sequence of SEQ ID NO: 3. In some cases, the glnA gene comprising the at least one genetic variation comprises a nucleic acid sequence selected from the group consisting of SEQ ID NOs 6-13 and 15-19. In some cases, expression of the glnA gene comprising the at least one genetic variation produces a GlnA protein comprising at least one amino acid substitution at amino acid position 66, 157, 172, 174, 189, 208, 218, 255, 268, 329 and/or 339 of a Klebsiella GlnA protein or at a homologous amino acid position in a homolog thereof. In some cases, expression of the glnA gene comprising the at least one genetic variation produces a GlnA protein comprising at least one amino acid substitution selected from the group consisting of M66I, M66V, G157C, H172L, P174L, S189F, E208K, G218S, T255I, G268S, A329V and/or N339D of a Klebsiella GlnA protein and identical amino acid substitutions at homologous amino acid positions in a homolog thereof. In some cases, the Klebsiella GlnA protein comprises an amino acid sequence of SEQ ID NO: 24. In some cases, the homolog of the Klebsiella GlnA protein is a Kosakonia GlnA protein. In some cases, the homolog of the Klebsiella GlnA protein comprises an amino acid sequence of SEQ ID NO: 25. In some cases, the homolog of the Klebsiella GlnA protein is a Metakosakonia GlnA protein. In some cases, the homolog of the Klebsiella GlnA protein comprises an amino acid sequence of SEQ ID NO: 26. In some cases, the GlnA protein comprises an amino acid sequence selected from the group consisting of SEQ ID NOs 29-36 and 38-42. In some cases, the at least one genetic variation in glnA comprises at least one nucleotide substitution at nucleotide position 308, 487, 511 and/or 657 of a Paraburkholderia glnA gene or in a homologous nucleotide position in a homolog thereof. In some cases, the Paraburkholderia glnA gene comprises a nucleic acid sequence of SEQ ID NO: 4. In some cases, the glnA gene comprising the at least one genetic variation comprises a nucleic acid sequence selected from the group consisting of SEQ ID NOs 20-23. In some cases, expression of the glnA gene comprising the at least one genetic variation produces a GlnA protein comprising at least one amino acid substitution at amino acid position 103, 163, 171 and/or 219 of a Paraburkholderia GlnA protein or in a homologous position in a homolog thereof. In some cases, expression of the glnA gene comprising the at least one genetic variation produces a GlnA protein comprising at least one amino acid substitution selected from the group consisting of Y103C, S163P, N171D and/or Q219H of a Paraburkholderia GlnA protein and identical amino acid substitutions in homologous amino acid positions in a homolog thereof. In some cases, the Paraburkholderia GlnA protein comprises an amino acid sequence of SEQ ID NO: 27. In some cases, the GlnA protein comprises an amino acid sequence selected from the group consisting of SEQ ID NOs 43-46. In some cases, the isolated bacterial mutants comprise at least one genetic variation in glnE. In some cases, the at least one genetic variation in glnE comprises at least one nucleotide substitution at nucleotide position 965 and/or 2838 of a Klebsiella glnE gene or at a homologous nucleotide position in a homolog thereof. In some cases, the at least one genetic variation in glnE comprises at least one nucleotide substitution at nucleotide position 965 and 2838 of a Klebsiella glnE gene or at a homologous nucleotide position in a homolog thereof. In some cases, the Klebsiella glnE gene comprises a nucleic acid sequence of SEQ ID NO: 5. In some cases, the glnE gene comprising the at least one genetic variation comprises a nucleic acid sequence of SEQ ID NO: 14. In some cases, expression of the glnE gene comprising the at least one genetic variation produces a GlnE protein comprising at least one amino acid substitution at amino acid position 322 and/or 746 of a Klebsiella GlnE protein or at a homologous amino acid position in a homolog thereof. In some cases, expression of the glnE gene comprising the at least one genetic variation produces a GlnE protein comprising at least one amino acid substitution at amino acid position 322 and 746 of a Klebsiella GlnE protein or at a homologous amino acid position in a homolog thereof. In some cases, the amino acid substitution at position 322 or the homologous amino acid position is a G to E substitution. In some cases, the amino acid substitution at position 746 or the homologous amino acid position is a G to D substitution. In some cases, the Klebsiella GlnE protein comprises an amino acid sequence of SEQ ID NO: 28. In some cases, the GlnE protein comprises an amino acid sequence of SEQ ID NO: 37. In some cases, the promoter or fragment thereof is selected from the nifHDK operon. In some cases, the promoter is a nifH promoter. In some cases, the inhibitory nitrogen fixation regulatory protein is NifL. In some cases, the positive nitrogen fixation regulatory protein is NifA. In some cases, the mutagen is selected from mitomycin C (MMC), N-methyl-N-nitrosourea (MNU), nitrous acid (NA), diepoxybutane (DEB), 1,2,7,8-diepoxyoctane (DEO), ethyl methane sulfonate (EMS), methyl methane sulfonate (MMS), N-methyl-N′-nitro-N-nitrosoguanidine (MNNG), 4-nitroquinoline 1-oxide (4-NQO), 2-methyloxy-6-chloro-9(3-[ethyl-2-chloroethyl]-aminopropylamin)-acridinedihydrochloride (ICR-170), 2-amino purine (2AP), and hydroxylamine (HA). In some cases, the mutagen is EMS. In some cases, the population of bacteria is selected from wild-type bacteria, transgenic mutant bacteria, non-intergeneric mutant bacteria and intergeneric mutant bacteria.

In still another aspect, provided herein is a microbial composition, comprising: one or more isolated bacteria selected from the group consisting of a bacterium deposited as PTA-126709, a bacterium deposited as PTA-126710, a bacterium deposited as PTA-126711, a bacterium deposited as PTA-126712, a bacterium deposited as PTA-126713, a bacterium deposited as PTA-126714, a bacterium deposited as PTA-126715, a bacterium deposited as PTA-126716, a bacterium deposited as PTA-126717, a bacterium deposited as PTA-126718, a bacterium deposited as PTA-126719, a bacterium deposited as PTA-126720, a bacterium deposited as PTA-126721, a bacterium deposited as PTA-126722, a bacterium deposited as PTA-126723, a bacterium deposited as PTA-126724, a bacterium deposited as PTA-126725 and a bacterium deposited as PTA-126726. In some cases, the composition further comprises an agriculturally acceptable carrier. In some cases, the one or more isolated bacteria fix atmospheric nitrogen at a rate higher than a wild type parental lineage bacteria.

In one aspect, provided herein is a nitrogen fixing bacterium comprising a mutant glnE gene comprising at least one nucleotide substitution at nucleotide position 965 and/or 2838 of a Klebsiella glnE gene or at a homologous nucleotide position in a homolog thereof. In some cases, the mutant glnE gene comprises a nucleotide substitution at nucleotide position 965 and 2838 of the Klebsiella glnE gene or in homologous nucleotide positions in the homolog thereof. In some cases, the mutant glnE gene shares at least 85%, 86%, 87%, 88%, 89%, 90%, 91%, 92%, 93%, 94%, 95%, 96%, 97%, 98%, or 99% identity with the Klebsiella glnE gene or the homolog thereof. In some cases, expression of the mutant glnE gene produces a mutant GlnE protein with at least one amino acid substitution at amino acid position 322 and/or 746 of a Klebsiella GlnE protein or at a homologous amino acid position in a homolog thereof. In some cases, expression of the mutant glnE gene produces a mutant GlnE protein with amino acid substitutions at amino acid positions 322 and 746 of the Klebsiella GlnE protein or at homologous amino acid positions in the homolog thereof. In some cases, the amino acid substitution at position 322 or the homologous amino acid position is a G to E substitution. In some cases, the amino acid substitution at position 746 or the homologous amino acid position is a G to D substitution. In some cases, the mutant GlnE protein shares at least 90%, 91%, 92%, 93%, 94%, 95%, 96%, 97%, 98%, or 99% identity with the Klebsiella GlnE protein or the homolog thereof. In some cases, the Klebsiella glnE gene comprises a nucleic acid sequence of SEQ ID NO: 5. In some cases, the mutant glnE gene comprises a nucleic acid sequence of SEQ ID NO: 14. In some cases, the Klebsiella GlnE protein comprises an amino acid sequence of SEQ ID NO: 28. In some cases, the mutant GlnE protein comprises an amino acid sequence of SEQ ID NO: 37. In some cases, the bacterium is genetically engineered.

In one aspect, provided herein is a nitrogen fixing bacterium comprising a mutant glnE gene encoding a GlnE protein comprising an amino acid sequence with at least one amino acid substitution at amino acid positions 322 and/or 746 of a Klebsiella GlnE protein or at a homologous amino acid position in a homolog thereof. In some cases, the GlnE protein comprises amino acid substitutions at amino acid positions 322 and 746 of the Klebsiella GlnE protein or at homologous amino acid positions in the homolog thereof. In some cases, the amino acid substitution at position 322 or the homologous amino acid position is a G to E substitution. In some cases, the amino acid substitution at position 746 or the homologous amino acid position is a G746D substitution. In some cases, the GlnE protein shares at least 90%, 91%, 92%, 93%, 94%, 95%, 96%, 97%, 98%, or 99% identity with the Klebsiella GlnE protein or the homolog thereof. In some cases, the Klebsiella GlnE protein comprises an amino acid sequence of SEQ ID NO: 20. In some cases, the mutant GlnE protein comprises an amino acid sequence of SEQ ID NO: 37. In some cases, the bacterium is genetically engineered.

In another aspect, provided herein is a nitrogen fixing bacterium comprising a mutant GlnE protein comprising an amino acid sequence with at least one amino acid substitution at amino acid positions 322 and/or 746 of a Klebsiella GlnE protein or at a homologous amino acid position in a homolog thereof. In some cases, the GlnE protein comprises amino acid substitutions at amino acid positions 322 and 746 of the Klebsiella GlnE protein or at homologous amino acid positions in the homolog thereof. In some cases, the amino acid substitution at position 322 or the homologous amino acid position is a G to E substitution. In some cases, the amino acid substitution at position 746 or the homologous amino acid position is a G to D substitution. In some cases, the GlnE protein shares at least 90%, 91%, 92%, 93%, 94%, 95%, 96%, 97%, 98%, or 99% identity with the Klebsiella GlnE protein or the homolog thereof. In some cases, the Klebsiella GlnE protein comprises an amino acid sequence of SEQ ID NO: 20. In some cases, the mutant GlnE protein comprises an amino acid sequence of SEQ ID NO: 37. In some cases, the nitrogen fixing bacterium is Klebsiella variicola CI4874. In some cases, the bacterium is genetically engineered.

In one aspect, provided herein is a nitrogen fixing bacterium comprising a mutant glnA gene comprising at least one nucleotide substitution at nucleotide position 198, 469, 515, 521, 566, 622, 652, 764, 802, 986 and/or 1015 of a Klebsiella glnA gene or at a homologous nucleotide position in a homolog thereof. In some cases, the mutant glnA gene shares at least 85%, 86%, 87%, 88%, 89%, 90%, 91%, 92%, 93%, 94%, 95%, 96%, 97%, 98%, or 99% identity with the Klebsiella glnA gene or the homolog thereof. In some cases, expression of the mutant glnA gene produces a mutant GlnA protein with at least one amino acid substitution at amino acid position 66, 157, 172, 174, 189, 208, 218, 255, 268, 329 and/or 339 of a Klebsiella GlnA protein or at a homologous amino acid position in a homolog thereof. In some cases, expression of the mutant glnA gene produces a mutant GlnA protein with at least one amino acid substitution selected from the group consisting of M66I, M66V, G157C, H172L, P174L, S189F, E208K, G218S, T255I, G268S, A329V and/or N339D of a Klebsiella GlnA protein and identical amino acid substitutions at homologous amino acid positions in a homolog thereof. In some cases, the mutant GlnA protein shares at least 90%, 91%, 92%, 93%, 94%, 95%, 96%, 97%, 98%, or 99% identity with the Klebsiella GlnA protein or the homolog thereof. In some cases, the Klebsiella glnA gene comprises a nucleic acid sequence of SEQ ID NO: 1. In some cases, the homolog of the Klebsiella glnA gene is a Kosakonia glnA gene. In some cases, the homolog of the Klebsiella glnA gene comprises a nucleic acid sequence of SEQ ID NO: 2. In some cases, the homolog of the Klebsiella glnA gene is a Metakosakonia glnA gene. In some cases, the homolog of the Klebsiella glnA gene comprises a nucleic acid sequence of SEQ ID NO: 3. In some cases, the mutant glnA gene comprises a nucleic acid sequence selected from the group consisting of SEQ ID NOs 6-13 and 15-19. In some cases, the Klebsiella GlnA protein comprises an amino acid sequence of SEQ ID NO: 24. In some cases, the homolog of the Klebsiella GlnA protein comprises an amino acid sequence of SEQ ID NO: 25. In some cases, the homolog of the Klebsiella GlnA protein comprises an amino acid sequence of SEQ ID NO: 26. In some cases, the mutant GlnA protein comprises an amino acid sequence selected from the group consisting of SEQ ID NOs 29-36 and 38-42. In some cases, the bacterium is genetically engineered.

In one aspect, provided herein is a nitrogen fixing bacterium comprising a mutant glnA gene encoding a GlnA protein comprising an amino acid sequence with at least one amino acid substitution at amino acid positions 66, 157, 172, 174, 189, 208, 218, 255, 268, 329 and/or 339 of a Klebsiella GlnA protein or at a homologous amino acid position in a homolog thereof. In some cases, the least one amino acid substitution is selected from the group consisting of M66I, M66V, G157C, H172L, P174L, S189F, E208K, G218S, T255I, G268S, A329V and N339D of the Klebsiella GlnA protein and identical amino acid substitutions at homologous positions in the homolog thereof. In some cases, the mutant GlnA protein shares at least 90%, 91%, 92%, 93%, 94%, 95%, 96%, 97%, 98%, or 99% identity with the Klebsiella GlnA protein or the homolog thereof. In some cases, the Klebsiella GlnA protein comprises an amino acid sequence of SEQ ID NO: 24. In some cases, the homolog of the Klebsiella GlnA protein is a Kosakonia GlnA protein. In some cases, the homolog of the Klebsiella GlnA protein comprises an amino acid sequence of SEQ ID NO: 25. In some cases, the homolog of the Klebsiella GlnA protein is a Metakosakonia GlnA protein. In some cases, the homolog of the Klebsiella GlnA protein comprises an amino acid sequence of SEQ ID NO: 26. In some cases, the GlnA protein comprises an amino acid sequence selected from the group consisting of SEQ ID NOs 29-36 and 38-42. In some cases, the bacterium is genetically engineered.

In another aspect, provided herein is a nitrogen fixing bacterium comprising a mutant GlnA protein comprising an amino acid sequence with at least one amino acid substitution at amino acid positions 66, 157, 172, 174, 189, 208, 218, 255, 268, 329 and/or 339 of a Klebsiella GlnA protein or at a homologous amino acid position in a homolog thereof. In some cases, the least one amino acid substitution is selected from the group consisting of M66I, M66V, G157C, H172L, P174L, S189F, E208K, G218S, T255I, G268S, A329V and N339D of the Klebsiella GlnA protein and identical amino acid substitutions in homologous amino acid positions in the homolog thereof. In some cases, the mutant GlnA protein shares at least 90%, 91%, 92%, 93%, 94%, 95%, 96%, 97%, 98%, or 99% identity with the Klebsiella GlnA protein or the homolog thereof. In some cases, the Klebsiella GlnA protein comprises an amino acid sequence of SEQ ID NO: 24. In some cases, the homolog of the Klebsiella GlnA protein is a Kosakonia GlnA protein. In some cases, the homolog of the Klebsiella GlnA protein comprises an amino acid sequence of SEQ ID NO: 25. In some cases, the homolog of the Klebsiella GlnA protein is a Metakosakonia GlnA protein. In some cases, the homolog of the Klebsiella GlnA protein comprises an amino acid sequence of SEQ ID NO: 26. In some cases, the GlnA protein comprises an amino acid sequence selected from the group consisting of SEQ ID NOs 29-36 and 38-42. In some cases, the nitrogen fixing bacterium is selected from the group consisting of Klebsiella variicola CI3296, Klebsiella variicola CI3936, Klebsiella variicola CI3933, Klebsiella variicola CI3927, Klebsiella variicola CI3925, Klebsiella variicola CI3943, Klebsiella variicola CI3940, Klebsiella variicola CI3938, Kosakonia sacchari CI4065, Metakosakonia intestini CI4875, Metakosakonia intestini CI4876, Metakosakonia intestini CI4877 and Metakosakonia intestini CI4878. In some cases, the bacterium is genetically engineered.

In one aspect, provided herein is a nitrogen fixing bacterium comprising a mutant glnA gene comprising at least one nucleotide substitution at nucleotide position 308, 487, 511 and/or 657 of a Paraburkholderia glnA gene or in a homologous nucleotide position in a homolog thereof. In some cases, the mutant glnA gene shares at least 85%, 86%, 87%, 88%, 89%, 90%, 91%, 92%, 93%, 94%, 95%, 96%, 97%, 98%, or 99% identity with the Paraburkholderia glnA gene or the homolog thereof. In some cases, expression of the mutant glnA gene produces a mutant GlnA protein with at least one amino acid substitution at amino acid position 103, 163, 171 and/or 219 of a Paraburkholderia GlnA protein or in a homologous position in a homolog thereof. In some cases, expression of the mutant glnA gene produces a mutant GlnA protein with at least one amino acid substitution selected from the group consisting of Y103C, S163P, N171D and/or Q219H of a Paraburkholderia GlnA protein and identical amino acid substitutions in homologous amino acid positions in a homolog thereof. In some cases, the mutant GlnA protein shares at least 90%, 91%, 92%, 93%, 94%, 95%, 96%, 97%, 98%, or 99% identity with the Paraburkholderia GlnA protein or the homolog thereof. In some cases, the Paraburkholderia glnA gene comprises a nucleic acid sequence of SEQ ID NO: 4. In some cases, the mutant glnA gene comprises a nucleic acid sequence selected from the group consisting of SEQ ID NOs 20-23. In some cases, the Paraburkholderia GlnA protein comprises an amino acid sequence of SEQ ID NO: 27. In some cases, the mutant GlnA protein comprises an amino acid sequence selected from the group consisting of SEQ ID NOs 43-46. In some cases, the bacterium is genetically engineered.

In one aspect, provided herein is a nitrogen fixing bacterium comprising a mutant glnA gene encoding a GlnA protein comprising an amino acid sequence with at least one amino acid substitution at amino acid positions 103, 163, 171 and/or 219 of a Paraburkholderia GlnA protein or in a homologous amino acid position in a homolog thereof. In some cases, the least one amino acid substitution is selected from the group consisting of Y103C, S163P, N171D and Q219H of the Paraburkholderia GlnA protein and identical amino acid substitutions in homologous amino acid positions in the homolog thereof. In some cases, the mutant GlnA protein shares at least 90%, 91%, 92%, 93%, 94%, 95%, 96%, 97%, 98%, or 99% identity with the Paraburkholderia GlnA protein or the homolog thereof. In some cases, the Paraburkholderia GlnA protein comprises an amino acid sequence of SEQ ID NO: 27. In some cases, the GlnA protein comprises an amino acid sequence selected from the group consisting of SEQ ID NOs 43-46. In some cases, the bacterium is genetically engineered.

In one aspect, provided herein is a nitrogen fixing bacterium comprising a mutant GlnA protein comprising an amino acid sequence with at least one amino acid substitution at amino acid positions 103, 163, 171 and/or 219 of a Paraburkholderia GlnA protein or in homologous amino acid positions in a homolog thereof. In some cases, the least one amino acid substitution is selected from the group consisting of Y103C, S163P, N171D and Q219H of the Paraburkholderia GlnA protein and identical amino acid substitutions in homologous amino acid positions in the homolog thereof. In some cases, the mutant GlnA protein shares at least 90%, 91%, 92%, 93%, 94%, 95%, 96%, 97%, 98%, or 99% identity with the Paraburkholderia GlnA protein or the homolog thereof. In some cases, the Paraburkholderia GlnA protein comprises an amino acid sequence of SEQ ID NO: 27. In some cases, the GlnA protein comprises an amino acid sequence selected from the group consisting of SEQ ID NOs 43-46. In some cases, the nitrogen fixing bacterium is selected from the group consisting of Paraburkholderia tropica CI4751, Paraburkholderia tropica CI4753, Paraburkholderia tropica CI4879 and Paraburkholderia tropica CI4752. In some cases, the bacterium is genetically engineered.

In one aspect provided herein is a microbial composition comprising the nitrogen fixing bacterium and a carrier. In some cases, the carrier is an agriculturally acceptable carrier. In some cases, the bacterium is genetically engineered. In some cases, the at least one nitrogen fixing bacterium is a nitrogen fixing bacterium comprising a mutant glnE gene comprising at least one nucleotide substitution at nucleotide position 965 and/or 2838 of a Klebsiella glnE gene or at a homologous nucleotide position in a homolog thereof. In some cases, the at least one nitrogen fixing bacterium is a nitrogen fixing bacterium comprising a mutant glnE gene encoding a GlnE protein comprising an amino acid sequence with at least one amino acid substitution at amino acid positions 322 and/or 746 of a Klebsiella GlnE protein or at a homologous amino acid position in a homolog thereof. In some cases, the at least one nitrogen fixing bacterium is a nitrogen fixing bacterium comprising a mutant GlnE protein comprising an amino acid sequence with at least one amino acid substitution at amino acid positions 322 and/or 746 of a Klebsiella GlnE protein or at a homologous amino acid position in a homolog thereof. In some cases, the at least one nitrogen fixing bacterium is a nitrogen fixing bacterium comprising a mutant glnA gene comprising at least one nucleotide substitution at nucleotide position 198, 469, 515, 521, 566, 622, 652, 764, 802, 986 and/or 1015 of a Klebsiella glnA gene or at a homologous nucleotide position in a homolog thereof. In some cases, the at least one nitrogen fixing bacterium is a nitrogen fixing bacterium comprising a mutant glnA gene encoding a GlnA protein comprising an amino acid sequence with at least one amino acid substitution at amino acid positions 66, 157, 172, 174, 189, 208, 218, 255, 268, 329 and/or 339 of a Klebsiella GlnA protein or at a homologous amino acid position in a homolog thereof. In some cases, the at least one nitrogen fixing bacterium is a nitrogen fixing bacterium comprising a mutant GlnA protein comprising an amino acid sequence with at least one amino acid substitution at amino acid positions 66, 157, 172, 174, 189, 208, 218, 255, 268, 329 and/or 339 of a Klebsiella GlnA protein or at a homologous amino acid position in a homolog thereof. In some cases, the at least one nitrogen fixing bacterium is a nitrogen fixing bacterium comprising a mutant glnA gene comprising at least one nucleotide substitution at nucleotide position 308, 487, 511 and/or 657 of a Paraburkholderia glnA gene or in a homologous nucleotide position in a homolog thereof. In some cases, the at least one nitrogen fixing bacterium is a nitrogen fixing bacterium comprising a mutant glnA gene encoding a GlnA protein comprising an amino acid sequence with at least one amino acid substitution at amino acid positions 103, 163, 171 and/or 219 of a Paraburkholderia GlnA protein or in a homologous amino acid position in a homolog thereof. In some cases, the at least one nitrogen fixing bacterium is a nitrogen fixing bacterium comprising a mutant GlnA protein comprising an amino acid sequence with at least one amino acid substitution at amino acid positions 103, 163, 171 and/or 219 of a Paraburkholderia GlnA protein or in homologous amino acid positions in a homolog thereof. In some cases, the at least one nitrogen fixing bacterium is a selected from the group consisting of a bacterium deposited as PTA-126709, a bacterium deposited as PTA-126710, a bacterium deposited as PTA-126711, a bacterium deposited as PTA-126712, a bacterium deposited as PTA-126713, a bacterium deposited as PTA-126714, a bacterium deposited as PTA-126715, a bacterium deposited as PTA-126716, a bacterium deposited as PTA-126717, a bacterium deposited as PTA-126718, a bacterium deposited as PTA-126719, a bacterium deposited as PTA-126720, a bacterium deposited as PTA-126721, a bacterium deposited as PTA-126722, a bacterium deposited as PTA-126723, a bacterium deposited as PTA-126724, a bacterium deposited as PTA-126725 and a bacterium deposited as PTA-126726. In some cases, the at least one nitrogen fixing bacterium is a selected from the group consisting of Klebsiella variicola CI3296, Klebsiella variicola CI3936, Klebsiella variicola CI3933, Klebsiella variicola CI3927, Klebsiella variicola CI3925, Klebsiella variicola CI3943, Klebsiella variicola CI3940, Klebsiella variicola CI3938, Klebsiella variicola CI4874, Kosakonia sacchari CI4065, Metakosakonia intestini CI4875, Metakosakonia intestini CI4876, Metakosakonia intestini CI4877, Metakosakonia intestini CI4878, Paraburkholderia tropica CI4751, Paraburkholderia tropica CI4753, Paraburkholderia tropica CI4879 and Paraburkholderia tropica CI4752.

In another aspect, provided herein is a method of providing fixed nitrogen to a plant comprising applying a microbial composition to a plant, a plant part, or a locus in which the plant is located, or a locus in which the plant will be grown, wherein the microbial composition comprising at least one of the nitrogen fixing bacterium provided herein. In some cases, the at least one nitrogen fixing bacterium is selected from the microbial strains found in Table 6. In some cases, the at least one nitrogen fixing bacterium is a nitrogen fixing bacterium comprising a mutant glnE gene comprising at least one nucleotide substitution at nucleotide position 965 and/or 2838 of a Klebsiella glnE gene or at a homologous nucleotide position in a homolog thereof. In some cases, the at least one nitrogen fixing bacterium is a nitrogen fixing bacterium comprising a mutant glnE gene encoding a GlnE protein comprising an amino acid sequence with at least one amino acid substitution at amino acid positions 322 and/or 746 of a Klebsiella GlnE protein or at a homologous amino acid position in a homolog thereof. In some cases, the at least one nitrogen fixing bacterium is a nitrogen fixing bacterium comprising a mutant GlnE protein comprising an amino acid sequence with at least one amino acid substitution at amino acid positions 322 and/or 746 of a Klebsiella GlnE protein or at a homologous amino acid position in a homolog thereof. In some cases, the at least one nitrogen fixing bacterium is a nitrogen fixing bacterium comprising a mutant glnA gene comprising at least one nucleotide substitution at nucleotide position 198, 469, 515, 521, 566, 622, 652, 764, 802, 986 and/or 1015 of a Klebsiella glnA gene or at a homologous nucleotide position in a homolog thereof. In some cases, the at least one nitrogen fixing bacterium is a nitrogen fixing bacterium comprising a mutant glnA gene encoding a GlnA protein comprising an amino acid sequence with at least one amino acid substitution at amino acid positions 66, 157, 172, 174, 189, 208, 218, 255, 268, 329 and/or 339 of a Klebsiella GlnA protein or at a homologous amino acid position in a homolog thereof. In some cases, the at least one nitrogen fixing bacterium is a nitrogen fixing bacterium comprising a mutant GlnA protein comprising an amino acid sequence with at least one amino acid substitution at amino acid positions 66, 157, 172, 174, 189, 208, 218, 255, 268, 329 and/or 339 of a Klebsiella GlnA protein or at a homologous amino acid position in a homolog thereof. In some cases, the at least one nitrogen fixing bacterium is a nitrogen fixing bacterium comprising a mutant glnA gene comprising at least one nucleotide substitution at nucleotide position 308, 487, 511 and/or 657 of a Paraburkholderia glnA gene or in a homologous nucleotide position in a homolog thereof. In some cases, the at least one nitrogen fixing bacterium is a nitrogen fixing bacterium comprising a mutant glnA gene encoding a GlnA protein comprising an amino acid sequence with at least one amino acid substitution at amino acid positions 103, 163, 171 and/or 219 of a Paraburkholderia GlnA protein or in a homologous amino acid position in a homolog thereof. In some cases, the at least one nitrogen fixing bacterium is a nitrogen fixing bacterium comprising a mutant GlnA protein comprising an amino acid sequence with at least one amino acid substitution at amino acid positions 103, 163, 171 and/or 219 of a Paraburkholderia GlnA protein or in homologous amino acid positions in a homolog thereof. In some cases, the at least one nitrogen fixing bacterium is a selected from the group consisting of a bacterium deposited as PTA-126709, a bacterium deposited as PTA-126710, a bacterium deposited as PTA-126711, a bacterium deposited as PTA-126712, a bacterium deposited as PTA-126713, a bacterium deposited as PTA-126714, a bacterium deposited as PTA-126715, a bacterium deposited as PTA-126716, a bacterium deposited as PTA-126717, a bacterium deposited as PTA-126718, a bacterium deposited as PTA-126719, a bacterium deposited as PTA-126720, a bacterium deposited as PTA-126721, a bacterium deposited as PTA-126722, a bacterium deposited as PTA-126723, a bacterium deposited as PTA-126724, a bacterium deposited as PTA-126725 and a bacterium deposited as PTA-126726. In some cases, the at least one nitrogen fixing bacterium is a selected from the group consisting of Klebsiella variicola CI3296, Klebsiella variicola CI3936, Klebsiella variicola CI3933, Klebsiella variicola CI3927, Klebsiella variicola CI3925, Klebsiella variicola CI3943, Klebsiella variicola CI3940, Klebsiella variicola CI3938, Klebsiella variicola CI4874, Kosakonia sacchari CI4065, Metakosakonia intestini CI4875, Metakosakonia intestini CI4876, Metakosakonia intestini CI4877, Metakosakonia intestini CI4878, Paraburkholderia tropica CI4751, Paraburkholderia tropica CI4753, Paraburkholderia tropica CI4879 and Paraburkholderia tropica CI4752. In some cases, the microbial composition produces 1% or more of fixed nitrogen in the plant. In some cases, the composition is a solid. In some cases, the composition is a liquid. In some cases, the applying comprises coating a seed or other plant propagation member with the microbial composition. In some cases, the at least one nitrogen fixing bacterium in the microbial composition has an average colonization ability per unit of plant root tissue of at least about 1.0×10⁴ cfu per gram of fresh weight of plant root tissue and produce fixed N of at least about 1×10⁻¹⁵ mmol N per bacterial cell per hour. In some cases, the applying comprises performing in-furrow treatment of the microbial composition. In some cases, the in-furrow treatment comprises applying the microbial composition at a concentration per acre of between about 1×10⁶ to about 3×10¹² cfu per acre. In some cases, the microbial composition is a liquid formulation comprising about 1×10⁶ to about 1×10¹¹ cfu of bacterial cells per milliliter.

### BRIEF DESCRIPTION OF THE FIGURES

FIGS. 1A-B depict an illustration of a molecular circuit for detecting the presence or absence of free ammonium. T=terminator, Pcon=constitutive promoter controlling the transcription of nifA and nifL, PnifH=promoter controlling the transcription of gfp (FIG. 1A), In the absence of free ammonium or below a given concentration threshold of free ammonium, the NifL disassociates with the NifA, which in turn acts upon the nifH promoter operably linked to the gfp, thus resulting in the expression of GFP. The figure further depicts molecular nitrogen (atmospheric nitrogen, N2) entering a nitrogen fixing bacterium and excreting free nitrogen that is sensed by a biosensor strain comprising the molecular circuit (FIG. 1B). Gln, glutamine; Glu, glutamate; 2-OG, 2-oxoglutarate; GS, glutamine synthetase; GOGAT, glutamate synthase.

FIG. 2 depicts a wild type Klebsiella variicola strain (right side of each plate) that exhibits diazotrophic growth versus a strain that cannot fix nitrogen (left side of each plate) under anaerobic conditions. The right plate differs from the left plate in that it contains EDA, which inhibits diazotrophic growth of the Klebsiella variicola 137 wild-type.

FIGS. 3A-B depict Klebsiella variicola 137 strains grown on solid media with 0.2% EDA (FIG. 3A) and Paraburkholderia tropica 8 strains grown on solid media with 100 mM methylammonium (FIG. 3B). Within each of FIG. 3A and FIG. 3B, the left plate was plated with Klebsiella 137 or Paraburkholderia 8 incubated without the mutagen such as 2-aminopurine (2AP) or EMS, and the right plate was plated with Klebsiella variicola 137 or Paraburkholderia 8 treated with the mutagen 2AP or EMS. The chemically mutagenized strains by 2AP or EMS that grew in the presence of ammonium analogues such as 0.2% EDA or 100 mM methylammonium exhibited higher survival rates on the plate

FIG. 4 depicts the fluorescence output of biosensor strains cultured with the supernatants of the EDA mutants, indicating that the EDA mutants excreted ammonium into the medium.

FIGS. 5A-B depicts a standardized response curve for the biosensor strain in the presence of varying concentrations of free ammonium (FIG. 5A) and glutamine (FIG. 5B) and the corresponding fluorescent output. The dissociation constant (Km) for ammonium and glutamine is ˜0.5 mM and ˜0.6 mM, respectively.

FIG. 6 depicts the range of fluorescence, in fluorescence arbitrary units (au), as a result of GFP expression and fluorescence, for an EDA resistant mutant library comprising 327 mutant Klebsiella variicola strains, indicating that approximately ⅕ of the EDA resistant mutant library comprises mutants capable of excreting fixed nitrogen at levels greater than the 137 wild-type strain does.

FIG. 7 depicts the nifH promoter activity in fluorescence arbitrary units (au), as a result of GFP expression and fluorescence, for 21 Klebsiella variicola EDA resistant mutant strains co-cultured with the biosensor. The black bars indicate the absence of ammonium and the gray bars indicate the presence of ammonium. In the 137 wild-type (left panel), the nifH promoter in the biosensor is repressed in the presence of ammonium, whereas many of the nifH promoters in the EDA resistant mutants are active or de-repressed in the presence of ammonium (right panel).

FIG. 8 depicts nitrogenase activity of 20 Klebsiella variicola EDA resistant mutant strains assessed by acetylene reduction assay. Ethylene production is used as a proxy for nitrogenase activity. All 20 mutant strains showed nitrogenase activity in the presence of 5 mM ammonium phosphate.

FIG. 9 depicts the amounts of ammonium excreted from 19 Klebsiella variicola EDA resistant mutant strains after 72 hr of incubation. Twelve (12) of Klebsiella variicola EDA resistant mutant strains excreted more than 20 mM ammonium.

FIGS. 10A-B depict ammonium excretion mutants in four different species identified by random mutagenesis and selection using ammonium analogues such as EDA and methylammonium along with the approximate positions of the mutations in GlnA in the four different species identified by genome-wide sequencing (FIG. 10A). (FIG. 10B), the amounts of ammonium excreted from Paraburkholderia tropica, Metakosakonia intestini, and Kosakonia sacchari are provided.

FIG. 11 illustrates the experiment described in Example 6 in which differential colonization of 137 mutant barcoded strains were determined across nine plants in terms of percent relative abundance.

### DETAILED DESCRIPTION OF THE DISCLOSURE

Development pipelines for constructing microbial products that are resistant to fixed nitrogen present in fertilizers, while also exhibiting improved ammonium excretion while balancing ammonium assimilation, have relied on rational approaches based on the previously identified candidate genes and pathways. Traditional genetic modifications that utilize heterologous constructs, genes, and vectors are considered genetically modified organisms in some jurisdictions. The nature of the random process of generating microbes that excrete ammonium in the presence of fertilizers poses the need to analyze large mutant libraries. However, the predominant technique utilizing the acetylene reduction assay to gauge nitrogenase activity and ammonium excretion is slow and unscalable. The biosensor strain of the present disclosure allows a large mutant library to be analyzed in a high-throughput manner.

Other co-culture methods rely on the growth of a co-culturing strain to address the nitrogen level. Increases in biomass under nitrogen fixing conditions often show little change and requires a long incubation time before any growth occurs due to the anaerobic nature of the assay conditions. This qualitative output necessarily precludes a high-throughput selection process.

While the following terms are believed to be well understood by one or ordinary skill in the art, the following definitions are set forth to facilitate explanation of the presently disclosed subject matter. Numerous variations, changes, and substitutions may occur to those skilled in the art without departing from the disclosure. It should be understood that various alternatives to the embodiments of the disclosure described herein may be employed.

### Definitions

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. For example, if the range 10-15 is disclosed, then 11, 12, 13, and 14 are also disclosed. All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

As used herein, the term “about” is used synonymously with the term “approximately.” Illustratively, the use of the term “about” with regard to an amount indicates that values slightly outside the cited values, e.g., plus or minus 0.1% to 10%.

Reference throughout this specification to “one embodiment”, “an embodiment”, “one aspect”, or “an aspect” means that a particular feature, structure or characteristic described in connection with the embodiment is included in at least one embodiment of the present disclosure. Thus, the appearances of the phrases “in one embodiment” or “in an embodiment” in various places throughout this specification are not necessarily all referring to the same embodiment. Furthermore, the particular features, structures, or characteristics can be combined in any suitable manner in one or more embodiments.

Fertilizers and exogenous nitrogen of the present disclosure may comprise the following nitrogen-containing molecules: ammonium, nitrate, nitrite, ammonia, glutamine, etc. Nitrogen sources of the present disclosure may include anhydrous ammonia, ammonia sulfate, urea, diammonium phosphate, urea-form, monoammonium phosphate, ammonium nitrate, nitrogen solutions, calcium nitrate, potassium nitrate, sodium nitrate, etc.

As used herein, “exogenous nitrogen” refers to non-atmospheric nitrogen readily available in the soil, field, or growth medium that is present under non-nitrogen limiting conditions, including ammonia, ammonium, nitrate, nitrite, urea, uric acid, ammonium acids, etc.

As used herein, “non-nitrogen limiting conditions” refers to non-atmospheric nitrogen available in the soil, field and media at concentrations greater than about 4 mM nitrogen, as disclosed by Kant et al. (2010. J. Exp. Biol. 62(4):1499-1509), which is incorporated herein by reference.

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

As used herein, a “control sequence” refers to an operator, promoter, silencer, or terminator.

As used herein, “introduced” refers to the introduction by means of modern biotechnology, and not a naturally occurring introduction.

As used herein, “introduced genetic material” means genetic material that is added to, and remains as a component of, the genome of the recipient.

In some embodiments, native or endogenous control sequences of genes of the present disclosure are replaced with one or more intrageneric control sequences.

As used herein, a “constitutive promoter” is a promoter, which is active under most conditions and/or during most development stages. There are several advantages to using constitutive promoters in expression vectors used in biotechnology, such as: high level of production of proteins used to select transgenic cells or organisms; high level of expression of reporter proteins or scorable markers, allowing easy detection and quantification; high level of production of a transcription factor that is part of a regulatory transcription system; production of compounds that requires ubiquitous activity in the organism; and production of compounds that are required during all stages of development. Non-limiting exemplary constitutive promoters include, CaMV 35S promoter, opine promoters, ubiquitin promoter, alcohol dehydrogenase promoter, etc.

As used herein, a “non-constitutive promoter” is a promoter which is active under certain conditions, in certain types of cells, and/or during certain development stages. For example, tissue specific, tissue preferred, cell type specific, cell type preferred, inducible promoters, and promoters under development control are non-constitutive promoters. Examples of promoters under developmental control include promoters that preferentially initiate transcription in certain tissues.

As used herein, “inducible” or “repressible” promoter is a promoter which is under chemical or environmental factors control. Examples of environmental conditions that may affect transcription by inducible promoters include anaerobic conditions, certain chemicals, the presence of light, acidic or basic conditions, etc.

As used herein, the term “operably linked” refers to the association of nucleic acid sequences on a single nucleic acid fragment so that the function of one is regulated by the other. For example, a promoter is operably linked with a coding sequence when it is capable of regulating the expression of that coding sequence (i.e., that the coding sequence is under the transcriptional control of the promoter). Coding sequences can be operably linked to regulatory sequences in a sense or antisense orientation. In another example, the complementary RNA regions of the disclosure can be operably linked, either directly or indirectly, 5′ to the target mRNA, or 3′ to the target mRNA, or within the target mRNA, or a first complementary region is 5′ and its complement is 3′ to the target mRNA.

As used herein, “recombinant construct”, “expression construct”, “chimeric construct”, “construct”, and “recombinant DNA construct” are used interchangeably herein. A recombinant construct comprises an artificial combination of nucleic acid fragments, e.g., regulatory and coding sequences that are not found together in nature. For example, a chimeric construct may comprise regulatory sequences and coding sequences that are derived from different sources, or regulatory sequences and coding sequences derived from the same source, but arranged in a manner different than that found in nature. Such construct may be used by itself or may be used in conjunction with a vector. If a vector is used then the choice of vector is dependent upon the method that will be used to transform host cells as is well known to those skilled in the art. For example, a plasmid vector can be used. The skilled artisan is well aware of the genetic elements that must be present on the vector in order to successfully transform, select and propagate host cells comprising any of the isolated nucleic acid fragments of the disclosure. The skilled artisan will also recognize that different independent transformation events will result in different levels and patterns of expression (Jones et al., (1985) EMBO J. 4:2411-2418; De Almeida et al., (1989) Mol. Gen. Genetics 218:78-86), and thus that multiple events must be screened in order to obtain lines displaying the desired expression level and pattern. Such screening may be accomplished by Southern analysis of DNA, Northern analysis of mRNA expression, immunoblotting analysis of protein expression, or phenotypic analysis, among others. Vectors can be plasmids, viruses, bacteriophages, pro-viruses, phagemids, transposons, artificial chromosomes, and the like, that replicate autonomously or can integrate into a chromosome of a host cell. A vector can also be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, that is not autonomously replicating. As used herein, the term “expression” refers to the production of a functional end-product e.g., an mRNA or a protein (precursor or mature).

The terms “polypeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” includes natural and/or unnatural or synthetic amino acids, including glycine and both the D or L optical isomers, and amino acid analogs and peptidomimetics.

As used herein the terms “microorganism” or “microbe” should be taken broadly. These terms, used interchangeably, include but are not limited to, the two prokaryotic domains, Bacteria and Archaea. The term may also encompass eukaryotic fungi and protists.

As used herein, “isolate,” “isolated,” “isolated microbe,” and like terms, are intended to mean that the one or more microorganisms has been separated from at least one of the materials with which it is associated in a particular environment (for example soil, water, plant tissue, etc.). Thus, an “isolated microbe” does not exist in its naturally occurring environment; rather, it is through the various techniques described herein that the microbe has been removed from its natural setting and placed into a non-naturally occurring state of existence. Thus, the isolated strain or isolated microbe may exist as, for example, a biologically pure culture, or as spores (or other forms of the strain). In aspects, the isolated microbe may be in association with an acceptable carrier, which may be an agriculturally acceptable carrier.

In certain aspects of the disclosure, the isolated microbes exist as “isolated and biologically pure cultures.” It will be appreciated by one of skill in the art that an isolated and biologically pure culture of a particular microbe, denotes that said culture is substantially free of other living organisms and contains only the individual microbe in question. The culture can contain varying concentrations of said microbe. The present disclosure notes that isolated and biologically pure microbes often “necessarily differ from less pure or impure materials.” See, e.g. In re Bergstrom, 427 F.2d 1394, (CCPA 1970)(discussing purified prostaglandins), see also, In re Bergy, 596 F.2d 952 (CCPA 1979)(discussing purified microbes), see also, Parke-Davis & Co. v. H. K. Mulford & Co., 189 F. 95 (S.D.N.Y. 1911) (Learned Hand discussing purified adrenaline), aff'd in part, rev'd in part, 196 F. 496 (2d Cir. 1912), each of which are incorporated herein by reference. Furthermore, in some aspects, the disclosure provides for certain quantitative measures of the concentration, or purity limitations, that must be found within an isolated and biologically pure microbial culture. The presence of these purity values, in certain embodiments, is a further attribute that distinguishes the presently disclosed microbes from those microbes existing in a natural state. See, e.g., Merck & Co. v. Olin Mathieson Chemical Corp., 253 F.2d 156 (4th Cir. 1958) (discussing purity limitations for vitamin B12 produced by microbes), incorporated herein by reference.

The term “biologically pure culture” or “substantially pure culture” refers to a culture of a bacterial species described herein containing no other bacterial species in quantities sufficient to interfere with the replication of the culture or be detected by normal bacteriological techniques.

In some embodiments, the microbes of the present disclosure have been modified such that they are not naturally occurring bacteria.

As used herein, an “intergeneric microorganism” is a microorganism that is formed by the deliberate combination of genetic material originally isolated from organisms of different taxonomic genera. An “intergeneric mutant” can be used interchangeably with “intergeneric microorganism”. An exemplary “intergeneric microorganism” includes a microorganism containing a mobile genetic element which was first identified in a microorganism in a genus different from the recipient microorganism. Further explanation can be found, inter alia, in 40 C.F.R. § 725.3.

In some aspects, microbes taught herein are “non-intergeneric,” which means that the microbes are not intergeneric.

A “non-intergeneric remodeled microorganism” has a synonymous meaning to “non-intergeneric engineered microorganism,” and will be utilized interchangeably.

Microbes of the present disclosure may include spores and/or vegetative cells. In some embodiments, microbes of the present disclosure include microbes in a viable but non-culturable (VBNC) state. As used herein, “spore” or “spores” refer to structures produced by bacteria and fungi that are adapted for survival and dispersal. Spores are generally characterized as dormant structures; however, spores are capable of differentiation through the process of germination. Germination is the differentiation of spores into vegetative cells that are capable of metabolic activity, growth, and reproduction. The germination of a single spore results in a single fungal or bacterial vegetative cell. Fungal spores are units of asexual reproduction, and in some cases are necessary structures in fungal life cycles. Bacterial spores are structures for surviving conditions that may ordinarily be nonconductive to the survival or growth of vegetative cells.

As used herein, “microbial composition” refers to a composition comprising one or more microbes of the present disclosure. In some embodiments, a microbial composition is administered to plants (including various plant parts) and/or in agricultural fields.

As used herein, “carrier,” “acceptable carrier,” or “agriculturally acceptable carrier” refers to a diluent, adjuvant, excipient, or vehicle with which the microbe can be administered, which does not detrimentally effect the microbe.

As used herein, “plant part” refers to any part of a plant such as a seed, root, stem, tissue, etc.

In some embodiments, the nitrogen fixation and assimilation genetic regulatory network comprises polynucleotides encoding genes and non-coding sequences that direct, modulate, and/or regulate microbial nitrogen fixation and/or assimilation and can comprise polynucleotide sequences of the nif cluster (e.g., nifA, nifB, nifC, . . . , nifZ), polynucleotides encoding nitrogen regulatory protein C, polynucleotides encoding nitrogen regulatory protein B, polynucleotide sequences of the gln cluster (e.g. glnA and glnD), draT, and ammonia transporters/permeases. In some cases, the Nif cluster may comprise NifB, NifH, NifD, NifK, NifE, NifN, NifX, hesa, and NifV. In some cases, the Nif cluster may comprise a subset of NifB, NifH, NifD, NifK, NifE, NifN, NifX, hesa, and NifV.

In some embodiments, the increase of nitrogen fixation in microbes and/or the production of 1% or more of the nitrogen in the microbes or media containing the microbes is measured relative to control microbes, which have not been exposed to the microbes of the present disclosure. All increases or decreases in microbes are measured relative to control microbes.

In some embodiments, the increase or decrease in the output of a reporter gene, such as a gfp, in the microbes or media containing the microbes is measured relative to control microbes, which either do not express the reporter gene or express a functionally deleted variant thereof. All increases or decreases in reporter genes in the microbes are measured relative to control microbes.

### Regulation of Nitrogen Fixation

In order to utilize elemental nitrogen (N) for chemical synthesis, life forms convert nitrogen gas (N₂) available in the atmosphere into ammonia in a process known as nitrogen fixation. Because of the energy-intensive nature of biological nitrogen fixation, diazotrophs (bacteria and archaea that fix atmospheric nitrogen gas) have evolved sophisticated and tight regulation of the nif gene cluster in response to environmental oxygen and available nitrogen. Nif genes encode enzymes involved in nitrogen fixation (such as the nitrogenase complex) and proteins that regulate nitrogen fixation. Shamseldin (2013. Global J. Biotechnol. Biochem. 8(4):84-94) discloses detailed descriptions of nif genes and their products, and is incorporated herein by reference.

In Proteobacteria, regulation of nitrogen fixation centers around the σ₅₄-dependent enhancer-binding protein NifA, the positive transcriptional regulator of the nif cluster. Intracellular levels of active NifA are controlled by two key factors: transcription of the nifLA operon and inhibition of NifA activity by protein-protein interaction with NifL. Both of these processes are responsive to intracellular glutamine levels via the PII protein signaling cascade. This cascade is mediated by GlnD, which directly senses glutamine and catalyzes the uridylylation or deuridylylation of two PII regulatory proteins—GlnB and GlnK—in response the absence or presence, respectively, of bound glutamine. Under conditions of nitrogen excess, unmodified GlnB signals the deactivation of the nifLA promoter. However, under conditions of nitrogen limitation, GlnB is post-translationally modified, which inhibits its activity and leads to transcription of the nifLA operon. In this way, nifLA transcription is tightly controlled in response to environmental nitrogen via the PII protein signaling cascade. On the post-translational level of NifA regulation, GlnK inhibits the NifL/NifA interaction in a matter dependent on the overall level of free GlnK within the cell.

NifA is transcribed from the nifLA operon, whose promoter is activated by phosphorylated NtrC, another σ₅₄-dependent regulator. The phosphorylation state of NtrC is mediated by the histidine kinase NtrB, which interacts with deuridylylated GlnB but not uridylylated GlnB. Under conditions of nitrogen excess, a high intracellular level of glutamine leads to deuridylylation of GlnB, which then interacts with NtrB to deactivate its phosphorylation activity and activate its phosphatase activity, resulting in dephosphorylation of NtrC and the deactivation of the nifLA promoter. However, under conditions of nitrogen limitation, a low level of intracellular glutamine results in uridylylation of GlnB, which inhibits its interaction with NtrB and allows the phosphorylation of NtrC and transcription of the nifLA operon. In this way, nifLA expression is tightly controlled in response to environmental nitrogen via the PII protein signaling cascade. nifA, ntrB, ntrC, and glnB, are all genes that can be mutated in the methods described herein. These processes may also be responsive to intracellular or extracellular levels of ammonia, urea or nitrates.

Although some endophytes have the ability to fix nitrogen in vitro, often the genetics are silenced in the field by high levels of exogenous chemical fertilizers. One can decouple the sensing of exogenous nitrogen from expression of the nitrogenase enzyme to facilitate field-based nitrogen fixation. Improving the integral of nitrogenase activity across time further serves to augment the production of nitrogen for utilization by the crop. Specific targets for genetic variation to facilitate field-based nitrogen fixation using the methods described herein include one or more genes selected from the group consisting of nifA, nifL, ntrB, ntrC, glnA, glnB, glnK, draT, amtB, glnD, glnE, nifJ, nifH, nifD, nifK, nifY, nifE, nifN, nifU, nifS, nifV, nifW, nifZ, nifM, nifF, nifB, and nifQ.

An additional target for genetic variation to facilitate field-based nitrogen fixation using the methods described herein is the NifA protein. The NifA protein is typically the activator for expression of nitrogen fixation genes. Increasing the production of NifA (either constitutively or during high ammonia condition) circumvents the native ammonia-sensing pathway. In addition, reducing the production of NifL proteins, a known inhibitor of NifA, also leads to an increased level of freely active NifA. In addition, increasing the transcription level of the nifLA operon (either constitutively or during high ammonia condition) also leads to an overall higher level of NifA proteins. Elevated level of nifLA expression is achieved by altering the promoter itself or by reducing the expression of NtrB (part of ntrB and ntrC signaling cascade that originally would result in the shutoff of nifLA operon during high nitrogen condition). High level of NifA achieved by these or any other methods described herein increases the nitrogen fixation activity of the endophytes.

### Biosensor

The biosensor microbe of the present disclosure enables the screening of ammonium-excreting microbes based on a co-culture system. The use of the biosensor has clear advantages, including: (i) excretion of ammonium from a mutant library can be visualized by fluorescence detection of the biosensor in liquid culture as well as on solid media; (ii) intracellular nitrogen level of the mutants that fixed atmospheric nitrogen into ammonium can be analyzed with a biosensor strain; (iii) as a biosensor utilizes ammonium fixed by the co-culture library, this system is more pertinent to the field environments in which microbes reside without artificial ammonium accumulation.

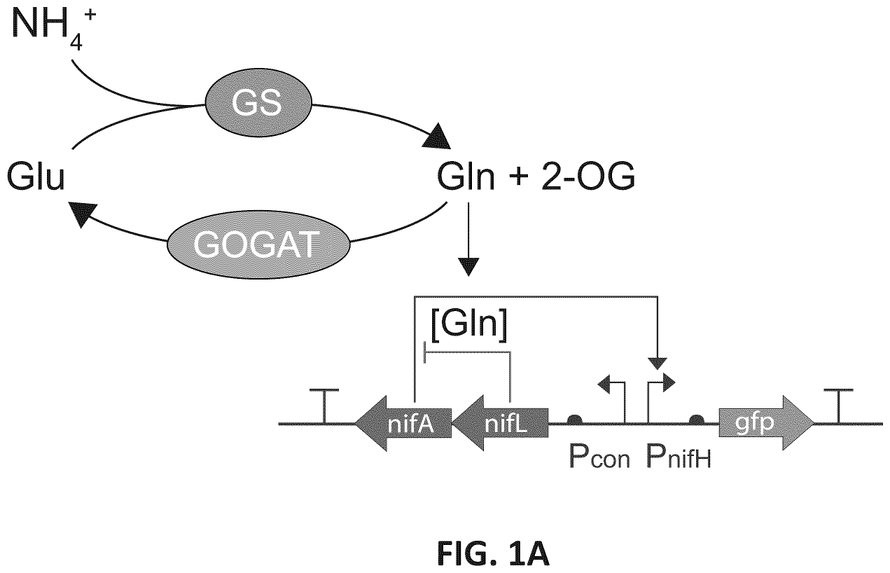

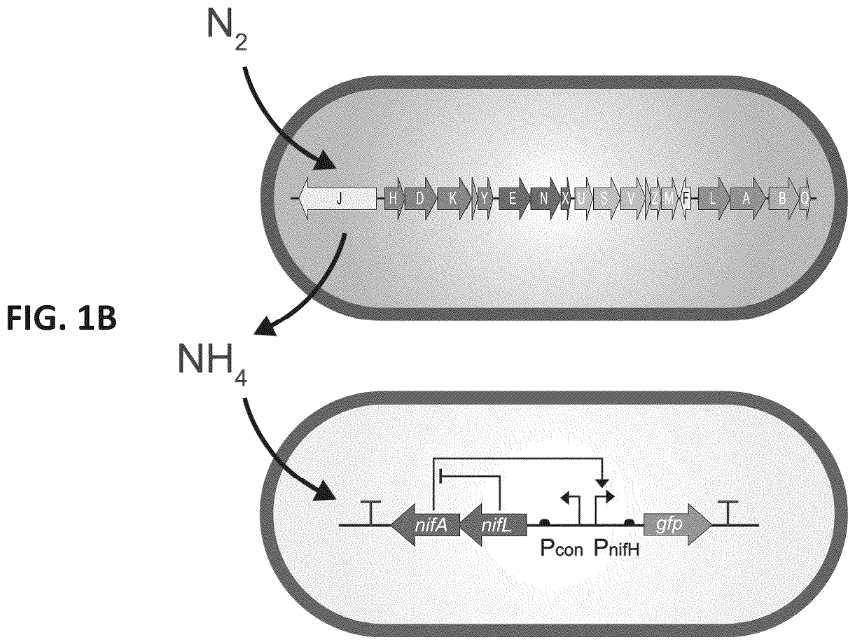

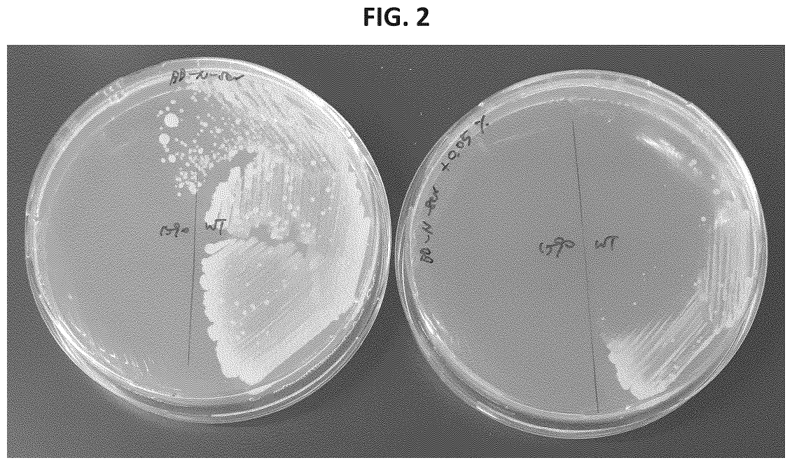

In some aspects, the biosensor comprises a heterologous sequence that acts as a circuit. See FIG. 1A-B. In some aspects, the circuit comprises both a nucleic acid sequence encoding an inhibitory nitrogen fixation regulatory protein under the control of a promoter and a nucleic acid sequence encoding a positive nitrogen fixation regulatory protein under the control of a promoter. In some aspects, the nucleic acid sequence encoding an inhibitory nitrogen fixation regulatory protein and the nucleic acid sequence encoding a positive nitrogen fixation regulatory protein are each under the control of a constitutive promoter. In some aspects, the nucleic acid sequence encoding an inhibitory nitrogen fixation regulatory protein and the nucleic acid sequence encoding a positive nitrogen fixation regulatory protein are present as an operon under the control of a constitutive promoter. In some aspects, the directionality of transcription of the nucleic acid sequence encoding an inhibitory nitrogen fixation regulatory protein and the nucleic acid sequence encoding a positive nitrogen fixation regulatory protein are each in the opposite direction of transcription from one or more other elements of the circuit. In some aspects, the directionality of transcription of the operon comprising the nucleic acid sequence encoding an inhibitory nitrogen fixation regulatory protein and the nucleic acid sequence encoding a positive nitrogen fixation regulatory protein is in the opposite direction of transcription from one or more other elements of the circuit. In some aspects, the circuit comprises a nifLA operon under the control of a promoter. In some aspects, the nifLA operon is under the control of a constitutive promoter. In some aspects, the directionality of transcription of the nifLA operon is in the opposite direction of transcription from one or more other elements of the circuit. In some aspects, the circuit comprises a nucleotide sequence encoding a fluorescent protein or a functional fragment and/or fusion thereof, or another reporter protein or molecule. In some aspects, the nucleotide sequence encoding a reporter protein is under the control of a promoter and a terminator. In some aspects, the nucleotide sequence encoding a fluorescent protein is under the control of a promoter and a terminator. In some aspects, the reporter protein is operably linked to or under the control of a promoter or fragment thereof selected from the Nif regulon. In some aspects, the fluorescent protein is operably linked to or under the control of a promoter or fragment thereof selected from the Nif regulon. In some aspects, the reporter protein is operably linked to or under the control of a promoter or fragment thereof selected from the nifHDK operon. In some aspects, the fluorescent protein is operably linked to or under the control of a promoter or fragment thereof selected from the nifHDK operon. In some aspects, the reporter protein is operably linked to or under the control of a nifH promoter. In some aspects, the fluorescent protein is operably linked to or under the control of a nifH promoter. In some aspects, the directionality of transcription of the reporter protein is in the opposite direction of transcription from one or more other elements of the circuit. In some aspects, the directionality of transcription of the fluorescent protein is in the opposite direction of transcription from one or more other elements of the circuit. In some aspects, the circuit comprises one or more constitutive promoters. In some aspects, the circuit comprises one or more terminators.

In some aspects, the nucleic acid sequence encoding an inhibitory nitrogen fixation regulatory protein is a nifL gene that encodes NifL. In some aspects, the nucleic acid sequence encoding a positive nitrogen fixation regulatory protein is a nifA gene that encodes NifA. In some aspects, NifL is bound to NifA in the presence of free ammonium or glutamine. In some aspects, in the absence of free ammonium or glutamine, the NifL is not bound to NifA. In some aspects, unbound NifA acts on the nifH promoter to initiate transcription and expression of the reporter protein (e.g., GFP). In some aspects, a microbe comprising the circuit does not express detectable amounts of reporter protein (e.g., GFP) in the presence of free ammonium or glutamine. In some aspects, a microbe comprising the circuit expresses detectable amounts of reporter protein (e.g., GFP) in the absence of free ammonium or glutamine.

In some aspects, a reporter element, such as a reporter protein, molecule or enzyme is produced or expressed by the biosensor. The reporter protein emits a detectable signal in response to certain predetermined changes in the cytosol of the living, engineered biosensor. In some aspects, the reporter protein is a bioluminescent photoprotein such as aequorin, which is derived from the hydrozoan Aequorea victoria. In some aspects, production of the reporter protein within the biosensor cell will then be controlled by expression of the introduced genetic material. In some aspects, other photoproteins or other types of reporter proteins, enzymes, and molecules may be incorporated into and utilized with various alternate embodiments of the present disclosure.

In some aspects, the reporter can be a protein that has fluorescent properties that undergo a detectable change in response to activation of the biosensor circuit. In some aspects, the reporter or reporter protein can be calcium-sensitive luminescent or fluorescent molecules, such as obelin, thalassicolin, mitrocomin (halistaurin), clytin (phialidin), mnemopsin, berovin, Indo-1, Fura-2, Quin-2, Fluo-3, Rhod-2, calcium green, BAPTA, cameleons, or similar molecules. In some aspects, the reporter protein can be a chimeric protein that includes a Ca′ binding domain and an associated fluorescent protein. In some aspects, the reporter can be an enzyme that is adapted to produce a luminescent or fluorescent signal. In some aspects, the reporter protein can be an enzyme such as luciferase or alkaline phosphatase that yields a luminescent or fluorescent signal respectively. In some aspects, the reporter can also be a fluorescent protein or can include fluorescent, charged, or magnetic nanoparticles, nanodots, or quantum dots. In some aspects, the reporter can be a dye that has fluorescent, ultraviolet, or visible properties, wherein the fluorescent, ultraviolet, or visible properties undergo a detectable change in response to the activation of the biosensor circuit.

In some aspects, the reporter gene is a fluorescent reporter gene. In some aspects, the fluorescent reporter gene encodes a fluorescent protein. In some aspects, the fluorescent reporter gene encodes an aequorin. In some aspects, the fluorescent protein is selected from the far-red class of fluorescent proteins. In some aspects, the far-red fluorescent protein is mPlum or a variant thereof.

In some aspects, the fluorescent protein is selected from the red class of fluorescent proteins. In some aspects the red fluorescent protein is selected from RFP, mCherry, tdTomato, mStrawberry, J-Red, DsRed-monomer, or a variant thereof. In some aspects, the fluorescent protein is selected from the orange class of fluorescent proteins. In some aspects, the orange fluorescent protein is selected from OFP, mOrange, mKO, or a variant thereof.

In some aspects, the fluorescent protein is selected from the yellow-green class of fluorescent proteins. In some aspects, the yellow-green fluorescent protein is selected from YFP, mCitrine, Venus, YPet, EYFP, or a variant thereof.

In some aspects, the fluorescent protein is selected from the green class of fluorescent proteins. In some aspects, the green fluorescent protein is selected from GFP, EGFP, Emerald, or a variant thereof. In some aspects, the fluorescent protein is selected from the cyan class of fluorescent proteins. In some aspects, the cyan fluorescent protein is selected from Cypet, mCFPm, Cerulean, CFP, or a variant thereof. In some aspects, the fluorescent protein is selected from the UV-excitable green class of fluorescent proteins. In some aspects the UV-excitable green fluorescent protein is selected from T-sapphire. In one embodiment, the GFP is a superfolder GFP.

In some aspects, the reporter gene is operably linked to promoter or a fragment thereof from the Nif regulon. The promoter is selected from the nifRLA operon, nifHDK operon, nifEN operon, nifBQ operon, nifJ operon, nifUSVM operon, or the nifWF operon. In some aspects, the promoter is selected from the nifHDK operon. In some aspects, the promoter is selected from nifH.

In some aspects, the oxygen sensor of nifL is modified such that the presence of oxygen cannot change the conformational shape of NifL between its oxidized or reduced form. In some aspects, the nifA and/or nifL genes are modified such that the concentration of oxygen has no effect on the ability of NifA to activate a promoter controlling the transcription of the reporter gene in the biosensor circuit. In some aspects, the nifA and/or nifL genes are modified such that the concentration of oxygen has no effect on the ability of NifA to activate the nifH promoter.

In some aspects, the nifA and the nifL genes utilized in the biosensor circuit are isolated from species of the following genera: Klebsiella, Kosakonia, Serratia, Rahnella, Pectobacterium, Dickeya, Vibrio, Pseudomonas, Azotobacter, Teredinibacter, Methylomonas, Methylovulum, Thiocystis, Thiodictyon, Thioflavicoccus, Aquaspirillum, Dechloromonas, Azoarcus, and Wolinella. In some aspects, the nifA and the nifL genes utilized in the biosensor circuit are isolated from any species within Enterobacteriaceae, Rhodocyclaceae, or Zoogloeaceae. In some aspects, the nifA is selected from one species of the listed genera and the nifL is selected from a different species of the listed genera. In some aspects, the nifA is selected from a species of the listed genera and the nifL is selected from a species of a different genus than the nifA.

In some aspects, the nifH promoter utilized in the biosensor circuit is isolated from species of the following genera: Klebsiella, Kosakonia, Serratia, Rahnella, Pectobacterium, Dickeya, Vibrio, Pseudomonas, Azotobacter, Teredinibacter, Methylomonas, Methylovulum, Thiocystis, Thiodictyon, Thioflavicoccus, Aquaspirillum, Dechloromonas, Azoarcus, and Wolinella. In some aspects, the nifH promoter utilized in the biosensor circuit is isolated from any species within Enterobacteriaceae, Rhodocyclaceae, or Zoogloeaceae.

In some aspects, the nifH promoter and the nifL and nifA genes are each isolated from a different species within the same genus. In some aspects, the nifH promoter and the nifL and nifA genes are each isolated from a different genera of bacteria. In some aspects, the nifH promoter and the nifL and nifA genes are each isolated from the same species.

In some aspects, the nifH promoter, the nifL gene, and the nifA gene are each isolated from a species of Klebsiella. In some aspects, at least one of the nifH promoter, the nifL gene, and the nifA gene are isolated from a species of Klebsiella.

In some aspects, the nifH promoter, the nifL gene, and the nifA gene are each isolated from a species of Kosakonia. In some aspects, at least one of the nifH promoter, the nifL gene, and the nifA gene are isolated from a species of Kosakonia.

In some aspects, the nifH promoter, the nifL gene, and the nifA gene are each isolated from a species of Azotobacter. In some aspects, at least one of the nifH promoter, the nifL gene, and the nifA gene are isolated from a species of Azotobacter.

In some aspects, the constitutive promoter controlling the nucleic acid sequence encoding the inhibitory nitrogen fixation regulatory protein and/or the nucleic acid sequence encoding the positive nitrogen fixation regulatory protein is selected from E. coli or Bacillus subtilis. In some aspects, the constitutive promoter controlling the nifLA is selected from E. coli or Bacillus subtilis. In some aspects, the constitutive promoter is selected from an E. coli constitutive promoter. In some aspects, the constitutive promoter is a constitutive σ⁷⁰ promoter. In some aspects, the constitutive promoter is a constitutive as promoter. In some aspects, the constitutive promoter is a constitutive σ³² promoter. In some aspects, the constitutive promoter is a constitutive σ⁵⁴ promoter. In some aspects, the constitutive promoter is selected from a B. subtilis constitutive promoter. In some aspects, the constitutive promoter is a constitutive σ^{A} promoter. In some aspects, the constitutive promoter is a constitutive σ^{B} promoter.

In some aspects, the one or more transcriptional terminators are selected from rho-independent terminators and rho-dependent terminators. In some aspects, the one of more transcriptional terminators are selected from any one or more bacterial terminator. In some aspects, the biosensor circuit comprises two or more transcriptional terminators. In some aspects, the two or more transcriptional terminators are from different species. In some aspects, the two or more transcriptional terminators are from the same genus. In some aspects, the two or more transcriptional terminators are from different genera.

In some aspects, the biosensor is a prokaryotic cell or a eukaryotic cell. In some aspects, the biosensor is a yeast or fungal cell. In some aspects, the biosensor is an insect cell, mammalian cell, animal cell, plant cell, or a bacterial cell. In some aspects, the biosensor is a fixed cell. In some aspects, the biosensor is an artificial cell. In some aspects, the biosensor is a synthetic cell. In some aspects, the insect cell is a sf9 cell or a Drosophila Schneider 2 cell. In some aspects, the mammalian cell is a HEK cell, a CHO cell, a COS cell, a 3t3 cell, or other cultured, immortalized, or passaged mammalian cell. In some aspects, the yeast or fungal cell is a Saccharomyces, Cryptococcus, or Candida cell.

In some aspects, the bacterial cell is a species of one or more of the following taxa: Achromobacter, Acidithiobacillus, Acidovorax, Acidovoraz, Acinetobacter, Actinoplanes, Adlercreutzia, Aerococcus, Aeromonas, Afipia, Agromyces, Ancylobacter, Arthrobacter, Atopostipes, Azospirillum, Bacillus, Bdellovibrio, Beijerinckia, Bosea, Bradyrhizobium, Brevibacillus, Brevundimonas, Burkholderia, Candidatus Haloredivivus, Caulobacter, Cellulomonas, Cellvibrio, Chryseobacterium, Citrobacter, Clostridium, Coraliomargarita, Corynebacterium, Cupriavidus, Curtobacterium, Curvibacter, Deinococcus, Delftia, Desemzia, Devosia, Dokdonella, Dyella, Enhydrobacter, Enterobacter, Enterococcus, Erwinia, Escherichia, Escherichia/Shigella, Exiguobacterium, Ferroglobus, Filimonas, Finegoldia, Flavisolibacter, Flavobacterium, Frigoribacterium, Gluconacetobacter, Hafnia, Halobaculum, Halomonas, Halosimplex, Herbaspirillum, Hymenobacter, Klebsiella, Kocuria, Kosakonia, Lactobacillus, Leclercia, Lentzea, Luteibacter, Luteimonas, Massilia, Mesorhizobium, Metakosakonia, Methylobacterium, Microbacterium, Micrococcus, Microvirga, Mycobacterium, Neisseria, Nocardia, Oceanibaculum, Ochrobactrum, Okibacterium, Oligotropha, Oryzihumus, Oxalophagus, Paenibacillus, Panteoa, Pantoea, Paraburkholderia Pelomonas, Perlucidibaca, Plantibacter, Polynucleobacter, Propionibacterium, Propioniciclava, Pseudoclavibacter, Pseudomonas, Pseudonocardia, Pseudoxanthomonas, Psychrobacter, Rahnella, Ralstonia, Rheinheimera, Rhizobium, Rhodococcus, Rhodopseudomonas, Roseateles, Ruminococcus, Sebaldella, Sediminibacillus, Sediminibacterium, Serratia, Shigella, Shinella, Sinorhizobium, Sinosporangium, Sphingobacterium, Sphingomonas, Sphingopyxis, Sphingosinicella, Staphylococcus, Stenotrophomonas, Strenotrophomonas, Streptococcus, Streptomyces, Stygiolobus, Sulfurisphaera, Tatumella, Tepidimonas, Thermomonas, Thiobacillus, Variovorax, WPS-2 genera incertae sedis, Xanthomonas, and Zimmermannella.

In some aspects, the bacterial cell is a species of one or more of the following taxa: Agrobacterium radiobacter, Bacillus acidocaldarius, Bacillus acidoterrestris, Bacillus agri, Bacillus aizawai, Bacillus albolactis, Bacillus alcalophilus, Bacillus alvei, Bacillus aminoglucosidicus, Bacillus aminovorans, Bacillus amylolyticus (also known as Paenibacillus amylolyticus), Bacillus amyloliquefaciens, Bacillus aneurinolyticus, Bacillus atrophaeus, Bacillus azotoformans, Bacillus badius, Bacillus cereus (synonyms: Bacillus endorhythmos, Bacillus medusa), Bacillus chitinosporus, Bacillus circulans, Bacillus coagulans, Bacillus endoparasiticus Bacillus fastidiosus, Bacillus firmus, Bacillus kurstaki, Bacillus lacticola, Bacillus lactimorbus, Bacillus lactis, Bacillus laterosporus (also known as Brevibacillus laterosporus), Bacillus lautus, Bacillus lentimorbus, Bacillus lentus, Bacillus lichenformis, Bacillus maroccanus, Bacillus megaterium, Bacillus metiens, Bacillus mycoides, Bacillus natto, Bacillus nematocida, Bacillus nigrifcans, Bacillus nigrum, Bacillus pantothenticus, Bacillus popillae, Bacillus psychrosaccharolyticus, Bacillus pumilus, Bacillus siamensis, Bacillus smithii, Bacillus sphaericus, Bacillus subtilis, Bacillus thuringiensis, Bacillus unflagellatus, Bradyrhizobium japonicum, Brevibacillus brevis Brevibacillus laterosporus (formerly Bacillus laterosporus), Chromobacterium subtsugae, Delftia acidovorans, Escherichia coli, Klebsiella variicola, Lactobacillus acidophilus, Lysobacter antibioticus, Lysobacter enzymogenes, Paenibacillus alvei, Paenibacillus polymyxa, Paenibacillus popilliae (formerly Bacillus popilliae), Pantoea agglomerans, Pasteuria penetrans (formerly Bacillus penetrans), Pasteuria usgae, Pectobacterium carotovorum (formerly Erwinia carotovora), Pseudomonas aeruginosa, Pseudomonas aureofaciens, Pseudomonas cepacia (formerly known as Burkholderia cepacia), Pseudomonas chlororaphis, Pseudomonas fluorescens, Pseudomonas proradix, Pseudomonas putida, Pseudomonas syringae, Serratia entomophila, Serratia marcescens, Streptomyces colombiensis, Streptomyces galbus, Streptomyces goshikiensis, Streptomyces griseoviridis, Streptomyces lavendulae, Streptomyces prasinus, Streptomyces saraceticus, Streptomyces venezuelae, Xanthomonas campestris, Xenorhabdus luminescens, and Xenorhabdus nematophila, In some cases the bacterium may be Azotobacter chroococcum, Methanosarcina barkeri, Klebsiella pneumoniae, Azotobacter vinelandii, Rhodobacter spharoides, Rhodobacter capsulatus, Rhodobcter palustris, Rhodosporillum rubrum, Rhizobium leguminosarum, or Rhizobium etli.

In some aspects, genetic manipulation of a cell to create the biosensor is desired. Genetic manipulation and modification of cells to create a biosensor typically involve the use of appropriately selected genetic delivery vehicles that function efficiently in the cell type of choice. In some aspects, a gene delivery vehicle such as, for example, bacterial plasmid vectors or viral vectors, for introducing the appropriate genetic material into the biosensor cells may be utilized.

In some aspects, it is useful to utilize a promoter element that directs high level expression of introduced genetic elements in the biosensor cell of choice. In some aspects, the promoter element is derived directly from the biosensor cell itself and then used to express one or more genes of interest from the biosensor circuit. In some aspects, the biosensor circuit may be introduced into the biosensor cell using standard techniques such as electroporation, transfection, etc. Other genetic engineering methods known to those or ordinary skill in the art are also compatible with the present disclosure.

In some aspects, the biosensor exhibits a high cooperativity, meaning that the biosensor has a narrow range of response to ammonium. In some instances, if the concentration or amount of ammonium is out of the narrow response range of the biosensor, it exhibits an on signal (high reporter protein, e.g., GFP) or an off signal (no reporter protein, e.g., GFP). In some aspects, the ammonium levels of the positive control can be quantified by the following: ˜1.4 mM (0.7 mM*2 from 2 times dilution (×0.5)) from the response curve. In some aspects, when the medium is diluted four (4) times (×0.25), the ammonium level is ˜0.35 mM for the biosensor which yields high reporter protein (e.g., GFP) signal. In embodiments wherein the reporter protein is a fluorescent protein, the reporter protein can be detected by applying light excitation sufficient to fluoresce the fluorescent protein, functional fragment, and/or fusions thereof. In embodiments wherein the reporter protein is a fluorescent protein, the fluorescence can be detected with a flow cytometer, plate reader or fluorescence-activated droplet sorting.

In some aspects, the non-biosensor microbes are grown in a medium and the non-biosensor microbes are then removed from the media. In some aspects, the resulting media are essentially free of the non-biosensor microbes. In some aspects, one or more biosensors are added to the media that are essentially free of the non-biosensor microbes.

In some aspects, the non-biosensor microbes are grown in a medium and the medium is diluted with a buffered solution or media. In some aspects, the medium is diluted at least 1:1, 1:2, 1:3, 1:4, 1:5, 1:6, 1:7, 1:8, 1:9, 1:10, 1:15, 1:20, 1:25, 1:30, 1:35, 1:40, 1:45, 1:50, 1:60, 1:70, 1:80, 1:90, 1:100, 1:200, 1:300, 1:400, 1:500, 1:600, 1:700, 1:800, 1:900, 1:1000, 1:1500, 1:2000, 1:2500, 1:3000, 1:3500, 1:4000, 1:4500, 1:5000, 1:5500, 1:6000, 1:6500, 1:7000, 1:7500, 1:8000, 1:8500, 1:9000, 1:9500, or 1:10000. In some aspects, the non-biosensor microbes are removed from the medium prior to the dilution such that the medium is essentially free of the non-biosensor microbes. In some aspects, one or more biosensors are added to the diluted medium. In some aspects, one or more biosensors are added to the medium that is essentially free of non-biosensor microbes. In some aspects, one or more biosensors are added to the diluted medium that is essentially free of non-biosensor microbes.

In some aspects, the medium is diluted prior to adding one or more biosensors due to the response curve the one or more biosensors operate within.

In some aspects, an ammonium transporter of the biosensor strain is modified. In some aspects, a regulatory sequence of an ammonium transporter of the biosensor strain is modified. In some aspects, the modification results in a modulation of the expression of the ammonium transporter. In some aspects, the modulation is an increase in the expression of the ammonium transporter. In some aspects, the modulation is a decrease in the expression of the ammonium transporter. In some aspects, the modulation results in a change in the ammonium and/or glutamine response curve in the biosensor strain. In some aspects, this change in the ammonium and/or glutamine response curve results in the biosensor strain being capable of effectively indicating the presence or absence of ammonium and/or glutamine in the medium in which the biosensor strain is placed. In some aspects, the ammonia transporter is AmtB.

In some aspects, the non-biosensor microbes grown in the medium are diluted such that the diluted medium comprises at least 1, at least 5, at least 10, at least 20, at least 30, at least 40, at least 50, at least 60, at least 70, at least 80, at least 90, at least 100, at least 150, at least 200, at least 300, at least 400, at least 500, at least 600, at least 700, at least 800, at least 900, at least 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸ biosensor microbes.

### Bacteria

Microbes useful in the methods and compositions disclosed herein may be obtained from any source. In some aspects, microbes may be bacteria, archaea, protozoa or fungi. The microbes of this disclosure may be nitrogen fixing microbes, for example a nitrogen fixing bacteria, nitrogen fixing archaea, nitrogen fixing fungi, nitrogen fixing yeast, or nitrogen fixing protozoa. Microbes useful in the methods and compositions disclosed herein may be spore-forming microbes, for example spore forming bacteria. In some aspects, bacteria useful in the methods and compositions disclosed herein may be Gram-positive bacteria or Gram-negative bacteria. In some aspects, the bacteria may be an endospore forming bacteria of the Firmicute phylum. In some aspects, the bacteria may be a diazatroph. In some aspects, the bacteria may not be a diazotroph.

The methods and compositions of this disclosure may be used with an archaea, such as, for example, Methanothermobacter thermoautotrophicus.

In some aspects, bacteria useful in the methods and compositions disclosed herein can be a species of one or more of the following taxa: Achromobacter, Acidithiobacillus, Acidovorax, Acidovoraz, Acinetobacter, Actinoplanes, Adlercreutzia, Aerococcus, Aeromonas, Afipia, Agromyces, Ancylobacter, Arthrobacter, Atopostipes, Azospirillum, Bacillus, Bdellovibrio, Beijerinckia, Bosea, Bradyrhizobium, Brevibacillus, Brevundimonas, Burkholderia, Candidatus Haloredivivus, Caulobacter, Cellulomonas, Cellvibrio, Chryseobacterium, Citrobacter, Clostridium, Coraliomargarita, Corynebacterium, Cupriavidus, Curtobacterium, Curvibacter, Deinococcus, Delftia, Desemzia, Devosia, Dokdonella, Dyella, Enhydrobacter, Enterobacter, Enterococcus, Erwinia, Escherichia, Escherichia/Shigella, Exiguobacterium, Ferroglobus, Filimonas, Finegoldia, Flavisolibacter, Flavobacterium, Frigoribacterium, Gluconacetobacter, Hafnia, Halobaculum, Halomonas, Halosimplex, Herbaspirillum, Hymenobacter, Klebsiella, Kocuria, Kosakonia, Lactobacillus, Leclercia, Lentzea, Luteibacter, Luteimonas, Massilia, Mesorhizobium, Metakosakonia, Methylobacterium, Microbacterium, Micrococcus, Microvirga, Mycobacterium, Neisseria, Nocardia, Oceanibaculum, Ochrobactrum, Okibacterium, Oligotropha, Oryzihumus, Oxalophagus, Paenibacillus, Panteoa, Pantoea, Paraburkholderia, Pelomonas, Perlucidibaca, Plantibacter, Polynucleobacter, Propionibacterium, Propioniciclava, Pseudoclavibacter, Pseudomonas, Pseudonocardia, Pseudoxanthomonas, Psychrobacter, Rahnella, Ralstonia, Rheinheimera, Rhizobium, Rhodococcus, Rhodopseudomonas, Roseateles, Ruminococcus, Sebaldella, Sediminibacillus, Sediminibacterium, Serratia, Shigella, Shinella, Sinorhizobium, Sinosporangium, Sphingobacterium, Sphingomonas, Sphingopyxis, Sphingosinicella, Staphylococcus, Stenotrophomonas, Strenotrophomonas, Streptococcus, Streptomyces, Stygiolobus, Sulfurisphaera, Tatumella, Tepidimonas, Thermomonas, Thiobacillus, Variovorax, WPS-2 genera incertae sedis, Xanthomonas, and Zimmermannella.

In some aspects, bacteria which may be useful include, but are not limited to, Agrobacterium radiobacter, Bacillus acidocaldarius, Bacillus acidoterrestris, Bacillus agri, Bacillus aizawai, Bacillus albolactis, Bacillus alcalophilus, Bacillus alvei, Bacillus aminoglucosidicus, Bacillus aminovorans, Bacillus amylolyticus (also known as Paenibacillus amylolyticus), Bacillus amyloliquefaciens, Bacillus aneurinolyticus, Bacillus atrophaeus, Bacillus azotoformans, Bacillus badius, Bacillus cereus (synonyms: Bacillus endorhythmos, Bacillus medusa), Bacillus chitinosporus, Bacillus circulans, Bacillus coagulans, Bacillus endoparasiticus Bacillus fastidiosus, Bacillus firmus, Bacillus kurstaki, Bacillus lacticola, Bacillus lactimorbus, Bacillus lactis, Bacillus laterosporus (also known as Brevibacillus laterosporus), Bacillus lautus, Bacillus lentimorbus, Bacillus lentus, Bacillus lichenformis, Bacillus maroccanus, Bacillus megaterium, Bacillus metiens, Bacillus mycoides, Bacillus natto, Bacillus nematocida, Bacillus nigrifcans, Bacillus nigrum, Bacillus pantothenticus, Bacillus popillae, Bacillus psychrosaccharolyticus, Bacillus pumilus, Bacillus siamensis, Bacillus smithii, Bacillus sphaericus, Bacillus subtilis, Bacillus thuringiensis, Bacillus unflagellatus, Bradyrhizobium japonicum, Brevibacillus brevis Brevibacillus laterosporus (formerly Bacillus laterosporus), Chromobacterium subtsugae, Delftia acidovorans, Escherichia coli, Klebsiella variicola, Lactobacillus acidophilus, Lysobacter antibioticus, Lysobacter enzymogenes, Paenibacillus alvei, Paenibacillus polymyxa, Paenibacillus popilliae (formerly Bacillus popilliae), Pantoea agglomerans, Pasteuria penetrans (formerly Bacillus penetrans), Pasteuria usgae, Pectobacterium carotovorum (formerly Erwinia carotovora), Pseudomonas aeruginosa, Pseudomonas aureofaciens, Pseudomonas cepacia (formerly known as Burkholderia cepacia), Pseudomonas chlororaphis, Pseudomonas fluorescens, Pseudomonas proradix, Pseudomonas putida, Pseudomonas syringae, Serratia entomophila, Serratia marcescens, Streptomyces colombiensis, Streptomyces galbus, Streptomyces goshikiensis, Streptomyces griseoviridis, Streptomyces lavendulae, Streptomyces prasinus, Streptomyces saraceticus, Streptomyces venezuelae, Xanthomonas campestris, Xenorhabdus luminescens, Kosakonia sacchari, Metakosakonia intestini, Paraburkholderia tropica and Xenorhabdus nematophila, In some cases the bacterium may be Azotobacter chroococcum, Methanosarcina barkeri, Klebsiella pneumoniae, Azotobacter vinelandii, Rhodobacter spharoides, Rhodobacter capsulatus, Rhodobcter palustris, Rhodosporillum rubrum, Rhizobium leguminosarum, or Rhizobium etli.

### Budapest Treaty on the International Recognition of the Deposit of Microorganisms for the Purpose of Patent Procedures

The microbial deposits of the present disclosure were made under the provisions of the Budapest Treaty on the International Recognition of the Deposit of Microorganisms for the Purpose of Patent Procedure (Budapest Treaty). See Table 7. The disclosure contemplates embodiments comprising any one of the microbes listed in Table 7, as well as derivatives, variants, and/or mutants thereof. Further, the disclosure contemplates agricultural compositions comprising any one of the microbes listed in Table 7, as well as derivatives, variants, and/or mutants thereof. Further, the disclosure contemplates methods of utilizing any one of the microbes listed in Table 7, as well as derivatives, variants, and/or mutants thereof. Methods of the disclosure may comprise applying said microbe to a plant or plant part (such as a seed), or to an area in which said plant or plant part is to be grown, in order to supply fixed atmospheric nitrogen to said plant.

Applicants state that pursuant to 37 C.F.R. § 1.808(a)(2) “all restrictions imposed by the depositor on the availability to the public of the deposited material will be irrevocably removed upon the granting of the patent.” This statement is subject to paragraph (b) of this section (i.e. 37 C.F.R. § 1.808(b)).

In some aspects, the bacteria is selected from Table 7. In some aspects, the bacteria is selected from Table 7 and was deposited with the Bigelow National Center for Marine Algae and Microbiota (NCMA), located at 60 Bigelow Drive, East Boothbay, Maine 04544, USA with the name designation, taxonomy, accession number and date of deposit as found in Table 7. In some aspects, the bacteria is selected from Table 7 and was deposited with the American Type Culture Collection (ATCC), located at 10801 University Boulevard, Manassas, Virginia 20110-2209, USA with the name designation, taxonomy, accession number and date of deposit as found in Table 7.

TABLE 7
[Microorganisms Deposited under the Budapest Treaty

| Depos- | Name | | Accession | Date of |
|---|---|---|---|---|
| itory | Designation | Taxonomy | Number | Deposit |
| ATCC | 137-3296, | Klebsiella variicola | PTA-126709 | Mar. 25, 2020 |
| | CI3296 | | | |
| ATCC | 137-3936, | Klebsiella variicola | PTA-126710 | Mar. 25, 2020 |
| | CI3936 | | | |
| ATCC | 137-3933, | Klebsiella variicola | PTA-126711 | Mar. 25, 2020 |
| | CI3933 | | | |
| ATCC | 137-3927, | Klebsiella variicola | PTA-126712 | Mar. 25, 2020 |
| | CI3927 | | | |
| ATCC | 137-3925, | Klebsiella variicola | PTA-126713 | Mar. 25, 2020 |
| | CI3925 | | | |
| ATCC | 137-3943, | Klebsiella variicola | PTA-126714 | Mar. 25, 2020 |
| | CI3943 | | | |
| ATCC | 137-3940, | Klebsiella variicola | PTA-126715 | Mar. 25, 2020 |
| | CI3940 | | | |
| ATCC | 137-3938, | Klebsiella variicola | PTA-126716 | Mar. 25, 2020 |
| | CI3938 | | | |
| ATCC | 137-4874, | Klebsiella variicola | PTA-126717 | Mar. 25, 2020 |
| | CI4874 | | | |
| ATCC | 6-4065, | Kosakonia sacchari | PTA-126718 | Mar. 25, 2020 |
| | CI4065 | | | |
| ATCC | 910-4875, | Metakosakonia | PTA-126719 | Mar. 25, 2020 |
| | CI4875 | intestini | | |
| ATCC | 910-4876, | Metakosakonia | PTA-126720 | Mar. 25, 2020 |
| | CI4876 | intestini | | |
| ATCC | 910-4877, | Metakosakonia | PTA-126721 | Mar. 25, 2020 |
| | CI4877 | intestini | | |
| ATCC | 910-4878, | Metakosakonia | PTA-126722 | Mar. 25, 2020 |
| | CI4878 | intestini | | |
| ATCC | 8-4751, | Paraburkholderia | PTA-126723 | Mar. 25, 2020 |
| | CI4751 | tropica | | |
| ATCC | 8-4753, | Paraburkholderia | PTA-126724 | Mar. 25, 2020 |
| | CI4753 | tropica | | |
| ATCC | 8-4879, | Paraburkholderia | PTA-126725 | Mar. 25, 2020 |
| | CI4879 | tropica | | |
| ATCC | 8-4752, | Paraburkholderia | PTA-126726 | Mar. 25, 2020 |
| | CI4752 | tropica | | |
| NCMA | CI137, | Klebsiella variicola | 201708001 | Aug. 11, 2017 |
| | 137, PB137 | (WT) | | |

### Random Mutagenesis

The present disclosure relates to methods of exposing a microbe or a population of microbes to a mutagen in order to randomly mutagenize the genomic and extragenomic DNA such that the DNA of the microbes accumulate one or more mutations. The goal is to expose the microbes to the mutagen at a dose high enough to cause genetic mutations, but also at a dose low enough not to cause too many genetic mutations that are deleterious. In some aspects, the one or more mutations result in a stop codon.

In some aspects, a population of microbes comprises microbes of the same genus or species. In some aspects, a population of bacteria comprises one or more different genera or species of microbes. In some aspects, a population of microbes comprises at least 10¹⁸, 10¹⁷, 10¹⁶, 10¹⁵, 10¹⁴, 10¹³, 10¹², 10¹¹, 10¹⁰, 10⁹, 10⁸, 10⁷, 10⁶, 105, 10⁴, 10³, or 10² microbial cells.

In some aspects, the population of microbes occurs on solid media. In some aspects, the population of microbes occurs in liquid media. In some aspects, the population of microbes occurs in semi-solid media.

In some aspects, the population of microbes are exposed to one or more mutagens. In some aspects, the population of microbes are exposed to one or more mutagens for a period of time sufficient to cause at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33 at least 34, or at least 35 mutations relative to the parent microbe.

In some aspects, a nucleotide sequence in the one or more microbes is inactivated. In some aspects, the nucleotide sequence is a coding region for a protein. In some aspects, the nucleotide sequence is a leader sequence, a signal peptide sequence. In some aspects, the nucleotide sequence is a regulatory or control sequence. In some aspects, the regulatory or control sequence is selected from a promoter, a terminator, an operator, a repressor, an activator, a trans-acting element, or a cis-acting element.

In some embodiments, the random mutagenesis is selected from chemical mutagenesis, and ultraviolet mutagenesis. In some aspects, chemical mutagens are selected from mitomycin C (MMC), N-methyl-N-nitrosourea (MNU), nitrous acid (NA), diepoxybutane (DEB), 1,2,7,8-diepoxyoctane (DEO), ethyl methane sulfonate (EMS), methyl methane sulfonate (MMS), N-methyl-N′-nitro-N-nitrosoguanidine (MNNG), 4-nitroquinoline 1-oxide (4-NQO), 2-methyloxy-6-chloro-9(3-[ethyl-2-chloroethyl]-aminopropylamin)-acridinedihydrochloride (ICR-170), 2-amino purine (2AP), and hydroxylamine (HA). In some aspects, EMS is selected. In some aspects, one or more chemical mutagen is selected.

In some aspects, the mutagen is applied to a population of microbes. In some aspects, the mutagen is applied to the media and the microbes are incubated under suitable conditions for growth. In some aspects, the microbes are washed one or more times to remove remaining traces of the mutagen. In some aspects, the resulting mutagenized cells are a mutant library that are capable of being screened for any number of phenotypic changes.

In some aspects, the concentration of the chemical mutagen applied to the population of microbes is 0.05%, 0.1%, 0.15%, 0.2%, 0.25%, 0.3%, 0.35%, 0.4%, 0.45%, 0.5%, 0.55%, 0.6%, 0.65%, 0.7%, 0.8%, 0.85%, 0.9%, 0.95%, 1%, 1.25%, 1.5%, 1.75%, 2%, 2.25%, 2.5%, 2.75%, 3%, 3.25%, 3.5%, 3.75%, 4%, 4.25%, 4.5%, 4.75%, 5%, 5.25%, 5.5%, 5.75%, 6%, 6.25%, 6.5%, 6.75%, 7%, 7.25%, 7.5%, 7.75%, 8%, 8.25%, 8.5%, 8.75%, 9%, 9.25%, 9.5%, 9.75%, 10%, 10.25%, 10.5%, 10.75%, 11%, 11.25%, 11.5%, 11.75%, 12%, 12.25%, 12.5%, 12.75%, 13%, 13.25%, 13.5%, 13.75%, 14%, 14.25%, 14.5%, 14.75%, 15%, 15.5%, 16%, 16.5%, 17%, 17.5%, 18%, 18.5%, 19%, 19.5%, or 20%.

In some aspects, the concentration of the chemical mutagen applied to the population of microbes is about 0.05%, about 0.1%, about 0.15%, about 0.2%, about 0.25%, about 0.3%, about 0.35%, about 0.4%, about 0.45%, about 0.5%, about 0.55%, about 0.6%, about 0.65%, about 0.7%, about 0.8%, about 0.85%, about 0.9%, about 0.95%, about 1%, about 1.25%, about 1.5%, about 1.75%, about 2%, about 2.25%, about 2.5%, about 2.75%, about 3%, about 3.25%, about 3.5%, about 3.75%, about 4%, about 4.25%, about 4.5%, about 4.75%, about 5%, about 5.25%, about 5.5%, about 5.75%, about 6%, about 6.25%, about 6.5%, about 6.75%, about 7%, about 7.25%, about 7.5%, about 7.75%, about 8%, about 8.25%, about 8.5%, about 8.75%, about 9%, about 9.25%, about 9.5%, about 9.75%, about 10%, about 10.25%, about 10.5%, about 10.75%, about 11%, about 11.25%, about 11.5%, about 11.75%, about 12%, about 12.25%, about 12.5%, about 12.75%, about 13%, about 13.25%, about 13.5%, about 13.75%, about 14%, about 14.25%, about 14.5%, about 14.75%, about 15%, about 15.5%, about 16%, about 16.5%, about 17%, about 17.5%, about 18%, about 18.5%, about 19%, about 19.5%, or about 20%.

In some aspects, mutagenesis libraries can be optimized by selecting for the ability to grow on an ammonium analog such as ethylenediamine (EDA) and methylammonium, which would inhibit growth in microbes that are otherwise not capable of fixing nitrogen while in the presence of an exogenous nitrogen source. Given that nitrogen fixation of diazotrophs is repressed in the presence of fixed nitrogen, ammonium assimilation by glutamine synthetase (GlnA) can be inhibited by the binding of an ammonium analog such as ethylenediamine (EDA) and methylammonium to the ammonium-binding site of GlnA. Thus, diazotrophy of microbes that possess a nitrogen fixing gene cluster can be selectively evolved while ammonium assimilation of the microbes is inhibited by EDA or methylammonium. In some embodiments, the use of EDA or methylammonium as a negative selection pressure provides for the ability to identify mutants capable of growing and thriving under what would otherwise be an inhibition in the presence of EDA, overcoming nitrogenase repression by fixed nitrogen in fertilizers and the like.

In some aspects, the selection pressure by EDA or methylammonium comprises culturing the microbes in the presence of a first EDA or methylammonium concentration, followed by culturing the microbes in the presence of a second EDA or methylammonium concentration. In some aspects, the selection pressure by EDA or methylammonium further comprises culturing the microbes in the presence of a third EDA or methylammonium concentration. In some aspects, the selection pressure by EDA or methylammonium further comprises culturing the microbes in the presence of a fourth EDA or methylammonium concentration.

In some aspects, the first EDA or methylammonium concentration is the lowest concentration. In some aspects, the second EDA or methylammonium concentration is higher than the first EDA or methylammonium concentration. In some aspects, the third EDA or methylammonium concentration is higher than the first and second EDA or methylammonium concentrations. In some aspects, the fourth EDA or methylammonium concentration is higher than the first, second, and third EDA or methylammonium concentrations.

In some aspects, the EDA concentration is 0.05%, 0.06%, 0.07%, 0.08%, 0.09%, 0.1%, 0.11%, 0.12%, 0.13%, 0.14%, 0.15%, 0.16%, 0.17%, 0.18%, 0.19%, 0.2%, 0.21%, 0.22%, 0.23%, 0.24%, 0.25%, 0.26%, 0.27%, 0.28%, 0.29%, 0.3%, 0.31%, 0.32%, 0.33%, 0.34%, 0.35%, 0.36%, 0.37%, 0.38%, 0.39%, 0.4%, 0.41%, 0.42%, 0.43%, 0.44%, 0.45%, 0.46%, 0.47%, 0.48%, 0.49%, 0.5%, 0.55%, 0.6%, 0.65%, 0.7%, 0.75%, 0.8%, 0.85%, 0.9%, 0.95%, 1%, 1.1%, 1.2%, 1.3%, 1.4%, 1.5%, 1.6%, 1.7%, 1.8%, or 1.9%.

In some aspects, the EDA concentration is about 0.05%, about 0.06%, about 0.07%, about 0.08%, about 0.09%, about 0.1%, about 0.11%, about 0.12%, about 0.13%, about 0.14%, about 0.15%, about 0.16%, about 0.17%, about 0.18%, about 0.19%, about 0.2%, about 0.21%, about 0.22%, about 0.23%, about 0.24%, about 0.25%, about 0.26%, about 0.27%, about 0.28%, about 0.29%, about 0.3%, about 0.31%, about 0.32%, about 0.33%, about 0.34%, about 0.35%, about 0.36%, about 0.37%, about 0.38%, about 0.39%, about 0.4%, about 0.41%, about 0.42%, about 0.43%, about 0.44%, about 0.45%, about 0.46%, about 0.47%, about 0.48%, about 0.49%, about 0.5%, about 0.55%, about 0.6%, about 0.65%, about 0.7%, about 0.75%, about 0.8%, about 0.85%, about 0.9%, about 0.95%, about 1%, about 1.1%, about 1.2%, about 1.3%, about 1.4%, about 1.5%, about 1.6%, about 1.7%, about 1.8%, or about 1.9%.

In some embodiments, the methylammonium concentration is 5 mM, 10 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, 100 mM, 110 mM, 120 mM, 130 mM, 140 mM, 150 mM, 160 mM, 170 mM, 180 mM, 190 mM, or 200 mM.

In some embodiments, the methylammonium concentration is about 5 mM, about 10 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, 80 about mM, 85 about mM, 90 about mM, 95 about mM, 100 about mM, 110 about mM, 120 about mM, 130 about mM, 140 about mM, 150 about mM, 160 about mM, 170 about mM, 180 about mM, 190 about mM, or 200 about mM.

In some embodiments, the mutagenesis methods are combined with EDA or methylammonium selection to further weed out a mutant library for false positives.

In one aspect, provided herein is a composition comprising one or more bacteria comprising at least one genetic variation in at least one gene, or non-coding polynucleotide, of the nitrogen fixation or assimilation genetic regulatory network. It should be recognized that the mutations can be made using the methods (e.g., random mutagenesis) provided herein or can be introduced, through the use of genetic engineering methods, or other methods known in the art. The composition can further comprise an agriculturally acceptable carrier. The agriculturally acceptable carrier can be any carrier known in the art.

In some aspects, one or more of the bacteria herein comprises at least one genetic variation in a member selected from the group consisting of: nifA, nifL, ntrB, ntrC, polynucleotide encoding glutamine synthetase, GlnA, GlnB, GlnK, Drat, AmtB, polynucleotide encoding glutaminase, GlnD, FlnE, NifJ, NifH, NifD, NifK, NifY, NifE, NifN, NifU, NifS, NifV, NifW, NifZ, NifM, NifF, NifB, NifQ, a gene associated with biosynthesis of a nitrogenase enzyme, or combinations thereof.

In some aspects, one or more of the bacteria comprises at least one genetic variation in at least one gene, or non-coding polynucleotide, of the nitrogen fixation or assimilation genetic regulatory network that results in one or more of: increased expression or activity of NifA or glutaminase; decreased expression or activity of NifL, NtrB, glutamine synthetase, GlnB, GlnK, DraT, AmtB; decreased adenylyl-removing activity of GlnE; or decreased uridylyl-removing activity of GlnD.

In some aspects, one or more of the bacteria are selected from the bacterial strains listed in Table 6.

In some aspects, one or more of the bacteria fix atmospheric nitrogen at a rate higher than a wild-type parental lineage bacteria.

In some aspects, one or more of the bacteria are selected from: Klebsiella variicola CI3296, Klebsiella variicola CI3936, Klebsiella variicola CI3933, Klebsiella variicola CI3927, Klebsiella variicola CI3925, Klebsiella variicola CI3943, Klebsiella variicola CI3940, Klebsiella variicola CI3938, Klebsiella variicola CI4874, Kosakonia sacchari CI4065, Metakosakonia intestini CI4875, Metakosakonia intestini CI4876, Metakosakonia intestini CI4877, Metakosakonia intestini CI4878, Paraburkholderia tropica CI4751, Paraburkholderia tropica CI4753, Paraburkholderia tropica CI4879 and Paraburkholderia tropica CI4752. The one or more bacteria can be genetically engineered. The one or more bacteria can be part of a composition as provided herein. The composition can comprise an agriculturally acceptable carrier as provided herein.

In some aspects, one or more of the bacteria are selected from: a bacterium deposited as PTA-126709, a bacterium deposited as PTA-126710, a bacterium deposited as PTA-126711, a bacterium deposited as PTA-126712, a bacterium deposited as PTA-126713, a bacterium deposited as PTA-126714, a bacterium deposited as PTA-126715, a bacterium deposited as PTA-126716, a bacterium deposited as PTA-126717, a bacterium deposited as PTA-126718, a bacterium deposited as PTA-126719, a bacterium deposited as PTA-126720, a bacterium deposited as PTA-126721, a bacterium deposited as PTA-126722, a bacterium deposited as PTA-126723, a bacterium deposited as PTA-126724, a bacterium deposited as PTA-126725, a bacterium deposited as PTA-126726 and combinations thereof. The one or more bacteria can be genetically engineered. The one or more bacteria can be part of a composition as provided herein. The composition can comprise an agriculturally acceptable carrier as provided herein.

In some aspects, one or more of the bacteria comprise at least one genetic variation in glnA. The one or more bacteria can be genetically engineered. The at least one genetic variation can be introduced via random mutagenesis as described herein. The one or more bacteria can be part of a composition as provided herein. The composition can comprise an agriculturally acceptable carrier as provided herein.

In some cases, the one or more bacteria can comprise a mutant glnA gene comprising at least one nucleotide substitution at nucleotide position 198, 469, 515, 521, 566, 622, 652, 764, 802, 986 and/or 1015 of a Klebsiella glnA gene or at a homologous nucleotide position in a homolog thereof. In some cases, the mutant glnA gene can share at least 85%, 86%, 87%, 88%, 89%, 90%, 91%, 92%, 93%, 94%, 95%, 96%, 97%, 98%, or 99% identity with the Klebsiella glnA gene or the homolog thereof. In some cases, outside of the at least nucleotide substitution, the remainder of the mutant glnA gene can share at least 85%, 86%, 87%, 88%, 89%, 90%, 91%, 92%, 93%, 94%, 95%, 96%, 97%, 98%, 99% or 100% identity with the Klebsiella glnA gene or the homolog thereof. In one embodiment, the Klebsiella glnA gene comprises a nucleic acid sequence of SEQ ID NO: 1. In some cases, the homolog of the Klebsiella glnA gene is a Kosakonia glnA gene. In some cases, the homolog of the Klebsiella glnA gene comprises a nucleic acid sequence of SEQ ID NO: 2. In some cases, the homolog of the Klebsiella glnA gene is a Metakosakonia glnA gene. In some cases, the homolog of the Klebsiella glnA gene comprises a nucleic acid sequence of SEQ ID NO: 3. In one embodiment, the mutant glnA gene comprising at least one genetic variation or mutation comprises a nucleic acid sequence selected from the group consisting of SEQ ID NOs 6-13 and 15-19.

In some cases, the one or more bacteria can comprise a mutant glnA gene comprising at least one nucleotide substitution at nucleotide position 308, 487, 511 and/or 657 of a Paraburkholderia glnA gene or in a homologous nucleotide position in a homolog thereof. In some cases, the mutant glnA gene can share at least 85%, 86%, 87%, 88%, 89%, 90%, 91%, 92%, 93%, 94%, 95%, 96%, 97%, 98%, or 99% identity with the Paraburkholderia glnA gene or the homolog thereof. In some cases, outside of the at least nucleotide substitution, the remainder of the mutant glnA gene can share at least 85%, 86%, 87%, 88%, 89%, 90%, 91%, 92%, 93%, 94%, 95%, 96%, 97%, 98%, 99% or 100% identity with the Paraburkholderia glnA gene or the homolog thereof. In one embodiment, the Paraburkholderia glnA gene comprises a nucleic acid sequence of SEQ ID NO: 4. In one embodiment, the mutant glnA gene comprising at least one genetic variation or mutation comprises a nucleic acid sequence selected from the group consisting of SEQ ID NOs 20-23.

In one embodiment, expression of a glnA gene comprising at least one genetic variation or mutation provided herein encodes a GlnA protein with at least one amino acid substitution at amino acid position 66, 157, 172, 174, 189, 208, 218, 255, 268, 329 and/or 339 of a Klebsiella GlnA protein or at a homologous amino acid position in a homolog thereof. In one embodiment, expression of a glnA gene comprising at least one genetic variation or mutation provided herein produces a GlnA protein with at least one amino acid substitution selected from the group consisting of M66I, M66V, G157C, H172L, P174L, S189F, E208K, G218S, T255I, G268S, A329V and N339D of a Klebsiella GlnA protein and identical amino acid substitutions at homologous amino acid positions in a homolog thereof. In some cases, the GlnA protein encoded by a glnA gene comprising at least one genetic variation or mutation herein shares at least 90%, 91%, 92%, 93%, 94%, 95%, 96%, 97%, 98%, or 99% identity with the Klebsiella GlnA protein or the homolog thereof. In some cases, the remainder of the GlnA protein not including the at least one amino acid substitution that is encoded by a glnA gene comprising at least one genetic variation or mutation provided herein shares at least 90%, 91%, 92%, 93%, 94%, 95%, 96%, 97%, 98%, 99% or 100% identity with the Klebsiella GlnA protein or the homolog thereof. The Klebsiella GlnA protein can comprise an amino acid sequence of SEQ ID NO: 24. In some cases, the homolog of the Klebsiella GlnA protein is a Kosakonia GlnA protein. In some cases, the homolog of the Klebsiella GlnA protein comprises an amino acid sequence of SEQ ID NO: 25. In some cases, the homolog of the Klebsiella GlnA protein is a Metakosakonia GlnA protein. In some cases, the homolog of the Klebsiella GlnA protein comprises an amino acid sequence of SEQ ID NO: 26. In one embodiment, a GlnA protein encoded by a glnA gene comprising at least one genetic variation or mutation provided herein comprises an amino acid sequence selected from the group consisting of SEQ ID NOs 29-36 and 38-42.

In one embodiment, expression of a glnA gene comprising at least one genetic variation or provided herein encodes a GlnA protein with at least one amino acid substitution at amino acid position 103, 163, 171 and/or 219 of a Paraburkholderia GlnA protein or at a homologous amino acid position in a homolog thereof. In one embodiment, expression of a glnA gene comprising at least one genetic variation or mutation provided herein produces a GlnA protein with at least one amino acid substitution selected from the group consisting of Y103C, S163P, N171D and Q219H of a Paraburkholderia GlnA protein and identical amino acid substitutions in homologous amino acid positions in a homolog thereof. In some cases, the GlnA protein encoded by a glnA gene comprising at least one genetic variation or mutation shares at least 90%, 91%, 92%, 93%, 94%, 95%, 96%, 97%, 98%, or 99% identity with the Paraburkholderia GlnA protein or the homolog thereof. In some cases, the remainder of the GlnA protein not including the at least one amino acid substitution that is encoded by a glnA gene comprising at least one genetic variation or mutation shares at least 90%, 91%, 92%, 93%, 94%, 95%, 96%, 97%, 98%, 99% or 100% identity with the Paraburkholderia GlnA protein or the homolog thereof. The Paraburkholderia GlnA protein can comprise an amino acid sequence of SEQ ID NO: 27. In one embodiment, a GlnA protein encoded by a glnA gene comprising at least one genetic variation or mutation provided herein comprises an amino acid sequence selected from the group consisting of SEQ ID NOs 43-46.

In some aspects, the one or more bacteria comprise a mutant GlnA protein comprising an amino acid sequence with at least one amino acid substitution at amino acid positions 66, 157, 172, 174, 189, 208, 218, 255, 268, 329 and/or 339 of a Klebsiella GlnA protein or at a homologous amino acid position in a homolog thereof. In some aspects, the one or more bacteria comprise a mutant GlnA protein comprising an amino acid sequence with at least one amino acid substitution selected from the group consisting of M66I, M66V, G157C, H172L, P174L, S189F, E208K, G218S, T255I, G268S, A329V and N339D of a Klebsiella GlnA protein and identical amino acid substitution at homologous amino acid positions in a homolog thereof. In some cases, the mutant GlnA protein shares at least 90%, 91%, 92%, 93%, 94%, 95%, 96%, 97%, 98%, or 99% identity with the Klebsiella GlnA protein or the homolog thereof. In some cases, the mutant GlnA protein shares at least 90%, 91%, 92%, 93%, 94%, 95%, 96%, 97%, 98%, 99% or 100% identity with the Klebsiella GlnA protein or the homolog thereof outside of the at least one amino acid substitution. The Klebsiella GlnA protein can comprise an amino acid sequence of SEQ ID NO: 24. In some cases, the homolog of the Klebsiella GlnA protein is a Kosakonia GlnA protein. In some cases, the homolog of the Klebsiella GlnA protein comprises an amino acid sequence of SEQ ID NO: 25. In some cases, the homolog of the Klebsiella GlnA protein is a Metakosakonia GlnA protein. In some cases, the homolog of the Klebsiella GlnA protein comprises an amino acid sequence of SEQ ID NO: 26. In one embodiment, the mutant GlnA protein comprises an amino acid sequence selected from the group consisting of SEQ ID NOs 29-36 and 38-42.

In some aspects, the one or more bacteria comprise a mutant GlnA protein comprising an amino acid sequence with at least one amino acid substitution at amino acid positions 103, 163, 171 and/or 219 of a Paraburkholderia GlnA protein or at a homologous amino acid position in a homolog thereof. In some aspects, the one or more bacteria comprise a mutant GlnA protein comprising an amino acid sequence with at least one amino acid substitution selected from the group consisting of Y103C, S163P, N171D and Q219H of a Paraburkholderia GlnA protein or at a homologous amino acid position in a homolog thereof. In some cases, the mutant GlnA protein shares at least 90%, 91%, 92%, 93%, 94%, 95%, 96%, 97%, 98%, or 99% identity with the Paraburkholderia GlnA protein or the homolog thereof. In some cases, the mutant GlnA protein shares at least 90%, 91%, 92%, 93%, 94%, 95%, 96%, 97%, 98%, 99% or 100% identity with the Paraburkholderia GlnA protein or the homolog thereof outside of the at least one amino acid substitution. The Paraburkholderia GlnA protein can comprise an amino acid sequence of SEQ ID NO: 27. In one embodiment, the mutant GlnA protein comprises an amino acid sequence selected from the group consisting of SEQ ID NOs 43-46.

In some aspects, the one or more bacteria comprise at least one genetic variation in glnE. The one or more bacteria can comprise a mutant glnE gene comprising at least one nucleotide substitution at nucleotide position 965 and/or 2838 of a Klebsiella glnE gene or at a homologous nucleotide position in a homolog thereof. The one or more bacteria can comprise a mutant glnE gene that comprises a nucleotide substitution at nucleotide position 965 and 2838 of a Klebsiella glnE gene or in homologous nucleotide positions in the homolog thereof. In some cases, the mutant glnE gene can share at least 85%, 86%, 87%, 88%, 89%, 90%, 91%, 92%, 93%, 94%, 95%, 96%, 97%, 98%, or 99% identity with the Klebsiella glnE gene or the homolog thereof. In some cases, outside of the at least nucleotide substitution, the remainder of the mutant glnE gene can share at least 85%, 86%, 87%, 88%, 89%, 90%, 91%, 92%, 93%, 94%, 95%, 96%, 97%, 98%, 99% or 100% identity with the Klebsiella glnE gene or the homolog thereof. In one embodiment, the Klebsiella glnE gene comprises a nucleic acid sequence of SEQ ID NO: 5. In one embodiment, the mutant glnE gene comprising at least one genetic variation or mutation comprises a nucleic acid sequence of SEQ ID NO: 14.

In one embodiment, expression of a glnE gene comprising at least one genetic variation or mutation encodes a GlnE protein with at least one amino acid substitution at amino acid position 322 and/or 746 of a Klebsiella GlnE protein or at a homologous amino acid position in a homolog thereof. In another embodiment, expression of a glnE gene comprising at least one genetic variation or mutation produces a mutant GlnE protein with amino acid substitutions at amino acid positions 322 and 746 of the Klebsiella GlnE protein or at homologous amino acid positions in the homolog thereof. The amino acid substitution at position 322 or the homologous amino acid position can be a G to E substitution. The amino acid substitution at position 746 or the homologous amino acid position can be a G to D substitution. In some cases, the GlnE protein encoded by a glnE gene comprising at least one genetic variation or mutation shares at least 90%, 91%, 92%, 93%, 94%, 95%, 96%, 97%, 98%, or 99% identity with the Klebsiella GlnE protein or the homolog thereof. In some cases, the remainder of the GlnE protein not including the at least one amino acid substitution that is encoded by a glnE gene comprising at least one genetic variation or mutation shares at least 90%, 91%, 92%, 93%, 94%, 95%, 96%, 97%, 98%, 99% or 100% identity with the Klebsiella GlnE protein or the homolog thereof. The Klebsiella GlnE protein can comprise an amino acid sequence of SEQ ID NO: 28. In one embodiment, a mutant GlnE protein encoded by a glnE gene comprising at least one genetic variation or mutation comprises an amino acid sequence of SEQ ID NO: 37.

In some aspects, the one or more bacteria comprise a mutant GlnE protein comprising an amino acid sequence with at least one amino acid substitution at amino acid positions 322 and/or 746 of a Klebsiella GlnE protein or at a homologous amino acid position in a homolog thereof. In other aspects, the one or more bacteria comprise a mutant GlnE protein comprising amino acid substitutions at amino acid positions 322 and 746 of a Klebsiella GlnE protein or at homologous amino acid positions in the homolog thereof. In some cases, the amino acid substitution at position 322 or the homologous amino acid position is a G to E substitution. In some cases, the amino acid substitution at position 746 or the homologous amino acid position is a G to D substitution. In some cases, the mutant GlnE protein shares at least 90%, 91%, 92%, 93%, 94%, 95%, 96%, 97%, 98%, or 99% identity with the Klebsiella GlnE protein or the homolog thereof. In some cases, the mutant GlnE protein shares at least 90%, 91%, 92%, 93%, 94%, 95%, 96%, 97%, 98%, 99% or 100% identity with the Klebsiella GlnE protein or the homolog thereof outside of the at least one amino acid substitution. The Klebsiella GlnE protein can comprise an amino acid sequence of SEQ ID NO: 28. In one embodiment, the mutant GlnE protein comprises an amino acid sequence of SEQ ID NO: 37.

It would be recognized by those of skill in the art, that nucleotide and protein sequence homologs may be of the same length or may contain insertions and/or deletions. In some cases, the homologous nucleotide and/or amino acid position in a homolog is identical to the nucleotide or amino acid position in a base sequence. In other cases, the homologous nucleotide and/or amino acid position in a homolog is a different nucleotide or amino acid position than in the base sequence. The homologous nucleotide or amino acid position in the homolog (the position in the homolog at which a substitution would occur based upon a substitution position disclosed herein) can be identified by aligning the homolog to a base sequence with a substitution disclosed herein and identifying the position of the nucleotide or the amino acid in the homolog that aligns with the position of the nucleotide or the amino acid in the base sequence that contains a substitution as disclosed herein. Such sequence alignment can be carried out by methods known to those of skill in the art.

### Applications

Methods of the present disclosure utilizing strains identified herein may be employed to introduce or improve one or more of a variety of desirable traits. Examples of traits that may introduced or improved include: root biomass, root length, height, shoot length, leaf number, water use efficiency, overall biomass, yield, fruit size, grain size, photosynthesis rate, tolerance to drought, heat tolerance, salt tolerance, resistance to nematode stress, resistance to a fungal pathogen, resistance to a bacterial pathogen, resistance to a viral pathogen, level of a metabolite, and proteome expression. The desirable traits, including height, overall biomass, root and/or shoot biomass, seed germination, seedling survival, photosynthetic efficiency, transpiration rate, seed/fruit number or mass, plant grain or fruit yield, leaf chlorophyll content, photosynthetic rate, root length, or any combination thereof, can be used to measure growth, and compared with the growth rate of reference agricultural plants (e.g., plants without the improved traits) grown under identical conditions.

A preferred trait to be introduced or improved is nitrogen fixation, as described herein. In some cases, a plant resulting from the methods described herein exhibits a difference in the trait that is at least about 5% greater, for example at least about 5%, at least about 8%, at least about 10%, at least about 15%, at least about 20%, at least about 25%, at least about 30%, at least about 40%, at least about 50%, at least about 60%, at least about 75%, at least about 80%, at least about 80%, at least about 90%, or at least 100%, at least about 200%, at least about 300%, at least about 400% or greater than a reference agricultural plant grown under the same conditions in the soil. In additional examples, a plant resulting from the methods described herein exhibits a difference in the trait that is at least about 5% greater, for example at least about 5%, at least about 8%, at least about 10%, at least about 15%, at least about 20%, at least about 25%, at least about 30%, at least about 40%, at least about 50%, at least about 60%, at least about 75%, at least about 80%, at least about 80%, at least about 90%, or at least 100%, at least about 200%, at least about 300%, at least about 400% or greater than a reference agricultural plant grown under similar conditions in the soil.

The trait to be improved utilizing microbial strains identified herein may be assessed under conditions including the application of one or more biotic or abiotic stressors. Examples of stressors include abiotic stresses (such as heat stress, salt stress, drought stress, cold stress, and low nutrient stress) and biotic stresses (such as nematode stress, insect herbivory stress, fungal pathogen stress, bacterial pathogen stress, and viral pathogen stress).

The trait improved by methods and compositions utilizing microbial strains identified herein may be nitrogen fixation, including in a plant not previously capable of nitrogen fixation. In some cases, bacteria isolated according to a method described herein produce 1% or more (e.g. 2%, 3%, 4%, 5%, 6%, 7%, 8%, 9%, 10%, 15%, 20%, or more) of a plant's nitrogen, which may represent an increase in nitrogen fixation capability of at least 2-fold (e.g. 3-fold, 4-fold, 5-fold, 6-fold, 7-fold, 8-fold, 9-fold, 10-fold, 20-fold, 50-fold, 100-fold, 1000-fold, or more) as compared to bacteria isolated from a plant before introducing any genetic variation. In some cases, the bacteria produce 5% or more of a plant's nitrogen. The desired level of nitrogen fixation may be achieved after repeating the steps of introducing genetic variation, exposure to a plurality of plants, and isolating bacteria from plants with an improved trait one or more times (e.g. 1, 2, 3, 4, 5, 10, 15, 25, or more times). In some cases, enhanced levels of nitrogen fixation are achieved in the presence of fertilizer supplemented with glutamine, ammonia, or other chemical source of nitrogen. Methods for assessing degree of nitrogen fixation are known, examples of which are described herein.

### Measuring Nitrogen Delivered in an Agriculturally Relevant Field Context

In the field, the amount of nitrogen delivered can be determined by the function of colonization multiplied by the activity.

$$Nitrogen delivered = {\int }_{Time \& Space} Colonization \times Activity$$

The above equation requires (1) the average colonization per unit of plant tissue, and (2) the activity as either the amount of nitrogen fixed or the amount of ammonia excreted by each microbial cell. To convert to pounds of nitrogen per acre, corn growth physiology is tracked over time, e.g., size of the plant and associated root system throughout the maturity stages.

The pounds of nitrogen delivered to a crop per acre-season can be calculated by the following equation:

$$Nitrogen delivered = \int Plant Tissue ( t ) \times Colonization ( t ) \times Activity ( t ) dt$$

The Plant Tissue(t) is the fresh weight of corn plant tissue over the growing time (t). Values for reasonably making the calculation are described in detail in the publication entitled Roots, Growth and Nutrient Uptake (Mengel. Dept. of Agronomy Pub. #AGRY-95-08 (Rev. May-95. p. 1-8.).

The Colonization (t) is the amount of the microbes of interest found within the plant tissue, per gram fresh weight of plant tissue, at any particular time, t, during the growing season. In the instance of only a single timepoint available, the single timepoint is normalized as the peak colonization rate over the season, and the colonization rate of the remaining timepoints are adjusted accordingly.

Activity(t) is the rate of which N is fixed by the microbes of interest per unit time, at any particular time, t, during the growing season. In the embodiments disclosed herein, this activity rate is approximated by in vitro acetylene reduction assay (ARA) in ARA media in the presence of 5 mM glutamine or Ammonium excretion assay in ARA media in the presence of 5 mM ammonium ions.

The Nitrogen delivered amount is then calculated by numerically integrating the above function. In cases where the values of the variables described above are discretely measured at set timepoints, the values in between those timepoints are approximated by performing linear interpolation.

### Nitrogen Fixation

Described herein are methods of increasing nitrogen fixation in a plant, comprising exposing the plant to one or more bacteria described herein, including through the application of compositions comprising the bacteria, as described herein. It should be understood that the bacteria may be generated and/or identified using the methods and compositions provided herein (e.g., through the use of a biosensor as provided herein) or through other methods known in the art. These bacteria comprise one or more genetic variations in one or more genes regulating nitrogen fixation, wherein the bacteria produce 1% or more (e.g. 2%, 3%, 4%, 5%, 6%, 7%, 8%, 9%, 10%, 15%, 20%, or more) of nitrogen in the plant, which may represent a nitrogen fixation capability of at least 2-fold (e.g. 3-fold, 4-fold, 5-fold, 6-fold, 7-fold, 8-fold, 9-fold, 10-fold, 20-fold, 50-fold, 100-fold, 1000-fold, or more) as compared to the plant in the absence of the bacteria. The bacteria may produce the nitrogen in the presence of fertilizer supplemented with glutamine, urea, nitrates or ammonia. Genetic variations can be any genetic variation described herein, including examples provided herein, in any number and any combination.

The amount of nitrogen fixation that occurs in the plants described herein may be measured in several ways, for example by an acetylene-reduction (AR) assay. An acetylene-reduction assay can be performed in vitro or in vivo. Evidence that a particular bacterium is providing fixed nitrogen to a plant can include: 1) total plant N significantly increases upon inoculation, preferably with a concomitant increase in N concentration in the plant; 2) nitrogen deficiency symptoms are relieved under N-limiting conditions upon inoculation (which should include an increase in dry matter); 3) N₂ fixation is documented through the use of an ¹⁵N approach (which can be isotope dilution experiments, ¹⁵N₂ reduction assays, or ¹⁵N natural abundance assays); 4) fixed N is incorporated into a plant protein or metabolite; and 5) all of these effects are not be seen in non-inoculated plants or in plants inoculated with a mutant of the inoculum strain.

### Agricultural Compositions

Compositions comprising bacteria or bacterial populations described herein can be in the form of a liquid, a foam, or a dry product. Compositions comprising bacteria or bacterial populations described herein may also be used to improve plant traits. Compositions comprising bacteria or bacterial populations can comprise engineered or randomly mutagenized microbes that comprise one or any combination of genetic variations in a glnA gene and/or glnE gene as provided herein. Any composition provided herein comprising one or more strains of microbes as provided herein can further comprise one or more strains from Table 6 as provided herein.

In some examples, a composition comprising bacterial populations may be in the form of a dry powder, a slurry of powder and water, or a flowable seed treatment. The compositions comprising bacterial populations may be coated on a surface of a seed, and may be in liquid form.

The composition can be fabricated in bioreactors such as continuous stirred tank reactors, batch reactors, and on the farm. In some examples, compositions can be stored in a container, such as a jug or in mini bulk. In some examples, compositions may be stored within an object selected from the group consisting of a bottle, jar, ampule, package, vessel, bag, box, bin, envelope, carton, container, silo, shipping container, truck bed, and/or case.

Compositions may also be used to improve plant traits. In some examples, one or more compositions may be coated onto a seed. In some examples, one or more compositions may be coated onto a seedling. In some examples, one or more compositions may be coated onto a surface of a seed. In some examples, one or more compositions may be coated as a layer above a surface of a seed. In some examples, a composition that is coated onto a seed may be in liquid form, in dry product form, in foam form, in a form of a slurry of powder and water, or in a flowable seed treatment. In some examples, one or more compositions may be applied to a seed and/or seedling by spraying, immersing, coating, encapsulating, and/or dusting the seed and/or seedling with the one or more compositions. In some examples, multiple bacteria or bacterial populations can be coated onto a seed and/or a seedling of the plant. In some examples, at least two, at least three, at least four, at least five, at least six, at least seven, at least eight, at least nine, at least ten, or more than ten bacteria of a bacterial combination can be selected from the strains of bacteria found in Table 6.

Examples of compositions may include seed coatings for commercially important agricultural crops, for example, sorghum, canola, tomato, strawberry, barley, rice, maize, and wheat. Examples of compositions can also include seed coatings for corn, soybean, canola, sorghum, potato, rice, vegetables, cereals, and oilseeds. Seeds as provided herein can be genetically modified organisms (GMO), non-GMO, organic, or conventional. In some examples, compositions may be sprayed on the plant aerial parts, or applied to the roots by inserting into furrows in which the plant seeds are planted, watering to the soil, or dipping the roots in a suspension of the composition. In some examples, compositions may be dehydrated in a suitable manner that maintains cell viability and the ability to artificially inoculate and colonize host plants. The bacterial species may be present in compositions at a concentration of between 10⁸ to 10¹⁰ CFU/ml. In some examples, compositions may be supplemented with trace metal ions, such as molybdenum ions, iron ions, manganese ions, or combinations of these ions. The concentration of ions in examples of compositions as described herein may between about 0.1 mM and about 50 mM. Some examples of compositions may also be formulated with a carrier, such as beta-glucan, carboxylmethyl cellulose (CMC), bacterial extracellular polymeric substance (EPS), sugar, animal milk, or other suitable carriers. In some examples, peat or planting materials can be used as a carrier, or biopolymers in which a composition is entrapped in the biopolymer can be used as a carrier. The compositions comprising the bacterial populations described herein can improve plant traits, such as promoting plant growth, maintaining high chlorophyll content in leaves, increasing fruit or seed numbers, and increasing fruit or seed unit weight.

The compositions comprising the bacterial populations described herein may be coated onto the surface of a seed. As such, compositions comprising a seed coated with one or more bacteria described herein are also contemplated. The seed coating can be formed by mixing the bacterial population with a porous, chemically inert granular carrier. Alternatively, the compositions may be inserted directly into the furrows into which the seed is planted or sprayed onto the plant leaves or applied by dipping the roots into a suspension of the composition. An effective amount of the composition can be used to populate the sub-soil region adjacent to the roots of the plant with viable bacterial growth, or populate the leaves of the plant with viable bacterial growth. In general, an effective amount is an amount sufficient to result in plants with improved traits (e.g. a desired level of nitrogen fixation).

Bacterial compositions described herein can be formulated using an agriculturally acceptable carrier. The formulation useful for these embodiments may include at least one member selected from the group consisting of a tackifier, a microbial stabilizer, a fungicide, an antibacterial agent, a preservative, a stabilizer, a surfactant, an anti-complex agent, an herbicide, a nematicide, an insecticide, a plant growth regulator, a fertilizer, a rodenticide, a desiccant, a bactericide, a nutrient, a hormone, or any combination thereof. In some examples, compositions may be shelf-stable. For example, any of the compositions described herein can include an agriculturally acceptable carrier (e.g., one or more of a fertilizer such as a non-naturally occurring fertilizer, an adhesion agent such as a non-naturally occurring adhesion agent, and a pesticide such as a non-naturally occurring pesticide). A non-naturally occurring adhesion agent can be, for example, a polymer, copolymer, or synthetic wax. For example, any of the coated seeds, seedlings, or plants described herein can contain such an agriculturally acceptable carrier in the seed coating. In any of the compositions or methods described herein, an agriculturally acceptable carrier can be or can include a non-naturally occurring compound (e.g., a non-naturally occurring fertilizer, a non-naturally occurring adhesion agent such as a polymer, copolymer, or synthetic wax, or a non-naturally occurring pesticide). Non-limiting examples of agriculturally acceptable carriers are described below. Additional examples of agriculturally acceptable carriers are known in the art.

In some cases, bacteria are mixed with an agriculturally acceptable carrier. The carrier can be a solid carrier or liquid carrier, and in various forms including microspheres, powders, emulsions and the like. The carrier may be any one or more of a number of carriers that confer a variety of properties, such as increased stability, wettability, or dispersability. Wetting agents such as natural or synthetic surfactants, which can be nonionic or ionic surfactants, or a combination thereof can be included in the composition. Water-in-oil emulsions can also be used to formulate a composition that includes the isolated bacteria (see, for example, U.S. Pat. No. 7,485,451). Suitable formulations that may be prepared include wettable powders, granules, gels, agar strips or pellets, thickeners, and the like, microencapsulated particles, and the like, liquids such as aqueous flowables, aqueous suspensions, water-in-oil emulsions, etc. The formulation may include grain or legume products, for example, ground grain or beans, broth or flour derived from grain or beans, starch, sugar, or oil.

In some embodiments, the agricultural carrier may be soil or a plant growth medium. Other agricultural carriers that may be used include water, fertilizers, plant-based oils, humectants, or combinations thereof. Alternatively, the agricultural carrier may be a solid, such as diatomaceous earth, loam, silica, alginate, clay, bentonite, vermiculite, seed cases, other plant and animal products, or combinations, including granules, pellets, or suspensions. Mixtures of any of the aforementioned ingredients are also contemplated as carriers, such as but not limited to, pesta (flour and kaolin clay), agar or flour-based pellets in loam, sand, or clay, etc. Formulations may include food sources for the bacteria, such as barley, rice, or other biological materials such as seed, plant parts, sugar cane bagasse, hulls or stalks from grain processing, ground plant material or wood from building site refuse, sawdust or small fibers from recycling of paper, fabric, or wood.

For example, a fertilizer can be used to help promote the growth or provide nutrients to a seed, seedling, or plant. Non-limiting examples of fertilizers include nitrogen, phosphorous, potassium, calcium, sulfur, magnesium, boron, chloride, manganese, iron, zinc, copper, molybdenum, and selenium (or a salt thereof). Additional examples of fertilizers include one or more amino acids, salts, carbohydrates, vitamins, glucose, NaCl, yeast extract, NH4H2PO4, (NH4)2SO4, glycerol, valine, L-leucine, lactic acid, propionic acid, succinic acid, malic acid, citric acid, KH tartrate, xylose, lyxose, and lecithin. In one embodiment, the formulation can include a tackifier or adherent (referred to as an adhesive agent) to help bind other active agents to a substance (e.g., a surface of a seed). Such agents are useful for combining bacteria with carriers that can contain other compounds (e.g., control agents that are not biologic), to yield a coating composition. Such compositions help create coatings around the plant or seed to maintain contact between the microbe and other agents with the plant or plant part. In one embodiment, adhesives are selected from the group consisting of: alginate, gums, starches, lecithins, formononetin, polyvinyl alcohol, alkali formononetinate, hesperetin, polyvinyl acetate, cephalins, Gum Arabic, Xanthan Gum, Mineral Oil, Polyethylene Glycol (PEG), Polyvinyl pyrrolidone (PVP), Arabino-galactan, Methyl Cellulose, PEG 400, Chitosan, Polyacrylamide, Polyacrylate, Polyacrylonitrile, Glycerol, Triethylene glycol, Vinyl Acetate, Gellan Gum, Polystyrene, Polyvinyl, Carboxymethyl cellulose, Gum Ghatti, and polyoxyethylene-polyoxybutylene block copolymers.

In some embodiments, the adhesives can be, e.g. a wax such as carnauba wax, beeswax, Chinese wax, shellac wax, spermaceti wax, candelilla wax, castor wax, ouricury wax, and rice bran wax, a polysaccharide (e.g., starch, dextrins, maltodextrins, alginate, and chitosans), a fat, oil, a protein (e.g., gelatin and zeins), gum arables, and shellacs. Adhesive agents can be non-naturally occurring compounds, e.g., polymers, copolymers, and waxes. For example, non-limiting examples of polymers that can be used as an adhesive agent include: polyvinyl acetates, polyvinyl acetate copolymers, ethylene vinyl acetate (EVA) copolymers, polyvinyl alcohols, polyvinyl alcohol copolymers, celluloses (e.g., ethylcelluloses, methylcelluloses, hydroxymethylcelluloses, hydroxypropylcelluloses, and carboxymethylcelluloses), polyvinylpyrolidones, vinyl chloride, vinylidene chloride copolymers, calcium lignosulfonates, acrylic copolymers, polyvinylacrylates, polyethylene oxide, acylamide polymers and copolymers, polyhydroxyethyl acrylate, methylacrylamide monomers, and polychloroprene.

In some examples, one or more of the adhesion agents, anti-fungal agents, growth regulation agents, and pesticides (e.g., insecticide) are non-naturally occurring compounds (e.g., in any combination). Additional examples of agriculturally acceptable carriers include dispersants (e.g., polyvinylpyrrolidone/vinyl acetate PVPIVA S-630), surfactants, binders, and filler agents.

The formulation can also contain a surfactant. Non-limiting examples of surfactants include nitrogen-surfactant blends such as Prefer 28 (Cenex), Surf-N(US), Inhance (Brandt), P-28 (Wilfarm) and Patrol (Helena); esterified seed oils include Sun-It II™ (adjuvant; AmCy), MSO (UAP), Scoil (Agsco), Hasten (Wilfarm) and Mes-100 (Drexel); and organo-silicone surfactants include Silwet® L77 (UAP), Silikin (Terra), Dyne-Amic (Helena), Kinetic® (nonionic organosilicone spray adjuvant; Helena), Sylgard® 309 (nonionic silicone polyether surfactant; Wilbur-Ellis) and Century (Precision). In one embodiment, the surfactant is present at a concentration of between 0.01% v/v to 10% v/v. In another embodiment, the surfactant is present at a concentration of between 0.1% v/v to 1% v/v.

In certain cases, the formulation includes a microbial stabilizer. Such an agent can include a desiccant, which can include any compound or mixture of compounds that can be classified as a desiccant regardless of whether the compound or compounds are used in such concentrations that they in fact have a desiccating effect on a liquid inoculant. Such desiccants are ideally compatible with the bacterial population used, and should promote the ability of the microbial population to survive application on the seeds and to survive desiccation. Examples of suitable desiccants include one or more of trehalose, sucrose, glycerol, and Methylene glycol. Other suitable desiccants include, but are not limited to, non reducing sugars and sugar alcohols (e.g., mannitol or sorbitol). The amount of desiccant introduced into the formulation can range from about 5% to about 50% by weight/volume, for example, between about 10% to about 40%, between about 15% to about 35%, or between about 20% to about 30%. In some cases, it is advantageous for the formulation to contain agents such as a fungicide, an antibacterial agent, an herbicide, a nematicide, an insecticide, a plant growth regulator, a rodenticide, bactericide, or a nutrient. In some examples, agents may include protectants that provide protection against seed surface-borne pathogens. In some examples, protectants may provide some level of control of soil-borne pathogens. In some examples, protectants may be effective predominantly on a seed surface.

In some examples, a fungicide may include a compound or agent, whether chemical or biological, that can inhibit the growth of a fungus or kill a fungus. In some examples, a fungicide may include compounds that may be fungistatic or fungicidal. In some examples, fungicide can be a protectant, or agents that are effective predominantly on the seed surface, providing protection against seed surface-borne pathogens and providing some level of control of soil-borne pathogens. Non-limiting examples of protectant fungicides include captan, maneb, thiram, or fludioxonil.

In some examples, fungicide can be a systemic fungicide, which can be absorbed into the emerging seedling and inhibit or kill the fungus inside host plant tissues. Systemic fungicides used for seed treatment include, but are not limited to the following: azoxystrobin, carboxin, mefenoxam, metalaxyl, thiabendazole, trifloxystrobin, and various triazole fungicides, including difenoconazole, ipconazole, tebuconazole, and triticonazole. Mefenoxam and metalaxyl are primarily used to target the water mold fungi Pythium and Phytophthora. Some fungicides are preferred over others, depending on the plant species, either because of subtle differences in sensitivity of the pathogenic fungal species, or because of the differences in the fungicide distribution or sensitivity of the plants. In some examples, fungicide can be a biological control agent, such as a bacterium or fungus. Such organisms may be parasitic to the pathogenic fungi, or secrete toxins or other substances that can kill or otherwise prevent the growth of fungi. Any type of fungicide, particularly ones that are commonly used on plants, can be used as a control agent in a seed composition.

In some examples, the seed coating composition comprises a control agent that has antibacterial properties. In one embodiment, the control agent with antibacterial properties is selected from the compounds described herein elsewhere. In another embodiment, the compound is Streptomycin, oxytetracycline, oxolinic acid, or gentamicin. Other examples of antibacterial compounds which can be used as part of a seed coating composition include those based on dichlorophene and benzylalcohol hemi formal (Proxel® from ICI or Acticide® RS from Thor Chemie and Kathon® MK 25 from Rohm & Haas) and isothiazolinone derivatives such as alkylisothiazolinones and benzisothiazolinones (Acticide® MBS from Thor Chemie).

In some examples, growth regulator is selected from the group consisting of: Abscisic acid, amidochlor, ancymidol, 6-benzylaminopurine, brassinolide, butralin, chlormequat (chlormequat chloride), choline chloride, cyclanilide, daminozide, dikegulac, dimethipin, 2,6-dimethylpuridine, ethephon, flumetralin, flurprimidol, fluthiacet, forchlorfenuron, gibberellic acid, inabenfide, indole-3-acetic acid, maleic hydrazide, mefluidide, mepiquat (mepiquat chloride), naphthaleneacetic acid, N-6-benzyladenine, paclobutrazol, prohexadione phosphorotrithioate, 2,3,5-tri-iodobenzoic acid, trinexapac-ethyl and uniconazole. Additional non-limiting examples of growth regulators include brassinosteroids, cytokinines (e.g., kinetin and zeatin), auxins (e.g., indolylacetic acid and indolylacetyl aspartate), flavonoids and isoflavanoids (e.g., formononetin and diosmetin), phytoaixins (e.g., glyceolline), and phytoalexin-inducing oligosaccharides (e.g., pectin, chitin, chitosan, polygalacuronic acid, and oligogalacturonic acid), and gibellerins. Such agents are ideally compatible with the agricultural seed or seedling onto which the formulation is applied (e.g., it should not be deleterious to the growth or health of the plant). Furthermore, the agent is ideally one which does not cause safety concerns for human, animal or industrial use (e.g., no safety issues, or the compound is sufficiently labile that the commodity plant product derived from the plant contains negligible amounts of the compound).

Some examples of nematode-antagonistic biocontrol agents include ARF18; 30 Arthrobotrys spp.; Chaetomium spp.; Cylindrocarpon spp.; Exophilia spp.; Fusarium spp.; Gliocladium spp.; Hirsutella spp.; Lecanicillium spp.; Monacrosporium spp.; Myrothecium spp.; Neocosmospora spp.; Paecilomyces spp.; Pochonia spp.; Stagonospora spp.; vesicular-arbuscular mycorrhizal fungi, Burkholderia spp.; Pasteuria spp., Brevibacillus spp.; Pseudomonas spp.; and Rhizobacteria. Particularly preferred nematode-antagonistic biocontrol agents include ARF18, Arthrobotrys oligospora, Arthrobotrys dactyloides, Chaetomium globosum, Cylindrocarpon heteronema, Exophilia jeanselmei, Exophilia pisciphila, Fusarium aspergilus, Fusarium solani, Gliocladium catenulatum, Gliocladium roseum, Gliocladium vixens, Hirsutella rhossiliensis, Hirsutella minnesotensis, Lecanicillium lecanii, Monacrosporium drechsleri, Monacrosporium gephyropagum, Myrotehcium verrucaria, Neocosmospora vasinfecta, Paecilomyces lilacinus, Pochonia chlamydosporia, Stagonospora heteroderae, Stagonospora phaseoli, vesicular-arbuscular mycorrhizal fungi, Burkholderia cepacia, Pasteuria penetrans, Pasteuria thornei, Pasteuria nishizawae, Pasteuria ramosa, Pastrueia usage, Brevibacillus laterosporus strain G4, Pseudomonas fluorescens and Rhizobacteria.

Some examples of nutrients can be selected from the group consisting of a nitrogen fertilizer including, but not limited to Urea, Ammonium nitrate, Ammonium sulfate, Non-pressure nitrogen solutions, Aqua ammonia, Anhydrous ammonia, Ammonium thiosulfate, Sulfur-coated urea, Urea-formaldehydes, IBDU, Polymer-coated urea, Calcium nitrate, Ureaform, and Methylene urea, phosphorous fertilizers such as Diammonium phosphate, Monoammonium phosphate, Ammonium polyphosphate, Concentrated superphosphate and Triple superphosphate, and potassium fertilizers such as Potassium chloride, Potassium sulfate, Potassium-magnesium sulfate, Potassium nitrate. Such compositions can exist as free salts or ions within the seed coat composition. Alternatively, nutrients/fertilizers can be complexed or chelated to provide sustained release over time.

Some examples of rodenticides may include selected from the group of substances consisting of 2-isovalerylindan-1,3-dione, 4-(quinoxalin-2-ylamino) benzenesulfonamide, alpha-chlorohydrin, aluminum phosphide, antu, arsenous oxide, barium carbonate, bisthiosemi, brodifacoum, bromadiolone, bromethalin, calcium cyanide, chloralose, chlorophacinone, cholecalciferol, coumachlor, coumafuryl, coumatetralyl, crimidine, difenacoum, difethialone, diphacinone, ergocalciferol, flocoumafen, fluoroacetamide, flupropadine, flupropadine hydrochloride, hydrogen cyanide, iodomethane, lindane, magnesium phosphide, methyl bromide, norbormide, phosacetim, phosphine, phosphorus, pindone, potassium arsenite, pyrinuron, scilliroside, sodium arsenite, sodium cyanide, sodium fluoroacetate, strychnine, thallium sulfate, warfarin and zinc phosphide.

In the liquid form, for example, solutions or suspensions, bacterial populations can be mixed or suspended in water or in aqueous solutions. Suitable liquid diluents or carriers include water, aqueous solutions, petroleum distillates, or other liquid carriers.

Solid compositions can be prepared by dispersing the bacterial populations in and on an appropriately divided solid carrier, such as peat, wheat, bran, vermiculite, clay, talc, bentonite, diatomaceous earth, fuller's earth, pasteurized soil, and the like. When such formulations are used as wettable powders, biologically compatible dispersing agents such as non-ionic, anionic, amphoteric, or cationic dispersing and emulsifying agents can be used.

The solid carriers used upon formulation include, for example, mineral carriers such as kaolin clay, pyrophyllite, bentonite, montmorillonite, diatomaceous earth, acid white soil, vermiculite, and pearlite, and inorganic salts such as ammonium sulfate, ammonium phosphate, ammonium nitrate, urea, ammonium chloride, and calcium carbonate. Also, organic fine powders such as wheat flour, wheat bran, and rice bran may be used. The liquid carriers include vegetable oils such as soybean oil and cottonseed oil, glycerol, ethylene glycol, polyethylene glycol, propylene glycol, polypropylene glycol, etc.

### Application of Bacterial Populations on Crops

The composition of the bacteria or bacterial population described herein can be applied in furrow, in talc, or as seed treatment. The composition can be applied to a seed package in bulk, mini bulk, in a bag, or in talc.

The planter can plant the treated seed and grows the crop according to conventional ways, twin row, or ways that do not require tilling. The seeds can be distributed using a control hopper or an individual hopper. Seeds can also be distributed using pressurized air or manually. Seed placement can be performed using variable rate technologies. Additionally, application of the bacteria or bacterial population described herein may be applied using variable rate technologies. In some examples, the bacteria can be applied to seeds of corn, soybean, canola, sorghum, potato, rice, vegetables, cereals, pseudocereals, and oilseeds. Examples of cereals may include barley, fonio, oats, palmer's grass, rye, pearl millet, sorghum, spelt, teff, triticale, and wheat. Examples of pseudocereals may include breadnut, buckwheat, cattail, chia, flax, grain amaranth, hanza, quinoa, and sesame. In some examples, seeds can be genetically modified organisms (GMO), non-GMO, organic or conventional.

Additives such as micro-fertilizer, PGR, herbicide, insecticide, and fungicide can be used additionally to treat the crops. Examples of additives include crop protectants such as insecticides, nematicides, fungicide, enhancement agents such as colorants, polymers, pelleting, priming, and disinfectants, and other agents such as inoculant, PGR, softener, and micronutrients. PGRs can be natural or synthetic plant hormones that affect root growth, flowering, or stem elongation. PGRs can include auxins, gibberellins, cytokinins, ethylene, and abscisic acid (ABA).

The composition can be applied in furrow in combination with liquid fertilizer. In some examples, the liquid fertilizer may be held in tanks. NPK fertilizers contain macronutrients of sodium, phosphorous, and potassium.

The composition, by providing fixed nitrogen, may improve plant traits, such as promoting plant growth, maintaining high chlorophyll content in leaves, increasing fruit or seed numbers, and increasing fruit or seed unit weight. Methods of the present disclosure may be employed to introduce or improve one or more of a variety of desirable traits. Examples of traits that may introduced or improved include: root biomass, root length, height, shoot length, leaf number, water use efficiency, overall biomass, yield, fruit size, grain size, photosynthesis rate, tolerance to drought, heat tolerance, salt tolerance, tolerance to low nitrogen stress, nitrogen use efficiency, resistance to nematode stress, resistance to a fungal pathogen, resistance to a bacterial pathogen, resistance to a viral pathogen, level of a metabolite, modulation in level of a metabolite, proteome expression. The desirable traits, including height, overall biomass, root and/or shoot biomass, seed germination, seedling survival, photosynthetic efficiency, transpiration rate, seed/fruit number or mass, plant grain or fruit yield, leaf chlorophyll content, photosynthetic rate, root length, or any combination thereof, can be used to measure growth, and compared with the growth rate of reference agricultural plants (e.g., plants without the introduced and/or improved traits) grown under identical conditions. In some examples, the desirable traits, including height, overall biomass, root and/or shoot biomass, seed germination, seedling survival, photosynthetic efficiency, transpiration rate, seed/fruit number or mass, plant grain or fruit yield, leaf chlorophyll content, photosynthetic rate, root length, or any combination thereof, can be used to measure growth, and compared with the growth rate of reference agricultural plants (e.g., plants without the introduced and/or improved traits) grown under similar conditions.

An agronomic trait to a host plant may include, but is not limited to, the following: altered oil content, altered protein content, altered seed carbohydrate composition, altered seed oil composition, and altered seed protein composition, chemical tolerance, cold tolerance, delayed senescence, disease resistance, drought tolerance, ear weight, growth improvement, health enhancement, heat tolerance, herbicide tolerance, herbivore resistance improved nitrogen fixation, improved nitrogen utilization, improved root architecture, improved water use efficiency, increased biomass, increased root length, increased seed weight, increased shoot length, increased yield, increased yield under water-limited conditions, kernel mass, kernel moisture content, metal tolerance, number of ears, number of kernels per ear, number of pods, nutrition enhancement, pathogen resistance, pest resistance, photosynthetic capability improvement, salinity tolerance, stay-green, vigor improvement, increased dry weight of mature seeds, increased fresh weight of mature seeds, increased number of mature seeds per plant, increased chlorophyll content, increased number of pods per plant, increased length of pods per plant, reduced number of wilted leaves per plant, reduced number of severely wilted leaves per plant, and increased number of non-wilted leaves per plant, a detectable modulation in the level of a metabolite, a detectable modulation in the level of a transcript, and a detectable modulation in the proteome, compared to an isoline plant grown from a seed without said seed treatment formulation.

In some cases, plants are inoculated with bacteria or bacterial populations that are isolated from the same species of plant as the plant element of the inoculated plant. For example, a bacteria or bacterial population that is normally found in one variety of Zea mays (corn) is associated with a plant element of a plant of another variety of Zea mays that in its natural state lacks said bacteria and bacterial populations. In one embodiment, the bacteria and bacterial populations is derived from a plant of a related species of plant as the plant element of the inoculated plant. For example, an bacteria and bacterial populations that is normally found in Zea diploperennis Iltis et al., (diploperennial teosinte) is applied to a Zea mays (corn), or vice versa. In some cases, plants are inoculated with bacteria and bacterial populations that are heterologous to the plant element of the inoculated plant. In one embodiment, the bacteria and bacterial populations is derived from a plant of another species. For example, a bacteria and bacterial populations that is normally found in dicots is applied to a monocot plant (e.g., inoculating corn with a soybean-derived bacteria and bacterial populations), or vice versa. In other cases, the bacteria and bacterial populations to be inoculated onto a plant is derived from a related species of the plant that is being inoculated. In one embodiment, the bacteria and bacterial populations is derived from a related taxon, for example, from a related species. The plant of another species can be an agricultural plant. In another embodiment, the bacteria and bacterial populations is part of a designed composition inoculated into any host plant element.

In some examples, the bacteria or bacterial population is exogenous wherein the bacteria and bacterial population is isolated from a different plant than the inoculated plant. For example, in one embodiment, the bacteria or bacterial population can be isolated from a different plant of the same species as the inoculated plant. In some cases, the bacteria or bacterial population can be isolated from a species related to the inoculated plant.

In some examples, the bacteria and bacterial populations described herein are capable of moving from one tissue type to another. For example, the present disclosure's detection and isolation of bacteria and bacterial populations within the mature tissues of plants after coating on the exterior of a seed demonstrates their ability to move from seed exterior into the vegetative tissues of a maturing plant. Therefore, in one embodiment, the population of bacteria and bacterial populations is capable of moving from the seed exterior into the vegetative tissues of a plant. In one embodiment, the bacteria and bacterial populations that is coated onto the seed of a plant is capable, upon germination of the seed into a vegetative state, of localizing to a different tissue of the plant. For example, bacteria and bacterial populations can be capable of localizing to any one of the tissues in the plant, including: the root, adventitious root, seminal 5 root, root hair, shoot, leaf, flower, bud, tassel, meristem, pollen, pistil, ovaries, stamen, fruit, stolon, rhizome, nodule, tuber, trichome, guard cells, hydathode, petal, sepal, glume, rachis, vascular cambium, phloem, and xylem. In one embodiment, the bacteria and bacterial populations is capable of localizing to the root and/or the root hair of the plant. In another embodiment, the bacteria and bacterial populations is capable of localizing to the photosynthetic tissues, for example, leaves and shoots of the plant. In other cases, the bacteria and bacterial populations is localized to the vascular tissues of the plant, for example, in the xylem and phloem. In still another embodiment, the bacteria and bacterial populations is capable of localizing to the reproductive tissues (flower, pollen, pistil, ovaries, stamen, fruit) of the plant. In another embodiment, the bacteria and bacterial populations is capable of localizing to the root, shoots, leaves and reproductive tissues of the plant. In still another embodiment, the bacteria and bacterial populations colonizes a fruit or seed tissue of the plant. In still another embodiment, the bacteria and bacterial populations is able to colonize the plant such that it is present in the surface of the plant (i.e., its presence is detectably present on the plant exterior, or the episphere of the plant). In still other embodiments, the bacteria and bacterial populations is capable of localizing to substantially all, or all, tissues of the plant. In certain embodiments, the bacteria and bacterial populations is not localized to the root of a plant. In other cases, the bacteria and bacterial populations is not localized to the photosynthetic tissues of the plant.

The effectiveness of the compositions can also be assessed by measuring the relative maturity of the crop or the crop heating unit (CHU). For example, the bacterial population can be applied to corn, and corn growth can be assessed according to the relative maturity of the corn kernel or the time at which the corn kernel is at maximum weight. The crop heating unit (CHU) can also be used to predict the maturation of the corn crop. The CHU determines the amount of heat accumulation by measuring the daily maximum temperatures on crop growth.

In examples, bacterial may localize to any one of the tissues in the plant, including: the root, adventitious root, seminal root, root hair, shoot, leaf, flower, bud tassel, meristem, pollen, pistil, ovaries, stamen, fruit, stolon, rhizome, nodule, tuber, trichome, guard cells, hydathode, petal, sepal, glume, rachis, vascular cambium, phloem, and xylem. In another embodiment, the bacteria or bacterial population is capable of localizing to the photosynthetic tissues, for example, leaves and shoots of the plant. In other cases, the bacteria and bacterial populations is localized to the vascular tissues of the plant, for example, in the xylem and phloem. In another embodiment, the bacteria or bacterial population is capable of localizing to reproductive tissues (flower, pollen, pistil, ovaries, stamen, or fruit) of the plant. In another embodiment, the bacteria and bacterial populations is capable of localizing to the root, shoots, leaves and reproductive tissues of the plant. In another embodiment, the bacteria or bacterial population colonizes a fruit or seed tissue of the plant. In still another embodiment, the bacteria or bacterial population is able to colonize the plant such that it is present in the surface of the plant. In another embodiment, the bacteria or bacterial population is capable of localizing to substantially all, or all, tissues of the plant. In certain embodiments, the bacteria or bacterial population is not localized to the root of a plant. In other cases, the bacteria and bacterial populations is not localized to the photosynthetic tissues of the plant.

The effectiveness of the bacterial compositions applied to crops can be assessed by measuring various features of crop growth including, but not limited to, planting rate, seeding vigor, root strength, drought tolerance, plant height, dry down, and test weight.

### Plant Species

The methods and bacteria described herein are suitable for any of a variety of plants, such as plants in the genera Hordeum, Oryza, Zea, and Triticeae. Other non-limiting examples of suitable plants include mosses, lichens, and algae. In some cases, the plants have economic, social and/or environmental value, such as food crops, fiber crops, oil crops, plants in the forestry or pulp and paper industries, feedstock for biofuel production and/or ornamental plants. In some examples, plants may be used to produce economically valuable products such as a grain, a flour, a starch, a syrup, a meal, an oil, a film, a packaging, a nutraceutical product, a pulp, an animal feed, a fish fodder, a bulk material for industrial chemicals, a cereal product, a processed human-food product, a sugar, an alcohol, and/or a protein. Non-limiting examples of crop plants include maize, rice, wheat, barley, sorghum, millet, oats, rye triticale, buckwheat, sweet corn, sugar cane, onions, tomatoes, strawberries, and asparagus. In some embodiments, the methods and bacteria described herein are suitable for any of a variety of transgenic plants, non-transgenic plants, and hybrid plants thereof.

In some examples, plants that may be obtained or improved using the methods and composition disclosed herein may include plants that are important or interesting for agriculture, horticulture, biomass for the production of biofuel molecules and other chemicals, and/or forestry. Some examples of these plants may include pineapple, banana, coconut, lily, grasspeas and grass; and dicotyledonous plants, such as, for example, peas, alfalfa, tomatillo, melon, chickpea, chicory, clover, kale, lentil, soybean, tobacco, potato, sweet potato, radish, cabbage, rape, apple trees, grape, cotton, sunflower, thale cress, canola, citrus (including orange, mandarin, kumquat, lemon, lime, grapefruit, tangerine, tangelo, citron, and pomelo), pepper, bean, lettuce, Panicum virgatum (switch), Sorghum bicolor (sorghum, sudan), Miscanthus giganteus (miscanthus), Saccharum sp. (energycane), Populus balsamifera (poplar), Zea mays (corn), Glycine max (soybean), Brassica napus (canola), Triticum aestivum (wheat), Gossypium hirsutum (cotton), Oryza sativa (rice), Helianthus annuus (sunflower), Medicago sativa (alfalfa), Beta vulgaris (sugarbeet), Pennisetum glaucum (pearl millet), Panicum spp. Sorghum spp., Miscanthus spp., Saccharum spp., Erianthus spp., Populus spp., Secale cereale (rye), Salix spp. (willow), Eucalyptus spp. (eucalyptus), Triticosecale spp. (triticum-25 wheat X rye), Bamboo, Carthamus tinctorius (safflower), Jatropha curcas (Jatropha), Ricinus communis (castor), Elaeis guineensis (oil palm), Phoenix dactylifera (date palm), Archontophoenix cunninghamiana (king palm), Syagrus romanzoffiana (queen palm), Linum usitatissimum (flax), Brassica juncea, Manihot esculenta (cassaya), Lycopersicon esculentum (tomato), Lactuca saliva (lettuce), Musa paradisiaca (banana), Solanum tuberosum (potato), Brassica oleracea (broccoli, cauliflower, brussel sprouts), Camellia sinensis (tea), Fragaria ananassa (strawberry), Theobroma cacao (cocoa), Coffea arabica (coffee), Vitis vinifera (grape), Ananas comosus (pineapple), Capsicum annum (hot & sweet pepper), Allium cepa (onion), Cucumis melo (melon), Cucumis sativus (cucumber), Cucurbita maxima (squash), Cucurbita moschata (squash), Spinacea oleracea (spinach), Citrullus lanatus (watermelon), Abelmoschus esculentus (okra), Solanum melongena (eggplant), Papaver somniferum (opium poppy), Papaver orientale, Taxus baccata, Taxus brevifolia, Artemisia annua, Cannabis saliva, Camptotheca acuminate, Catharanthus roseus, Vinca rosea, Cinchona officinalis, Coichicum autumnale, Veratrum californica, Digitalis lanata, Digitalis purpurea, Dioscorea 5 spp., Andrographis paniculata, Atropa belladonna, Datura stomonium, Berberis spp., Cephalotaxus spp., Ephedra sinica, Ephedra spp., Erythroxylum coca, Galanthus wornorii, Scopolia spp., Lycopodium serratum (Huperzia serrata), Lycopodium spp., Rauwolfia serpentina, Rauwolfia spp., Sanguinaria canadensis, Hyoscyamus spp., Calendula officinalis, Chrysanthemum parthenium, Coleus forskohlii, Tanacetum parthenium, Parthenium argentatum (guayule), Hevea spp. (rubber), Mentha spicata (mint), Mentha piperita (mint), Bixa orellana, Alstroemeria spp., Rosa spp. (rose), Dianthus caryophyllus (carnation), Petunia spp. (petunia), Poinsettia pulcherrima (poinsettia), Nicotiana tabacum (tobacco), Lupinus albus (lupin), Uniola paniculata (oats), Hordeum vulgare (barley), and Lolium spp. (rye).

In some examples, a monocotyledonous plant may be used. Monocotyledonous plants belong to the orders of the Alismatales, Arales, Arecales, Bromeliales, Commelinales, Cyclanthales, Cyperales, Eriocaulales, Hydrocharitales, Juncales, Lilliales, Najadales, Orchidales, Pandanales, Poales, Restionales, Triuridales, Typhales, and Zingiberales. Plants belonging to the class of the Gymnospermae are Cycadales, Ginkgoales, Gnetales, and Pinales. In some examples, the monocotyledonous plant can be selected from the group consisting of a maize, rice, wheat, barley, and sugarcane.

In some examples, a dicotyledonous plant may be used, including those belonging to the orders of the Aristochiales, Asterales, Batales, Campanulales, Capparales, Caryophyllales, Casuarinales, Celastrales, Cornales, Diapensales, Dilleniales, Dipsacales, Ebenales, Ericales, Eucomiales, Euphorbiales, Fabales, Fagales, Gentianales, Geraniales, Haloragales, Hamamelidales, Middles, Juglandales, Lamiales, Laurales, Lecythidales, Leitneriales, Magniolales, Malvales, Myricales, Myrtales, Nymphaeales, Papeverales, Piperales, Plantaginales, Plumb aginales, Podostemales, Polemoniales, Polygalales, Polygonales, Primulales, Proteales, Rafflesiales, Ranunculales, Rhamnales, Rosales, Rubiales, Salicales, Santales, Sapindales, Sarraceniaceae, Scrophulariales, Theales, Trochodendrales, Umbellales, Urticales, and Violates. In some examples, the dicotyledonous plant can be selected from the group consisting of cotton, soybean, pepper, and tomato.

In some cases, the plant to be improved is not readily amenable to experimental conditions. For example, a crop plant may take too long to grow enough to practically assess an improved trait serially over multiple iterations. Accordingly, a first plant from which bacteria are initially isolated, and/or the plurality of plants to which genetically manipulated bacteria are applied may be a model plant, such as a plant more amenable to evaluation under desired conditions. Non-limiting examples of model plants include Setaria, Brachypodium, and Arabidopsis. Ability of bacteria isolated according to a method of the disclosure using a model plant may then be applied to a plant of another type (e.g. a crop plant) to confirm conferral of the improved trait.

Traits that may be improved by the methods disclosed herein include any observable characteristic of the plant, including, for example, growth rate, height, weight, color, taste, smell, changes in the production of one or more compounds by the plant (including for example, metabolites, proteins, drugs, carbohydrates, oils, and any other compounds). Selecting plants based on genotypic information is also envisaged (for example, including the pattern of plant gene expression in response to the bacteria, or identifying the presence of genetic markers, such as those associated with increased nitrogen fixation). Plants may also be selected based on the absence, suppression or inhibition of a certain feature or trait (such as an undesirable feature or trait) as opposed to the presence of a certain feature or trait (such as a desirable feature or trait).

### Concentrations and Rates of Application of Agricultural Compositions

As aforementioned, the agricultural compositions of the present disclosure, which comprise a generated and/or identified microbe, can be applied to plants in a multitude of ways. In two particular aspects, the disclosure contemplates an in-furrow treatment or a seed treatment

For seed treatment embodiments, the microbes of the disclosure can be present on the seed in a variety of concentrations. For example, the microbes can be found in a seed treatment at a cfu concentration, per seed of: 1×10¹, 1×10², 1×10³, 1×10⁴, 1×10⁵, 1×10⁶, 1×10⁷, 1×10⁸, 1×10⁹, 1×10¹⁰, or more. In particular aspects, the seed treatment compositions comprise about 1×10⁴ to about 1×10⁸ cfu per seed. In other particular aspects, the seed treatment compositions comprise about 1×10⁵ to about 1×10⁷ cfu per seed. In other aspects, the seed treatment compositions comprise about 1×10⁶ cfu per seed. In general, the one or more bacteria present in an agricultural or microbial composition provided herein can have an average colonization ability per unit of plant root tissue of at least about 1.0×10⁴ bacterial cells per gram of fresh weight of plant root tissue and can produce fixed N of at least about 1×10⁻¹⁷ mmol N per bacterial cell per hour.

In the United States, about 10% of corn acreage is planted at a seed density of above about 36,000 seeds per acre; ⅓ of the corn acreage is planted at a seed density of between about 33,000 to 36,000 seeds per acre; ⅓ of the corn acreage is planted at a seed density of between about 30,000 to 33,000 seeds per acre, and the remainder of the acreage is variable. See, “Corn Seeding Rate Considerations,” written by Steve Butzen, available at: www.pioneer.com/home/site/us/agronomy/library/corn-seeding-rate-considerations/

Table 8 below utilizes various cfu concentrations per seed in a contemplated seed treatment embodiment (rows across) and various seed acreage planting densities (1^{st} column: 15K-41K) to calculate the total amount of cfu per acre, which would be utilized in various agricultural scenarios (i.e. seed treatment concentration per seed×seed density planted per acre). Thus, if one were to utilize a seed treatment with 1×10⁶ cfu per seed and plant 30,000 seeds per acre, then the total cfu content per acre would be 3×10¹⁰ (i.e. 30K*1×10⁶)

TABLE 8
Total CFU Per Acre Calculation for Seed Treatment Embodiments

| Corn | | | | | | | | |
|---|---|---|---|---|---|---|---|---|
| Population | | | | | | | | |
| (i.e. seeds | | | | | | | | |
| per acre) | 1.00E+02 | 1.00E+03 | 1.00E+04 | 1.00E+05 | 1.00E+06 | 1.00E+07 | 1.00E+08 | 1.00E+09 |
| 15,000 | 1.50E+06 | 1.50E+07 | 1.50E+08 | 1.50E+09 | 1.50E+10 | 1.50E+11 | 1.50E+12 | 1.50E+13 |
| 16,000 | 1.60E+06 | 1.60E+07 | 1.60E+08 | 1.60E+09 | 1.60E+10 | 1.60E+11 | 1.60E+12 | 1.60E+13 |
| 17,000 | 1.70E+06 | 1.70E+07 | 1.70E+08 | 1.70E+09 | 1.70E+10 | 1.70E+11 | 1.70E+12 | 1.70E+13 |
| 18,000 | 1.80E+06 | 1.80E+07 | 1.80E+08 | 1.80E+09 | 1.80E+10 | 1.80E+11 | 1.80E+12 | 1.80E+13 |
| 19,000 | 1.90E+06 | 1.90E+07 | 1.90E+08 | 1.90E+09 | 1.90E+10 | 1.90E+11 | 1.90E+12 | 1.90E+13 |
| 20,000 | 2.00E+06 | 2.00E+07 | 2.00E+08 | 2.00E+09 | 2.00E+10 | 2.00E+11 | 2.00E+12 | 2.00E+13 |
| 21,000 | 2.10E+06 | 2.10E+07 | 2.10E+08 | 2.10E+09 | 2.10E+10 | 2.10E+11 | 2.10E+12 | 2.10E+13 |
| 22,000 | 2.20E+06 | 2.20E+07 | 2.20E+08 | 2.20E+09 | 2.20E+10 | 2.20E+11 | 2.20E+12 | 2.20E+13 |
| 23,000 | 2.30E+06 | 2.30E+07 | 2.30E+08 | 2.30E+09 | 2.30E+10 | 2.30E+11 | 2.30E+12 | 2.30E+13 |
| 24,000 | 2.40E+06 | 2.40E+07 | 2.40E+08 | 2.40E+09 | 2.40E+10 | 2.40E+11 | 2.40E+12 | 2.40E+13 |
| 25,000 | 2.50E+06 | 2.50E+07 | 2.50E+08 | 2.50E+09 | 2.50E+10 | 2.50E+11 | 2.50E+12 | 2.50E+13 |
| 26,000 | 2.60E+06 | 2.60E+07 | 2.60E+08 | 2.60E+09 | 2.60E+10 | 2.60E+11 | 2.60E+12 | 2.60E+13 |
| 27,000 | 2.70E+06 | 2.70E+07 | 2.70E+08 | 2.70E+09 | 2.70E+10 | 2.70E+11 | 2.70E+12 | 2.70E+13 |
| 28,000 | 2.80E+06 | 2.80E+07 | 2.80E+08 | 2.80E+09 | 2.80E+10 | 2.80E+11 | 2.80E+12 | 2.80E+13 |
| 29,000 | 2.90E+06 | 2.90E+07 | 2.90E+08 | 2.90E+09 | 2.90E+10 | 2.90E+11 | 2.90E+12 | 2.90E+13 |
| 30,000 | 3.00E+06 | 3.00E+07 | 3.00E+08 | 3.00E+09 | 3.00E+10 | 3.00E+11 | 3.00E+12 | 3.00E+13 |
| 31,000 | 3.10E+06 | 3.10E+07 | 3.10E+08 | 3.10E+09 | 3.10E+10 | 3.10E+11 | 3.10E+12 | 3.10E+13 |
| 32,000 | 3.20E+06 | 3.20E+07 | 3.20E+08 | 3.20E+09 | 3.20E+10 | 3.20E+11 | 3.20E+12 | 3.20E+13 |
| 33,000 | 3.30E+06 | 3.30E+07 | 3.30E+08 | 3.30E+09 | 3.30E+10 | 3.30E+11 | 3.30E+12 | 3.30E+13 |
| 34,000 | 3.40E+06 | 3.40E+07 | 3.40E+08 | 3.40E+09 | 3.40E+10 | 3.40E+11 | 3.40E+12 | 3.40E+13 |
| 35,000 | 3.50E+06 | 3.50E+07 | 3.50E+08 | 3.50E+09 | 3.50E+10 | 3.50E+11 | 3.50E+12 | 3.50E+13 |
| 36,000 | 3.60E+06 | 3.60E+07 | 3.60E+08 | 3.60E+09 | 3.60E+10 | 3.60E+11 | 3.60E+12 | 3.60E+13 |
| 37,000 | 3.70E+06 | 3.70E+07 | 3.70E+08 | 3.70E+09 | 3.70E+10 | 3.70E+11 | 3.70E+12 | 3.70E+13 |
| 38,000 | 3.80E+06 | 3.80E+07 | 3.80E+08 | 3.80E+09 | 3.80E+10 | 3.80E+11 | 3.80E+12 | 3.80E+13 |
| 39,000 | 3.90E+06 | 3.90E+07 | 3.90E+08 | 3.90E+09 | 3.90E+10 | 3.90E+11 | 3.90E+12 | 3.90E+13 |
| 40,000 | 4.00E+06 | 4.00E+07 | 4.00E+08 | 4.00E+09 | 4.00E+10 | 4.00E+11 | 4.00E+12 | 4.00E+13 |
| 41,000 | 4.10E+06 | 4.10E+07 | 4.10E+08 | 4.10E+09 | 4.10E+10 | 4.10E+11 | 4.10E+12 | 4.10E+13 |

For in-furrow embodiments, the microbes of the disclosure can be applied at a cfu concentration per acre of: 1×10⁶, 3.20×10¹⁰, 1.60×10¹¹, 3.20×10¹¹, 8.0×10¹¹, 1.6×10¹², 3.20×10¹², or more. Therefore, in aspects, the liquid in-furrow compositions can be applied at a concentration of between about 1×10⁶ to about 3×10¹² cfu per acre.

In some aspects, the in-furrow compositions are contained in a liquid formulation. In the liquid in-furrow embodiments, the microbes can be present at a cfu concentration per milliliter of: 1×10¹, 1×10², 1×10³, 1×10⁴ 1×10⁵, 1×10⁶, 1×10⁷ 1×10⁸, 1×10⁹ 1×10¹⁰, 1×10¹¹, 1×10¹², 1×10¹³, or more. In certain aspects, the liquid in-furrow compositions comprise microbes at a concentration of about 1×10⁶ to about 1×10¹¹ cfu per milliliter. In other aspects, the liquid in-furrow compositions comprise microbes at a concentration of about 1×10⁷ to about 1×10¹⁰ cfu per milliliter. In other aspects, the liquid in-furrow compositions comprise microbes at a concentration of about 1×10⁸ to about 1×10⁹ cfu per milliliter. In other aspects, the liquid in-furrow compositions comprise microbes at a concentration of up to about 1×10¹³ cfu per milliliter.

### Biosensor Applications

In some aspects, provided herein are methods of utilizing a biosensor as provided herein to detect the presence of ammonium in a composition or medium. The method can comprise: (a) inoculating a composition with a biosensor, wherein the biosensor comprises a bacterium expressing a reporter protein, wherein the bacterium expresses the reporter molecule in the absence of ammonium; (b) exposing the inoculated composition to a stimulus sufficient to activate the reporter molecule; and (c) detecting reporter molecule output of the inoculated composition following (b), as compared to a control composition. In some cases, the method further comprises quantifying the amount of ammonium in the composition based upon the detected reporter molecule output.

In some aspects, provided herein are methods of utilizing a biosensor as provided herein to identify bacteria comprising at least one genetic variation introduced into at least one gene, or non-coding polynucleotide, of the nitrogen fixation or assimilation genetic regulatory network. The at least one genetic variation can be introduced via the random mutagenesis methods described herein.

In one embodiment, provided herein are methods for identification of bacterial mutants capable of fixing atmospheric nitrogen. The method for the identification of bacterial mutants capable of fixing atmospheric nitrogen can comprise: (a) exposing a population of bacteria to a mutagen in a microbial composition; (b) co-culturing the microbial composition with a biosensor, wherein the biosensor comprises a bacterium expressing a reporter protein, wherein the bacterium expresses the reporter molecule in the absence of ammonium; (c) exposing the co-cultured composition to a stimulus sufficient to activate the reporter molecule; and (d) identifying bacterial mutants capable of fixing atmospheric nitrogen as those that result in decreased or no reporter molecule output as compared to a control. The control can be a co-culture comprising the biosensor comprising bacterium and a bacterial strain known to be capable of fixing atmospheric nitrogen. The control can be a co-culture comprising the biosensor comprising bacterium and a bacterial strain known to be incapable of fixing atmospheric nitrogen. In some cases, the method further comprises isolating the bacterial mutants capable of fixing atmospheric nitrogen identified in (d).

The method for identification of bacterial mutants capable of fixing atmospheric nitrogen can comprise: (a) exposing a population of bacteria to a mutagen; (b) exposing the population of bacteria exposed to the mutagen in (a) to a diazotrophic growth inhibitor in a microbial composition; (c) co-culturing the microbial composition with a biosensor, wherein the biosensor comprises a bacterium expressing a reporter protein; (d) exposing the co-cultured composition to a stimulus sufficient to activate the reporter molecule, wherein the bacterium expresses the reporter molecule in the absence of ammonium; and (e) identifying bacterial mutants capable of fixing atmospheric nitrogen as those that result in decreased or no reporter molecule output as compared to a control. The control can be a co-culture comprising the biosensor comprising bacterium and a bacterial strain known to be capable of fixing atmospheric nitrogen. The control can be a co-culture comprising the biosensor comprising bacterium and a bacterial strain known to be incapable of fixing atmospheric nitrogen. In some cases, the method further comprises isolating the bacterial mutants capable of fixing atmospheric nitrogen identified in (e). The diazotrophic growth inhibitor can be ethylenediamine (EDA) or methylammonium.

Any of the methods provided herein that utilize a biosensor as provided herein can be high-throughput in nature.

In one embodiment, the method for identification of bacterial mutants capable of fixing atmospheric nitrogen is high-throughput and comprises: (a) exposing a population of bacteria to a mutagen; (b) transferring the population of bacteria in (a) into one or more samples comprising a medium; (c) co-culturing a biosensor within each of the one or more samples in (b), wherein the biosensor comprises a bacterium expressing a reporter protein, wherein the bacterium expresses the reporter molecule in the absence of ammonium; (d) exposing each of the one or more samples in (c) to a stimulus sufficient to activate the reporter molecule; and (e) identifying from the one or more samples in (d) one or more bacterial mutants capable of fixing atmospheric nitrogen as those that result in decreased output of the reporter molecule as compared to a control. The control can be a co-culture comprising the biosensor comprising bacterium and a bacterial strain known to be capable of fixing atmospheric nitrogen. The control can be a co-culture comprising the biosensor comprising bacterium and a bacterial strain known to be incapable of fixing atmospheric nitrogen. In some cases, the method further comprises isolating the bacterial mutants capable of fixing atmospheric nitrogen identified in (e).

Further to any of the above methods utilizing a biosensor as provided herein, the output of the reporter protein can be measured or detected following exposure of compositions or co-cultures comprising the biosensor comprising bacterium to the stimulus sufficient to activate the reporter protein as compared to a control composition. The control composition can comprise microbes that do not express the reporter protein. The control composition can comprise microbes that express a functionally deleted variant of the reporter protein.

In some aspects, provided herein are methods of utilizing a biosensor as provided herein to identify a genetic variation introduced into at least one gene, or non-coding polynucleotide, of the nitrogen fixation or assimilation genetic regulatory network of a mutant bacterial cell. The at least one genetic variation can be introduced via the random mutagenesis methods described herein. In some cases, further to any of the above embodiments for identifying bacterial mutants capable of fixing atmospheric nitrogen, said methods further comprise sequencing the genome(s) of the bacteria identified as capable of fixing atmospheric nitrogen following isolation of said identified bacteria; and identifying genes, pathways, and/or regulatory elements that contain mutations from the sequenced genomes. In some cases, the methods further comprise determining whether the genes, pathways, and/or regulatory elements contain mutations known to be associated with the fixation of atmospheric nitrogen.

In one embodiment, the biosensor for use in any of the methods employing said biosensor comprises a bacterium comprising: (i) a nucleic acid sequence encoding a reporter molecule, (ii) a promoter or fragment thereof selected from the Nif regulon operably linked to the nucleic acid sequence of (i), (iii) a nucleic acid sequence encoding an inhibitory nitrogen fixation regulatory protein, and (iv) a nucleic acid sequence encoding a positive nitrogen fixation regulatory protein, wherein the bacterium expresses the reporter molecule in the absence of ammonium. The promoter or fragment thereof for use in the biosensor can be selected from the nifHDK operon. In one embodiment, the promoter is a nifH promoter. The inhibitory nitrogen fixation regulatory protein can be NifL. The positive nitrogen fixation regulatory protein can be NifA.

The population of bacteria for use in any of the methods provided herein can be a population of any bacteria known in the art and/or provided herein. In one embodiment, the population of bacteria is selected from wild-type bacteria, transgenic mutant bacteria, non-intergeneric mutant bacteria and intergeneric mutant bacteria.

In one embodiment, the stimulus is selected from the group consisting of a chemical stimulus, a physical stimulus, a genetic stimulus and an energy stimulus. In one embodiment, the stimulus is light.

Further to any of the above embodiments, the reporter protein or molecule can be any reporter protein known in the art and/or provided herein. In some aspects, the reporter can be an enzyme that is adapted to produce a luminescent or fluorescent signal. In some aspects, the reporter protein can be an enzyme such as luciferase or alkaline phosphatase that yields a luminescent or fluorescent signal respectively. In some aspects, the reporter can also be a fluorescent protein or can include fluorescent, charged, or magnetic nanoparticles, nanodots, or quantum dots. In some aspects, the reporter can be a dye that has fluorescent, ultraviolet, or visible properties, wherein the fluorescent, ultraviolet, or visible properties undergo a detectable change in response to the activation of the biosensor circuit. In one embodiment, the reporter protein or molecule is a fluorescent protein, functional fragment, and/or fusions thereof. The fluorescent protein can be any fluorescent protein known in the art and/or provided herein such as, for example, GFP, RFP, YFP, CFP, or functional variants or fragments thereof. In one embodiment, the fluorescent protein is GFP. In one embodiment, the GFP is a superfolder GFP. In one embodiment, the reporter protein is a fluorescent protein and the stimulus is light such that exposing the co-cultures comprising biosensor comprising bacterium entails exposing said co-cultures to light excitation sufficient to fluoresce the fluorescent protein, functional fragment, and/or fusions thereof. The fluorescence output (e.g., intensity) can then be detected and/or measured with a flow cytometer, a plate reader, or fluorescence-activated droplet sorting.

The mutagen used in any of the above embodiments can be any mutagen known in the art and/or provided herein. The mutagen can be selected from mitomycin C (MMC), N-methyl-N-nitrosourea (MNU), nitrous acid (NA), diepoxybutane (DEB), 1,2,7,8-diepoxyoctane (DEO), ethyl methane sulfonate (EMS), methyl methane sulfonate (MMS), N-methyl-N′-nitro-N-nitrosoguanidine (MNNG), 4-nitroquinoline 1-oxide (4-NQO), 2-methyloxy-6-chloro-9(3-[ethyl-2-chloroethyl]-aminopropylamin)-acridinedihydrochloride (ICR-170), 2-amino purine (2AP), and hydroxylamine (HA). In one embodiment, the mutagen is EMS.

### High-Throughput Method

In some aspects, the high-throughput method is capable of performing the method on at least 100, at least 1,000, at least 10,000, at least 100,000, at least 1,000,000, or at least 10,000,000 bacteria. In some aspects, the high-throughput method is capable of being performed in less than 1 hour, less than 2 hours, less than 3 hours, less than 4 hours, less than 5 hours, less than 6 hours, less than 7 hours, less than 8 hours, less than 9 hours, less than 10 hours, less than 11 hours, less than 12 hours, less than 24 hours, less than 36 hours, less than 48 hours, less than 60 hours, or less than 72 hours. In some aspects, the high-throughput method is capable of being performed in less than 1 day, less than 2 days, less than 3 days, less than 4 days, less than 5 days, less than 6 days, or less than 7 days.

In some aspects, the high-throughput method is capable of performing the method on at least 100, at least 1,000, at least 10,000, at least 100,000, at least 1,000,000, or at least 10,000,000 bacteria in less than 1 hour, less than 2 hours, less than 3 hours, less than 4 hours, less than 5 hours, less than 6 hours, less than 7 hours, less than 8 hours, less than 9 hours, less than 10 hours, less than 11 hours, less than 12 hours, less than 24 hours, less than 36 hours, less than 48 hours, less than 60 hours, or less than 72 hours.

In some aspects, the methods of the disclosure include the use of minute volumes of media or another liquid, in which mutagenized microbes can be placed along with a biosensor. These small volumes of liquid amount to microdroplets that can be utilized with the biosensor strain to rapidly screen random microbial mutants and sort and collect the droplets via fluorescence-activated droplet sorting in embodiments where the reporter protein is a fluorescent protein.

In some aspects, the microdroplets comprise less than 2 μL, less than 3 μL, less than 4 μL, less than 5 μL, less than 6 μL, less than 7 μL, less than 8 μL, less than 9 μL, less than 10 μL, less than 11 μL, less than 12 μL, less than 13 μL, less than 14 μL, less than 15 μL, less than 16 μL, less than 17 μL, less than 18 μL, less than 19 μL, less than 20 μL, less than 21 μL, less than 22 μL, less than 23 μL, less than 24 μL, less than 25 μL, less than 30 μL, less than 35 μL, less than 40 μL, less than 45 μL, less than 50 μL, less than 55 μL, less than 60 μL, less than 65 μL, less than 70 μL, less than 75 μL, less than 80 μL, less than 85 μL, less than 90 μL, less than 95 μL, less than 100 μL, less than 125 μL, less than 150 μL, less than 175 μL, less than 200 μL, less than 225 μL, less than 250 μL, less than 275 μL, less than 300 μL, less than 325 μL, less than 350 μL, less than 375 μL, less than 400 μL, less than 425 μL, less than 450 μL, less than 475 μL, or less than 500 μL of liquid.

In some aspects, the microdroplets are less than 1 M, less than 2 M, less than 3 M, less than 4 μM, less than 5 μM, less than 6 μM, less than 7 μM, less than 8 μM, less than 9 μM, less than 10 μM, less than 15 μM, less than 20 μM, less than 25 μM, less than 30 μM, less than 35 μM, less than 40 μM, less than 45 μM, less than 50 μM, less than 55 μM, less than 60 μM, less than 65 M, less than 70 μM, less than 75 μM, less than 80 μM, less than 85 μM, less than 90 μM, less than 95 μM, less than 100 μM, less than 125 μM, less than 150 μM, less than 175 μM, less than 200 μM, less than 225 μM, less than 250 μM, less than 275 μM, less than 300 μM, less than 350 M, less than 400 μM, less than 450 μM, less than 500 μM, less than 550 μM, less than 600 μM, less than 650 μM, less than 700 μM, less than 750 μM, less than 800 μM, less than 850 μM, less than 900 μM, less than 950 μM, or less than 1000 μM in diameter.

The sorted droplets may be subjected to sequencing to identify genetic modifications that are responsible for improve ammonium excretion. The high-throughput methods provide for a method to target genetic engineering to enhance nitrogen fixing and ammonium excreting microbes.

Following cultivation of a mutant library under the nitrogen-fixing conditions, the level of excreted ammonium in the media is quantitatively measured or detected by analyzing GFP from the biosensor by flow cytometer, plate-reader, or other fluorescent output detection method. As the biosensor harbors an antibiotic selection marker, we can lyse the nitrogen fixing microbes with application of antibiotics to which the biosensor is resistant, further enabling the analysis of intracellular levels of nitrogen fixed by the mutated microbes using the biosensor.

In some aspects, the mutants are pooled together prior to the addition of the one or more biosensor microbes or prior to dilution of the medium. In some aspects, the pooled mutants comprise at least 2, at least 3, at least 4, at least 5, at least 6, at least 7, at least 8, at least 9, at least 10, at least 20, at least 25, at least 30, at least 35, at least 40, at least 45, at least 50, at least 55, at least 60, at least 65, at least 70, at least 75, at least 80, at least 85, at least 90, at least 95, or at least 100 different mutants.

### EXAMPLES

### Example 1. Biosensor Assay for Klebsiella, Kosakonia, and Metakosakonia

Day 1—Cultures for ammonium excretion assay are initiated by inoculating a single colony into a 96-deep well plate containing 1 ml of rich medium (e.g., Lennox LB or SOB). Incubate the cultures with shaking at 900 rpm at 30° C. overnight.

Day 2—The overnight cultures are diluted 100-fold in ARA minimal medium containing 17.1 mM ammonium acetate into 96-well plates. The dilution may be 2 μL of the overnight culture to 200 μL of fresh minimal medium. The ammonium acetate may be substituted with other nitrogen sources. The 96-well plates are incubated with shaking at 900 rpm at 30° C. for 24 hours. Cultures for biosensor strains are initiated by inoculating a single colony into 1 ml of LB medium supplemented with 15 g/ml gentamicin in a 15 ml culture tube for plasmid and incubated at 250 rpm at 37° C. overnight.

Day 3—The cultures for the ammonium excretion assay are diluted by adding 50 μL of the overnight culture into 450 μL of ammonium-free ARA minimal medium in a 96-deep well plate. The 96-well plate is transferred into an anaerobic Coy chamber and grown with shaking at 900 rpm at 30° C. for 24 hours (the incubation time can be adjusted). The biosensor cultures are diluted into ARA minimal medium containing 17.1 mM ammonium acetate supplemented with 15 g/mL gentamicin in a 15 mL culture tube by 100-fold and incubated at 250 rpm at 37° C. for ˜24 hours.

Day 4—Add 20 μL of 10×ARA salt medium supplemented with 150 g/ml gentamicin and 20 μL of the overnight grown biosensor into a new 96-well reaction plate. The cultures for the ammonium excretion assay are retrieved from the chamber and centrifuged at 4,000 g for 20 minutes to separate the cell pellets. Add 160 μL of the supernatant into the reaction plate to make 200 μL total. The dilution can be adjusted for supernatant based on the levels of ammonium excretion. If less than 160 μL of the supernatant is used, fill 1×ARA buffer to the remainder of the reaction to bring volume to 200 μL. The 96-well plate is transferred into an anaerobic Coy chamber and grown with shaking at 900 rpm at 30° C. for 15-18 hours.

Day 5—The plate is removed from the anaerobic chamber and incubated at room temperature for approximately one hour for GFP maturation before reading. The fluorescence signals can be analyzed either by a plate reader or a flow cytometer. Translation of the biosensor can be arrested by diluting cells in ice-cold phosphate-buffered saline (PBS) supplemented with 2 mg/ml kanamycin, but the fluorescent signals are stable over 12 hours at room temperature, and for several days at 4° C.

The inclusion of both positive and negative controls is necessary. This experiment utilizes Klebsiella variicola strains 137-2084 and 138-1590 as a positive and a negative control, respectively. Strains 137-2084 and 138-1590 are derived from the Klebsiella variicola 137. A standard curve for fixed nitrogen (e.g., ammonium or glutamine) can be generated by differing fixed nitrogen sources in the 4^{th} day reaction plate in which the supernatant containing excreted ammonium is replaced with 1×ammonium-free ARA buffer.

```
TABLE 1
10× ARA Salt Medium (per L)
200 g sucrose
    0.25 g MgSO₄•7H₂0
10 g NaCl
      1 g CaCl₂•2H₂O
  29 mg FeCl₃
          2.5 mg Na₂MoO₄•2H₂O
If adding nitrogen: 6 μL/mL of 22%
ammonium acetate (final 17.1 mM)
```

TABLE 2
ARA Buffer (per L)
25 g Na₂HPO₄
3 g KH₂PO₄
If adding nitrogen: 6 μL/mL of 22%
ammonium acetate (final 17.1 mM)

### Example 2: Biosensor Assay for Paraburkholderia and Herbaspirillum

Day 1—Cultures for ammonium excretion assay are initiated by inoculating a single colony into a 96-deep well plate containing 1 ml of rich medium (e.g., Lennox LB or SOB). Incubate the cultures with shaking at 900 rpm at 30° C. overnight.

Day 2—The overnight cultures are diluted 100-fold in JMV minimal medium containing 10 mM ammonium chloride into 96-well plates. The dilution may be 2 μL of the overnight culture to 200 μL of fresh minimal medium. The ammonium chloride may be substituted with other nitrogen sources. The 96-well plates are incubated with shaking at 900 rpm at 30° C. for 24 hours.

Day 3—The cultures for the ammonium excretion assay are diluted by adding 50 μL of the overnight culture into 450 μL of ammonium-free JMV minimal medium in a 96-deep well plate. The 96-well plate is transferred into a hypoxic Coy chamber and grown with shaking at 900 rpm at 30° C. for 48 hours (the incubation time can be adjusted) under hypoxic conditions (1˜3% oxygen). Cultures for biosensor strains are initiated by inoculating a single colony into 1 ml of LB medium supplemented with 15 g/ml gentamicin in a 15 ml culture tube for plasmid and incubated at 250 rpm at 37° C. overnight.

Day 4—The biosensor cultures are diluted into ARA minimal medium containing 17.1 mM ammonium acetate supplemented with 15 g/mL gentamicin in a 15 mL culture tube by 100-fold and incubated at 250 rpm at 37° C. for ˜24 hours.

Day 5—Add 20 μL of 10×ARA salt medium supplemented with 150 g/ml gentamicin and 20 μL of the overnight grown biosensor into a new 96-well reaction plate. The cultures for the ammonium excretion assay are retrieved from the chamber and centrifuged at 4,000 g for 20 minutes to separate the cell pellets. Add 160 μL of the supernatant into the reaction plate to make 200 μL total. The dilution can be adjusted for supernatant based on the levels of ammonium excretion. If less than 160 μL of the supernatant is used, fill 1×JMV buffer to the remainder of the reaction to bring volume to 200 μL. The 96-well plate is transferred into an anaerobic Coy chamber and grown with shaking at 900 rpm at 30° C. for 15-18 hours.

Day 6—The plate is removed from the anaerobic chamber and incubated at room temperature for approximately one hour for GFP maturation before reading. The fluorescence signals can be analyzed either by a plate reader or a flow cytometer. Translation of the biosensor can be arrested by diluting cells in ice-cold phosphate-buffered saline (PBS) supplemented with 2 mg/ml kanamycin, but the fluorescent signals are stable over 12 hours at room temperature, and for several days at 4° C.

The inclusion of both positive and negative controls is necessary. This experiment utilizes Paraburkholderia tropica wild-type and Paraburkholderia tropica 8-4752 as a negative control and a positive control, respectively. Strains 137-4752 is derived from the Paraburkholderia tropica 8. A standard curve for fixed nitrogen (e.g., ammonium or glutamine) can be generated by differing fixed nitrogen sources in the 5^{th} day reaction plate in which the supernatant containing excreted ammonium is replaced with 1×ammonium-free JMV buffer.

```
TABLE 3
10× JMV Salt Medium (per L)
50 g mannitol
18.02 g glucose
    2 g MgSO₄•7H₂0
1 g NaCl
    1 g CaCl₂•2H₂O
0.65 g Fe-EDTA
10 ml of Trace element
If adding nitrogen: 10 μL/mL of
1M ammonium chloride (final 10 mM)
```

TABLE 4
Trace element (per L)
0.04 g CuSO₄•5H₂O
0.12 g ZnSO₄•7H₂O
1.4 g H₃BO₃
1 g Na₂MoO₄•2H₂O
1.175 g MnSO₄•H₂O

TABLE 5
JMV Buffer (per L)
0.6 g K₂HPO₄
1.8 g KH₂PO₄
Adjust pH to 7.0 using KOH

### Example 3: Joint EDA-2AP Selection Assay

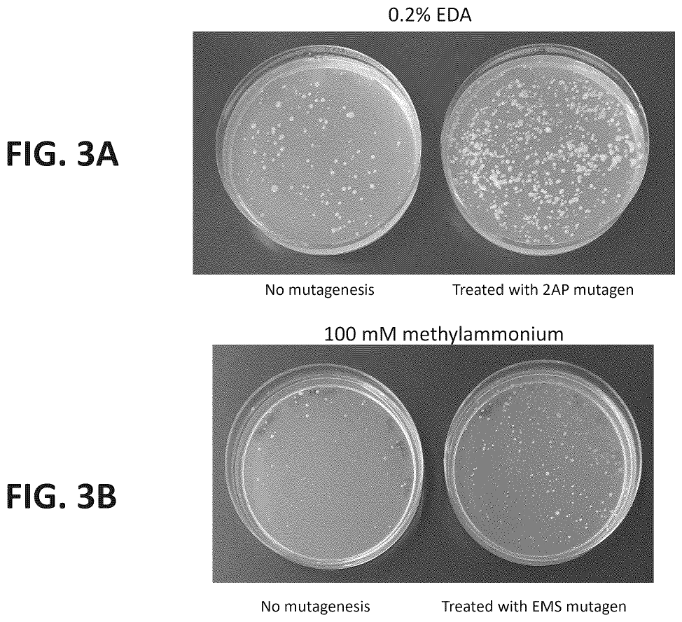

Single colonies of Klebsiella, Kosakonia, Metakosakonia, Paraburkholderia, or Herbaspirillum were inoculated into rich media and grown overnight at 30° C. Aliquots (5 L) of overnight cultures were diluted into 1 mL of rich media supplemented with between 0.5 and 1 mg/mL 2-aminopurine and grown for 24 h at 30° C. At the conclusion of the 24 h period the cultures were washed with PBS twice and plated on ammonium-free minimal medium supplemented with EDA from about 0.1% EDA to about 0.2% EDA or methylammonium from about 25 mM to about 200 mM methylammonium, and the plates were incubated at 30° C. under anaerobic conditions or hypoxic conditions for facultative anaerobes such as Klebsiella and obligate aerobes such as Paraburkholderia, respectively (FIG. 3A). The resulting individual colonies were indicated as resistant mutants to EDA or methylammonium and selected, grown in liquid media, and stored.

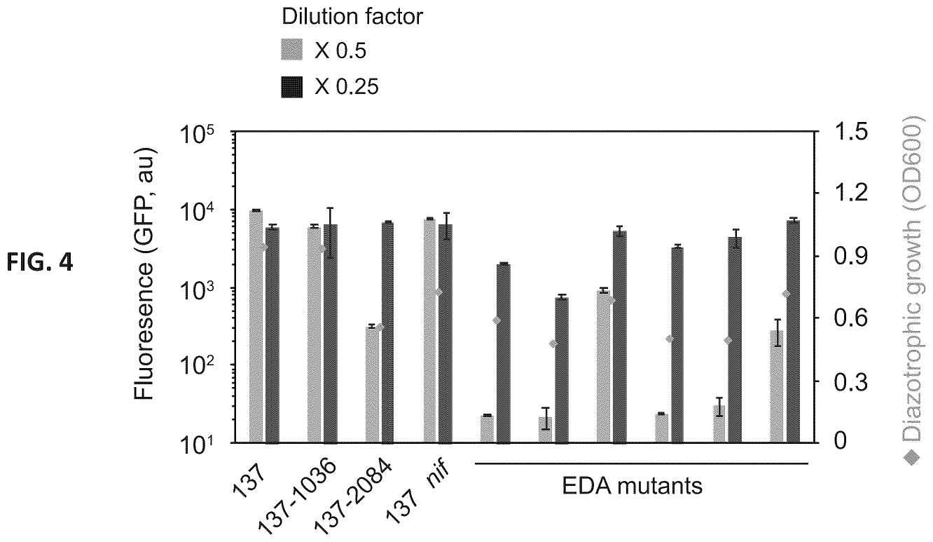

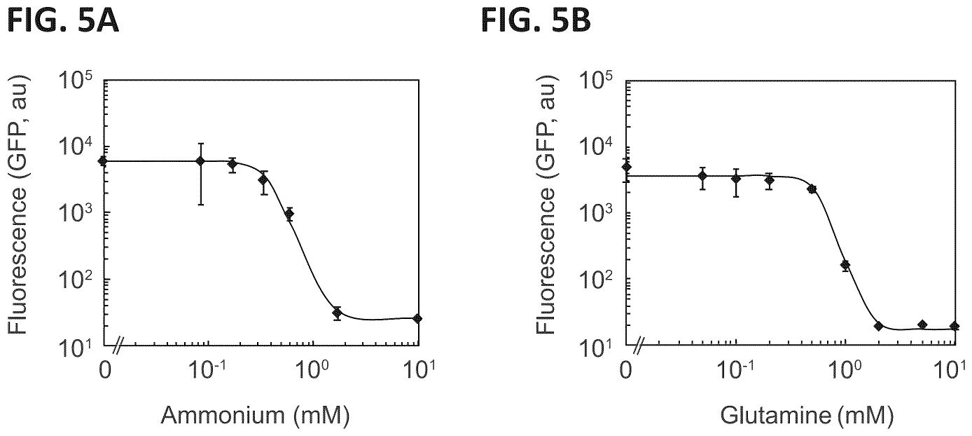

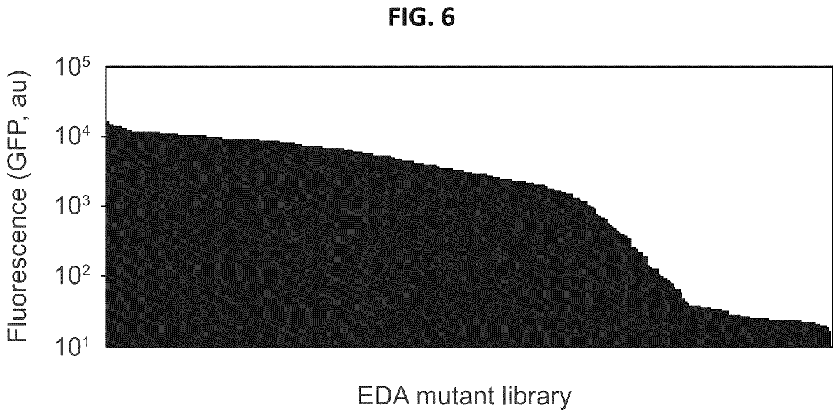

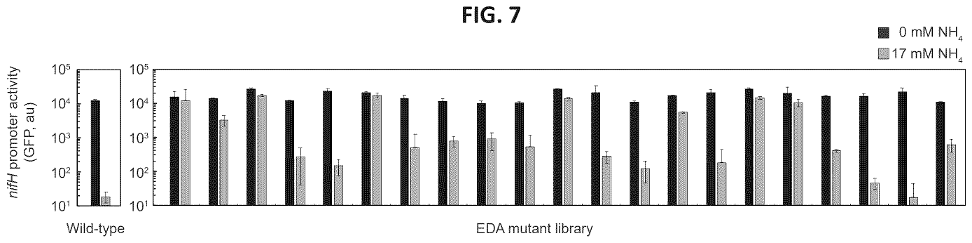

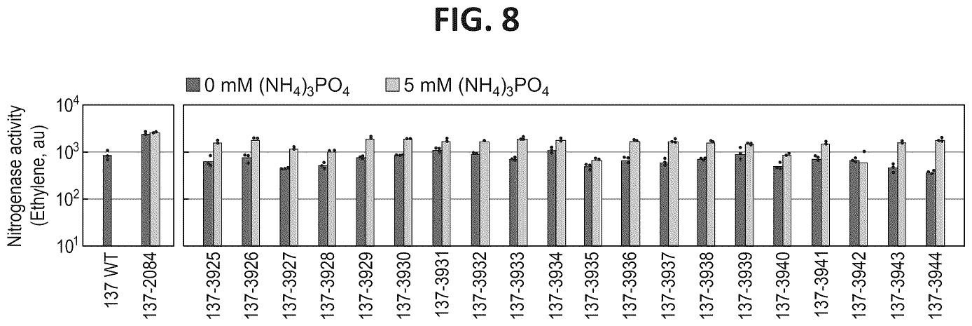

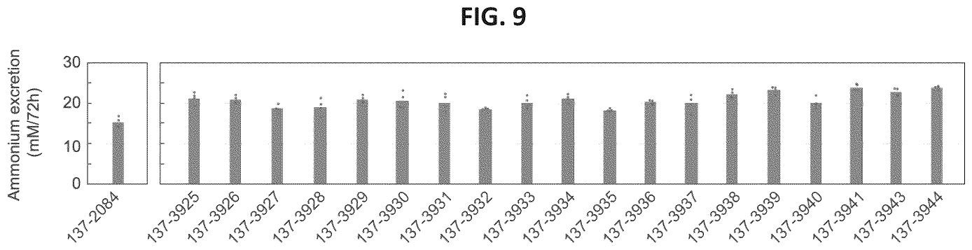

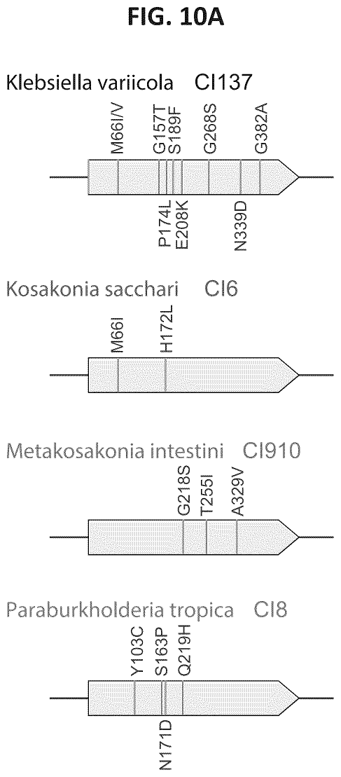

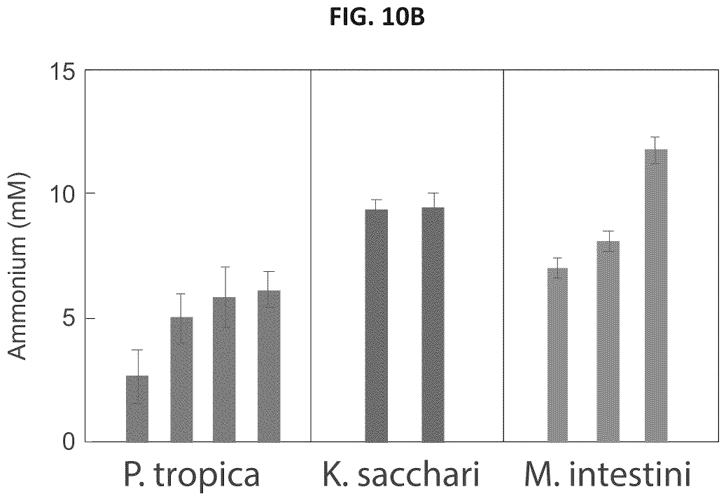

Combining the biosensor strain with the EDA- or methylammonium-resistant mutants for co-culture of the biosensor strain with the individual resistant mutants and various control strains allowed for a determination of the degree of ammonium excretion from nitrogen fixation activity of the resistant mutants as determined by the presence/absence, or the degree thereof, of GFP fluorescence from the biosensor strain. The biosensor can detect higher ammonium excretion from the 5 selected EDA-resistant mutants than from the 137 wild-type and its derivatives remodeled by targeted engineering (FIG. 4). In addition, the biosensor can detect higher ammonium excretion from the selected methylammonium-resistant mutants than from their own wild-type as ammonium excretion from the wild-type strains is undetectable (FIG. 10A-B). These methods provide low false positive rates. The library composed of 327 mutants and 20% of EDA-resistant mutants from Klebsiella variicola showed improved ammonium excretion. (FIG. 6). Additionally, we can detect nitrogenase expression when mutants are exposed to ammonium. We introduced a plasmid containing GFP under the regulation of the nifH promoter. In the wild-type background, the nifH expression is repressed by 17 mM ammonium acetate, whereas some of the mutants exhibited the nifH expression even in the presence of 17 mM ammonium acetate, indicating ammonium repression of nitrogenase expression is relieved in those mutants (FIG. 7). These mutants also showed nitrogenase activity in the presence of ammonium (FIG. 8) and higher ammonium excretion up to 22.7 mM ammonium after 72 hr of incubation (FIG. 9).

### Example 4: Joint Methylammonium-EMS Selection Assay

Single colonies of Klebsiella, Kosakonia, Metakosakonia, Paraburkholderia, or Herbaspirillum were inoculated into rich media and grown overnight at 30° C. Aliquots (10 L) of the overnight cultures were diluted into 1 mL of rich media and grown for 5 hr at 30° C. to an optical density of about 1. The cell pellets collected by centrifugation were washed twice with 1 mL of ARA minimal medium. The cell pellets were resuspended in 1 mL of ARA minimal medium plus 14 μl of EMS and incubated for 2 hr at 30° C. The cell pellets collected by centrifugation were washed twice with 1 mL of ARA minimal medium and resuspended in 1 mL of ARA minimal medium. Aliquots (50 μl) of the cells were diluted into 950 μL of rich media, incubated for overnight at 30° C. and plated on ammonium-free minimal medium supplemented with EDA from about 0.1% EDA to about 0.2% EDA or methylammonium from about 25 mM to about 200 mM methylammonium, and the plates were incubated at 30° C. under anaerobic conditions or hypoxic conditions for facultative anaerobes such as Klebsiella and obligate aerobes such as Paraburkholderia, respectively (FIG. 3B). The resulting individual colonies were indicated as resistant mutants to EDA or methylammonium and selected, grown in liquid media, and stored.

Combining the biosensor strain with the EDA- or methylammonium-resistant mutants for co-culture of the biosensor strain with the individual resistant mutants and various control strains allowed for a determination of the degree of ammonium excretion from nitrogen fixation activity of the resistant mutants as determined by the presence/absence, or the degree thereof, of GFP fluorescence from the biosensor strain. The biosensor can detect higher ammonium excretion from the selected EDA-resistant mutants than from their wild-type (FIG. 4).

### Example 5: Identification of Single Nucleotide Polymorphism (SNP)

In order to identify mutations in the ammonium excreting mutants generated from Klebsiella variicola, Kosakonia sacchari, Metakosakonia intestini and Paraburkholderia tropica their genome sequences were analyzed using whole-genome sequencing. The genomic DNAs were isolated from each mutant and sequenced with an Illumina MiSeq® (next-generation sequencing (NGS)) v3. The sequence reads were aligned to reference sequences using Bowtie 2. (FIG. 10A and Table 6). Changes in the protein sequences and ammonium excretion levels from each mutant across four species were listed in Table 6 and depicted in FIG. 10B. Most of the SNPs were identified in glnA that encodes glutamine synthetase across four species. In addition, we also identified mutations in glnE of Klebsiella variicola that affects glutamine synthetase activity by regulating adenylylation of glutamine synthetase. It is noted that the mutagenized microbes with the edits in Table 6 have been deposited as illustrated in Table 7.

TABLE 6
SNPs in ammonium excreting mutants and their levels of
ammonium excretion.

| | | | | Nucleic | Amino |
|---|---|---|---|---|---|
| | | | Ammonium | Acid | Acid |
| | | Amino acid | excretion | SEQ ID | Seq ID |
| Gene | Species | substitution | (mM) | NO: | NO: |
| glnA | Klebsiella variicola | M66I | 18.58 ± 1.58 | 6 | 29 |
| | CI3296 | | | | |
| glnA | Klebsiella variicola | M66V | 19.98 ± 2.04 | 7 | 30 |
| | CI3936 | | | | |
| glnA | Klebsiella variicola | G157C | 20.12 ± 1.49 | 8 | 31 |
| | CI3933 | | | | |
| glnA | Klebsiella variicola | P174L | 18.59 ± 1.01 | 9 | 32 |
| | CI3927 | | | | |
| glnA | Klebsiella variicola | S189F | 21.26 ± 1.42 | 10 | 33 |
| | CI3925 | | | | |
| glnA | Klebsiella variicola | E208K | 22.73 ± 0.97 | 11 | 34 |
| | CI3943 | | | | |
| glnA | Klebsiella variicola | G268S | 19.98 ± 1.40 | 12 | 35 |
| | CI3940 | | | | |
| glnA | Klebsiella variicola | N339D | 22.19 ± 1.01 | 13 | 36 |
| | CI3938 | | | | |
| glnE | Klebsiella variicola | G322E/ | 2.34 ± 0.34 | 14 | 37 |
| | CI4874 | G746D | | | |
| glnA | Kosakonia sacchari | M66I | 9.30 ± 0.18 | 15 | 38 |
| | CI4065 | | | | |
| glnA | Metakosakonia | H172L | 10.01 ± 1.21 | 16 | 39 |
| | intestini CI4875 | | | | |
| glnA | Metakosakonia | G218S | 11.76 ± 0.54 | 17 | 40 |
| | intestini CI4876 | | | | |
| glnA | Metakosakonia | T255I | 10.28 ± 0.25 | 18 | 41 |
| | intestini CI4877 | | | | |
| glnA | Metakosakonia | A329V | 10.33 ± 0.19 | 19 | 42 |
| | intestini CI4878 | | | | |
| glnA | Paraburkholderia | Y103C | 6.16 ± 0.71 | 20 | 43 |
| | tropica CI4751 | | | | |
| glnA | Paraburkholderia | S163P | 5.03 ± 0.99 | 21 | 44 |
| | tropica CI4753 | | | | |
| glnA | Paraburkholderia | N171D | 5.86 ± 1.22 | 22 | 45 |
| | tropica CI4879 | | | | |
| glnA | Paraburkholderia | Q219H | 2.65 ± 1.09 | 23 | 46 |
| | tropica CI4752 | | | | |

### Example 6: Determining Relative Abundance of Microbial Strains

In order to determine the colonization capacity of some of the strains identified and isolated as described in the Examples provided herein, the percent relative abundance was calculated for a series of bacterial strains derived from K. variicola parental strain 137 using the cocoseq assay as described in PCT/US2020/012564, filed on Jan. 7, 2020.

In particular, unique naturally occurring cocoseq barcodes were inserted into the following strains: 137, 137-1036, 137-3933, 137-3944, 137-4073, 137-4074, 137-2285. All resulting barcoded strains were pooled in equal ratio to create an inoculum (final optical density of 1.0) which was then applied to ten seedlings (1 mL of inoculum per seedlings) in sterile sand. Successfully germinated plants were harvested three weeks after inoculation and processed similarly to previous colonization experiments to result in purified gDNA of the root rhizosphere. Purified gDNA was used as template (3 ul of gDNA per reaction) for cocoseq PCR as described in PCT/US2020/012564, filed on Jan. 7, 2020, which is incorporated by reference herein. Subsequent PCR products were sequenced on the Illumina MiSeq® (next-generation sequencing (NGS)) platform (2×75 bp) and resulting data was analyzed via in-house software to identify and enumerate cocoseq barcodes.

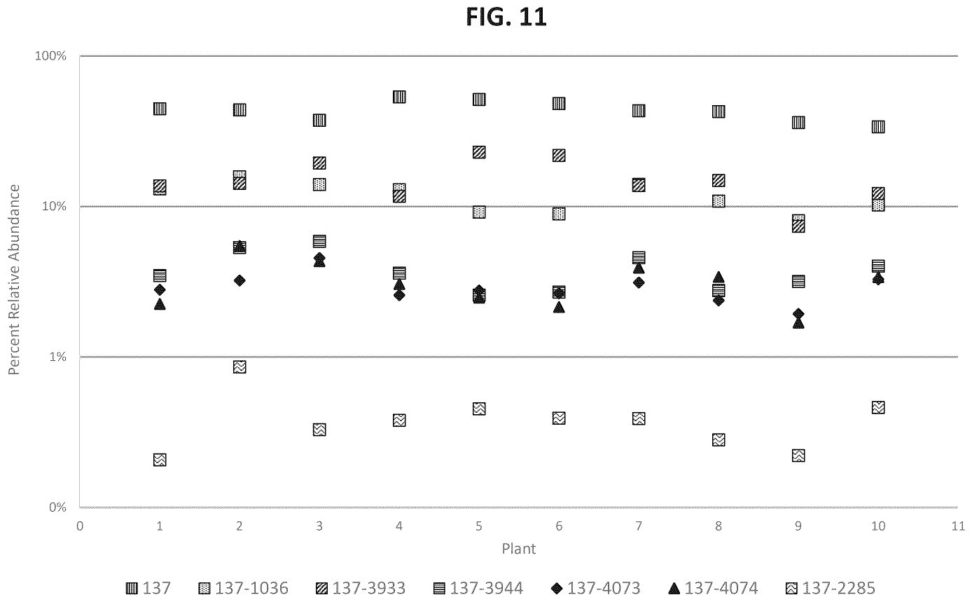

As can be seen in FIG. 11, strains identified in the Examples provided herein (e.g., 137-3933), had colonization capacities similar to the parental 137 strain.

### Example 7: Trials with Mutagenized Strains of the Disclosure

An experiment will be conducted utilizing each of the deposited mutant microbes described in Table 6 and their corresponding parental strain in order to evaluate the ability of the mutant microbes to fix nitrogen. The mutant microbe and respective parental strain will be added to a liquid composition comprising an agriculturally acceptable carrier and applied to a plant, a plant part (e.g., seed), or a locus in which the plant is located, or a locus in which the plant will be grown. The plants to be tested will include corn, soybean, canola, sorghum, potato, rice, vegetables, cereals (e.g., barley, rye, sorghum), pseudocereals (e.g., breadnut, buckwheat, sesame), and oilseeds.

In one set of experiments, the microbial compositions will be applied in furrow at a concentration of about 1×10⁶ to about 1×10¹¹ cfu of bacterial cells per milliliter or at a concentration per acre of between about 1×10⁶ to about 3×10¹² cfu per acre for each plant to be tested.

In another set of experiments, the microbial compositions will be applied by coating a seed of the specific plant at a concentration of about 1×10⁴ to about 1×10⁸ cfu per seed.

The relative and/or absolute abundance of each microbe will be assessed using the cocoseq assay as described in PCT/US2020/012564, filed on Jan. 7, 2020. The N-fixation activity of each of the microbes tested in this Example (i.e., microbes from Table 6 and their respective parental strains) will be assessed using an in vitro ARA assay at 5 mM glutamine or ammonium phosphate.

### NUMBERED EMBODIMENTS OF THE DISCLOSURE

Other subject matter contemplated by the present disclosure is set out in the following numbered embodiments:
1) A biosensor capable of detecting the presence of ammonium in a composition, the biosensor comprising: a bacterium comprising: (a) a nucleic acid sequence encoding a reporter molecule, (b) a promoter or fragment thereof selected from the Nif regulon operably linked to the nucleic acid sequence of (a), (c) a nucleic acid sequence encoding an inhibitory nitrogen fixation regulatory protein, and (d) a nucleic acid sequence encoding a positive nitrogen fixation regulatory protein; wherein the bacterium expresses the reporter molecule in the absence of ammonium.
2) The biosensor of embodiment 1, wherein the reporter molecule is a fluorescent protein, functional fragment, and/or fusions thereof.
3) The biosensor of embodiment 2, wherein the fluorescent protein is a GFP, RFP, YFP, CFP, or functional variant or fragment thereof.
4) The biosensor of embodiment 3, wherein the fluorescent protein is GFP.
5) The biosensor of embodiment 4, wherein the GFP is a superfolder GFP.
6) The biosensor of any one of the above embodiments, wherein the promoter or fragment thereof is selected from the nifHDK operon.
7) The biosensor of any one of the above embodiments, wherein the promoter is a nifH promoter.
8) The biosensor of any one of the above embodiments, wherein the inhibitory nitrogen fixation regulatory protein is NifL.
9) The biosensor of any one of the above embodiments, wherein the positive nitrogen fixation regulatory protein is NifA.
10) The biosensor of any one of the above embodiments, wherein the bacterium in Escherichia coli.
11) A method of detecting the presence of ammonium in a composition, the method comprising: (a) inoculating a composition with a biosensor, wherein the biosensor comprises a bacterium, which comprises: (i) a nucleic acid sequence encoding a reporter molecule, (ii) a promoter or fragment thereof selected from the Nif regulon operably linked to the nucleic acid sequence of (i), (iii) a nucleic acid sequence encoding an inhibitory nitrogen fixation regulatory protein, and (iv) a nucleic acid sequence encoding a positive nitrogen fixation regulatory protein; wherein the bacterium expresses the reporter molecule in the absence of ammonium; (b) exposing the inoculated composition to a stimulus sufficient to activate the reporter molecule; and (c) detecting reporter molecule output of the inoculated composition following (b), as compared to a control composition.
12) The method of embodiment 11, further comprising quantifying the amount of ammonium in the composition based upon the detected reporter molecule output.
13) The method of embodiment 11 or 12, wherein the stimulus is selected from the group consisting of a chemical stimulus, a physical stimulus, a genetic stimulus and an energy stimulus.
14) The method of any one of embodiments 11-13, wherein the stimulus is light.
15) The method of embodiment 14, wherein the reporter molecule is a fluorescent protein, functional fragment, and/or fusions thereof.
16) The method of embodiment 15, wherein the fluorescent protein is a GFP, RFP, YFP, CFP, or functional variant or fragment thereof.
17) The method of embodiment 15 or 16, wherein the fluorescent protein is GFP.
18) The method of embodiment 17, wherein the GFP is a superfolder GFP.
19) The method of any one of embodiments 15-18, wherein step (b) entails exposing the inoculated composition to light excitation sufficient to fluoresce the fluorescent protein, functional fragment, and/or fusions thereof.
20) The method of any one of embodiments 15-19, wherein step (c) entails detecting intensity of fluorescent output of the inoculated composition following (b), as compared to the control composition.
21) The method of any one of embodiments 15-20, wherein the fluorescence is detected with a flow cytometer, a plate reader, or fluorescence-activated droplet sorting.
22) A method for identification of bacterial mutants capable of fixing atmospheric nitrogen, the method comprising: (a) exposing a population of bacteria to a mutagen in a microbial composition; (b) co-culturing the microbial composition with a biosensor, wherein the biosensor comprises a bacterium, which comprises: (i) a nucleic acid sequence encoding a reporter molecule, (ii) a promoter or fragment thereof selected from the Nif regulon operably linked to the nucleic acid sequence of (i), (iii) a nucleic acid sequence encoding an inhibitory nitrogen fixation regulatory protein, and (iv) a nucleic acid sequence encoding a positive nitrogen fixation regulatory protein, wherein the bacterium expresses the reporter molecule in the absence of ammonium; (c) exposing the co-cultured composition to a stimulus sufficient to activate the reporter molecule; and (d) identifying bacterial mutants capable of fixing atmospheric nitrogen as those that result in decreased or no reporter molecule output as compared to a control.
23) The method of embodiment 22, wherein the reporter molecule output of the co-cultured composition is detected following (c), as compared to a control composition.
24) The method of embodiment 23, wherein the control composition comprises microbes that do not express the reporter molecule.
25) The method of embodiment 23, wherein the control composition comprises microbes that express a functionally deleted variant of the reporter molecule.
26) The method of any one of embodiment 22-25, wherein the stimulus is selected from the group consisting of a chemical stimulus, a physical stimulus, a genetic stimulus and an energy stimulus.
27) The method of any one of embodiments 22-25, wherein the stimulus is light.
28) The method of embodiment 27, wherein the reporter molecule is a fluorescent protein, functional fragment, and/or fusions thereof.
29) The method of embodiment 28, wherein the fluorescent protein is a GFP, RFP, YFP, CFP, or functional variant or fragment thereof.
30) The method of embodiment 28 or 29, wherein the fluorescent protein is GFP.
31) The method of embodiment 30, wherein the GFP is a superfolder GFP.
32) The method of any one of embodiments 28-31, wherein step (c) entails exposing the inoculated composition to light excitation sufficient to fluoresce the fluorescent protein, functional fragment, and/or fusions thereof.
33) The method of any one of embodiments 28-32, wherein the fluorescence is detected with a flow cytometer, a plate reader, or fluorescence-activated droplet sorting.
34) The method of any one of embodiments 22-33, further comprising isolating the bacterial mutants capable of fixing atmospheric nitrogen identified in (d).
35) The method of embodiment 34, wherein the isolated bacterial mutants are comprise a bacterium selected from a bacterium deposited as PTA-126709, a bacterium deposited as PTA-126710, a bacterium deposited as PTA-126711, a bacterium deposited as PTA-126712, a bacterium deposited as PTA-126713, a bacterium deposited as PTA-126714, a bacterium deposited as PTA-126715, a bacterium deposited as PTA-126716, a bacterium deposited as PTA-126717, a bacterium deposited as PTA-126718, a bacterium deposited as PTA-126719, a bacterium deposited as PTA-126720, a bacterium deposited as PTA-126721, a bacterium deposited as PTA-126722, a bacterium deposited as PTA-126723, a bacterium deposited as PTA-126724, a bacterium deposited as PTA-126725, a bacterium deposited as PTA-126726 and combinations thereof.
36) The method of embodiment 34, wherein the isolated bacterial mutants comprise a bacterium comprising at least one genetic variation introduced into a member selected from the group consisting of: nifA, nifL, ntrB, ntrC, polynucleotide encoding glutamine synthetase, glnA, glnB, glnK, drat, amtB, polynucleotide encoding glutaminase, glnD, glnE, nifJ, nifH, nifD, nifK, nifY, nifE, nif, nifU, nifS, nifV, nifW, nifZ, nifM, nifF, nifB, nifQ, a gene associated with biosynthesis of a nitrogenase enzyme, or combinations thereof.
37) The method of embodiment 34 or 36, wherein the isolated bacterial mutants comprise a bacterium comprising at least one genetic variation in glnA.
38) The method of embodiment 37, wherein the at least one genetic variation in glnA comprises at least one nucleotide substitution at nucleotide position 198, 469, 515, 521, 566, 622, 652, 764, 802, 986 and/or 1015 of a Klebsiella glnA gene or at a homologous nucleotide position in a homolog thereof.
39) The method of embodiment 38, wherein the Klebsiella glnA gene comprises a nucleic acid sequence of SEQ ID NO: 1.
40) The method of embodiment 38, wherein the homolog of the Klebsiella glnA gene is a Kosakonia glnA gene.
41) The method of embodiment 38 or 40, wherein the homolog of the Klebsiella glnA gene comprises a nucleic acid sequence of SEQ ID NO: 2.
42) The method of embodiment 38, wherein the homolog of the Klebsiella glnA gene is a Metakosakonia glnA gene.
43) The method of embodiment 38 or 42, wherein the homolog of the Klebsiella glnA gene comprises a nucleic acid sequence of SEQ ID NO: 3.
44) The method of any one of embodiments 37-43, wherein the glnA gene comprising the at least one genetic variation comprises a nucleic acid sequence selected from the group consisting of SEQ ID NOs 6-13 and 15-19.
45) The method of any one of embodiments 37-44, wherein expression of the glnA gene comprising the at least one genetic variation produces a GlnA protein comprising at least one amino acid substitution at amino acid position 66, 157, 172, 174, 189, 208, 218, 255, 268, 329 and/or 339 of a Klebsiella GlnA protein or at a homologous amino acid position in a homolog thereof.
46) The method of any one of embodiments 37-44, wherein expression of the glnA gene comprising the at least one genetic variation produces a GlnA protein comprising at least one amino acid substitution selected from the group consisting of M66I, M66V, G157C, H172L, P174L, S189F, E208K, G218S, T255I, G268S, A329V and/or N339D of a Klebsiella GlnA protein and identical amino acid substitutions at homologous amino acid positions in a homolog thereof.
47) The method of embodiment 45 or 46, wherein the Klebsiella GlnA protein comprises an amino acid sequence of SEQ ID NO: 24.
48) The method of embodiment 45 or 46, wherein the homolog of the Klebsiella GlnA protein is a Kosakonia GlnA protein.
49) The method of any one of embodiment 45, 46 or 48, wherein the homolog of the Klebsiella GlnA protein comprises an amino acid sequence of SEQ ID NO: 25.
50) The method of embodiment 45 or 46, wherein the homolog of the Klebsiella GlnA protein is a Metakosakonia GlnA protein.
51) The method of any one of embodiment 45, 46 or 50, wherein the homolog of the Klebsiella GlnA protein comprises an amino acid sequence of SEQ ID NO: 26.
52) The method of any one of embodiments 45-51, wherein the GlnA protein comprises an amino acid sequence selected from the group consisting of SEQ ID NOs 29-36 and 38-42.
53) The method of embodiment 37, wherein the at least one genetic variation in glnA comprises at least one nucleotide substitution at nucleotide position 308, 487, 511 and/or 657 of a Paraburkholderia glnA gene or in a homologous nucleotide position in a homolog thereof.
54) The method of embodiment 53, wherein the Paraburkholderia glnA gene comprises a nucleic acid sequence of SEQ ID NO: 4.
55) The method of any one of embodiments 37, 53 or 54, wherein the glnA gene comprising the at least one genetic variation comprises a nucleic acid sequence selected from the group consisting of SEQ ID NOs 20-23.
56) The method of any one of embodiments 37 or 53-55, wherein expression of the glnA gene comprising the at least one genetic variation produces a GlnA protein comprising at least one amino acid substitution at amino acid position 103, 163, 171 and/or 219 of a Paraburkholderia GlnA protein or in a homologous position in a homolog thereof.
57) The method of any one of embodiments 37 or 53-55, wherein expression of the glnA gene comprising the at least one genetic variation produces a GlnA protein comprising at least one amino acid substitution selected from the group consisting of Y103C, S163P, N171D and/or Q219H of a Paraburkholderia GlnA protein and identical amino acid substitutions in homologous amino acid positions in a homolog thereof.
58) The method of embodiment 56 or 57, wherein the Paraburkholderia GlnA protein comprises an amino acid sequence of SEQ ID NO: 27.
59) The method of any one of embodiments 56-58, wherein the GlnA protein comprises an amino acid sequence selected from the group consisting of SEQ ID NOs 43-46.
60) The method of embodiment 34 or 36, wherein the isolated bacterial mutants comprise a bacterium comprising at least one genetic variation in glnE.
61) The method of embodiment 60, wherein the at least one genetic variation in glnE comprises at least one nucleotide substitution at nucleotide position 965 and/or 2838 of a Klebsiella glnE gene or at a homologous nucleotide position in a homolog thereof.
62) The method of embodiment 60 or 61, wherein the at least one genetic variation in glnE comprises at least one nucleotide substitution at nucleotide position 965 and 2838 of a Klebsiella glnE gene or at a homologous nucleotide position in a homolog thereof.
63) The method of embodiment 61 or 62, wherein the Klebsiella glnE gene comprises a nucleic acid sequence of SEQ ID NO: 5.
64) The method of any one of embodiments 60-63, wherein the glnE gene comprising the at least one genetic variation comprises a nucleic acid sequence of SEQ ID NO: 14.
65) The method of any one of embodiments 60-64, wherein expression of the glnE gene comprising the at least one genetic variation produces a GlnE protein comprising at least one amino acid substitution at amino acid position 322 and/or 746 of a Klebsiella GlnE protein or at a homologous amino acid position in a homolog thereof.
66) The method of any one of embodiments 60-64, wherein expression of the glnE gene comprising the at least one genetic variation produces a GlnE protein comprising at least one amino acid substitution at amino acid position 322 and 746 of a Klebsiella GlnE protein or at a homologous amino acid position in a homolog thereof.
67) The method of embodiment 65 or 66, wherein the amino acid substitution at position 322 or the homologous amino acid position is a G to E substitution.
68) The method of any one of embodiments 65-67, wherein the amino acid substitution at position 746 or the homologous amino acid position is a G to D substitution.
69) The method of any one of embodiments 65-68, wherein the Klebsiella GlnE protein comprises an amino acid sequence of SEQ ID NO: 28.
70) The method of any one of embodiments 65-69, wherein the GlnE protein comprises an amino acid sequence of SEQ ID NO: 37.
71) A method for high-throughput identification of one or more bacterial mutants capable of fixing atmospheric nitrogen, the method comprising: (a) exposing a population of bacteria to a mutagen; (b) transferring the population of bacteria in (a) into one or more samples comprising a medium; (c) co-culturing a biosensor within each of the one or more samples in (b), wherein the biosensor comprises a bacterium, which comprises: (i) a nucleic acid sequence encoding a reporter molecule, (ii) a promoter or fragment thereof selected from the Nif regulon operably linked to the nucleic acid sequence of (i), (iii) a nucleic acid sequence encoding an inhibitory nitrogen fixation regulatory protein, and(iv) a nucleic acid sequence encoding a positive nitrogen fixation regulatory protein; wherein the bacterium expresses the reporter molecule in the absence of ammonium; (d) exposing each of the one or more samples in (c) to a stimulus sufficient to activate the reporter molecule; and (e) identifying from the one or more samples in (d) one or more bacterial mutants capable of fixing atmospheric nitrogen as those that result in decreased output of the reporter molecule as compared to a control.
72) The method of embodiment 71, wherein the output of the reporter molecule of the one or more samples in (d) is measured following (d), as compared to a control composition.
73) The method of embodiment 72, wherein the control composition comprises microbes that do not express the reporter molecule.
74) The method of embodiment 72, wherein the control composition comprises microbes that express a functionally deleted variant of the reporter molecule.
75) The method of any one of embodiments 71-74, wherein the stimulus is selected from the group consisting of a chemical stimulus, a physical stimulus, a genetic stimulus and an energy stimulus.
76) The method of any one of embodiments 71-75, wherein the stimulus is light.
77) The method of embodiment 76, wherein the reporter molecule is a fluorescent protein, functional fragment, and/or fusions thereof.
78) The method of embodiment 77, wherein the fluorescent protein is a GFP, RFP, YFP, CFP, or functional variant or fragment thereof.
79) The method of embodiment 77 or 78, wherein the fluorescent protein is GFP.
80) The method of embodiment 79, wherein the GFP is a superfolder GFP.
81) The method of any one of embodiments 77-80, wherein step (d) entails exposing the inoculated composition to light excitation sufficient to fluoresce the fluorescent protein, functional fragment, and/or fusions thereof.
82) The method of any one of embodiments 77-81, wherein the fluorescence is detected with a flow cytometer, a plate reader, or fluorescence-activated droplet sorting.
83) The method of any one of embodiments 71-80, further comprising isolating the bacterial mutants capable of fixing atmospheric nitrogen.
84) The method of embodiment 83, wherein the isolated bacterial mutants comprise a bacterium are selected from a bacterium deposited as PTA-126709, a bacterium deposited as PTA-126710, a bacterium deposited as PTA-126711, a bacterium deposited as PTA-126712, a bacterium deposited as PTA-126713, a bacterium deposited as PTA-126714, a bacterium deposited as PTA-126715, a bacterium deposited as PTA-126716, a bacterium deposited as PTA-126717, a bacterium deposited as PTA-126718, a bacterium deposited as PTA-126719, a bacterium deposited as PTA-126720, a bacterium deposited as PTA-126721, a bacterium deposited as PTA-126722, a bacterium deposited as PTA-126723, a bacterium deposited as PTA-126724, a bacterium deposited as PTA-126725, a bacterium deposited as PTA-126726 and combinations thereof.
85) The method of embodiment 83, wherein the isolated bacterial mutants comprise a bacterium comprising at least one genetic variation introduced into a member selected from the group consisting of: nifA, nifL, ntrB, ntrC, polynucleotide encoding glutamine synthetase, glnA, glnB, glnK, drat, amtB, polynucleotide encoding glutaminase, glnD, glnE, nifJ, nifH, nifD, nifK, nifY, nifE, nif, nif, nifS, nifV, nifW, nifZ, nifM, nif, nifB, nifQ, a gene associated with biosynthesis of a nitrogenase enzyme, or combinations thereof.
86) The method of embodiment 83 or 85, wherein the isolated bacterial mutants comprise a bacterium comprising at least one genetic variation in glnA.
87) The method of embodiment 86, wherein the at least one genetic variation in glnA comprises at least one nucleotide substitution at nucleotide position 198, 469, 515, 521, 566, 622, 652, 764, 802, 986 and/or 1015 of a Klebsiella glnA gene or at a homologous nucleotide position in a homolog thereof.
88) The method of embodiment 87, wherein the Klebsiella glnA gene comprises a nucleic acid sequence of SEQ ID NO: 1.
89) The method of embodiment 87, wherein the homolog of the Klebsiella glnA gene is a Kosakonia glnA gene.
90) The method of embodiment 87 or 89, wherein the homolog of the Klebsiella glnA gene comprises a nucleic acid sequence of SEQ ID NO: 2.
91) The method of embodiment 87, wherein the homolog of the Klebsiella glnA gene is a Metakosakonia glnA gene.
92) The method of embodiment 87 or 91, wherein the homolog of the Klebsiella glnA gene comprises a nucleic acid sequence of SEQ ID NO: 3.
93) The method of any one of embodiments 86-92, wherein the glnA gene comprising the at least one genetic variation comprises a nucleic acid sequence selected from the group consisting of SEQ ID NOs 6-13 and 15-19.
94) The method of any one of embodiments 86-93, wherein expression of the glnA gene comprising the at least one genetic variation produces a GlnA protein comprising at least one amino acid substitution at amino acid position 66, 157, 172, 174, 189, 208, 218, 255, 268, 329 and/or 339 of a Klebsiella GlnA protein or at a homologous amino acid position in a homolog thereof.
95) The method of any one of embodiments 86-93, wherein expression of the glnA gene comprising the at least one genetic variation produces a GlnA protein comprising at least one amino acid substitution selected from the group consisting of M66I, M66V, G157C, H172L, P174L, S189F, E208K, G218S, T255I, G268S, A329V and/or N339D of a Klebsiella GlnA protein and identical amino acid substitutions at homologous amino acid positions in a homolog thereof.
96) The method of embodiment 94 or 95, wherein the Klebsiella GlnA protein comprises an amino acid sequence of SEQ ID NO: 24.
97) The method of embodiment 94 or 95, wherein the homolog of the Klebsiella GlnA protein is a Kosakonia GlnA protein.
98) The method of any one of embodiment 94, 95 or 97, wherein the homolog of the Klebsiella GlnA protein comprises an amino acid sequence of SEQ ID NO: 25.
99) The method of embodiment 94 or 95, wherein the homolog of the Klebsiella GlnA protein is a Metakosakonia GlnA protein.
100) The method of any one of embodiment 94, 95 or 99, wherein the homolog of the Klebsiella GlnA protein comprises an amino acid sequence of SEQ ID NO: 26.
101) The method of any one of embodiments 94-100, wherein the GlnA protein comprises an amino acid sequence selected from the group consisting of SEQ ID NOs 29-36 and 38-42.
102) The method of embodiment 86, wherein the at least one genetic variation in glnA comprises at least one nucleotide substitution at nucleotide position 308, 487, 511 and/or 657 of a Paraburkholderia glnA gene or in a homologous nucleotide position in a homolog thereof.
103) The method of embodiment 102, wherein the Paraburkholderia glnA gene comprises a nucleic acid sequence of SEQ ID NO: 4.
104) The method of any one of embodiments 86, 102 or 103, wherein the glnA gene comprising the at least one genetic variation comprises a nucleic acid sequence selected from the group consisting of SEQ ID NOs 20-23.
105) The method of any one of embodiments 86 or 102-104, wherein expression of the glnA gene comprising the at least one genetic variation produces a GlnA protein comprising at least one amino acid substitution at amino acid position 103, 163, 171 and/or 219 of a Paraburkholderia GlnA protein or in a homologous position in a homolog thereof.
106) The method of any one of embodiments 86 or 102-104, wherein expression of the glnA gene comprising the at least one genetic variation produces a GlnA protein comprising at least one amino acid substitution selected from the group consisting of Y103C, S163P, N171D and/or Q219H of a Paraburkholderia GlnA protein and identical amino acid substitutions in homologous amino acid positions in a homolog thereof.
107) The method of embodiment 105 or 106, wherein the Paraburkholderia GlnA protein comprises an amino acid sequence of SEQ ID NO: 27.
108) The method of any one of embodiments 105-107, wherein the GlnA protein comprises an amino acid sequence selected from the group consisting of SEQ ID NOs 43-46.
109) The method of embodiment 83 or 85, wherein the isolated bacterial mutants comprise at least one genetic variation in glnE.
110) The method of embodiment 109, wherein the at least one genetic variation in glnE comprises at least one nucleotide substitution at nucleotide position 965 and/or 2838 of a Klebsiella glnE gene or at a homologous nucleotide position in a homolog thereof.
111) The method of embodiment 109 or 110, wherein the at least one genetic variation in glnE comprises at least one nucleotide substitution at nucleotide position 965 and 2838 of a Klebsiella glnE gene or at a homologous nucleotide position in a homolog thereof.
112) The method of embodiment 110 or 111, wherein the Klebsiella glnE gene comprises a nucleic acid sequence of SEQ ID NO: 5.
113) The method of any one of embodiments 109-112, wherein the glnE gene comprising the at least one genetic variation comprises a nucleic acid sequence of SEQ ID NO: 14.
114) The method of any one of embodiments 109-113, wherein expression of the glnE gene comprising the at least one genetic variation produces a GlnE protein comprising at least one amino acid substitution at amino acid position 322 and/or 746 of a Klebsiella GlnE protein or at a homologous amino acid position in a homolog thereof.
115) The method of any one of embodiments 109-113, wherein expression of the glnE gene comprising the at least one genetic variation produces a GlnE protein comprising at least one amino acid substitution at amino acid position 322 and 746 of a Klebsiella GlnE protein or at a homologous amino acid position in a homolog thereof.
116) The method of embodiment 114 or 115, wherein the amino acid substitution at position 322 or the homologous amino acid position is a G to E substitution.
117) The method of any one of embodiments 114-116, wherein the amino acid substitution at position 746 or the homologous amino acid position is a G to D substitution.
118) The method of any one of embodiments 114-117, wherein the Klebsiella GlnE protein comprises an amino acid sequence of SEQ ID NO: 28.
119) The method of any one of embodiments 114-118, wherein the GlnE protein comprises an amino acid sequence of SEQ ID NO: 37.
120) The method of any one of embodiments 71-119, wherein the population of bacteria comprises at least 100,000 bacteria, and wherein the method for high-throughput identification is completed in less than four hours.
121) A method for identifying novel bacterial genes, pathways, and/or regulatory elements involved in fixing atmospheric nitrogen, the method comprising: (a) exposing a population of bacteria to a mutagen in a microbial composition; (b) inoculating the microbial composition with a biosensor, wherein the biosensor comprises a bacterium, which comprises: (i) a nucleic acid sequence encoding a reporter molecule, (ii) a promoter or fragment thereof selected from the Nif regulon operably linked to the nucleic acid sequence of (i), (iii) a nucleic acid sequence encoding an inhibitory nitrogen fixation regulatory protein, and (iv) a nucleic acid sequence encoding a positive nitrogen fixation regulatory protein; wherein the bacterium expresses the reporter molecule in the absence of ammonium; (c) exposing the inoculated composition to a stimulus sufficient to activate the reporter molecule, (d) measuring the output of the reporter molecule of the inoculated composition following (c), as compared to a control composition; (e) isolating bacteria from inoculated compositions that exhibit a decreased output of the reporter molecule as compared to the control composition as bacteria capable of fixing atmospheric nitrogen; (f) sequencing the genome(s) of the bacteria capable of fixing atmospheric nitrogen from (e); and (g) identifying genes, pathways, and/or regulatory elements that contain mutations from the sequenced genomes.
122) The method of embodiment 121, wherein the control composition comprises microbes that do not express the reporter molecule.
123) The method of embodiment 121, wherein the control composition comprises microbes that express a functionally deleted variant of the reporter molecule.
124) The method of any one of embodiments 121-123, wherein the stimulus is selected from the group consisting of a chemical stimulus, a physical stimulus, a genetic stimulus and an energy stimulus.
125) The method of any one of embodiments 121-124, wherein the stimulus is light.
126) The method of embodiment 125, wherein the reporter molecule is a fluorescent protein, functional fragment, and/or fusions thereof.
127) The method of embodiment 126, wherein the fluorescent protein is a GFP, RFP, YFP, CFP, or functional variant or fragment thereof.
128) The method of embodiment 126 or 127, wherein the fluorescent protein is GFP.
129) The method of embodiment 128, wherein the GFP is a superfolder GFP.
130) The method of any one of embodiments 126-129, wherein step (c) entails exposing the inoculated composition to light excitation sufficient to fluoresce the fluorescent protein, functional fragment, and/or fusions thereof.
131) The method of any one of embodiments 126-129, wherein the fluorescence is detected with a flow cytometer, a plate reader, or fluorescence-activated droplet sorting.
132) The method of any one of embodiments 121-131, further comprising determining whether the genes, pathways, and/or regulatory elements contain mutations known to be associated with the fixation of atmospheric nitrogen.
133) The method of any one of embodiments 121-132, wherein the identified mutations comprise at least one mutation in glnA or glnE.
134) The method of embodiment 133, wherein the at least one mutation in glnA comprises at least one nucleotide substitution at nucleotide position 198, 469, 515, 521, 566, 622, 652, 764, 802, 986 and/or 1015 of a Klebsiella glnA gene or at a homologous nucleotide position in a homolog thereof.
135) The method of embodiment 134, wherein the Klebsiella glnA gene comprises a nucleic acid sequence of SEQ ID NO: 1.
136) The method of embodiment 134, wherein the homolog of the Klebsiella glnA gene is a Kosakonia glnA gene.
137) The method of embodiment 134 or 136, wherein the homolog of the Klebsiella glnA gene comprises a nucleic acid sequence of SEQ ID NO: 2.
138) The method of embodiment 134, wherein the homolog of the Klebsiella glnA gene is a Metakosakonia glnA gene.
139) The method of embodiment 134 or 138, wherein the homolog of the Klebsiella glnA gene comprises a nucleic acid sequence of SEQ ID NO: 3.
140) The method of any one of embodiments 133-139, wherein the glnA gene comprising the at least one genetic variation comprises a nucleic acid sequence selected from the group consisting of SEQ ID NOs 6-13 and 15-19.
141) The method of any one of embodiments 133-140, wherein expression of the glnA gene comprising the at least one genetic variation produces a GlnA protein comprising at least one amino acid substitution at amino acid position 66, 157, 172, 174, 189, 208, 218, 255, 268, 329 and/or 339 of a Klebsiella GlnA protein or at a homologous amino acid position in a homolog thereof.
142) The method of any one of embodiments 133-140, wherein expression of the glnA gene comprising the at least one genetic variation produces a GlnA protein comprising at least one amino acid substitution selected from the group consisting of M66I, M66V, G157C, H172L, P174L, S189F, E208K, G218S, T255I, G268S, A329V and/or N339D of a Klebsiella GlnA protein and identical amino acid substitutions at homologous amino acid positions in a homolog thereof.
143) The method of embodiment 141 or 142, wherein the Klebsiella GlnA protein comprises an amino acid sequence of SEQ ID NO: 24.
144) The method of embodiment 141 or 142, wherein the homolog of the Klebsiella GlnA protein is a Kosakonia GlnA protein.
145) The method of any one of embodiment 141, 142 or 144, wherein the homolog of the Klebsiella GlnA protein comprises an amino acid sequence of SEQ ID NO: 25.
146) The method of embodiment 141 or 142, wherein the homolog of the Klebsiella GlnA protein is a Metakosakonia GlnA protein.
147) The method of any one of embodiment 141, 142 or 146, wherein the homolog of the Klebsiella GlnA protein comprises an amino acid sequence of SEQ ID NO: 26.
148) The method of any one of embodiments 141-147, wherein the GlnA protein comprises an amino acid sequence selected from the group consisting of SEQ ID NOs 29-36 and 38-42.
149) The method of embodiment 133, wherein the at least one genetic variation in glnA comprises at least one nucleotide substitution at nucleotide position 308, 487, 511 and/or 657 of a Paraburkholderia glnA gene or in a homologous nucleotide position in a homolog thereof.
150) The method of embodiment 149, wherein the Paraburkholderia glnA gene comprises a nucleic acid sequence of SEQ ID NO: 4.
151) The method of any one of embodiments 133, 149 or 150, wherein the glnA gene comprising the at least one genetic variation comprises a nucleic acid sequence selected from the group consisting of SEQ ID NOs 20-23.
152) The method of any one of embodiments 133 or 147-151, wherein expression of the glnA gene comprising the at least one genetic variation produces a GlnA protein comprising at least one amino acid substitution at amino acid position 103, 163, 171 and/or 219 of a Paraburkholderia GlnA protein or in a homologous position in a homolog thereof.
153) The method of any one of embodiments 133 or 147-151, wherein expression of the glnA gene comprising the at least one genetic variation produces a GlnA protein comprising at least one amino acid substitution selected from the group consisting of Y103C, S163P, N171D and/or Q219H of a Paraburkholderia GlnA protein and identical amino acid substitutions in homologous amino acid positions in a homolog thereof.
154) The method of embodiment 152 or 153, wherein the Paraburkholderia GlnA protein comprises an amino acid sequence of SEQ ID NO: 27.
155) The method of any one of embodiments 152-154, wherein the GlnA protein comprises an amino acid sequence selected from the group consisting of SEQ ID NOs 43-46.
156) The method of embodiment 133, wherein the at least one genetic variation in glnE comprises at least one nucleotide substitution at nucleotide position 965 and/or 2838 of a Klebsiella glnE gene or at a homologous nucleotide position in a homolog thereof.
157) The method of embodiment 133 or 156, wherein the at least one genetic variation in glnE comprises at least one nucleotide substitution at nucleotide position 965 and 2838 of a Klebsiella glnE gene or at a homologous nucleotide position in a homolog thereof.
158) The method of embodiment 156 or 157, wherein the Klebsiella glnE gene comprises a nucleic acid sequence of SEQ ID NO: 5.
159) The method of any one of embodiments 133 or 156-158, wherein the glnE gene comprising the at least one genetic variation comprises a nucleic acid sequence of SEQ ID NO: 14.
160) The method of any one of embodiments 133 or 156-159, wherein expression of the glnE gene comprising the at least one genetic variation produces a GlnE protein comprising at least one amino acid substitution at amino acid position 322 and/or 746 of a Klebsiella GlnE protein or at a homologous amino acid position in a homolog thereof.
161) The method of any one of embodiments 133 or 156-159, wherein expression of the glnE gene comprising the at least one genetic variation produces a GlnE protein comprising at least one amino acid substitution at amino acid position 322 and 746 of a Klebsiella GlnE protein or at a homologous amino acid position in a homolog thereof.
162) The method of embodiment 160 or 161, wherein the amino acid substitution at position 322 or the homologous amino acid position is a G to E substitution.
163) The method of any one of embodiments 160-162, wherein the amino acid substitution at position 746 or the homologous amino acid position is a G to D substitution.
164) The method of any one of embodiments 160-163, wherein the Klebsiella GlnE protein comprises an amino acid sequence of SEQ ID NO: 28.
165) The method of any one of embodiments 160-164, wherein the GlnE protein comprises an amino acid sequence of SEQ ID NO: 37.
166) A method for identification of bacterial mutants capable of fixing atmospheric nitrogen, the method comprising: (a) exposing a population of bacteria to a mutagen; (b) exposing the population of bacteria exposed to the mutagen in (a) to a diazotrophic growth inhibitor in a microbial composition; (c) co-culturing the microbial composition with a biosensor, wherein the biosensor comprises a bacterium, which comprises: (i) a nucleic acid sequence encoding a reporter molecule, (ii) a promoter or fragment thereof selected from the Nif regulon operably linked to the nucleic acid sequence of (i), (iii) a nucleic acid sequence encoding an inhibitory nitrogen fixation regulatory protein, and (iv) a nucleic acid sequence encoding a positive nitrogen fixation regulatory protein, wherein the bacterium expresses the reporter molecule in the absence of ammonium; (d) exposing the co-cultured composition to a stimulus sufficient to activate the reporter molecule; and (e) identifying bacterial mutants capable of fixing atmospheric nitrogen as those that result in decreased or no output of the reporter molecule, as compared to a control.
167) The method of embodiment 166, wherein the diazotrophic growth inhibitor is ethylenediamine (EDA) or methylammonium.
168) The method of embodiment 166 or 167, wherein the output of the reporter molecule of the one or more samples in (d) is measured following (d), as compared to a control composition.
169) The method of embodiment 168, wherein the control composition comprises microbes that do not express the reporter molecule.
170) The method of embodiment 168, wherein the control composition comprises microbes that express a functionally deleted variant of the reporter molecule.
171) The method of any one of embodiments 166-170, wherein the stimulus is selected from the group consisting of a chemical stimulus, a physical stimulus, a genetic stimulus and an energy stimulus.
172) The method of any one of embodiments 166-171, wherein the stimulus is light.
173) The method of embodiment 172, wherein the reporter molecule is a fluorescent protein, functional fragment, and/or fusions thereof.
174) The method of embodiment 173, wherein the fluorescent protein is a GFP, RFP, YFP, CFP, or functional variant or fragment thereof.
175) The method of embodiment 173 or 174, wherein the fluorescent protein is GFP.
176) The method of embodiment 175, wherein the GFP is a superfolder GFP.
177) The method of any one of embodiments 173-176, wherein step (d) entails exposing the inoculated composition to light excitation sufficient to fluoresce the fluorescent protein, functional fragment, and/or fusions thereof.
178) The method of any one of embodiments 173-177, wherein the fluorescence is detected with a flow cytometer, a plate reader, or fluorescence-activated droplet sorting.
179) The method of any one of embodiments 166-178, further comprising isolating the bacterial mutants identified as capable of fixing atmospheric nitrogen.
180) The method of embodiment 179, wherein the isolated bacterial mutants are comprise a bacterium selected from a bacterium deposited as PTA-126709, a bacterium deposited as PTA-126710, a bacterium deposited as PTA-126711, a bacterium deposited as PTA-126712, a bacterium deposited as PTA-126713, a bacterium deposited as PTA-126714, a bacterium deposited as PTA-126715, a bacterium deposited as PTA-126716, a bacterium deposited as PTA-126717, a bacterium deposited as PTA-126718, a bacterium deposited as PTA-126719, a bacterium deposited as PTA-126720, a bacterium deposited as PTA-126721, a bacterium deposited as PTA-126722, a bacterium deposited as PTA-126723, a bacterium deposited as PTA-126724, a bacterium deposited as PTA-126725, a bacterium deposited as PTA-126726 and combinations thereof.
181) The method of embodiment 179, wherein the isolated bacterial mutants comprise bacteria which comprise at least one genetic variation introduced into a member selected from the group consisting of: nifA, nifL, ntrB, ntrC, polynucleotide encoding glutamine synthetase, glnA, glnB, glnK, drat, amtB, polynucleotide encoding glutaminase, glnD, glnE, nifJ, nifH, nifD, nifK, nifY, nifE, nif, nifU, nifS, nifV, nifW, nifZ, nifM, nifF, nifB, nifQ, a gene associated with biosynthesis of a nitrogenase enzyme, or combinations thereof.
182) The method of embodiment 179 or 181, wherein the isolated bacterial mutants comprise a bacterium comprising a genetic variation in glnA.
183) The method of embodiment 182, wherein the at least one genetic variation in glnA comprises at least one nucleotide substitution at nucleotide position 198, 469, 515, 521, 566, 622, 652, 764, 802, 986 and/or 1015 of a Klebsiella glnA gene or at a homologous nucleotide position in a homolog thereof.
184) The method of embodiment 183, wherein the Klebsiella glnA gene comprises a nucleic acid sequence of SEQ ID NO: 1.
185) The method of embodiment 183, wherein the homolog of the Klebsiella glnA gene is a Kosakonia glnA gene.
186) The method of embodiment 183 or 185, wherein the homolog of the Klebsiella glnA gene comprises a nucleic acid sequence of SEQ ID NO: 2.
187) The method of embodiment 183, wherein the homolog of the Klebsiella glnA gene is a Metakosakonia glnA gene.
188) The method of embodiment 183 or 187, wherein the homolog of the Klebsiella glnA gene comprises a nucleic acid sequence of SEQ ID NO: 3.
189) The method of any one of embodiments 182-188, wherein the glnA gene comprising the at least one genetic variation comprises a nucleic acid sequence selected from the group consisting of SEQ ID NOs 6-13 and 15-19.
190) The method of any one of embodiments 182-189, wherein expression of the glnA gene comprising the at least one genetic variation produces a GlnA protein comprising at least one amino acid substitution at amino acid position 66, 157, 172, 174, 189, 208, 218, 255, 268, 329 and/or 339 of a Klebsiella GlnA protein or at a homologous amino acid position in a homolog thereof.
191) The method of any one of embodiments 182-189, wherein expression of the glnA gene comprising the at least one genetic variation produces a GlnA protein comprising at least one amino acid substitution selected from the group consisting of M66I, M66V, G157C, H172L, P174L, S189F, E208K, G218S, T255I, G268S, A329V and/or N339D of a Klebsiella GlnA protein and identical amino acid substitutions at homologous amino acid positions in a homolog thereof.
192) The method of embodiment 190 or 191, wherein the Klebsiella GlnA protein comprises an amino acid sequence of SEQ ID NO: 24.
193) The method of embodiment 190 or 191, wherein the homolog of the Klebsiella GlnA protein is a Kosakonia GlnA protein.
194) The method of any one of embodiment 190, 191 or 193, wherein the homolog of the Klebsiella GlnA protein comprises an amino acid sequence of SEQ ID NO: 25.
195) The method of embodiment 190 or 191, wherein the homolog of the Klebsiella GlnA protein is a Metakosakonia GlnA protein.
196) The method of any one of embodiment 190, 191 or 195, wherein the homolog of the Klebsiella GlnA protein comprises an amino acid sequence of SEQ ID NO: 26.
197) The method of any one of embodiments 190-196, wherein the GlnA protein comprises an amino acid sequence selected from the group consisting of SEQ ID NOs 29-36 and 38-42.
198) The method of embodiment 182, wherein the at least one genetic variation in glnA comprises at least one nucleotide substitution at nucleotide position 308, 487, 511 and/or 657 of a Paraburkholderia glnA gene or in a homologous nucleotide position in a homolog thereof.
199) The method of embodiment 198, wherein the Paraburkholderia glnA gene comprises a nucleic acid sequence of SEQ ID NO: 4.
200) The method of any one of embodiments 182, 198 or 199, wherein the glnA gene comprising the at least one genetic variation comprises a nucleic acid sequence selected from the group consisting of SEQ ID NOs 20-23.
201) The method of any one of embodiments 182 or 198-200, wherein expression of the glnA gene comprising the at least one genetic variation produces a GlnA protein comprising at least one amino acid substitution at amino acid position 103, 163, 171 and/or 219 of a Paraburkholderia GlnA protein or in a homologous position in a homolog thereof.
202) The method of any one of embodiments 182 or 198-200, wherein expression of the glnA gene comprising the at least one genetic variation produces a GlnA protein comprising at least one amino acid substitution selected from the group consisting of Y103C, S163P, N171D and/or Q219H of a Paraburkholderia GlnA protein and identical amino acid substitutions in homologous amino acid positions in a homolog thereof.
203) The method of embodiment 201 or 202, wherein the Paraburkholderia GlnA protein comprises an amino acid sequence of SEQ ID NO: 27.
204) The method of any one of embodiments 201-203, wherein the GlnA protein comprises an amino acid sequence selected from the group consisting of SEQ ID NOs 43-46.
205) The method of embodiment 179 or 181, wherein the isolated bacterial mutants comprise at least one genetic variation in glnE.
206) The method of embodiment 205, wherein the at least one genetic variation in glnE comprises at least one nucleotide substitution at nucleotide position 965 and/or 2838 of a Klebsiella glnE gene or at a homologous nucleotide position in a homolog thereof.
207) The method of embodiment 205 or 206, wherein the at least one genetic variation in glnE comprises at least one nucleotide substitution at nucleotide position 965 and 2838 of a Klebsiella glnE gene or at a homologous nucleotide position in a homolog thereof.
208) The method of embodiment 206 or 207, wherein the Klebsiella glnE gene comprises a nucleic acid sequence of SEQ ID NO: 5.
209) The method any one of embodiments 205-208, wherein the glnE gene comprising the at least one genetic variation comprises a nucleic acid sequence of SEQ ID NO: 14.
210) The method of any one of embodiments 205-209, wherein expression of the glnE gene comprising the at least one genetic variation produces a GlnE protein comprising at least one amino acid substitution at amino acid position 322 and/or 746 of a Klebsiella GlnE protein or at a homologous amino acid position in a homolog thereof.
211) The method of any one of embodiments 205-209, wherein expression of the glnE gene comprising the at least one genetic variation produces a GlnE protein comprising at least one amino acid substitution at amino acid position 322 and 746 of a Klebsiella GlnE protein or at a homologous amino acid position in a homolog thereof.
212) The method of embodiment 210 or 211, wherein the amino acid substitution at position 322 or the homologous amino acid position is a G to E substitution.
213) The method of any one of embodiments 210-212, wherein the amino acid substitution at position 746 or the homologous amino acid position is a G to D substitution.
214) The method of any one of embodiments 210-213, wherein the Klebsiella GlnE protein comprises an amino acid sequence of SEQ ID NO: 28.
215) The method of any one of embodiments 210-214, wherein the GlnE protein comprises an amino acid sequence of SEQ ID NO: 37.
216) The method of any one of embodiments 11-215, wherein the promoter or fragment thereof is selected from the nifHDK operon.
217) The method of any one of embodiments 11-216, wherein the promoter is a nifH promoter.
218) The method of any one of embodiments 11-217, wherein the inhibitory nitrogen fixation regulatory protein is NifL.
219) The method of any one of embodiments 11-218, wherein the positive nitrogen fixation regulatory protein is NifA.
220) The method of any one of embodiments 22-219, wherein the population of bacteria is selected from wild-type bacteria, transgenic mutant bacteria, non-intergeneric mutant bacteria and intergeneric mutant bacteria.
221) The method of any one of embodiments 22-220, wherein the mutagen is selected from mitomycin C (MMC), N-methyl-N-nitrosourea (MNU), nitrous acid (NA), diepoxybutane (DEB), 1,2,7,8-diepoxyoctane (DEO), ethyl methane sulfonate (EMS), methyl methane sulfonate (MMS), N-methyl-N′-nitro-N-nitrosoguanidine (MNNG), 4-nitroquinoline 1-oxide (4-NQO), 2-methyloxy-6-chloro-9(3-[ethyl-2-chloroethyl]-aminopropylamin)-acridinedihydrochloride (ICR-170), 2-amino purine (2AP), and hydroxylamine (HA).
222) The method of embodiment 95221, wherein the mutagen is EMS.
223) A microbial composition, comprising: one or more isolated bacteria selected from the group consisting of a bacterium deposited as PTA-126709, a bacterium deposited as PTA-126710, a bacterium deposited as PTA-126711, a bacterium deposited as PTA-126712, a bacterium deposited as PTA-126713, a bacterium deposited as PTA-126714, a bacterium deposited as PTA-126715, a bacterium deposited as PTA-126716, a bacterium deposited as PTA-126717, a bacterium deposited as PTA-126718, a bacterium deposited as PTA-126719, a bacterium deposited as PTA-126720, a bacterium deposited as PTA-126721, a bacterium deposited as PTA-126722, a bacterium deposited as PTA-126723, a bacterium deposited as PTA-126724, a bacterium deposited as PTA-126725 and a bacterium deposited as PTA-126726.
224) The microbial composition of embodiment 223, further comprising an agriculturally acceptable carrier.
225) The microbial composition of embodiment 223, wherein the one or more isolated bacteria fix atmospheric nitrogen at a rate higher than a wild type parental lineage bacteria.
226) A nitrogen fixing bacterium comprising a mutant glnE gene comprising at least one nucleotide substitution at nucleotide position 965 and/or 2838 of a Klebsiella glnE gene or at a homologous nucleotide position in a homolog thereof.
227) The nitrogen fixing bacterium of embodiment 226, wherein the mutant glnE gene comprises a nucleotide substitution at nucleotide position 965 and 2838 of the Klebsiella glnE gene or in homologous nucleotide positions in the homolog thereof.
228) The nitrogen fixing bacterium of embodiment 226 or 227, wherein the mutant glnE gene shares at least 85%, 86%, 87%, 88%, 89%, 90%, 91%, 92%, 93%, 94%, 95%, 96%, 97%, 98%, or 99% identity with the Klebsiella glnE gene or the homolog thereof.
229) The nitrogen fixing bacterium of embodiment 226, wherein expression of the mutant glnE gene produces a mutant GlnE protein with at least one amino acid substitution at amino acid position 322 and/or 746 of a Klebsiella GlnE protein or at a homologous amino acid position in a homolog thereof.
230) The nitrogen fixing bacterium of embodiment 227, wherein expression of the mutant glnE gene produces a mutant GlnE protein with amino acid substitutions at amino acid positions 322 and 746 of the Klebsiella GlnE protein or at homologous amino acid positions in the homolog thereof.
231) The nitrogen fixing bacterium of embodiment 229 or 230, wherein the amino acid substitution at position 322 or the homologous amino acid position is a G to E substitution.
232) The nitrogen fixing bacterium of any one of embodiments 229-231, wherein the amino acid substitution at position 746 or the homologous amino acid position is a G to D substitution.
233) The nitrogen fixing bacterium of any one of embodiments 229-232, wherein the mutant GlnE protein shares at least 90%, 91%, 92%, 93%, 94%, 95%, 96%, 97%, 98%, or 99% identity with the Klebsiella GlnE protein or the homolog thereof.
234) The nitrogen fixing bacterium of any one of embodiments 226-233, wherein the Klebsiella glnE gene comprises a nucleic acid sequence of SEQ ID NO: 5.
235) The nitrogen fixing bacterium of any one of embodiments 226-234, wherein the mutant glnE gene comprises a nucleic acid sequence of SEQ ID NO: 14.
236) The nitrogen fixing bacterium of any one of embodiments 226-235, wherein the Klebsiella GlnE protein comprises an amino acid sequence of SEQ ID NO: 28.
237) The nitrogen fixing bacterium of any one of embodiments 229-236, wherein the mutant GlnE protein comprises an amino acid sequence of SEQ ID NO: 37.
238) A nitrogen fixing bacterium comprising a mutant glnE gene encoding a GlnE protein comprising an amino acid sequence with at least one amino acid substitution at amino acid positions 322 and/or 746 of a Klebsiella GlnE protein or at a homologous amino acid position in a homolog thereof.
239) The nitrogen fixing bacterium of embodiment 238, wherein the GlnE protein comprises amino acid substitutions at amino acid positions 322 and 746 of the Klebsiella GlnE protein or at homologous amino acid positions in the homolog thereof.
240) The nitrogen fixing bacterium of embodiment 238 or 239, wherein the amino acid substitution at position 322 or the homologous amino acid position is a G to E substitution.
241) The nitrogen fixing bacterium of any one of embodiments 238-240, wherein the amino acid substitution at position 746 or the homologous amino acid position is a G746D substitution.
242) The nitrogen fixing bacterium of any one of embodiments 238-241, wherein the GlnE protein shares at least 90%, 91%, 92%, 93%, 94%, 95%, 96%, 97%, 98%, or 99% identity with the Klebsiella GlnE protein or the homolog thereof.
243) The nitrogen fixing bacterium of any one of embodiments 238-242, wherein the Klebsiella GlnE protein comprises an amino acid sequence of SEQ ID NO: 28.
244) The nitrogen fixing bacterium of any one of embodiments 238-243, wherein the mutant GlnE protein comprises an amino acid sequence of SEQ ID NO: 37.
245) A nitrogen fixing bacterium comprising a mutant GlnE protein comprising an amino acid sequence with at least one amino acid substitution at amino acid positions 322 and/or 746 of a Klebsiella GlnE protein or at a homologous amino acid position in a homolog thereof.
246) The nitrogen fixing bacterium of embodiment 245, wherein the GlnE protein comprises amino acid substitutions at amino acid positions 322 and 746 of the Klebsiella GlnE protein or at homologous amino acid positions in the homolog thereof.
247) The nitrogen fixing bacterium of embodiment 245 or 246, wherein the amino acid substitution at position 322 or the homologous amino acid position is a G to E substitution.
248) The nitrogen fixing bacterium of any one of embodiments 245-247, wherein the amino acid substitution at position 746 or the homologous amino acid position is a G to D substitution.
249) The nitrogen fixing bacterium of any one of embodiments 245-248, wherein the GlnE protein shares at least 90%, 91%, 92%, 93%, 94%, 95%, 96%, 97%, 98%, or 99% identity with the Klebsiella GlnE protein or the homolog thereof.
250) The nitrogen fixing bacterium of any one of embodiments 245-249, wherein the Klebsiella GlnE protein comprises an amino acid sequence of SEQ ID NO: 28.
251) The nitrogen fixing bacterium of any one of embodiments 245-250, wherein the mutant GlnE protein comprises an amino acid sequence of SEQ ID NO: 37.
252) The nitrogen fixing bacterium of any one of embodiments 226-251, wherein the nitrogen fixing bacterium is Klebsiella variicola CI4874.
253) A nitrogen fixing bacterium comprising a mutant glnA gene comprising at least one nucleotide substitution at nucleotide position 198, 469, 515, 521, 566, 622, 652, 764, 802, 986 and/or 1015 of a Klebsiella glnA gene or at a homologous nucleotide position in a homolog thereof.
254) The nitrogen fixing bacterium of embodiment 253, wherein the mutant glnA gene shares at least 85%, 86%, 87%, 88%, 89%, 90%, 91%, 92%, 93%, 94%, 95%, 96%, 97%, 98%, or 99% identity with the Klebsiella glnA gene or the homolog thereof.
255) The nitrogen fixing bacterium of embodiment 253 or 254, wherein expression of the mutant glnA gene produces a mutant GlnA protein with at least one amino acid substitution at amino acid position 66, 157, 172, 174, 189, 208, 218, 255, 268, 329 and/or 339 of a Klebsiella GlnA protein or at a homologous amino acid position in a homolog thereof.
256) The nitrogen fixing bacterium of embodiment 253 or 254, wherein expression of the mutant glnA gene produces a mutant GlnA protein with at least one amino acid substitution selected from the group consisting of M66I, M66V, G157C, H172L, P174L, S189F, E208K, G218S, T255I, G268S, A329V and/or N339D of a Klebsiella GlnA protein and identical amino acid substitutions at homologous amino acid positions in a homolog thereof.
257) The nitrogen fixing bacterium of embodiment 255 or 256, wherein the mutant GlnA protein shares at least 90%, 91%, 92%, 93%, 94%, 95%, 96%, 97%, 98%, or 99% identity with the Klebsiella GlnA protein or the homolog thereof.
258) The nitrogen fixing bacterium of any one of embodiments 253-257, wherein the Klebsiella glnA gene comprises a nucleic acid sequence of SEQ ID NO: 1.
259) The nitrogen fixing bacterium of any one of embodiments 253-257, wherein the homolog of the Klebsiella glnA gene is a Kosakonia glnA gene.
260) The nitrogen fixing bacterium of any one of embodiments 253-257 or 259, wherein the homolog of the Klebsiella glnA gene comprises a nucleic acid sequence of SEQ ID NO: 2.
261) The nitrogen fixing bacterium of any one of embodiments 253-257, wherein the homolog of the Klebsiella glnA gene is a Metakosakonia glnA gene.
262) The nitrogen fixing bacterium of any one of embodiments 253-257 or 261, wherein the homolog of the Klebsiella glnA gene comprises a nucleic acid sequence of SEQ ID NO: 3.
263) The nitrogen fixing bacterium of any one of embodiments 253-262, wherein the mutant glnA gene comprises a nucleic acid sequence selected from the group consisting of SEQ ID NOs 6-13 and 15-19.
264) The nitrogen fixing bacterium of any one of embodiments 255-258, wherein the Klebsiella GlnA protein comprises an amino acid sequence of SEQ ID NO: 24.
265) The nitrogen fixing bacterium of any one of embodiments 255-257, 259-260 or 263, wherein the homolog of the Klebsiella GlnA protein comprises an amino acid sequence of SEQ ID NO: 25.
266) The nitrogen fixing bacterium of any one of embodiments 255-257 or 261-263, wherein the homolog of the Klebsiella GlnA protein comprises an amino acid sequence of SEQ ID NO: 26.
267) The nitrogen fixing bacterium of any one of embodiments 255-266, wherein the mutant GlnA protein comprises an amino acid sequence selected from the group consisting of SEQ ID NOs 29-36 and 38-42.
268) A nitrogen fixing bacterium comprising a mutant glnA gene encoding a GlnA protein comprising an amino acid sequence with at least one amino acid substitution at amino acid positions 66, 157, 172, 174, 189, 208, 218, 255, 268, 329 and/or 339 of a Klebsiella GlnA protein or at a homologous amino acid position in a homolog thereof.
269) The nitrogen fixing bacterium of embodiment 268, wherein the least one amino acid substitution is selected from the group consisting of M66I, M66V, G157C, H172L, P174L, S189F, E208K, G218S, T255I, G268S, A329V and N339D of the Klebsiella GlnA protein and identical amino acid substitutions at homologous positions in the homolog thereof.
270) The nitrogen fixing bacterium of embodiment 268 or 269, wherein the mutant GlnA protein shares at least 90%, 91%, 92%, 93%, 94%, 95%, 96%, 97%, 98%, or 99% identity with the Klebsiella GlnA protein or the homolog thereof.
271) The nitrogen fixing bacterium of any one of embodiments 268-270, wherein the Klebsiella GlnA protein comprises an amino acid sequence of SEQ ID NO: 24.
272) The nitrogen fixing bacterium of any one of embodiments 268-270, wherein the homolog of the Klebsiella GlnA protein is a Kosakonia GlnA protein.
273) The nitrogen fixing bacterium of any one of embodiments 268-270 or 272, wherein the homolog of the Klebsiella GlnA protein comprises an amino acid sequence of SEQ ID NO: 25.
274) The nitrogen fixing bacterium of any one of embodiments 268-270, wherein the homolog of the Klebsiella GlnA protein is a Metakosakonia GlnA protein.
275) The nitrogen fixing bacterium of any one of embodiments 268-270 or 274, wherein the homolog of the Klebsiella GlnA protein comprises an amino acid sequence of SEQ ID NO: 26.
276) The nitrogen fixing bacterium of any one of embodiments 268-275, wherein the GlnA protein comprises an amino acid sequence selected from the group consisting of SEQ ID NOs 29-36 and 38-42.
277) A nitrogen fixing bacterium comprising a mutant GlnA protein comprising an amino acid sequence with at least one amino acid substitution at amino acid positions 66, 157, 172, 174, 189, 208, 218, 255, 268, 329 and/or 339 of a Klebsiella GlnA protein or at a homologous amino acid position in a homolog thereof.
278) The nitrogen fixing bacterium of embodiment 277, wherein the least one amino acid substitution is selected from the group consisting of M66I, M66V, G157C, H172L, P174L, S189F, E208K, G218S, T255I, G268S, A329V and N339D of the Klebsiella GlnA protein and identical amino acid substitutions in homologous amino acid positions in the homolog thereof.
279) The nitrogen fixing bacterium of embodiment 277 or 278, wherein the mutant GlnA protein shares at least 90%, 91%, 92%, 93%, 94%, 95%, 96%, 97%, 98%, or 99% identity with the Klebsiella GlnA protein or the homolog thereof.
280) The nitrogen fixing bacterium of any one of embodiments 277-279, wherein the Klebsiella GlnA protein comprises an amino acid sequence of SEQ ID NO: 24.
281) The nitrogen fixing bacterium of any one of embodiments 277-279, wherein the homolog of the Klebsiella GlnA protein is a Kosakonia GlnA protein.
282) The nitrogen fixing bacterium of any one of embodiments 277-279 or 281, wherein the homolog of the Klebsiella GlnA protein comprises an amino acid sequence of SEQ ID NO: 25.
283) The nitrogen fixing bacterium of any one of embodiments 277-279, wherein the homolog of the Klebsiella GlnA protein is a Metakosakonia GlnA protein.
284) The nitrogen fixing bacterium of any one of embodiments 277-279 or 283, wherein the homolog of the Klebsiella GlnA protein comprises an amino acid sequence of SEQ ID NO: 26.
285) The nitrogen fixing bacterium of any one of embodiments 277-284, wherein the GlnA protein comprises an amino acid sequence selected from the group consisting of SEQ ID NOs 29-36 and 38-42.
286) The nitrogen fixing bacterium of any one of embodiments 253-285, wherein the nitrogen fixing bacterium is selected from the group consisting of Klebsiella variicola CI3296, Klebsiella variicola CI3936, Klebsiella variicola CI3933, Klebsiella variicola CI3927, Klebsiella variicola CI3925, Klebsiella variicola CI3943, Klebsiella variicola CI3940, Klebsiella variicola CI3938, Kosakonia sacchari CI4065, Metakosakonia intestini CI4875, Metakosakonia intestini CI4876, Metakosakonia intestini CI4877 and Metakosakonia intestini CI4878.
287) A nitrogen fixing bacterium comprising a mutant glnA gene comprising at least one nucleotide substitution at nucleotide position 308, 487, 511 and/or 657 of a Paraburkholderia glnA gene or in a homologous nucleotide position in a homolog thereof.
288) The nitrogen fixing bacterium of embodiment 287, wherein the mutant glnA gene shares at least 85%, 86%, 87%, 88%, 89%, 90%, 91%, 92%, 93%, 94%, 95%, 96%, 97%, 98%, or 99% identity with the Paraburkholderia glnA gene or the homolog thereof.
289) The nitrogen fixing bacterium of embodiment 287 or 288, wherein expression of the mutant glnA gene produces a mutant GlnA protein with at least one amino acid substitution at amino acid position 103, 163, 171 and/or 219 of a Paraburkholderia GlnA protein or in a homologous position in a homolog thereof.
290) The nitrogen fixing bacterium of embodiment 287 or 288, wherein expression of the mutant glnA gene produces a mutant GlnA protein with at least one amino acid substitution selected from the group consisting of Y103C, S163P, N171D and/or Q219H of a Paraburkholderia GlnA protein and identical amino acid substitutions in homologous amino acid positions in a homolog thereof.
291) The nitrogen fixing bacterium of embodiment 289 or 290, wherein the mutant GlnA protein shares at least 90%, 91%, 92%, 93%, 94%, 95%, 96%, 97%, 98%, or 99% identity with the Paraburkholderia GlnA protein or the homolog thereof.
292) The nitrogen fixing bacterium of any one of embodiments 287-291, wherein the Paraburkholderia glnA gene comprises a nucleic acid sequence of SEQ ID NO: 4.
293) The nitrogen fixing bacterium of any one of embodiments 287-292, wherein the mutant glnA gene comprises a nucleic acid sequence selected from the group consisting of SEQ ID NOs 20-23.
294) The nitrogen fixing bacterium of any one of embodiments 289-293, wherein the Paraburkholderia GlnA protein comprises an amino acid sequence of SEQ ID NO: 27.
295) The nitrogen fixing bacterium of any one of embodiments 289-294, wherein the mutant GlnA protein comprises an amino acid sequence selected from the group consisting of SEQ ID NOs 43-46.
296) A nitrogen fixing bacterium comprising a mutant glnA gene encoding a GlnA protein comprising an amino acid sequence with at least one amino acid substitution at amino acid positions 103, 163, 171 and/or 219 of a Paraburkholderia GlnA protein or in a homologous amino acid position in a homolog thereof.
297) The nitrogen fixing bacterium of embodiment 296, wherein the least one amino acid substitution is selected from the group consisting of Y103C, S163P, N171D and Q219H of the Paraburkholderia GlnA protein and identical amino acid substitutions in homologous amino acid positions in the homolog thereof.
298) The nitrogen fixing bacterium of embodiment 296 or 297, wherein the mutant GlnA protein shares at least 90%, 91%, 92%, 93%, 94%, 95%, 96%, 97%, 98%, or 99% identity with the Paraburkholderia GlnA protein or the homolog thereof.
299) The nitrogen fixing bacterium of any one of embodiments 296-298, wherein the Paraburkholderia GlnA protein comprises an amino acid sequence of SEQ ID NO: 27.
300) The nitrogen fixing bacterium of any one of embodiments 296-299, wherein the GlnA protein comprises an amino acid sequence selected from the group consisting of SEQ ID NOs 43-46.
301) A nitrogen fixing bacterium comprising a mutant GlnA protein comprising an amino acid sequence with at least one amino acid substitution at amino acid positions 103, 163, 171 and/or 219 of a Paraburkholderia GlnA protein or in homologous amino acid positions in a homolog thereof.
302) The nitrogen fixing bacterium of embodiment 301, wherein the least one amino acid substitution is selected from the group consisting of Y103C, S163P, N171D and Q219H of the Paraburkholderia GlnA protein and identical amino acid substitutions in homologous amino acid positions in the homolog thereof.
303) The nitrogen fixing bacterium of embodiment 301 or 302, wherein the mutant GlnA protein shares at least 90%, 91%, 92%, 93%, 94%, 95%, 96%, 97%, 98%, or 99% identity with the Paraburkholderia GlnA protein or the homolog thereof.
304) The nitrogen fixing bacterium of any one of embodiments 301-303, wherein the Paraburkholderia GlnA protein comprises an amino acid sequence of SEQ ID NO: 27.
305) The nitrogen fixing bacterium of any one of embodiments 301-304, wherein the GlnA protein comprises an amino acid sequence selected from the group consisting of SEQ ID NOs 43-46.
306) The nitrogen fixing bacterium of any one of embodiments 287-305, wherein the nitrogen fixing bacterium is selected from the group consisting of Paraburkholderia tropica CI4751, Paraburkholderia tropica CI4753, Paraburkholderia tropica CI4879 and Paraburkholderia tropica CI4752.
307) The nitrogen fixing bacterium of any one of embodiments 226-306, wherein the bacterium is genetically engineered.
308) A microbial composition comprising the nitrogen fixing bacterium of any one of embodiments 226-307 and a carrier.
309) A method of providing fixed nitrogen to a plant comprising applying a microbial composition to a plant, a plant part, or a locus in which the plant is located, or a locus in which the plant will be grown, wherein the microbial composition comprises at least one nitrogen fixing bacterium of any one of embodiments 226-306.
310) The method of embodiment 309, wherein the microbial composition produces 1% or more of fixed nitrogen in the plant.
311) The method of any one of embodiments 309-310, wherein the composition is a solid.
312) The method of any one of embodiments 309-310, wherein the composition is a liquid.
313) The method of any one of embodiments 309-312, wherein the applying comprises coating a seed or other plant propagation member with the microbial composition.
314) The method of embodiment 313, wherein the at least one nitrogen fixing bacterium in the microbial composition has an average colonization ability per unit of plant root tissue of at least about 1.0×10⁴ cfu per gram of fresh weight of plant root tissue and produce fixed N of at least about 1×10¹⁵ mmol N per bacterial cell per hour.
315) The method of embodiment 313, wherein the applying comprises performing in-furrow treatment of the microbial composition.
316) The method of embodiment 315, wherein the in-furrow treatment comprises applying the microbial composition at a concentration per acre of between about 1×10⁶ to about 3×10¹² cfu per acre.
317) The method of embodiment 315 or 316, wherein the microbial composition is a liquid formulation comprising about 1×10⁶ to about 1×10¹¹ cfu of bacterial cells per milliliter.

SEQUENCES OF THE DISCLOSURE WITH SEQ ID NO IDENTIFIERS

| | Nucleic Acid | Amino Acid | |
|---|---|---|---|
| Gene Name | SEQ ID NO. | SEQ ID NO: | Strain |
| glnA | 1 | 24 | K. variicola CI137, 137 |
| glnA | 2 | 25 | K. sacchari 6 |
| glnA | 3 | 26 | M. intestini 910 |
| glnA | 4 | 27 | P. tropica 8 |
| glnE | 5 | 28 | K. variicola CI137, 137 |
| glnA | 6 | 29 | K. variicola CI3296, 137-3296 |
| glnA | 7 | 30 | K. variicola CI3936, 137-3936 |
| glnA | 8 | 31 | K. variicola CI3933, 137-3933 |
| glnA | 9 | 32 | K. variicola CI3927, 137-3927 |
| glnA | 10 | 33 | K. variicola CI3925, 137-3925 |
| glnA | 11 | 34 | K. variicola CI3943, 137-3943 |
| glnA | 12 | 35 | K. variicola CI3940, 137-3940 |
| glnA | 13 | 36 | K. variicola CI3938, 137-3938 |
| glnE | 14 | 37 | K. variicola CI4874, 137-4874 |
| glnA | 15 | 38 | K. sacchari CI4065, 6-4065 |
| glnA | 16 | 39 | M. intestini CI4875, 910-4875 |
| glnA | 17 | 40 | M. intestini CI4876, 910-4876 |
| glnA | 18 | 41 | M. intestini CI4877, 910-4877 |
| glnA | 19 | 42 | M. intestini CI4878, 910-4878 |
| glnA | 20 | 43 | P. tropica CI4751, 8-4751 |
| glnA | 21 | 44 | P. tropica CI4753, 8-4753 |
| glnA | 22 | 45 | P. tropica CI4879, 8-4879 |
| glnA | 23 | 46 | P. tropica CI4752, 8-4752 |

### INCORPORATION BY REFERENCE

All references, articles, publications, patents, patent publications, and patent applications cited herein are incorporated by reference in their entireties for all purposes.

However, mention of any reference, article, publication, patent, patent publication, and patent application cited herein is not, and should not be taken as, an acknowledgment or any form of suggestion that they constitute valid prior art or form part of the common general knowledge in any country in the world.

What is claimed:

1) A biosensor capable of detecting the presence of ammonium in a composition, the biosensor comprising: a bacterium comprising:
a) a nucleic acid sequence encoding a reporter molecule;
b) a promoter or fragment thereof selected from the Nif regulon operably linked to the nucleic acid sequence of (a);
c) a nucleic acid sequence encoding an inhibitory nitrogen fixation regulatory protein; and
d) a nucleic acid sequence encoding a positive nitrogen fixation regulatory protein; wherein the bacterium expresses the reporter molecule in the absence of ammonium.

2) The biosensor of claim 1, wherein the promoter or fragment thereof is selected from the nifHDK operon.

3) The biosensor of claim 1, wherein the promoter is a nifH promoter.

4) The biosensor of claim 1, wherein the inhibitory nitrogen fixation regulatory protein is NifL.

5) The biosensor of claim 1, wherein the positive nitrogen fixation regulatory protein is NifA.

6) A method for high-throughput identification of one or more bacterial mutants capable of fixing atmospheric nitrogen, the method comprising:
a) exposing a population of bacteria to a mutagen;
b) transferring the population of bacteria in (a) into one or more samples comprising a medium;
c) co-culturing a biosensor within each of the one or more samples in (b), wherein the biosensor comprises a bacterium, which comprises:
i) a nucleic acid sequence encoding a reporter molecule,
ii) a promoter or fragment thereof selected from the Nif regulon operably linked to the nucleic acid sequence of (i),
iii) a nucleic acid sequence encoding an inhibitory nitrogen fixation regulatory protein, and
iv) a nucleic acid sequence encoding a positive nitrogen fixation regulatory protein; wherein the bacterium expresses the reporter molecule in the absence of ammonium;
d) exposing each of the one or more samples in (c) to a stimulus sufficient to activate the reporter molecule; and
e) identifying from the one or more samples in (d) one or more bacterial mutants capable of fixing atmospheric nitrogen as those that result in decreased output of the reporter molecule as compared to a control.

7) The method of claim 6, wherein the stimulus is selected from the group consisting of a chemical stimulus, a physical stimulus, a genetic stimulus and an energy stimulus.

8) The method of claim 6, wherein the stimulus is light and the reporter molecule is a fluorescent protein.

9) The method of claim 8, wherein step (d) entails exposing the inoculated composition to light excitation sufficient to fluoresce the fluorescent protein, functional fragment, and/or fusions thereof.

10) The method of claim 9, wherein the fluorescence is detected with a flow cytometer, a plate reader, or fluorescence-activated droplet sorting.

11) The method of claim 6, further comprising isolating the bacterial mutants capable of fixing atmospheric nitrogen.

12) The method of claim 11, wherein the isolated bacterial mutants comprise a bacterium selected from a bacterium deposited as PTA-126709, a bacterium deposited as PTA-126710, a bacterium deposited as PTA-126711, a bacterium deposited as PTA-126712, a bacterium deposited as PTA-126713, a bacterium deposited as PTA-126714, a bacterium deposited as PTA-126715, a bacterium deposited as PTA-126716, a bacterium deposited as PTA-126717, a bacterium deposited as PTA-126718, a bacterium deposited as PTA-126719, a bacterium deposited as PTA-126720, a bacterium deposited as PTA-126721, a bacterium deposited as PTA-126722, a bacterium deposited as PTA-126723, a bacterium deposited as PTA-126724, a bacterium deposited as PTA-126725, a bacterium deposited as PTA-126726 and combinations thereof.

13) The method of claim 11, wherein the isolated bacterial mutants comprise a bacterium comprising at least one genetic variation in glnA.

14) The method of claim 13, wherein the at least one genetic variation in glnA comprises at least one nucleotide substitution at nucleotide position 198, 469, 515, 521, 566, 622, 652, 764, 802, 986 and/or 1015 of a Klebsiella glnA gene or at a homologous nucleotide position in a homolog thereof.

15) The method of claim 14, wherein the Klebsiella glnA gene comprises a nucleic acid sequence of SEQ ID NO: 1.

16) The method of claim 13, wherein the glnA gene comprising the at least one genetic variation comprises a nucleic acid sequence selected from the group consisting of SEQ ID NOs 6-13 and 15-19.

17) The method of claim 13, wherein expression of the glnA gene comprising the at least one genetic variation produces a GlnA protein comprising at least one amino acid substitution at amino acid position 66, 157, 172, 174, 189, 208, 218, 255, 268, 329 and/or 339 of a Klebsiella GlnA protein or at a homologous amino acid position in a homolog thereof.

18) The method of claim 13, wherein expression of the glnA gene comprising the at least one genetic variation produces a GlnA protein comprising at least one amino acid substitution selected from the group consisting of M66I, M66V, G157C, H172L, P174L, S189F, E208K, G218S, T255I, G268S, A329V and/or N339D of a Klebsiella GlnA protein and identical amino acid substitutions at homologous amino acid positions in a homolog thereof.

19) The method of claim 17, wherein the Klebsiella GlnA protein comprises an amino acid sequence of SEQ ID NO: 24.

20) The method of claim 17, wherein the GlnA protein comprises an amino acid sequence selected from the group consisting of SEQ ID NOs 29-36 and 38-42.

21) The method of claim 13, wherein the glnA gene comprising the at least one genetic variation comprises a nucleic acid sequence selected from the group consisting of SEQ ID NOs 20-23.

22) The method of claim 6, wherein the population of bacteria is selected from wild-type bacteria, transgenic mutant bacteria, non-intergeneric mutant bacteria and intergeneric mutant bacteria.

23) The method of claim 6, wherein the mutagen is selected from mitomycin C (MMC), N-methyl-N-nitrosourea (MNU), nitrous acid (NA), diepoxybutane (DEB), 1,2,7,8-diepoxyoctane (DEO), ethyl methane sulfonate (EMS), methyl methane sulfonate (MMS), N-methyl-N′-nitro-N-nitrosoguanidine (MNNG), 4-nitroquinoline 1-oxide (4-NQO), 2-methyloxy-6-chloro-9(3-[ethyl-2-chloroethyl]-aminopropylamin)-acridinedihydrochloride (ICR-170), 2-amino purine (2AP), and hydroxylamine (HA).

24) The method of claim 23, wherein the mutagen is EMS.


SEQUENCE LISTING (46 sequences)

SEQ ID NO: 1 - DNA, 1410 bp, Klebsiella variicola
  source 1..1410: mol_type = genomic DNA
atgtccgctgaacacgttttgacgatgctgaacgagcacgaagtgaagtttgtcgatctgcgcttcaccgataccaaaggtaaagaacagcacgtcacgattccgtctcatcaggtaaatgccgaattcttcgaagaaggcaaaatgtttgatggctcctcgatcggtggctggaaaggtatcaacgaatctgacatggtcctgatgccggacgcttccaccgcggtcattgacccgttctacgaagaaccgaccctgatcatccgctgcgatatcctcgagccaggcaccctgcagggctatgaccgtgacccgcgctccatcgcgaaacgcgctgaagagtacctgcgcgccaccggcatcgctgacaccgtcctgttcgggccagaaccagaattcttcctgttcgacgacatccgttttggcgcctctatctccggctcccatgtcgcgatcgatgacatcgaaggtgcgtggaactcctccaccaaatacgaaggcggtaacaaaggtcaccgtccgggcgtgaaagggggttacttcccggttccgccagtagactcttctcaggatatccgttccaccatgtgtatgatcatggaagagatgggcctggttgttgaagctcaccaccacgaagtggctaccgcaggtcagaacgaagtggcaacccgcttcaacaccatgaccaaaaaagcggacgaaattcagatttacaaatacgttgttcacaacgtcgctcaccgcttcggtaaaaccgcgacctttatgccgaaaccaatgttcggcgataacggctccggcatgcactgccatatgtctctggcgaaaaacgggactaacctgttctccggcgacaaatacgcaggtctgtctgagcaggcgctgttctacatcggcggcgtaatcaaacacgctaaagctatcaacgccctggcgaacccgaccaccaactcctacaagcgtctggtcccgggttacgaagcgccggttatgctggcttactctgcgcgtaaccgctctgcctccatccgtattccggtggtgacctctccgaaagcgcgtcgtatcgaagtgcgcttcccggacccggctgccaacccgtacctgtgctttgcggcgctgctgatggctggccttgatggtatcaagaacaaaatccatccgggcgaagcaatggacaaaaacctgtatgacctgccgccggaagaagcgaaagagatcccgcaggttgcaggctctctggaagaagccctgcaggccctggatgctgaccgcgagttcctgacggctggcggcgtgttcaccaatgacgctatcgatgcttacatcgccctgcgtctggaagagaacgaccgcgtacgcatgactccgcatccggttgagttcgaactgtactacagcgtctaa

SEQ ID NO: 2 - DNA, 1410 bp, Kosakonia sacchari
  source 1..1410: mol_type = genomic DNA
atgtccgctgaacacgttttgacgatgctgaatgagcacgaagtgaagtttgtcgatctgcgcttcaccgataccaaaggtaaagaacagcacgtcactatcccagcccatcaagtaaacgctgacttctttgaagaaggtaaaatgtttgatggttcttcgattggtggctggaaaggcatcaacgaatcagacatggtgctgatgccggacgcgactaccgctgtcattgacccgttctacgaagagtctacgctgatcattcgttgcgatatcctcgaaccaggcaccatgcagggctacgatcgtgacccgcgctccatcgctaaacgcgctgaagaatacctgcgctccaccggtctggcagacaccgttctgtttgggccagagccggaattcttcctgttcgacgacgtgcgcttcggcagctccatttccggttccagcgtcgctatcgacgatatcgaaggcgcatggaacacctccaccaaatacgaaggtggtaacaaaggtcaccgtccggcagtgaaaggcggttacttcccggttccgccggtcgattcatcacaggatctgcgttccaccatgtgtctggtgatggaagagatgggcctggttgttgaagctcaccaccacgaagtggcaacggctggtcagaacgaagtggctacccgcttcaacaccatgaccaaaaaagcggacgaaattcagatttacaaatacgtggtgcacaacgttgctcaccgctttggtaaaaccgcgacctttatgccgaaaccgatgttcggtgataacggttccggtatgcactgccacatgtctctgtccaagaacggtaccaacctgttctctggcgacaaatacgctggcctgtctgaaatggcgctgtactacattggcggcgtaatcaaacacgcaaaagcgatcaacgccctgtctaacccgaccaccaactcctacaagcgtctggtcccgggttatgaagcgccggtaatgctggcttactctgcacgtaaccgttctgcatctatccgtattccggtagtggcttctccgaaagcgcgtcgtatcgaagtgcgcttcccggacccggctgctaacccgtatctgtgcttcgcagcgctgttgatggctggtctggatggcattaaaaacaaaatccacccgggcgaagcgatggacaaaaacctgtatgacctgccggctgaagaagcgaaagaaatcccgcaggttgcaggctctctggaagaagcgctcaacgcgctggatgctgaccgcgagttcctgactgctggtggcgtattcaccgacgacgctatcgatgcttacatcgcgctgcgtatcgaagagaacgaccgtgttcgcatgacgccgcacccggtagagttcgaactgtactacagcgtttaa

SEQ ID NO: 3 - DNA, 1410 bp, Metakosakonia intestini
  source 1..1410: mol_type = genomic DNA
atgtccgctgaacacgttttgacgatgctgaacgagcacgaagtcaagtttgttgatttgcgcttcaccgacactaaaggtaaagagcagcacgtcacgatcccagctcatcaggtaaatgctgacttctttgaagaaggcaaaatgtttgatggctcctcgattggtggctggaaaggcattaacgaatctgacatggtactgatgccggacgcctctaccgctgtcattgacccgttctacgaagaaccgactctgatcatccgttgcgatatcctcgagccgggcactatgcagggctacgatcgcgacccgcgctccatcgcaaaacgcgctgaagaatacctgcgcgcaacaggcatcgcagacaccgttctgttcgggcctgagccagagttcttcctgttcgacgacgtacgtttcggcagctctatttccggttcccacgtcgctatcgatgatatcgaaggcgcatggaactcctccaccaaatacgaaggcggtaacaaaggtcaccgtccggctgttaaaggtggttacttcccggttccgccagtggactccgcacaagacctgcgttccactatgtgtctggtaatggaagagatgggcctggttgttgaagctcatcaccacgaagttgcaactgctggtcagaacgaagtggcaacccgcttcaataccatgaccaaaaaagcagacgaaattcagatctacaaatatgtggttcacaacgttgctcaccgcttcggtaaaaccgcaacctttatgccaaaaccgatgttcggtgataacggttccggtatgcactgccacatgtctctgtccaagaacggcgtaaacctgttctctggcgacaaatatgctggtctgtctgagcaggcgctgtactacatcggcggcgttattaaacacgctaaagcgatcaacgccctgtccaacccgactaccaactcctacaaacgtctggtcccgggttacgaagcaccggtcatgctggcttactctgcccgtaaccgttcagcttccatccgtattccggtggttgcatctccgaaagcgcgtcgtattgaagtgcgtttcccggacccggctgctaacccgtacctgtgcttcgcagcgctgctgatggctggtcttgatggcatcaagaacaaaatccatccgggcgaagcgatggacaaaaacctgtatgacctgccagctgaagaagcgaaagaaatcccgcaggttgcaggctctctggaagaagcactgaactgcctgaacgaagaccgcgagttcctgactgcgggtggcgtattcaccgacgacgcaattgatgcttacatcgctctgcgtatcgaagaaaacgaccgcgtacgcatgacgccgcacccggtagagttcgagctgtactacagcgtttaa

SEQ ID NO: 4 - DNA, 1416 bp, Paraburkholderia tropica
  source 1..1416: mol_type = genomic DNA
atgagtaaatccgtggccgacgtcatgcaactcgtcaaggacgaagacgtcaagtttgtcgacttccgctttaccgacacgcgcggcaaggagcaacacgtctcggttccgctgtcgcacttcgacgaagacaagttcgagtcgggtcatgcatttgacggttcgtcgatcgccggctggaagggcatcgaagcctcggacatgctgctcgttccggatgcgaacacggccttcatcgacccgttctacgaagagtcgacgctggttctgacctgcgacgtggtcgagccggctgacggcaagggctacgagcgcgacccgcgctcgctcgccaagcgcgctgaagcgtacctgaagagcacgggcctcggcgacacggccttcttcggtccggaacccgaattcttcattttcgactcggtccagtggaacacggacatgtcgggctcgttcgtcaagatcggctcggaagaagcaccgtggtcgtcgagcaaggaattcgaaggcggcaacacgggtcaccgtccgggcgtcaagggcggctacttcccggtcgcgccggtcgacacgttccaggacatccgttcggaaatgtgtctgctgctcgaacagatcggcatcccggtcgaagtgcaccaccacgaagtcgctggccagggccagaacgaaatcggcacgaagttctcgacgctggttcagcgcgcggactggacgcagcagatgaagtacatcatccataacgtggcgcacacgtatggcaagacggcgacgttcatgccgaagccgatcgttggcgacaacggttcgggcatgcacgttcaccagtcgatctggaaggacggccagaacctgttcgcaggcaacggctacgcaggtctgtcggaattcgcgctgttctacatcggcggcatcatcaagcacgctcgcgcgctgaacgccatcacgaacccgtcgacgaactcgtacaagcgtctggttccgcacttcgaagcaccggtcaagctggcttactcggcacgcaaccgttcggcgtcgatccgtattccgcacgtctcgaacccgaagggtcgccgtatcgaaacgcgcttcccggacccgatggccaacccgtacctgtgcttctcggcactgatgatggcaggtctggacggcgtgcagaacaagatccatccgggcgaagccgccgacaagaacctgtacgacctgccgccggaagaggatgcaaagatcccgaccgtttgcgccggcctcgaccaggctctggaagcgcttgacaaggatcgcgagttcctgacgcgcggtggcgtgttcacggattcgatgatcgacgcctacctcgcactgaaggaaggcgaactgcaacgcgtgcgcatgacgacgcacccggtcgagttcgaactgtactactcgctgtaa

SEQ ID NO: 5 - DNA, 2838 bp, Klebsiella variicola
  source 1..2838: mol_type = genomic DNA
atgatgccgctttctccgcaattacagcagcactggcagacggtcgctgaccgtctgccagcggattttcccattgccgaactgagcccacaggccaggtcggtcatggcgttcagcgattttgtcgaacagagtgtgatcgcccagccgggctggctgaatgagcttgcggactcctcgccggaggcggaagagtggcggcattacgaggcctggctgcaggatcgcctgcaggccgtcactgacgaagcggggttgatgcgagagctgcgtctcttccgccgccagatgatggtccgcatcgcctgggcgcaggcgctgtcgctggtgagcgaagaagagactctgcagcagctgagcgtcctggcggagaccctgattgtcgccgcccgcgactggctgtacgccgcctgctgtaaggagtggggaacgccatgcaatgccgagggccagccgcagccgctgctgatcctcgggatgggaaagctgggcggcggcgagctgaacttctcttccgatatcgatctgatctttgcctggcctgagcatggcgccacccgcggcggccgccgcgagctggataacgcccagttctttacccgtctggggcagcggctgatcaaggcccttgaccagccgacgcaggacggctttgtctatcgggttgacatgcgcctgcggccgtttggcgacagtgggccgctggtactcagttttgcggcgctggaagattattaccaggagcagggtcgggactgggaacgctatgcgatggtgaaagcgcggatcatgggcgataacgacggcgtgtacgccagcgagttgcgcgcgatgctccgtcctttcgtcttccgccgttatatcgacttcagcgtgatccagtcgctgcgtaacatgaaaggcatgatcgcccgcgaagtgcggcgtcgcgggctgaaagacaacatcaagctcggcgccggcgggatccgtgaaattgagtttatcgttcaggtctttcaactgatccgcggtggtcgcgaacctgcactgcagcagcgcgccctgctgccgacgctggcggcgattgatgagctacatctgctgccggaaggcgacgcggcgctgctgcgcgaggcctatctgttcctgcgccggctggaaaacctgctgcaaagcatcaacgatgagcagacccagaccctgccgcaggatgaacttaaccgcgccaggctggcgtgggggatgcataccgaagactgggagacgctgagcgcgcagctggcgagccagatggccaacgtgcggcgagtgtttaatgaactgatcggcgatgatgaggatcagtccccggatgagcaactggccgagtactggcgcgagctgtggcaggatgcgctggaagaagatgacgccagcccggcgctggcgcatttaaacgataccgaccgccgtagcgtgctggcgctgattgccgattttcgtaaagagctggatcggcgcaccatcggcccgcgcggccgccaggtgctggatcagctgatgccgcatctgctgagcgaaatctgctcgcgcgccgatgcgccgctgcctctggcgcggatcacgccgctgttgaccgggatcgtcacccgtaccacctatcttgagctgctgagcgaattccccggcgcgctgaagcacctgatcacgctctgcgcggcgtcgccgatggtcgccagccagctggcgcgccacccgctgctgctggatgagctgctggatcccaacaccctctatcagccgacggcgaccgatgcctatcgcgacgagctgcgccagtacctgctgcgcgtgccggaagaggatgaagagcagcagctggaggcgttgcgccagtttaagcaggcgcagcagctgcatatcgcggcggcggatatcgctggtaccctgccggtgatgaaggtcagcgatcacttaacctggcttgccgaagcgatcctcgacgcggtggtgcagcaggcatgggggcagatggtcgctcgctacggccagccgacccacctgcacgatcgccagggtcgcggcttcgccgtcgtcggctacggtaagcttggcggctgggagctgggctacagctccgatctcgatctggtgttcctccatgactgcccggcggaggtgatgaccgacggcgagcgggagattgacggccgtcagttctacctgcggctggcccagcggatcatgcacctgttcagcacccgcacctcgtccggtattctctacgaagtggacgcccggctgcgtccttctggcgcggcggggatgctggtcaccaccgccgacgcgtttgctgactatcagcagaacgaagcctggacgtgggaacatcaggcgctggtgcgcgcccgcgtggtctatggcgacccggcgctgcaggcgcgctttgacgccattcgtcgcgatatcctgaccaccccgcgggaggggatgaccctgcagaccgaggttcgcgagatgcgcgagaagatgcgcgcccaccttggcaacaaacatcccgatcgttttgatatcaaagccgatgccggcgggatcaccgatattgaatttattactcagtatctggtcctacgctatgccagtgacaagccgaagctgacccgctggtctgacaacgtgcgtattcttgagctgctggcgcagaacgacatcatggacgaggaggaggcgcgcgccttaacgcatgcgtacaccaccttgcgtgatgcgctccatcacctggccctgcaggagcagccgggacacgtggcgccagaggccttcagccgggagcgtcagcaggtcagcgccagctggcagaagtggctgatggcttaa

SEQ ID NO: 6 - DNA, 1410 bp, Klebsiella variicola
  source 1..1410: mol_type = genomic DNA
atgtccgctgaacacgttttgacgatgctgaacgagcacgaagtgaagtttgtcgatctgcgcttcaccgataccaaaggtaaagaacagcacgtcacgattccgtctcatcaggtaaatgccgaattcttcgaagaaggcaaaatgtttgatggctcctcgatcggtggctggaaaggtatcaacgaatctgacatagtcctgatgccggacgcttccaccgcggtcattgacccgttctacgaagaaccgaccctgatcatccgctgcgatatcctcgagccaggcaccctgcagggctatgaccgtgacccgcgctccatcgcgaaacgcgctgaagagtacctgcgcgccaccggcatcgctgacaccgtcctgttcgggccagaaccagaattcttcctgttcgacgacatccgttttggcgcctctatctccggctcccatgtcgcgatcgatgacatcgaaggtgcgtggaactcctccaccaaatacgaaggcggtaacaaaggtcaccgtccgggcgtgaaagggggttacttcccggttccgccagtagactcttctcaggatatccgttccaccatgtgtatgatcatggaagagatgggcctggttgttgaagctcaccaccacgaagtggctaccgcaggtcagaacgaagtggcaacccgcttcaacaccatgaccaaaaaagcggacgaaattcagatttacaaatacgttgttcacaacgtcgctcaccgcttcggtaaaaccgcgacctttatgccgaaaccaatgttcggcgataacggctccggcatgcactgccatatgtctctggcgaaaaacgggactaacctgttctccggcgacaaatacgcaggtctgtctgagcaggcgctgttctacatcggcggcgtaatcaaacacgctaaagctatcaacgccctggcgaacccgaccaccaactcctacaagcgtctggtcccgggttacgaagcgccggttatgctggcttactctgcgcgtaaccgctctgcctccatccgtattccggtggtgacctctccgaaagcgcgtcgtatcgaagtgcgcttcccggacccggctgccaacccgtacctgtgctttgcggcgctgctgatggctggccttgatggtatcaagaacaaaatccatccgggcgaagcaatggacaaaaacctgtatgacctgccgccggaagaagcgaaagagatcccgcaggttgcaggctctctggaagaagccctgcaggccctggatgctgaccgcgagttcctgacggctggcggcgtgttcaccaatgacgctatcgatgcttacatcgccctgcgtctggaagagaacgaccgcgtacgcatgactccgcatccggttgagttcgaactgtactacagcgtctaa

SEQ ID NO: 7 - DNA, 1410 bp, Klebsiella variicola
  source 1..1410: mol_type = genomic DNA
atgtccgctgaacacgttttgacgatgctgaacgagcacgaagtgaagtttgtcgatctgcgcttcaccgataccaaaggtaaagaacagcacgtcacgattccgtctcatcaggtaaatgccgaattcttcgaagaaggcaaaatgtttgatggctcctcgatcggtggctggaaaggtatcaacgaatctgacgtggtcctgatgccggacgcttccaccgcggtcattgacccgttctacgaagaaccgaccctgatcatccgctgcgatatcctcgagccaggcaccctgcagggctatgaccgtgacccgcgctccatcgcgaaacgcgctgaagagtacctgcgcgccaccggcatcgctgacaccgtcctgttcgggccagaaccagaattcttcctgttcgacgacatccgttttggcgcctctatctccggctcccatgtcgcgatcgatgacatcgaaggtgcgtggaactcctccaccaaatacgaaggcggtaacaaaggtcaccgtccgggcgtgaaagggggttacttcccggttccgccagtagactcttctcaggatatccgttccaccatgtgtatgatcatggaagagatgggcctggttgttgaagctcaccaccacgaagtggctaccgcaggtcagaacgaagtggcaacccgcttcaacaccatgaccaaaaaagcggacgaaattcagatttacaaatacgttgttcacaacgtcgctcaccgcttcggtaaaaccgcgacctttatgccgaaaccaatgttcggcgataacggctccggcatgcactgccatatgtctctggcgaaaaacgggactaacctgttctccggcgacaaatacgcaggtctgtctgagcaggcgctgttctacatcggcggcgtaatcaaacacgctaaagctatcaacgccctggcgaacccgaccaccaactcctacaagcgtctggtcccgggttacgaagcgccggttatgctggcttactctgcgcgtaaccgctctgcctccatccgtattccggtggtgacctctccgaaagcgcgtcgtatcgaagtgcgcttcccggacccggctgccaacccgtacctgtgctttgcggcgctgctgatggctggccttgatggtatcaagaacaaaatccatccgggcgaagcaatggacaaaaacctgtatgacctgccgccggaagaagcgaaagagatcccgcaggttgcaggctctctggaagaagccctgcaggccctggatgctgaccgcgagttcctgacggctggcggcgtgttcaccaatgacgctatcgatgcttacatcgccctgcgtctggaagagaacgaccgcgtacgcatgactccgcatccggttgagttcgaactgtactacagcgtctaa

SEQ ID NO: 8 - DNA, 1410 bp, Klebsiella variicola
  source 1..1410: mol_type = genomic DNA
atgtccgctgaacacgttttgacgatgctgaacgagcacgaagtgaagtttgtcgatctgcgcttcaccgataccaaaggtaaagaacagcacgtcacgattccgtctcatcaggtaaatgccgaattcttcgaagaaggcaaaatgtttgatggctcctcgatcggtggctggaaaggtatcaacgaatctgacatggtcctgatgccggacgcttccaccgcggtcattgacccgttctacgaagaaccgaccctgatcatccgctgcgatatcctcgagccaggcaccctgcagggctatgaccgtgacccgcgctccatcgcgaaacgcgctgaagagtacctgcgcgccaccggcatcgctgacaccgtcctgttcgggccagaaccagaattcttcctgttcgacgacatccgttttggcgcctctatctccggctcccatgtcgcgatcgatgacatcgaatgtgcgtggaactcctccaccaaatacgaaggcggtaacaaaggtcaccgtccgggcgtgaaagggggttacttcccggttccgccagtagactcttctcaggatatccgttccaccatgtgtatgatcatggaagagatgggcctggttgttgaagctcaccaccacgaagtggctaccgcaggtcagaacgaagtggcaacccgcttcaacaccatgaccaaaaaagcggacgaaattcagatttacaaatacgttgttcacaacgtcgctcaccgcttcggtaaaaccgcgacctttatgccgaaaccaatgttcggcgataacggctccggcatgcactgccatatgtctctggcgaaaaacgggactaacctgttctccggcgacaaatacgcaggtctgtctgagcaggcgctgttctacatcggcggcgtaatcaaacacgctaaagctatcaacgccctggcgaacccgaccaccaactcctacaagcgtctggtcccgggttacgaagcgccggttatgctggcttactctgcgcgtaaccgctctgcctccatccgtattccggtggtgacctctccgaaagcgcgtcgtatcgaagtgcgcttcccggacccggctgccaacccgtacctgtgctttgcggcgctgctgatggctggccttgatggtatcaagaacaaaatccatccgggcgaagcaatggacaaaaacctgtatgacctgccgccggaagaagcgaaagagatcccgcaggttgcaggctctctggaagaagccctgcaggccctggatgctgaccgcgagttcctgacggctggcggcgtgttcaccaatgacgctatcgatgcttacatcgccctgcgtctggaagagaacgaccgcgtacgcatgactccgcatccggttgagttcgaactgtactacagcgtctaa

SEQ ID NO: 9 - DNA, 1410 bp, Klebsiella variicola
  source 1..1410: mol_type = genomic DNA
atgtccgctgaacacgttttgacgatgctgaacgagcacgaagtgaagtttgtcgatctgcgcttcaccgataccaaaggtaaagaacagcacgtcacgattccgtctcatcaggtaaatgccgaattcttcgaagaaggcaaaatgtttgatggctcctcgatcggtggctggaaaggtatcaacgaatctgacatggtcctgatgccggacgcttccaccgcggtcattgacccgttctacgaagaaccgaccctgatcatccgctgcgatatcctcgagccaggcaccctgcagggctatgaccgtgacccgcgctccatcgcgaaacgcgctgaagagtacctgcgcgccaccggcatcgctgacaccgtcctgttcgggccagaaccagaattcttcctgttcgacgacatccgttttggcgcctctatctccggctcccatgtcgcgatcgatgacatcgaaggtgcgtggaactcctccaccaaatacgaaggcggtaacaaaggtcaccgtctgggcgtgaaagggggttacttcccggttccgccagtagactcttctcaggatatccgttccaccatgtgtatgatcatggaagagatgggcctggttgttgaagctcaccaccacgaagtggctaccgcaggtcagaacgaagtggcaacccgcttcaacaccatgaccaaaaaagcggacgaaattcagatttacaaatacgttgttcacaacgtcgctcaccgcttcggtaaaaccgcgacctttatgccgaaaccaatgttcggcgataacggctccggcatgcactgccatatgtctctggcgaaaaacgggactaacctgttctccggcgacaaatacgcaggtctgtctgagcaggcgctgttctacatcggcggcgtaatcaaacacgctaaagctatcaacgccctggcgaacccgaccaccaactcctacaagcgtctggtcccgggttacgaagcgccggttatgctggcttactctgcgcgtaaccgctctgcctccatccgtattccggtggtgacctctccgaaagcgcgtcgtatcgaagtgcgcttcccggacccggctgccaacccgtacctgtgctttgcggcgctgctgatggctggccttgatggtatcaagaacaaaatccatccgggcgaagcaatggacaaaaacctgtatgacctgccgccggaagaagcgaaagagatcccgcaggttgcaggctctctggaagaagccctgcaggccctggatgctgaccgcgagttcctgacggctggcggcgtgttcaccaatgacgctatcgatgcttacatcgccctgcgtctggaagagaacgaccgcgtacgcatgactccgcatccggttgagttcgaactgtactacagcgtctaa

SEQ ID NO: 10 - DNA, 1410 bp, Klebsiella variicola
  source 1..1410: mol_type = genomic DNA
atgtccgctgaacacgttttgacgatgctgaacgagcacgaagtgaagtttgtcgatctgcgcttcaccgataccaaaggtaaagaacagcacgtcacgattccgtctcatcaggtaaatgccgaattcttcgaagaaggcaaaatgtttgatggctcctcgatcggtggctggaaaggtatcaacgaatctgacatggtcctgatgccggacgcttccaccgcggtcattgacccgttctacgaagaaccgaccctgatcatccgctgcgatatcctcgagccaggcaccctgcagggctatgaccgtgacccgcgctccatcgcgaaacgcgctgaagagtacctgcgcgccaccggcatcgctgacaccgtcctgttcgggccagaaccagaattcttcctgttcgacgacatccgttttggcgcctctatctccggctcccatgtcgcgatcgatgacatcgaaggtgcgtggaactcctccaccaaatacgaaggcggtaacaaaggtcaccgtccgggcgtgaaagggggttacttcccggttccgccagtagactcttttcaggatatccgttccaccatgtgtatgatcatggaagagatgggcctggttgttgaagctcaccaccacgaagtggctaccgcaggtcagaacgaagtggcaacccgcttcaacaccatgaccaaaaaagcggacgaaattcagatttacaaatacgttgttcacaacgtcgctcaccgcttcggtaaaaccgcgacctttatgccgaaaccaatgttcggcgataacggctccggcatgcactgccatatgtctctggcgaaaaacgggactaacctgttctccggcgacaaatacgcaggtctgtctgagcaggcgctgttctacatcggcggcgtaatcaaacacgctaaagctatcaacgccctggcgaacccgaccaccaactcctacaagcgtctggtcccgggttacgaagcgccggttatgctggcttactctgcgcgtaaccgctctgcctccatccgtattccggtggtgacctctccgaaagcgcgtcgtatcgaagtgcgcttcccggacccggctgccaacccgtacctgtgctttgcggcgctgctgatggctggccttgatggtatcaagaacaaaatccatccgggcgaagcaatggacaaaaacctgtatgacctgccgccggaagaagcgaaagagatcccgcaggttgcaggctctctggaagaagccctgcaggccctggatgctgaccgcgagttcctgacggctggcggcgtgttcaccaatgacgctatcgatgcttacatcgccctgcgtctggaagagaacgaccgcgtacgcatgactccgcatccggttgagttcgaactgtactacagcgtctaa

SEQ ID NO: 11 - DNA, 1410 bp, Klebsiella variicola
  source 1..1410: mol_type = genomic DNA
atgtccgctgaacacgttttgacgatgctgaacgagcacgaagtgaagtttgtcgatctgcgcttcaccgataccaaaggtaaagaacagcacgtcacgattccgtctcatcaggtaaatgccgaattcttcgaagaaggcaaaatgtttgatggctcctcgatcggtggctggaaaggtatcaacgaatctgacatggtcctgatgccggacgcttccaccgcggtcattgacccgttctacgaagaaccgaccctgatcatccgctgcgatatcctcgagccaggcaccctgcagggctatgaccgtgacccgcgctccatcgcgaaacgcgctgaagagtacctgcgcgccaccggcatcgctgacaccgtcctgttcgggccagaaccagaattcttcctgttcgacgacatccgttttggcgcctctatctccggctcccatgtcgcgatcgatgacatcgaaggtgcgtggaactcctccaccaaatacgaaggcggtaacaaaggtcaccgtccgggcgtgaaagggggttacttcccggttccgccagtagactcttctcaggatatccgttccaccatgtgtatgatcatggaagagatgggcctggttgttaaagctcaccaccacgaagtggctaccgcaggtcagaacgaagtggcaacccgcttcaacaccatgaccaaaaaagcggacgaaattcagatttacaaatacgttgttcacaacgtcgctcaccgcttcggtaaaaccgcgacctttatgccgaaaccaatgttcggcgataacggctccggcatgcactgccatatgtctctggcgaaaaacgggactaacctgttctccggcgacaaatacgcaggtctgtctgagcaggcgctgttctacatcggcggcgtaatcaaacacgctaaagctatcaacgccctggcgaacccgaccaccaactcctacaagcgtctggtcccgggttacgaagcgccggttatgctggcttactctgcgcgtaaccgctctgcctccatccgtattccggtggtgacctctccgaaagcgcgtcgtatcgaagtgcgcttcccggacccggctgccaacccgtacctgtgctttgcggcgctgctgatggctggccttgatggtatcaagaacaaaatccatccgggcgaagcaatggacaaaaacctgtatgacctgccgccggaagaagcgaaagagatcccgcaggttgcaggctctctggaagaagccctgcaggccctggatgctgaccgcgagttcctgacggctggcggcgtgttcaccaatgacgctatcgatgcttacatcgccctgcgtctggaagagaacgaccgcgtacgcatgactccgcatccggttgagttcgaactgtactacagcgtctaa

SEQ ID NO: 12 - DNA, 1410 bp, Klebsiella variicola
  source 1..1410: mol_type = genomic DNA
atgtccgctgaacacgttttgacgatgctgaacgagcacgaagtgaagtttgtcgatctgcgcttcaccgataccaaaggtaaagaacagcacgtcacgattccgtctcatcaggtaaatgccgaattcttcgaagaaggcaaaatgtttgatggctcctcgatcggtggctggaaaggtatcaacgaatctgacatggtcctgatgccggacgcttccaccgcggtcattgacccgttctacgaagaaccgaccctgatcatccgctgcgatatcctcgagccaggcaccctgcagggctatgaccgtgacccgcgctccatcgcgaaacgcgctgaagagtacctgcgcgccaccggcatcgctgacaccgtcctgttcgggccagaaccagaattcttcctgttcgacgacatccgttttggcgcctctatctccggctcccatgtcgcgatcgatgacatcgaaggtgcgtggaactcctccaccaaatacgaaggcggtaacaaaggtcaccgtccgggcgtgaaagggggttacttcccggttccgccagtagactcttctcaggatatccgttccaccatgtgtatgatcatggaagagatgggcctggttgttgaagctcaccaccacgaagtggctaccgcaggtcagaacgaagtggcaacccgcttcaacaccatgaccaaaaaagcggacgaaattcagatttacaaatacgttgttcacaacgtcgctcaccgcttcggtaaaaccgcgacctttatgccgaaaccaatgttcggcgataacggctccagcatgcactgccatatgtctctggcgaaaaacgggactaacctgttctccggcgacaaatacgcaggtctgtctgagcaggcgctgttctacatcggcggcgtaatcaaacacgctaaagctatcaacgccctggcgaacccgaccaccaactcctacaagcgtctggtcccgggttacgaagcgccggttatgctggcttactctgcgcgtaaccgctctgcctccatccgtattccggtggtgacctctccgaaagcgcgtcgtatcgaagtgcgcttcccggacccggctgccaacccgtacctgtgctttgcggcgctgctgatggctggccttgatggtatcaagaacaaaatccatccgggcgaagcaatggacaaaaacctgtatgacctgccgccggaagaagcgaaagagatcccgcaggttgcaggctctctggaagaagccctgcaggccctggatgctgaccgcgagttcctgacggctggcggcgtgttcaccaatgacgctatcgatgcttacatcgccctgcgtctggaagagaacgaccgcgtacgcatgactccgcatccggttgagttcgaactgtactacagcgtctaa

SEQ ID NO: 13 - DNA, 1410 bp, Klebsiella variicola
  source 1..1410: mol_type = genomic DNA
atgtccgctgaacacgttttgacgatgctgaacgagcacgaagtgaagtttgtcgatctgcgcttcaccgataccaaaggtaaagaacagcacgtcacgattccgtctcatcaggtaaatgccgaattcttcgaagaaggcaaaatgtttgatggctcctcgatcggtggctggaaaggtatcaacgaatctgacatggtcctgatgccggacgcttccaccgcggtcattgacccgttctacgaagaaccgaccctgatcatccgctgcgatatcctcgagccaggcaccctgcagggctatgaccgtgacccgcgctccatcgcgaaacgcgctgaagagtacctgcgcgccaccggcatcgctgacaccgtcctgttcgggccagaaccagaattcttcctgttcgacgacatccgttttggcgcctctatctccggctcccatgtcgcgatcgatgacatcgaaggtgcgtggaactcctccaccaaatacgaaggcggtaacaaaggtcaccgtccgggcgtgaaagggggttacttcccggttccgccagtagactcttctcaggatatccgttccaccatgtgtatgatcatggaagagatgggcctggttgttgaagctcaccaccacgaagtggctaccgcaggtcagaacgaagtggcaacccgcttcaacaccatgaccaaaaaagcggacgaaattcagatttacaaatacgttgttcacaacgtcgctcaccgcttcggtaaaaccgcgacctttatgccgaaaccaatgttcggcgataacggctccggcatgcactgccatatgtctctggcgaaaaacgggactaacctgttctccggcgacaaatacgcaggtctgtctgagcaggcgctgttctacatcggcggcgtaatcaaacacgctaaagctatcaacgccctggcgaacccgaccaccaactcctacaagcgtctggtcccgggttacgaagcgccggttatgctggcttactctgcgcgtgaccgctctgcctccatccgtattccggtggtgacctctccgaaagcgcgtcgtatcgaagtgcgcttcccggacccggctgccaacccgtacctgtgctttgcggcgctgctgatggctggccttgatggtatcaagaacaaaatccatccgggcgaagcaatggacaaaaacctgtatgacctgccgccggaagaagcgaaagagatcccgcaggttgcaggctctctggaagaagccctgcaggccctggatgctgaccgcgagttcctgacggctggcggcgtgttcaccaatgacgctatcgatgcttacatcgccctgcgtctggaagagaacgaccgcgtacgcatgactccgcatccggttgagttcgaactgtactacagcgtctaa

SEQ ID NO: 14 - DNA, 2838 bp, Klebsiella variicola
  source 1..2838: mol_type = genomic DNA
atgatgccgctttctccgcaattacagcagcactggcagacggtcgctgaccgtctgccagcggattttcccattgccgaactgagcccacaggccaggtcggtcatggcgttcagcgattttgtcgaacagagtgtgatcgcccagccgggctggctgaatgagcttgcggactcctcgccggaggcggaagagtggcggcattacgaggcctggctgcaggatcgcctgcaggccgtcactgacgaagcggggttgatgcgagagctgcgtctcttccgccgccagatgatggtccgcatcgcctgggcgcaggcgctgtcgctggtgagcgaagaagagactctgcagcagctgagcgtcctggcggagaccctgattgtcgccgcccgcgactggctgtacgccgcctgctgtaaggagtggggaacgccatgcaatgccgagggccagccgcagccgctgctgatcctcgggatgggaaagctgggcggcggcgagctgaacttctcttccgatatcgatctgatctttgcctggcctgagcatggcgccacccgcggcggccgccgcgagctggataacgcccagttctttacccgtctggggcagcggctgatcaaggcccttgaccagccgacgcaggacggctttgtctatcgggttgacatgcgcctgcggccgtttggcgacagtgggccgctggtactcagttttgcggcgctggaagattattaccaggagcagggtcgggactgggaacgctatgcgatggtgaaagcgcggatcatgggcgataacgacggcgtgtacgccagcgagttgcgcgcgatgctccgtcctttcgtcttccgccgttatatcgacttcagcgtgatccagtcgctgcgtaacatgaaaggcatgatcgcccgcgaagtgcggcgtcgcgggctgaaagacaacatcaagctcggcgccggcgagatccgtgaaattgagtttatcgttcaggtctttcaactgatccgcggtggtcgcgaacctgcactgcagcagcgcgccctgctgccgacgctggcggcgattgatgagctacatctgctgccggaaggcgacgcggcgctgctgcgcgaggcctatctgttcctgcgccggctggaaaacctgctgcaaagcatcaacgatgagcagacccagaccctgccgcaggatgaacttaaccgcgccaggctggcgtgggggatgcataccgaagactgggagacgctgagcgcgcagctggcgagccagatggccaacgtgcggcgagtgtttaatgaactgatcggcgatgatgaggatcagtccccggatgagcaactggccgagtactggcgcgagctgtggcaggatgcgctggaagaagatgacgccagcccggcgctggcgcatttaaacgataccgaccgccgtagcgtgctggcgctgattgccgattttcgtaaagagctggatcggcgcaccatcggcccgcgcggccgccaggtgctggatcagctgatgccgcatctgctgagcgaaatctgctcgcgcgccgatgcgccgctgcctctggcgcggatcacgccgctgttgaccgggatcgtcacccgtaccacctatcttgagctgctgagcgaattccccggcgcgctgaagcacctgatcacgctctgcgcggcgtcgccgatggtcgccagccagctggcgcgccacccgctgctgctggatgagctgctggatcccaacaccctctatcagccgacggcgaccgatgcctatcgcgacgagctgcgccagtacctgctgcgcgtgccggaagaggatgaagagcagcagctggaggcgttgcgccagtttaagcaggcgcagcagctgcatatcgcggcggcggatatcgctggtaccctgccggtgatgaaggtcagcgatcacttaacctggcttgccgaagcgatcctcgacgcggtggtgcagcaggcatgggggcagatggtcgctcgctacggccagccgacccacctgcacgatcgccagggtcgcggcttcgccgtcgtcggctacggtaagcttggcggctgggagctgggctacagctccgatctcgatctggtgttcctccatgactgcccggcggaggtgatgaccgacggcgagcgggagattgacggccgtcagttctacctgcggctggcccagcggatcatgcacctgttcagcacccgcacctcgtccgatattctctacgaagtggacgcccggctgcgtccttctggcgcggcggggatgctggtcaccaccgccgacgcgtttgctgactatcagcagaacgaagcctggacgtgggaacatcaggcgctggtgcgcgcccgcgtggtctatggcgacccggcgctgcaggcgcgctttgacgccattcgtcgcgatatcctgaccaccccgcgggaggggatgaccctgcagaccgaggttcgcgagatgcgcgagaagatgcgcgcccaccttggcaacaaacatcccgatcgttttgatatcaaagccgatgccggcgggatcaccgatattgaatttattactcagtatctggtcctacgctatgccagtgacaagccgaagctgacccgctggtctgacaacgtgcgtattcttgagctgctggcgcagaacgacatcatggacgaggaggaggcgcgcgccttaacgcatgcgtacaccaccttgcgtgatgcgctccatcacctggccctgcaggagcagccgggacacgtggcgccagaggccttcagccgggagcgtcagcaggtcagcgccagctggcagaagtggctgatggcttaa

SEQ ID NO: 15 - DNA, 1410 bp, Kosakonia sacchari
  source 1..1410: mol_type = genomic DNA
atgtccgctgaacacgttttgacgatgctgaatgagcacgaagtgaagtttgtcgatctgcgcttcaccgataccaaaggtaaagaacagcacgtcactatcccagcccatcaagtaaacgctgacttctttgaagaaggtaaaatgtttgatggttcttcgattggtggctggaaaggcatcaacgaatcagacatagtgctgatgccggacgcgactaccgctgtcattgacccgttctacgaagagtctacgctgatcattcgttgcgatatcctcgaaccaggcaccatgcagggctacgatcgtgacccgcgctccatcgctaaacgcgctgaagaatacctgcgctccaccggtctggcagacaccgttctgtttgggccagagccggaattcttcctgttcgacgacgtgcgcttcggcagctccatttccggttccagcgtcgctatcgacgatatcgaaggcgcatggaacacctccaccaaatacgaaggtggtaacaaaggtcaccgtccggcagtgaaaggcggttacttcccggttccgccggtcgattcatcacaggatctgcgttccaccatgtgtctggtgatggaagagatgggcctggttgttgaagctcaccaccacgaagtggcaacggctggtcagaacgaagtggctacccgcttcaacaccatgaccaaaaaagcggacgaaattcagatttacaaatacgtggtgcacaacgttgctcaccgctttggtaaaaccgcgacctttatgccgaaaccgatgttcggtgataacggttccggtatgcactgccacatgtctctgtccaagaacggtaccaacctgttctctggcgacaaatacgctggcctgtctgaaatggcgctgtactacattggcggcgtaatcaaacacgcaaaagcgatcaacgccctgtctaacccgaccaccaactcctacaagcgtctggtcccgggttatgaagcgccggtaatgctggcttactctgcacgtaaccgttctgcatctatccgtattccggtagtggcttctccgaaagcgcgtcgtatcgaagtgcgcttcccggacccggctgctaacccgtatctgtgcttcgcagcgctgttgatggctggtctggatggcattaaaaacaaaatccacccgggcgaagcgatggacaaaaacctgtatgacctgccggctgaagaagcgaaagaaatcccgcaggttgcaggctctctggaagaagcgctcaacgcgctggatgctgaccgcgagttcctgactgctggtggcgtattcaccgacgacgctatcgatgcttacatcgcgctgcgtatcgaagagaacgaccgtgttcgcatgacgccgcacccggtagagttcgaactgtactacagcgtttaa

SEQ ID NO: 16 - DNA, 1410 bp, Metakosakonia intestini
  source 1..1410: mol_type = genomic DNA
atgtccgctgaacacgttttgacgatgctgaacgagcacgaagtcaagtttgttgatttgcgcttcaccgacactaaaggtaaagagcagcacgtcacgatcccagctcatcaggtaaatgctgacttctttgaagaaggcaaaatgtttgatggctcctcgattggtggctggaaaggcattaacgaatctgacatggtactgatgccggacgcctctaccgctgtcattgacccgttctacgaagaaccgactctgatcatccgttgcgatatcctcgagccgggcactatgcagggctacgatcgcgacccgcgctccatcgcaaaacgcgctgaagaatacctgcgcgcaacaggcatcgcagacaccgttctgttcgggcctgagccagagttcttcctgttcgacgacgtacgtttcggcagctctatttccggttcccacgtcgctatcgatgatatcgaaggcgcatggaactcctccaccaaatacgaaggcggtaacaaaggtctccgtccggctgttaaaggtggttacttcccggttccgccagtggactccgcacaagacctgcgttccactatgtgtctggtaatggaagagatgggcctggttgttgaagctcatcaccacgaagttgcaactgctggtcagaacgaagtggcaacccgcttcaataccatgaccaaaaaagcagacgaaattcagatctacaaatatgtggttcacaacgttgctcaccgcttcggtaaaaccgcaacctttatgccaaaaccgatgttcggtgataacggttccggtatgcactgccacatgtctctgtccaagaacggcgtaaacctgttctctggcgacaaatatgctggtctgtctgagcaggcgctgtactacatcggcggcgttattaaacacgctaaagcgatcaacgccctgtccaacccgactaccaactcctacaaacgtctggtcccgggttacgaagcaccggtcatgctggcttactctgcccgtaaccgttcagcttccatccgtattccggtggttgcatctccgaaagcgcgtcgtattgaagtgcgtttcccggacccggctgctaacccgtacctgtgcttcgcagcgctgctgatggctggtcttgatggcatcaagaacaaaatccatccgggcgaagcgatggacaaaaacctgtatgacctgccagctgaagaagcgaaagaaatcccgcaggttgcaggctctctggaagaagcactgaactgcctgaacgaagaccgcgagttcctgactgcgggtggcgtattcaccgacgacgcaattgatgcttacatcgctctgcgtatcgaagaaaacgaccgcgtacgcatgacgccgcacccggtagagttcgagctgtactacagcgtttaa

SEQ ID NO: 17 - DNA, 1410 bp, Metakosakonia intestini
  source 1..1410: mol_type = genomic DNA
atgtccgctgaacacgttttgacgatgctgaacgagcacgaagtcaagtttgttgatttgcgcttcaccgacactaaaggtaaagagcagcacgtcacgatcccagctcatcaggtaaatgctgacttctttgaagaaggcaaaatgtttgatggctcctcgattggtggctggaaaggcattaacgaatctgacatggtactgatgccggacgcctctaccgctgtcattgacccgttctacgaagaaccgactctgatcatccgttgcgatatcctcgagccgggcactatgcagggctacgatcgcgacccgcgctccatcgcaaaacgcgctgaagaatacctgcgcgcaacaggcatcgcagacaccgttctgttcgggcctgagccagagttcttcctgttcgacgacgtacgtttcggcagctctatttccggttcccacgtcgctatcgatgatatcgaaggcgcatggaactcctccaccaaatacgaaggcggtaacaaaggtcaccgtccggctgttaaaggtggttacttcccggttccgccagtggactccgcacaagacctgcgttccactatgtgtctggtaatggaagagatgggcctggttgttgaagctcatcaccacgaagttgcaactgctagtcagaacgaagtggcaacccgcttcaataccatgaccaaaaaagcagacgaaattcagatctacaaatatgtggttcacaacgttgctcaccgcttcggtaaaaccgcaacctttatgccaaaaccgatgttcggtgataacggttccggtatgcactgccacatgtctctgtccaagaacggcgtaaacctgttctctggcgacaaatatgctggtctgtctgagcaggcgctgtactacatcggcggcgttattaaacacgctaaagcgatcaacgccctgtccaacccgactaccaactcctacaaacgtctggtcccgggttacgaagcaccggtcatgctggcttactctgcccgtaaccgttcagcttccatccgtattccggtggttgcatctccgaaagcgcgtcgtattgaagtgcgtttcccggacccggctgctaacccgtacctgtgcttcgcagcgctgctgatggctggtcttgatggcatcaagaacaaaatccatccgggcgaagcgatggacaaaaacctgtatgacctgccagctgaagaagcgaaagaaatcccgcaggttgcaggctctctggaagaagcactgaactgcctgaacgaagaccgcgagttcctgactgcgggtggcgtattcaccgacgacgcaattgatgcttacatcgctctgcgtatcgaagaaaacgaccgcgtacgcatgacgccgcacccggtagagttcgagctgtactacagcgtttaa

SEQ ID NO: 18 - DNA, 1410 bp, Metakosakonia intestini
  source 1..1410: mol_type = genomic DNA
atgtccgctgaacacgttttgacgatgctgaacgagcacgaagtcaagtttgttgatttgcgcttcaccgacactaaaggtaaagagcagcacgtcacgatcccagctcatcaggtaaatgctgacttctttgaagaaggcaaaatgtttgatggctcctcgattggtggctggaaaggcattaacgaatctgacatggtactgatgccggacgcctctaccgctgtcattgacccgttctacgaagaaccgactctgatcatccgttgcgatatcctcgagccgggcactatgcagggctacgatcgcgacccgcgctccatcgcaaaacgcgctgaagaatacctgcgcgcaacaggcatcgcagacaccgttctgttcgggcctgagccagagttcttcctgttcgacgacgtacgtttcggcagctctatttccggttcccacgtcgctatcgatgatatcgaaggcgcatggaactcctccaccaaatacgaaggcggtaacaaaggtcaccgtccggctgttaaaggtggttacttcccggttccgccagtggactccgcacaagacctgcgttccactatgtgtctggtaatggaagagatgggcctggttgttgaagctcatcaccacgaagttgcaactgctggtcagaacgaagtggcaacccgcttcaataccatgaccaaaaaagcagacgaaattcagatctacaaatatgtggttcacaacgttgctcaccgcttcggtaaaaccgcaatctttatgccaaaaccgatgttcggtgataacggttccggtatgcactgccacatgtctctgtccaagaacggcgtaaacctgttctctggcgacaaatatgctggtctgtctgagcaggcgctgtactacatcggcggcgttattaaacacgctaaagcgatcaacgccctgtccaacccgactaccaactcctacaaacgtctggtcccgggttacgaagcaccggtcatgctggcttactctgcccgtaaccgttcagcttccatccgtattccggtggttgcatctccgaaagcgcgtcgtattgaagtgcgtttcccggacccggctgctaacccgtacctgtgcttcgcagcgctgctgatggctggtcttgatggcatcaagaacaaaatccatccgggcgaagcgatggacaaaaacctgtatgacctgccagctgaagaagcgaaagaaatcccgcaggttgcaggctctctggaagaagcactgaactgcctgaacgaagaccgcgagttcctgactgcgggtggcgtattcaccgacgacgcaattgatgcttacatcgctctgcgtatcgaagaaaacgaccgcgtacgcatgacgccgcacccggtagagttcgagctgtactacagcgtttaa

SEQ ID NO: 19 - DNA, 1410 bp, Metakosakonia intestini
  source 1..1410: mol_type = genomic DNA
atgtccgctgaacacgttttgacgatgctgaacgagcacgaagtcaagtttgttgatttgcgcttcaccgacactaaaggtaaagagcagcacgtcacgatcccagctcatcaggtaaatgctgacttctttgaagaaggcaaaatgtttgatggctcctcgattggtggctggaaaggcattaacgaatctgacatggtactgatgccggacgcctctaccgctgtcattgacccgttctacgaagaaccgactctgatcatccgttgcgatatcctcgagccgggcactatgcagggctacgatcgcgacccgcgctccatcgcaaaacgcgctgaagaatacctgcgcgcaacaggcatcgcagacaccgttctgttcgggcctgagccagagttcttcctgttcgacgacgtacgtttcggcagctctatttccggttcccacgtcgctatcgatgatatcgaaggcgcatggaactcctccaccaaatacgaaggcggtaacaaaggtcaccgtccggctgttaaaggtggttacttcccggttccgccagtggactccgcacaagacctgcgttccactatgtgtctggtaatggaagagatgggcctggttgttgaagctcatcaccacgaagttgcaactgctggtcagaacgaagtggcaacccgcttcaataccatgaccaaaaaagcagacgaaattcagatctacaaatatgtggttcacaacgttgctcaccgcttcggtaaaaccgcaacctttatgccaaaaccgatgttcggtgataacggttccggtatgcactgccacatgtctctgtccaagaacggcgtaaacctgttctctggcgacaaatatgctggtctgtctgagcaggcgctgtactacatcggcggcgttattaaacacgctaaagcgatcaacgccctgtccaacccgactaccaactcctacaaacgtctggtcccgggttacgaagtaccggtcatgctggcttactctgcccgtaaccgttcagcttccatccgtattccggtggttgcatctccgaaagcgcgtcgtattgaagtgcgtttcccggacccggctgctaacccgtacctgtgcttcgcagcgctgctgatggctggtcttgatggcatcaagaacaaaatccatccgggcgaagcgatggacaaaaacctgtatgacctgccagctgaagaagcgaaagaaatcccgcaggttgcaggctctctggaagaagcactgaactgcctgaacgaagaccgcgagttcctgactgcgggtggcgtattcaccgacgacgcaattgatgcttacatcgctctgcgtatcgaagaaaacgaccgcgtacgcatgacgccgcacccggtagagttcgagctgtactacagcgtttaa

SEQ ID NO: 20 - DNA, 1416 bp, Paraburkholderia tropica
  source 1..1416: mol_type = genomic DNA
atgagtaaatccgtggccgacgtcatgcaactcgtcaaggacgaagacgtcaagtttgtcgacttccgctttaccgacacgcgcggcaaggagcaacacgtctcggttccgctgtcgcacttcgacgaagacaagttcgagtcgggtcatgcatttgacggttcgtcgatcgccggctggaagggcatcgaagcctcggacatgctgctcgttccggatgcgaacacggccttcatcgacccgttctacgaagagtcgacgctggttctgacctgcgacgtggtcgagccggctgacggcaagggctgcgagcgcgacccgcgctcgctcgccaagcgcgctgaagcgtacctgaagagcacgggcctcggcgacacggccttcttcggtccggaacccgaattcttcattttcgactcggtccagtggaacacggacatgtcgggctcgttcgtcaagatcggctcggaagaagcaccgtggtcgtcgagcaaggaattcgaaggcggcaacacgggtcaccgtccgggcgtcaagggcggctacttcccggtcgcgccggtcgacacgttccaggacatccgttcggaaatgtgtctgctgctcgaacagatcggcatcccggtcgaagtgcaccaccacgaagtcgctggccagggccagaacgaaatcggcacgaagttctcgacgctggttcagcgcgcggactggacgcagcagatgaagtacatcatccataacgtggcgcacacgtatggcaagacggcgacgttcatgccgaagccgatcgttggcgacaacggttcgggcatgcacgttcaccagtcgatctggaaggacggccagaacctgttcgcaggcaacggctacgcaggtctgtcggaattcgcgctgttctacatcggcggcatcatcaagcacgctcgcgcgctgaacgccatcacgaacccgtcgacgaactcgtacaagcgtctggttccgcacttcgaagcaccggtcaagctggcttactcggcacgcaaccgttcggcgtcgatccgtattccgcacgtctcgaacccgaagggtcgccgtatcgaaacgcgcttcccggacccgatggccaacccgtacctgtgcttctcggcactgatgatggcaggtctggacggcgtgcagaacaagatccatccgggcgaagccgccgacaagaacctgtacgacctgccgccggaagaggatgcaaagatcccgaccgtttgcgccggcctcgaccaggctctggaagcgcttgacaaggatcgcgagttcctgacgcgcggtggcgtgttcacggattcgatgatcgacgcctacctcgcactgaaggaaggcgaactgcaacgcgtgcgcatgacgacgcacccggtcgagttcgaactgtactactcgctgtaa

SEQ ID NO: 21 - DNA, 1416 bp, Paraburkholderia tropica
  source 1..1416: mol_type = genomic DNA
atgagtaaatccgtggccgacgtcatgcaactcgtcaaggacgaagacgtcaagtttgtcgacttccgctttaccgacacgcgcggcaaggagcaacacgtctcggttccgctgtcgcacttcgacgaagacaagttcgagtcgggtcatgcatttgacggttcgtcgatcgccggctggaagggcatcgaagcctcggacatgctgctcgttccggatgcgaacacggccttcatcgacccgttctacgaagagtcgacgctggttctgacctgcgacgtggtcgagccggctgacggcaagggctacgagcgcgacccgcgctcgctcgccaagcgcgctgaagcgtacctgaagagcacgggcctcggcgacacggccttcttcggtccggaacccgaattcttcattttcgactcggtccagtggaacacggacatgtcgggctcgttcgtcaagatcggctcggaagaagcaccgtggtcgccgagcaaggaattcgaaggcggcaacacgggtcaccgtccgggcgtcaagggcggctacttcccggtcgcgccggtcgacacgttccaggacatccgttcggaaatgtgtctgctgctcgaacagatcggcatcccggtcgaagtgcaccaccacgaagtcgctggccagggccagaacgaaatcggcacgaagttctcgacgctggttcagcgcgcggactggacgcagcagatgaagtacatcatccataacgtggcgcacacgtatggcaagacggcgacgttcatgccgaagccgatcgttggcgacaacggttcgggcatgcacgttcaccagtcgatctggaaggacggccagaacctgttcgcaggcaacggctacgcaggtctgtcggaattcgcgctgttctacatcggcggcatcatcaagcacgctcgcgcgctgaacgccatcacgaacccgtcgacgaactcgtacaagcgtctggttccgcacttcgaagcaccggtcaagctggcttactcggcacgcaaccgttcggcgtcgatccgtattccgcacgtctcgaacccgaagggtcgccgtatcgaaacgcgcttcccggacccgatggccaacccgtacctgtgcttctcggcactgatgatggcaggtctggacggcgtgcagaacaagatccatccgggcgaagccgccgacaagaacctgtacgacctgccgccggaagaggatgcaaagatcccgaccgtttgcgccggcctcgaccaggctctggaagcgcttgacaaggatcgcgagttcctgacgcgcggtggcgtgttcacggattcgatgatcgacgcctacctcgcactgaaggaaggcgaactgcaacgcgtgcgcatgacgacgcacccggtcgagttcgaactgtactactcgctgtaa

SEQ ID NO: 22 - DNA, 1416 bp, Paraburkholderia tropica
  source 1..1416: mol_type = genomic DNA
atgagtaaatccgtggccgacgtcatgcaactcgtcaaggacgaagacgtcaagtttgtcgacttccgctttaccgacacgcgcggcaaggagcaacacgtctcggttccgctgtcgcacttcgacgaagacaagttcgagtcgggtcatgcatttgacggttcgtcgatcgccggctggaagggcatcgaagcctcggacatgctgctcgttccggatgcgaacacggccttcatcgacccgttctacgaagagtcgacgctggttctgacctgcgacgtggtcgagccggctgacggcaagggctacgagcgcgacccgcgctcgctcgccaagcgcgctgaagcgtacctgaagagcacgggcctcggcgacacggccttcttcggtccggaacccgaattcttcattttcgactcggtccagtggaacacggacatgtcgggctcgttcgtcaagatcggctcggaagaagcaccgtggtcgtcgagcaaggaattcgaaggcggcgacacgggtcaccgtccgggcgtcaagggcggctacttcccggtcgcgccggtcgacacgttccaggacatccgttcggaaatgtgtctgctgctcgaacagatcggcatcccggtcgaagtgcaccaccacgaagtcgctggccagggccagaacgaaatcggcacgaagttctcgacgctggttcagcgcgcggactggacgcagcagatgaagtacatcatccataacgtggcgcacacgtatggcaagacggcgacgttcatgccgaagccgatcgttggcgacaacggttcgggcatgcacgttcaccagtcgatctggaaggacggccagaacctgttcgcaggcaacggctacgcaggtctgtcggaattcgcgctgttctacatcggcggcatcatcaagcacgctcgcgcgctgaacgccatcacgaacccgtcgacgaactcgtacaagcgtctggttccgcacttcgaagcaccggtcaagctggcttactcggcacgcaaccgttcggcgtcgatccgtattccgcacgtctcgaacccgaagggtcgccgtatcgaaacgcgcttcccggacccgatggccaacccgtacctgtgcttctcggcactgatgatggcaggtctggacggcgtgcagaacaagatccatccgggcgaagccgccgacaagaacctgtacgacctgccgccggaagaggatgcaaagatcccgaccgtttgcgccggcctcgaccaggctctggaagcgcttgacaaggatcgcgagttcctgacgcgcggtggcgtgttcacggattcgatgatcgacgcctacctcgcactgaaggaaggcgaactgcaacgcgtgcgcatgacgacgcacccggtcgagttcgaactgtactactcgctgtaa

SEQ ID NO: 23 - DNA, 1416 bp, Paraburkholderia tropica
  source 1..1416: mol_type = genomic DNA
atgagtaaatccgtggccgacgtcatgcaactcgtcaaggacgaagacgtcaagtttgtcgacttccgctttaccgacacgcgcggcaaggagcaacacgtctcggttccgctgtcgcacttcgacgaagacaagttcgagtcgggtcatgcatttgacggttcgtcgatcgccggctggaagggcatcgaagcctcggacatgctgctcgttccggatgcgaacacggccttcatcgacccgttctacgaagagtcgacgctggttctgacctgcgacgtggtcgagccggctgacggcaagggctacgagcgcgacccgcgctcgctcgccaagcgcgctgaagcgtacctgaagagcacgggcctcggcgacacggccttcttcggtccggaacccgaattcttcattttcgactcggtccagtggaacacggacatgtcgggctcgttcgtcaagatcggctcggaagaagcaccgtggtcgtcgagcaaggaattcgaaggcggcaacacgggtcaccgtccgggcgtcaagggcggctacttcccggtcgcgccggtcgacacgttccaggacatccgttcggaaatgtgtctgctgctcgaacagatcggcatcccggtcgaagtgcaccaccacgaagtcgctggccacggccagaacgaaatcggcacgaagttctcgacgctggttcagcgcgcggactggacgcagcagatgaagtacatcatccataacgtggcgcacacgtatggcaagacggcgacgttcatgccgaagccgatcgttggcgacaacggttcgggcatgcacgttcaccagtcgatctggaaggacggccagaacctgttcgcaggcaacggctacgcaggtctgtcggaattcgcgctgttctacatcggcggcatcatcaagcacgctcgcgcgctgaacgccatcacgaacccgtcgacgaactcgtacaagcgtctggttccgcacttcgaagcaccggtcaagctggcttactcggcacgcaaccgttcggcgtcgatccgtattccgcacgtctcgaacccgaagggtcgccgtatcgaaacgcgcttcccggacccgatggccaacccgtacctgtgcttctcggcactgatgatggcaggtctggacggcgtgcagaacaagatccatccgggcgaagccgccgacaagaacctgtacgacctgccgccggaagaggatgcaaagatcccgaccgtttgcgccggcctcgaccaggctctggaagcgcttgacaaggatcgcgagttcctgacgcgcggtggcgtgttcacggattcgatgatcgacgcctacctcgcactgaaggaaggcgaactgcaacgcgtgcgcatgacgacgcacccggtcgagttcgaactgtactactcgctgtaa

SEQ ID NO: 24 - protein, 469 aa, Klebsiella variicola
MSAEHVLTMLNEHEVKFVDLRFTDTKGKEQHVTIPSHQVNAEFFEEGKMFDGSSIGGWKGINESDMVLMPDASTAVIDPFYEEPTLIIRCDILEPGTLQGYDRDPRSIAKRAEEYLRATGIADTVLFGPEPEFFLFDDIRFGASISGSHVAIDDIEGAWNSSTKYEGGNKGHRPGVKGGYFPVPPVDSSQDIRSTMCMIMEEMGLVVEAHHHEVATAGQNEVATRFNTMTKKADEIQIYKYVVHNVAHRFGKTATFMPKPMFGDNGSGMHCHMSLAKNGTNLFSGDKYAGLSEQALFYIGGVIKHAKAINALANPTTNSYKRLVPGYEAPVMLAYSARNRSASIRIPVVTSPKARRIEVRFPDPAANPYLCFAALLMAGLDGIKNKIHPGEAMDKNLYDLPPEEAKEIPQVAGSLEEALQALDADREFLTAGGVFTNDAIDAYIALRLEENDRVRMTPHPVEFELYYSV

SEQ ID NO: 25 - protein, 469 aa, Kosakonia sacchari
MSAEHVLTMLNEHEVKFVDLRFTDTKGKEQHVTIPAHQVNADFFEEGKMFDGSSIGGWKGINESDMVLMPDATTAVIDPFYEESTLIIRCDILEPGTMQGYDRDPRSIAKRAEEYLRSTGLADTVLFGPEPEFFLFDDVRFGSSISGSSVAIDDIEGAWNTSTKYEGGNKGHRPAVKGGYFPVPPVDSSQDLRSTMCLVMEEMGLVVEAHHHEVATAGQNEVATRFNTMTKKADEIQIYKYVVHNVAHRFGKTATFMPKPMFGDNGSGMHCHMSLSKNGTNLFSGDKYAGLSEMALYYIGGVIKHAKAINALSNPTTNSYKRLVPGYEAPVMLAYSARNRSASIRIPVVASPKARRIEVRFPDPAANPYLCFAALLMAGLDGIKNKIHPGEAMDKNLYDLPAEEAKEIPQVAGSLEEALNALDADREFLTAGGVFTDDAIDAYIALRIEENDRVRMTPHPVEFELYYSV

SEQ ID NO: 26 - protein, 469 aa, Metakosakonia intestini
MSAEHVLTMLNEHEVKFVDLRFTDTKGKEQHVTIPAHQVNADFFEEGKMFDGSSIGGWKGINESDMVLMPDASTAVIDPFYEEPTLIIRCDILEPGTMQGYDRDPRSIAKRAEEYLRATGIADTVLFGPEPEFFLFDDVRFGSSISGSHVAIDDIEGAWNSSTKYEGGNKGHRPAVKGGYFPVPPVDSAQDLRSTMCLVMEEMGLVVEAHHHEVATAGQNEVATRFNTMTKKADEIQIYKYVVHNVAHRFGKTATFMPKPMFGDNGSGMHCHMSLSKNGVNLFSGDKYAGLSEQALYYIGGVIKHAKAINALSNPTTNSYKRLVPGYEAPVMLAYSARNRSASIRIPVVASPKARRIEVRFPDPAANPYLCFAALLMAGLDGIKNKIHPGEAMDKNLYDLPAEEAKEIPQVAGSLEEALNCLNEDREFLTAGGVFTDDAIDAYIALRIEENDRVRMTPHPVEFELYYSV

SEQ ID NO: 27 - protein, 471 aa, Paraburkholderia tropica
MSKSVADVMQLVKDEDVKFVDFRFTDTRGKEQHVSVPLSHFDEDKFESGHAFDGSSIAGWKGIEASDMLLVPDANTAFIDPFYEESTLVLTCDVVEPADGKGYERDPRSLAKRAEAYLKSTGLGDTAFFGPEPEFFIFDSVQWNTDMSGSFVKIGSEEAPWSSSKEFEGGNTGHRPGVKGGYFPVAPVDTFQDIRSEMCLLLEQIGIPVEVHHHEVAGQGQNEIGTKFSTLVQRADWTQQMKYIIHNVAHTYGKTATFMPKPIVGDNGSGMHVHQSIWKDGQNLFAGNGYAGLSEFALFYIGGIIKHARALNAITNPSTNSYKRLVPHFEAPVKLAYSARNRSASIRIPHVSNPKGRRIETRFPDPMANPYLCFSALMMAGLDGVQNKIHPGEAADKNLYDLPPEEDAKIPTVCAGLDQALEALDKDREFLTRGGVFTDSMIDAYLALKEGELQRVRMTTHPVEFELYYSL

SEQ ID NO: 28 - protein, 945 aa, Klebsiella variicola
MMPLSPQLQQHWQTVADRLPADFPIAELSPQARSVMAFSDFVEQSVIAQPGWLNELADSSPEAEEWRHYEAWLQDRLQAVTDEAGLMRELRLFRRQMMVRIAWAQALSLVSEEETLQQLSVLAETLIVAARDWLYAACCKEWGTPCNAEGQPQPLLILGMGKLGGGELNFSSDIDLIFAWPEHGATRGGRRELDNAQFFTRLGQRLIKALDQPTQDGFVYRVDMRLRPFGDSGPLVLSFAALEDYYQEQGRDWERYAMVKARIMGDNDGVYASELRAMLRPFVFRRYIDFSVIQSLRNMKGMIAREVRRRGLKDNIKLGAGGIREIEFIVQVFQLIRGGREPALQQRALLPTLAAIDELHLLPEGDAALLREAYLFLRRLENLLQSINDEQTQTLPQDELNRARLAWGMHTEDWETLSAQLASQMANVRRVFNELIGDDEDQSPDEQLAEYWRELWQDALEEDDASPALAHLNDTDRRSVLALIADFRKELDRRTIGPRGRQVLDQLMPHLLSEICSRADAPLPLARITPLLTGIVTRTTYLELLSEFPGALKHLITLCAASPMVASQLARHPLLLDELLDPNTLYQPTATDAYRDELRQYLLRVPEEDEEQQLEALRQFKQAQQLHIAAADIAGTLPVMKVSDHLTWLAEAILDAVVQQAWGQMVARYGQPTHLHDRQGRGFAVVGYGKLGGWELGYSSDLDLVFLHDCPAEVMTDGEREIDGRQFYLRLAQRIMHLFSTRTSSGILYEVDARLRPSGAAGMLVTTADAFADYQQNEAWTWEHQALVRARVVYGDPALQARFDAIRRDILTTPREGMTLQTEVREMREKMRAHLGNKHPDRFDIKADAGGITDIEFITQYLVLRYASDKPKLTRWSDNVRILELLAQNDIMDEEEARALTHAYTTLRDALHHLALQEQPGHVAPEAFSRERQQVSASWQKWLMA

SEQ ID NO: 29 - protein, 469 aa, Klebsiella variicola
MSAEHVLTMLNEHEVKFVDLRFTDTKGKEQHVTIPSHQVNAEFFEEGKMFDGSSIGGWKGINESDIVLMPDASTAVIDPFYEEPTLIIRCDILEPGTLQGYDRDPRSIAKRAEEYLRATGIADTVLFGPEPEFFLFDDIRFGASISGSHVAIDDIEGAWNSSTKYEGGNKGHRPGVKGGYFPVPPVDSSQDIRSTMCMIMEEMGLVVEAHHHEVATAGQNEVATRFNTMTKKADEIQIYKYVVHNVAHRFGKTATFMPKPMFGDNGSGMHCHMSLAKNGTNLFSGDKYAGLSEQALFYIGGVIKHAKAINALANPTTNSYKRLVPGYEAPVMLAYSARNRSASIRIPVVTSPKARRIEVRFPDPAANPYLCFAALLMAGLDGIKNKIHPGEAMDKNLYDLPPEEAKEIPQVAGSLEEALQALDADREFLTAGGVFTNDAIDAYIALRLEENDRVRMTPHPVEFELYYSV

SEQ ID NO: 30 - protein, 469 aa, Klebsiella variicola
MSAEHVLTMLNEHEVKFVDLRFTDTKGKEQHVTIPSHQVNAEFFEEGKMFDGSSIGGWKGINESDVVLMPDASTAVIDPFYEEPTLIIRCDILEPGTLQGYDRDPRSIAKRAEEYLRATGIADTVLFGPEPEFFLFDDIRFGASISGSHVAIDDIEGAWNSSTKYEGGNKGHRPGVKGGYFPVPPVDSSQDIRSTMCMIMEEMGLVVEAHHHEVATAGQNEVATRFNTMTKKADEIQIYKYVVHNVAHRFGKTATFMPKPMFGDNGSGMHCHMSLAKNGTNLFSGDKYAGLSEQALFYIGGVIKHAKAINALANPTTNSYKRLVPGYEAPVMLAYSARNRSASIRIPVVTSPKARRIEVRFPDPAANPYLCFAALLMAGLDGIKNKIHPGEAMDKNLYDLPPEEAKEIPQVAGSLEEALQALDADREFLTAGGVFTNDAIDAYIALRLEENDRVRMTPHPVEFELYYSV

SEQ ID NO: 31 - protein, 469 aa, Klebsiella variicola
MSAEHVLTMLNEHEVKFVDLRFTDTKGKEQHVTIPSHQVNAEFFEEGKMFDGSSIGGWKGINESDMVLMPDASTAVIDPFYEEPTLIIRCDILEPGTLQGYDRDPRSIAKRAEEYLRATGIADTVLFGPEPEFFLFDDIRFGASISGSHVAIDDIECAWNSSTKYEGGNKGHRPGVKGGYFPVPPVDSSQDIRSTMCMIMEEMGLVVEAHHHEVATAGQNEVATRFNTMTKKADEIQIYKYVVHNVAHRFGKTATFMPKPMFGDNGSGMHCHMSLAKNGTNLFSGDKYAGLSEQALFYIGGVIKHAKAINALANPTTNSYKRLVPGYEAPVMLAYSARNRSASIRIPVVTSPKARRIEVRFPDPAANPYLCFAALLMAGLDGIKNKIHPGEAMDKNLYDLPPEEAKEIPQVAGSLEEALQALDADREFLTAGGVFTNDAIDAYIALRLEENDRVRMTPHPVEFELYYSV

SEQ ID NO: 32 - protein, 469 aa, Klebsiella variicola
MSAEHVLTMLNEHEVKFVDLRFTDTKGKEQHVTIPSHQVNAEFFEEGKMFDGSSIGGWKGINESDMVLMPDASTAVIDPFYEEPTLIIRCDILEPGTLQGYDRDPRSIAKRAEEYLRATGIADTVLFGPEPEFFLFDDIRFGASISGSHVAIDDIEGAWNSSTKYEGGNKGHRLGVKGGYFPVPPVDSSQDIRSTMCMIMEEMGLVVEAHHHEVATAGQNEVATRFNTMTKKADEIQIYKYVVHNVAHRFGKTATFMPKPMFGDNGSGMHCHMSLAKNGTNLFSGDKYAGLSEQALFYIGGVIKHAKAINALANPTTNSYKRLVPGYEAPVMLAYSARNRSASIRIPVVTSPKARRIEVRFPDPAANPYLCFAALLMAGLDGIKNKIHPGEAMDKNLYDLPPEEAKEIPQVAGSLEEALQALDADREFLTAGGVFTNDAIDAYIALRLEENDRVRMTPHPVEFELYYSV

SEQ ID NO: 33 - protein, 469 aa, Klebsiella variicola
MSAEHVLTMLNEHEVKFVDLRFTDTKGKEQHVTIPSHQVNAEFFEEGKMFDGSSIGGWKGINESDMVLMPDASTAVIDPFYEEPTLIIRCDILEPGTLQGYDRDPRSIAKRAEEYLRATGIADTVLFGPEPEFFLFDDIRFGASISGSHVAIDDIEGAWNSSTKYEGGNKGHRPGVKGGYFPVPPVDSFQDIRSTMCMIMEEMGLVVEAHHHEVATAGQNEVATRFNTMTKKADEIQIYKYVVHNVAHRFGKTATFMPKPMFGDNGSGMHCHMSLAKNGTNLFSGDKYAGLSEQALFYIGGVIKHAKAINALANPTTNSYKRLVPGYEAPVMLAYSARNRSASIRIPVVTSPKARRIEVRFPDPAANPYLCFAALLMAGLDGIKNKIHPGEAMDKNLYDLPPEEAKEIPQVAGSLEEALQALDADREFLTAGGVFTNDAIDAYIALRLEENDRVRMTPHPVEFELYYSV

SEQ ID NO: 34 - protein, 469 aa, Klebsiella variicola
MSAEHVLTMLNEHEVKFVDLRFTDTKGKEQHVTIPSHQVNAEFFEEGKMFDGSSIGGWKGINESDMVLMPDASTAVIDPFYEEPTLIIRCDILEPGTLQGYDRDPRSIAKRAEEYLRATGIADTVLFGPEPEFFLFDDIRFGASISGSHVAIDDIEGAWNSSTKYEGGNKGHRPGVKGGYFPVPPVDSSQDIRSTMCMIMEEMGLVVKAHHHEVATAGQNEVATRFNTMTKKADEIQIYKYVVHNVAHRFGKTATFMPKPMFGDNGSGMHCHMSLAKNGTNLFSGDKYAGLSEQALFYIGGVIKHAKAINALANPTTNSYKRLVPGYEAPVMLAYSARNRSASIRIPVVTSPKARRIEVRFPDPAANPYLCFAALLMAGLDGIKNKIHPGEAMDKNLYDLPPEEAKEIPQVAGSLEEALQALDADREFLTAGGVFTNDAIDAYIALRLEENDRVRMTPHPVEFELYYSV

SEQ ID NO: 35 - protein, 469 aa, Klebsiella variicola
MSAEHVLTMLNEHEVKFVDLRFTDTKGKEQHVTIPSHQVNAEFFEEGKMFDGSSIGGWKGINESDMVLMPDASTAVIDPFYEEPTLIIRCDILEPGTLQGYDRDPRSIAKRAEEYLRATGIADTVLFGPEPEFFLFDDIRFGASISGSHVAIDDIEGAWNSSTKYEGGNKGHRPGVKGGYFPVPPVDSSQDIRSTMCMIMEEMGLVVEAHHHEVATAGQNEVATRFNTMTKKADEIQIYKYVVHNVAHRFGKTATFMPKPMFGDNGSSMHCHMSLAKNGTNLFSGDKYAGLSEQALFYIGGVIKHAKAINALANPTTNSYKRLVPGYEAPVMLAYSARNRSASIRIPVVTSPKARRIEVRFPDPAANPYLCFAALLMAGLDGIKNKIHPGEAMDKNLYDLPPEEAKEIPQVAGSLEEALQALDADREFLTAGGVFTNDAIDAYIALRLEENDRVRMTPHPVEFELYYSV

SEQ ID NO: 36 - protein, 469 aa, Klebsiella variicola
MSAEHVLTMLNEHEVKFVDLRFTDTKGKEQHVTIPSHQVNAEFFEEGKMFDGSSIGGWKGINESDMVLMPDASTAVIDPFYEEPTLIIRCDILEPGTLQGYDRDPRSIAKRAEEYLRATGIADTVLFGPEPEFFLFDDIRFGASISGSHVAIDDIEGAWNSSTKYEGGNKGHRPGVKGGYFPVPPVDSSQDIRSTMCMIMEEMGLVVEAHHHEVATAGQNEVATRFNTMTKKADEIQIYKYVVHNVAHRFGKTATFMPKPMFGDNGSGMHCHMSLAKNGTNLFSGDKYAGLSEQALFYIGGVIKHAKAINALANPTTNSYKRLVPGYEAPVMLAYSARDRSASIRIPVVTSPKARRIEVRFPDPAANPYLCFAALLMAGLDGIKNKIHPGEAMDKNLYDLPPEEAKEIPQVAGSLEEALQALDADREFLTAGGVFTNDAIDAYIALRLEENDRVRMTPHPVEFELYYSV

SEQ ID NO: 37 - protein, 945 aa, Klebsiella variicola
MMPLSPQLQQHWQTVADRLPADFPIAELSPQARSVMAFSDFVEQSVIAQPGWLNELADSSPEAEEWRHYEAWLQDRLQAVTDEAGLMRELRLFRRQMMVRIAWAQALSLVSEEETLQQLSVLAETLIVAARDWLYAACCKEWGTPCNAEGQPQPLLILGMGKLGGGELNFSSDIDLIFAWPEHGATRGGRRELDNAQFFTRLGQRLIKALDQPTQDGFVYRVDMRLRPFGDSGPLVLSFAALEDYYQEQGRDWERYAMVKARIMGDNDGVYASELRAMLRPFVFRRYIDFSVIQSLRNMKGMIAREVRRRGLKDNIKLGAGEIREIEFIVQVFQLIRGGREPALQQRALLPTLAAIDELHLLPEGDAALLREAYLFLRRLENLLQSINDEQTQTLPQDELNRARLAWGMHTEDWETLSAQLASQMANVRRVFNELIGDDEDQSPDEQLAEYWRELWQDALEEDDASPALAHLNDTDRRSVLALIADFRKELDRRTIGPRGRQVLDQLMPHLLSEICSRADAPLPLARITPLLTGIVTRTTYLELLSEFPGALKHLITLCAASPMVASQLARHPLLLDELLDPNTLYQPTATDAYRDELRQYLLRVPEEDEEQQLEALRQFKQAQQLHIAAADIAGTLPVMKVSDHLTWLAEAILDAVVQQAWGQMVARYGQPTHLHDRQGRGFAVVGYGKLGGWELGYSSDLDLVFLHDCPAEVMTDGEREIDGRQFYLRLAQRIMHLFSTRTSSDILYEVDARLRPSGAAGMLVTTADAFADYQQNEAWTWEHQALVRARVVYGDPALQARFDAIRRDILTTPREGMTLQTEVREMREKMRAHLGNKHPDRFDIKADAGGITDIEFITQYLVLRYASDKPKLTRWSDNVRILELLAQNDIMDEEEARALTHAYTTLRDALHHLALQEQPGHVAPEAFSRERQQVSASWQKWLMA

SEQ ID NO: 38 - protein, 469 aa, Kosakonia sacchari
MSAEHVLTMLNEHEVKFVDLRFTDTKGKEQHVTIPAHQVNADFFEEGKMFDGSSIGGWKGINESDIVLMPDATTAVIDPFYEESTLIIRCDILEPGTMQGYDRDPRSIAKRAEEYLRSTGLADTVLFGPEPEFFLFDDVRFGSSISGSSVAIDDIEGAWNTSTKYEGGNKGHRPAVKGGYFPVPPVDSSQDLRSTMCLVMEEMGLVVEAHHHEVATAGQNEVATRFNTMTKKADEIQIYKYVVHNVAHRFGKTATFMPKPMFGDNGSGMHCHMSLSKNGTNLFSGDKYAGLSEMALYYIGGVIKHAKAINALSNPTTNSYKRLVPGYEAPVMLAYSARNRSASIRIPVVASPKARRIEVRFPDPAANPYLCFAALLMAGLDGIKNKIHPGEAMDKNLYDLPAEEAKEIPQVAGSLEEALNALDADREFLTAGGVFTDDAIDAYIALRIEENDRVRMTPHPVEFELYYSV

SEQ ID NO: 39 - protein, 469 aa, Metakosakonia intestini
MSAEHVLTMLNEHEVKFVDLRFTDTKGKEQHVTIPAHQVNADFFEEGKMFDGSSIGGWKGINESDMVLMPDASTAVIDPFYEEPTLIIRCDILEPGTMQGYDRDPRSIAKRAEEYLRATGIADTVLFGPEPEFFLFDDVRFGSSISGSHVAIDDIEGAWNSSTKYEGGNKGLRPAVKGGYFPVPPVDSAQDLRSTMCLVMEEMGLVVEAHHHEVATAGQNEVATRFNTMTKKADEIQIYKYVVHNVAHRFGKTATFMPKPMFGDNGSGMHCHMSLSKNGVNLFSGDKYAGLSEQALYYIGGVIKHAKAINALSNPTTNSYKRLVPGYEAPVMLAYSARNRSASIRIPVVASPKARRIEVRFPDPAANPYLCFAALLMAGLDGIKNKIHPGEAMDKNLYDLPAEEAKEIPQVAGSLEEALNCLNEDREFLTAGGVFTDDAIDAYIALRIEENDRVRMTPHPVEFELYYSV

SEQ ID NO: 40 - protein, 469 aa, Metakosakonia intestini
MSAEHVLTMLNEHEVKFVDLRFTDTKGKEQHVTIPAHQVNADFFEEGKMFDGSSIGGWKGINESDMVLMPDASTAVIDPFYEEPTLIIRCDILEPGTMQGYDRDPRSIAKRAEEYLRATGIADTVLFGPEPEFFLFDDVRFGSSISGSHVAIDDIEGAWNSSTKYEGGNKGHRPAVKGGYFPVPPVDSAQDLRSTMCLVMEEMGLVVEAHHHEVATASQNEVATRFNTMTKKADEIQIYKYVVHNVAHRFGKTATFMPKPMFGDNGSGMHCHMSLSKNGVNLFSGDKYAGLSEQALYYIGGVIKHAKAINALSNPTTNSYKRLVPGYEAPVMLAYSARNRSASIRIPVVASPKARRIEVRFPDPAANPYLCFAALLMAGLDGIKNKIHPGEAMDKNLYDLPAEEAKEIPQVAGSLEEALNCLNEDREFLTAGGVFTDDAIDAYIALRIEENDRVRMTPHPVEFELYYSV

SEQ ID NO: 41 - protein, 469 aa, Metakosakonia intestini
MSAEHVLTMLNEHEVKFVDLRFTDTKGKEQHVTIPAHQVNADFFEEGKMFDGSSIGGWKGINESDMVLMPDASTAVIDPFYEEPTLIIRCDILEPGTMQGYDRDPRSIAKRAEEYLRATGIADTVLFGPEPEFFLFDDVRFGSSISGSHVAIDDIEGAWNSSTKYEGGNKGHRPAVKGGYFPVPPVDSAQDLRSTMCLVMEEMGLVVEAHHHEVATAGQNEVATRFNTMTKKADEIQIYKYVVHNVAHRFGKTAIFMPKPMFGDNGSGMHCHMSLSKNGVNLFSGDKYAGLSEQALYYIGGVIKHAKAINALSNPTTNSYKRLVPGYEAPVMLAYSARNRSASIRIPVVASPKARRIEVRFPDPAANPYLCFAALLMAGLDGIKNKIHPGEAMDKNLYDLPAEEAKEIPQVAGSLEEALNCLNEDREFLTAGGVFTDDAIDAYIALRIEENDRVRMTPHPVEFELYYSV

SEQ ID NO: 42 - protein, 469 aa, Metakosakonia intestini
MSAEHVLTMLNEHEVKFVDLRFTDTKGKEQHVTIPAHQVNADFFEEGKMFDGSSIGGWKGINESDMVLMPDASTAVIDPFYEEPTLIIRCDILEPGTMQGYDRDPRSIAKRAEEYLRATGIADTVLFGPEPEFFLFDDVRFGSSISGSHVAIDDIEGAWNSSTKYEGGNKGHRPAVKGGYFPVPPVDSAQDLRSTMCLVMEEMGLVVEAHHHEVATAGQNEVATRFNTMTKKADEIQIYKYVVHNVAHRFGKTATFMPKPMFGDNGSGMHCHMSLSKNGVNLFSGDKYAGLSEQALYYIGGVIKHAKAINALSNPTTNSYKRLVPGYEVPVMLAYSARNRSASIRIPVVASPKARRIEVRFPDPAANPYLCFAALLMAGLDGIKNKIHPGEAMDKNLYDLPAEEAKEIPQVAGSLEEALNCLNEDREFLTAGGVFTDDAIDAYIALRIEENDRVRMTPHPVEFELYYSV

SEQ ID NO: 43 - protein, 471 aa, Paraburkholderia tropica
MSKSVADVMQLVKDEDVKFVDFRFTDTRGKEQHVSVPLSHFDEDKFESGHAFDGSSIAGWKGIEASDMLLVPDANTAFIDPFYEESTLVLTCDVVEPADGKGCERDPRSLAKRAEAYLKSTGLGDTAFFGPEPEFFIFDSVQWNTDMSGSFVKIGSEEAPWSSSKEFEGGNTGHRPGVKGGYFPVAPVDTFQDIRSEMCLLLEQIGIPVEVHHHEVAGQGQNEIGTKFSTLVQRADWTQQMKYIIHNVAHTYGKTATFMPKPIVGDNGSGMHVHQSIWKDGQNLFAGNGYAGLSEFALFYIGGIIKHARALNAITNPSTNSYKRLVPHFEAPVKLAYSARNRSASIRIPHVSNPKGRRIETRFPDPMANPYLCFSALMMAGLDGVQNKIHPGEAADKNLYDLPPEEDAKIPTVCAGLDQALEALDKDREFLTRGGVFTDSMIDAYLALKEGELQRVRMTTHPVEFELYYSL

SEQ ID NO: 44 - protein, 471 aa, Paraburkholderia tropica
MSKSVADVMQLVKDEDVKFVDFRFTDTRGKEQHVSVPLSHFDEDKFESGHAFDGSSIAGWKGIEASDMLLVPDANTAFIDPFYEESTLVLTCDVVEPADGKGYERDPRSLAKRAEAYLKSTGLGDTAFFGPEPEFFIFDSVQWNTDMSGSFVKIGSEEAPWSPSKEFEGGNTGHRPGVKGGYFPVAPVDTFQDIRSEMCLLLEQIGIPVEVHHHEVAGQGQNEIGTKFSTLVQRADWTQQMKYIIHNVAHTYGKTATFMPKPIVGDNGSGMHVHQSIWKDGQNLFAGNGYAGLSEFALFYIGGIIKHARALNAITNPSTNSYKRLVPHFEAPVKLAYSARNRSASIRIPHVSNPKGRRIETRFPDPMANPYLCFSALMMAGLDGVQNKIHPGEAADKNLYDLPPEEDAKIPTVCAGLDQALEALDKDREFLTRGGVFTDSMIDAYLALKEGELQRVRMTTHPVEFELYYSL

SEQ ID NO: 45 - protein, 471 aa, Paraburkholderia tropica
MSKSVADVMQLVKDEDVKFVDFRFTDTRGKEQHVSVPLSHFDEDKFESGHAFDGSSIAGWKGIEASDMLLVPDANTAFIDPFYEESTLVLTCDVVEPADGKGYERDPRSLAKRAEAYLKSTGLGDTAFFGPEPEFFIFDSVQWNTDMSGSFVKIGSEEAPWSSSKEFEGGDTGHRPGVKGGYFPVAPVDTFQDIRSEMCLLLEQIGIPVEVHHHEVAGQGQNEIGTKFSTLVQRADWTQQMKYIIHNVAHTYGKTATFMPKPIVGDNGSGMHVHQSIWKDGQNLFAGNGYAGLSEFALFYIGGIIKHARALNAITNPSTNSYKRLVPHFEAPVKLAYSARNRSASIRIPHVSNPKGRRIETRFPDPMANPYLCFSALMMAGLDGVQNKIHPGEAADKNLYDLPPEEDAKIPTVCAGLDQALEALDKDREFLTRGGVFTDSMIDAYLALKEGELQRVRMTTHPVEFELYYSL

SEQ ID NO: 46 - protein, 471 aa, Paraburkholderia tropica
MSKSVADVMQLVKDEDVKFVDFRFTDTRGKEQHVSVPLSHFDEDKFESGHAFDGSSIAGWKGIEASDMLLVPDANTAFIDPFYEESTLVLTCDVVEPADGKGYERDPRSLAKRAEAYLKSTGLGDTAFFGPEPEFFIFDSVQWNTDMSGSFVKIGSEEAPWSSSKEFEGGNTGHRPGVKGGYFPVAPVDTFQDIRSEMCLLLEQIGIPVEVHHHEVAGHGQNEIGTKFSTLVQRADWTQQMKYIIHNVAHTYGKTATFMPKPIVGDNGSGMHVHQSIWKDGQNLFAGNGYAGLSEFALFYIGGIIKHARALNAITNPSTNSYKRLVPHFEAPVKLAYSARNRSASIRIPHVSNPKGRRIETRFPDPMANPYLCFSALMMAGLDGVQNKIHPGEAADKNLYDLPPEEDAKIPTVCAGLDQALEALDKDREFLTRGGVFTDSMIDAYLALKEGELQRVRMTTHPVEFELYYSL